SEQUENCE LISTING (32 sequences)

SEQ ID NO: 1 - DNA, 6973 bp, Moraxella catarrhalis
ccatggatatgggcaggtgtgctcgcctgccgtatgatggcgatgacaccccatttgccccatatctgtacgatttgacatgtgatatgatttaacatgtgacatgatttaacattgtttaatactgttgccatcattaccataatttagtaacgcatttagtaacgcatttgtaaaaatcattgcgcccctttatgtgtatcatatgaatagaatattatgattgtatctgattattgtatcagaatggtgatgctatatgatgatgcctacgagttgatttgggttaatcactctatgatttgatatattttgaaactaatctattgacttaaatcaccatatggttataatttagcataatggtaggctttttgtaaaaatcacatcgcaatattgttctactgttactaccatgcttgaatgacgatcccaatcaccagattcattcaagtgatgtgtttgtatacgcaccatttaccctaattatttcaatcaaatgcctatgtcagcatgtatcatttttttaaggtaaaccaccatgaatcacatctataaagtcatctttaacaaagccacaggcacatttatggcagtggcagagtacgccaaatcccacagcacggggggggggtagctgtgctacagggcaagttggcagtgtatgcactctgagctttgcccgtattgccgcgctcgctgtcctcgtgatcggtgcaacgctcagtggcagtgcttatgctcaaaaaaaagataccaaacatatcgcaattggtgaacaaaaccagccaagacgctcaggcactgccaaggcggacggtgatcgagccattgctattggtgaaaatgctaacgcacagggcggtcaagccatcgccatcggtagtagtaataaaactgtcaatggaagcagtttggataagataggtaccgatgctacgggtcaagagtccatcgccatcggtggtgatgtaaaggctagtggtgatgcctcgattgccatcggtagtgatgacttacatttgcttgatcagcatggtaatcctaaacatccgaaaggtactctgattaacgatcttattaacggccatgcagtattaaaagaaatacgaagctcaaaggataatgatgtaaaatatagacgcacaaccgcaagcggacacgccagtactgcagtgggagccatgtcatatgcacagggtcatttttccaacgcctttggtacacgggcaacagctaaaagtgcctattccttggcagtgggtcttgccgccacagccgagggccaatctacaatcgctattggttctgatgcaacatctagctcgttgggagcgatagcccttggtgcaggtactcgtgctcagctacagggcagtattgccctaggtcaaggttctgttgtcactcagagtgataataattctagaccggcctatacaccaaatacccaggcactagaccccaagtttcaagccaccaataatacgaaggcgggtccactttccattggtagtaactctatcaaacgtaaaatcatcaatgtcggtgcaggtgttaataaaaccgatgcggtcaatgtggcacagctagaagcggtggtgaagtgggctaaggagcgtagaattacttttcagggtgatgataacagtactgacgtaaaaataggtttggataatactttaactattaaaggtggtgcagagaccaacgcattaaccgataataatatcggtgtggtaaaagaggctgataatagtggtctgaaagttaaacttgctaaaactttaaacaatcttactgaggtgaatacaactacattaaatgccacaaccacagttaaggtaggtagtagtagtagtactacagctgaattattgagtgatagtttaacctttacccagcccaatacaggcagtcaaagcacaagcaaaaccgtctatggcgttaatggggtgaagtttactaataatgcagaaacaacagcagcaatcggcactactcgtattaccagagataaaattggctttgctcgagatggtgatgttgatgaaaaacaagcaccatatttggataaaaaacaacttaaagtgggtagtgttgcaattaccatagacaatggcattgatgcaggtaataaaaagatcagtaatcttgccaaaggtagcagtgctaacgatgcggttaccatcgaacagctcaaagccgccaagcctactttaaacgcaggcgctggcatcagtgtcacacctactgaaatatcagttgatgctaagagtggcaatgttaccgccccaacttacaacattggcgtgaaaaccaccgagcttaacagtgatggcactagtgataaatttagtgttaagggtagtggtacgaacaatagcttagttaccgccgaacatttggcaagctatctaaatgaagtcaatcgaacggctgacagtgctctacaaagctttaccgttaaagaagaagacgatgatgacgccaacgctatcaccgtggctaaagatacgacaaaaaatgccggcgcagtcagcatcttaaaactcaaaggtaaaaacggtctaacggttgctaccaaaaaagatggtacggttacctttgggcttagccaagatagcggtctgaccattggcaaaagcaccctaaacaacgatggcttgactgttaaagataccaacgaacaaatccaagtcggtgctaatggcattaaatttactaatgtgaatggtagtaatccaggtactggcattgcaaataccgctcgcattaccagagataaaattggctttgctggttctgatggtgcagttgatacaaacaaaccttatcttgatcaagacaagctacaagttggcaatgttaagattaccaacactggcattaacgcaggtggtaaagccatcacagggctgtccccaacactgcctagcattgccgatcaaagtagccgcaacatagaactgggcaatacaatccaagacaaagacaaatccaacgctgccagcattaatgatatattaaatacaggctttaacctaaaaaataataacaaccccattgactttgtctccacttatgacattgttgactttgccaatggcaatgccaccaccgccacagtaacccatgataccgctaacaaaaccagtaaagtggtatatgatgtgaatgtggatgatacaaccattcatctaacaggcactgatgacaataaaaaacttggcgtcaaaaccaccaaactgaacaaaacaagtgctaatggtaatacagcaactaactttaatgttaactctagtgatgaagatgcccttgttaacgccaaagacatcgccgaaaatctaaacaccctagccaaggaaattcacaccaccaaaggcacagcagacaccgccctacaaacctttaccgttaaaaaggtagatgaaaataataatgctgatgacgccaacgccatcaccgtgggtcaaaagaacgcaaataatcaagtcaacaccctaacactcaaaggtgaaaacggtcttaatattaaaaccgacaaaaatggtacggttacctttggcattaacaccacaagcggtcttaaagccggcaaaagcaccctaaacgacggtggcttgtctattaaaaaccccactggtagcgaacaaatccaagtcggtgctgatggcgtgaagtttgccaaggttaataataatggtgttgtaggtgctggcattgatggcacaactcgcattaccagagatgaaattggctttactgggactaatggctcacttgataaaagcaaaccccacctaagcaaagacggcattaacgcaggtggtaaaaagattaccaacattcaatcaggtgagattgcccaaaacagccatgatgctgtgacaggcggcaagatttatgatttaaaaaccgaacttgaaaacaaaatcagcagtactgccaaaacagcacaaaactcattacacgaattctcagtagcagatgaacaaggtaataactttacggttagtaacccttactccagttatgacacctcaaagacctctgatgtcatcacctttgcaggtgaaaacggcattaccaccaaggtaaataaaggtgtggtgcgtgtgggcattgaccaaaccaaaggcttaaccacgcctaagctgaccgtgggtaataataatggcaaaggcattgtcattgacagccaaaatggtcaaaataccatcacaggactaagcaacactctagctaatgttaccaatgataaaggtagcgtacgcaccacagaacagggcaatataatcaaagacgaagacaaaacccgtgccgccagcattgttgatgtgctaagcgcaggctttaacttgcaaggcaatggtgaagcggttgactttgtctccacttatgacaccgtcaactttgccgatggcaatgccaccaccgctaaggtgacctatgatgacacaagcaaaaccagtaaagtggtctatgatgtcaatgtggatgatacaaccattgaagttaaagataaaaaacttggcgtaaaaaccaccacattgaccagtactggcacaggtgctaataaatttgccctaagcaatcaagctactggcgatgcgcttgtcaaggccagtgatatcgttgctcatctaaacaccttatctggcgacatccaaactgccaaaggggcaagccaagcgaacaactcagcaggctatgtggatgctgatggcaataaggtcatctatgacagtaccgataacaagtactatcaagccaaaaatgatggcacagttgataaaaccaaagaagttgccaaagacaaactggtcgcccaagcccaaaccccagatggcacattggctcaaatgaatgtcaaatcagtcattaacaaagaacaagtaaatgatgccaataaaaagcaaggcatcaatgaagacaacgcctttgttaaaggacttgaaaaagccgcttctgataacaaaaccaaaaacgccgcagtaactgtgggtgatttaaatgccgttgcccaaacaccgctgacctttgcaggggatacaggcacaacggctaaaaaactgggcgagactttgaccatcaaaggtgggcaaacagacaccaataagctaaccgataataacatcggtgtggtagcaggtactgatggcttcactgtcaaacttgccaaagacctaaccaatcttaacagcgttaatgcaggtggcaccaaaattgatgacaaaggcgtgtcttttgtagactcaagcggtcaagccaaagcaaacacccctgtgctaagtgccaatgggctggacctgggtggcaaggtcatcagtaatgtgggcaaaggcacaaaagataccgacgctgccaatgtacaacagttaaacgaagtacgcaacttgttgggtcttggtaatgctggtaatgataacgctgacggcaatcaggtaaacattgccgacatcaaaaaagacccaaattcaggttcatcatctaaccgcactgtcatcaaagcaggcacggtacttggcggtaaaggtaataacgataccgaaaaacttgccactggtggtatacaagtgggcgtggataaagacggcaacgctaacggcgatttaagcaatgtttgggtcaaaacccaaaaagatggcagcaaaaaagccctgctcgccacttataacgccgcaggtcagaccaactatttgaccaacaaccccgcagaagccattgacagaataaatgaacaaggtatccgcttcttccatgtcaacgatggcaatcaagagcctgtggtacaagggcgtaacggcattgactcaagtgcctcaggcaagcactcagtggcgataggtttccaggccaaggcagatggtgaagccgccgttgccataggcagacaaacccaagcaggcaaccaatccatcgccatcggtgataacgcacaagccacgggcgatcaatccatcgccatcggtacaggcaatgtggtagcaggtaagcactctggtgccatcggcgacccaagcactgttaaggctgataacagttacagtgtgggtaataacaaccagtttaccgatgccactcaaaccgatgtctttggtgtgggcaataacatcaccgtgaccgaaagtaactcggttgccttaggttcaaactctgccatcagtgcaggcacacacgcaggcacacaagccaaaaaatctgacggcacagcaggtacaaccaccacagcaggtgcaaccggtacggttaaaggctttgctggacaaacggcggttggtgcggtctccgtgggtgcctcaggtgctgaacgccgtatccaaaatgtggcagcaggtgaggtcagtgccaccagcaccgatgcggtcaatggtagccagttgtacaaagccacccaaagcattgccaacgcaaccaatgagcttgaccatcgtatccaccaaaacgaaaataaggccaatgcagggatttcatcagcgatggcgatggcgtccatgccacaagcctacattcctggcagatccatggttaccgggggtattgccacccacaacggtcaaggtgcggtggcagtgggactgtcgaagctgtcggataatggtcaatgggtatttaaaatcaatggttcagccgatacccaaggccatgtaggggcggcagttggtgcaggttttcacttttaagccataaatcgcaagattttacttaaaaatcaatctcaccatagttgtataaaacagcatcagcatcagtcatattactgatgctgatgttttttatcacttaaaccattttaccgctcaagtgattctctttcaccatgaccaaatcgccattgatcataggtaaacttattgagtaaattttatcaatgtagttgttagatatggttaaaattgtgccattgaccaaaaaatgaccgatttatcccgaaaatttctgattatgatccgttgacctgcaggtcgac

SEQ ID NO: 2 - DNA, 5976 bp, Moraxella catarrhalis
gtgatcggtgcaacgctcagtggcagtgcttatgctcaaaaaaaagataccaaacatatcgcaattggtgaacaaaaccagccaagacgctcaggcactgccaaggcggacggtgatcgagccattgctattggtgaaaatgctaacgcacagggcggtcaagccatcgccatcggtagtagtaataaaactgtcaatggaagcagtttggataagataggtaccgatgctacgggtcaagagtccatcgccatcggtggtgatgtaaaggctagtggtgatgcctcgattgccatcggtagtgatgacttacatttgcttgatcagcatggtaatcctaaacatccgaaaggtactctgattaacgatcttattaacggccatgcagtattaaaagaaatacgaagctcaaaggataatgatgtaaaatatagacgcacaaccgcaagcggacacgccagtactgcagtgggagccatgtcatatgcacagggtcatttttccaacgcctttggtacacgggcaacagctaaaagtgcctattccttggcagtgggtcttgccgccacagccgagggccaatctacaatcgctattggttctgatgcaacatctagctcgttgggagcgatagcccttggtgcaggtactcgtgctcagctacagggcagtattgccctaggtcaaggttctgttgtcactcagagtgataataattctagaccggcctatacaccaaatacccaggcactagaccccaagtttcaagccaccaataatacgaaggcgggtccactttccattggtagtaactctatcaaacgtaaaatcatcaatgtcggtgcaggtgttaataaaaccgatgcggtcaatgtggcacagctagaagcggtggtgaagtgggctaaggagcgtagaattacttttcagggtgatgataacagtactgacgtaaaaataggtttggataatactttaactattaaaggtggtgcagagaccaacgcattaaccgataataatatcggtgtggtaaaagaggctgataatagtggtctgaaagttaaacttgctaaaactttaaacaatcttactgaggtgaatacaactacattaaatgccacaaccacagttaaggtaggtagtagtagtagtactacagctgaattattgagtgatagtttaacctttacccagcccaatacaggcagtcaaagcacaagcaaaaccgtctatggcgttaatggggtgaagtttactaataatgcagaaacaacagcagcaatcggcactactcgtattaccagagataaaattggctttgctcgagatggtgatgttgatgaaaaacaagcaccatatttggataaaaaacaacttaaagtgggtagtgttgcaattaccatagacaatggcattgatgcaggtaataaaaagatcagtaatcttgccaaaggtagcagtgctaacgatgcggttaccatcgaacagctcaaagccgccaagcctactttaaacgcaggcgctggcatcagtgtcacacctactgaaatatcagttgatgctaagagtggcaatgttaccgccccaacttacaacattggcgtgaaaaccaccgagcttaacagtgatggcactagtgataaatttagtgttaagggtagtggtacgaacaatagcttagttaccgccgaacatttggcaagctatctaaatgaagtcaatcgaacggctgacagtgctctacaaagctttaccgttaaagaagaagacgatgatgacgccaacgctatcaccgtggctaaagatacgacaaaaaatgccggcgcagtcagcatcttaaaactcaaaggtaaaaacggtctaacggttgctaccaaaaaagatggtacggttacctttgggcttagccaagatagcggtctgaccattggcaaaagcaccctaaacaacgatggcttgactgttaaagataccaacgaacaaatccaagtcggtgctaatggcattaaatttactaatgtgaatggtagtaatccaggtactggcattgcaaataccgctcgcattaccagagataaaattggctttgctggttctgatggtgcagttgatacaaacaaaccttatcttgatcaagacaagctacaagttggcaatgttaagattaccaacactggcattaacgcaggtggtaaagccatcacagggctgtccccaacactgcctagcattgccgatcaaagtagccgcaacatagaactgggcaatacaatccaagacaaagacaaatccaacgctgccagcattaatgatatattaaatacaggctttaacctaaaaaataataacaaccccattgactttgtctccacttatgacattgttgactttgccaatggcaatgccaccaccgccacagtaacccatgataccgctaacaaaaccagtaaagtggtatatgatgtgaatgtggatgatacaaccattcatctaacaggcactgatgacaataaaaaacttggcgtcaaaaccaccaaactgaacaaaacaagtgctaatggtaatacagcaactaactttaatgttaactctagtgatgaagatgcccttgttaacgccaaagacatcgccgaaaatctaaacaccctagccaaggaaattcacaccaccaaaggcacagcagacaccgccctacaaacctttaccgttaaaaaggtagatgaaaataataatgctgatgacgccaacgccatcaccgtgggtcaaaagaacgcaaataatcaagtcaacaccctaacactcaaaggtgaaaacggtcttaatattaaaaccgacaaaaatggtacggttacctttggcattaacaccacaagcggtcttaaagccggcaaaagcaccctaaacgacggtggcttgtctattaaaaaccccactggtagcgaacaaatccaagtcggtgctgatggcgtgaagtttgccaaggttaataataatggtgttgtaggtgctggcattgatggcacaactcgcattaccagagatgaaattggctttactgggactaatggctcacttgataaaagcaaaccccacctaagcaaagacggcattaacgcaggtggtaaaaagattaccaacattcaatcaggtgagattgcccaaaacagccatgatgctgtgacaggcggcaagatttatgatttaaaaaccgaacttgaaaacaaaatcagcagtactgccaaaacagcacaaaactcattacacgaattctcagtagcagatgaacaaggtaataactttacggttagtaacccttactccagttatgacacctcaaagacctctgatgtcatcacctttgcaggtgaaaacggcattaccaccaaggtaaataaaggtgtggtgcgtgtgggcattgaccaaaccaaaggcttaaccacgcctaagctgaccgtgggtaataataatggcaaaggcattgtcattgacagccaaaatggtcaaaataccatcacaggactaagcaacactctagctaatgttaccaatgataaaggtagcgtacgcaccacagaacagggcaatataatcaaagacgaagacaaaacccgtgccgccagcattgttgatgtgctaagcgcaggctttaacttgcaaggcaatggtgaagcggttgactttgtctccacttatgacaccgtcaactttgccgatggcaatgccaccaccgctaaggtgacctatgatgacacaagcaaaaccagtaaagtggtctatgatgtcaatgtggatgatacaaccattgaagttaaagataaaaaacttggcgtaaaaaccaccacattgaccagtactggcacaggtgctaataaatttgccctaagcaatcaagctactggcgatgcgcttgtcaaggccagtgatatcgttgctcatctaaacaccttatctggcgacatccaaactgccaaaggggcaagccaagcgaacaactcagcaggctatgtggatgctgatggcaataaggtcatctatgacagtaccgataacaagtactatcaagccaaaaatgatggcacagttgataaaaccaaagaagttgccaaagacaaactggtcgcccaagcccaaaccccagatggcacattggctcaaatgaatgtcaaatcagtcattaacaaagaacaagtaaatgatgccaataaaaagcaaggcatcaatgaagacaacgcctttgttaaaggacttgaaaaagccgcttctgataacaaaaccaaaaacgccgcagtaactgtgggtgatttaaatgccgttgcccaaacaccgctgacctttgcaggggatacaggcacaacggctaaaaaactgggcgagactttgaccatcaaaggtgggcaaacagacaccaataagctaaccgataataacatcggtgtggtagcaggtactgatggcttcactgtcaaacttgccaaagacctaaccaatcttaacagcgttaatgcaggtggcaccaaaattgatgacaaaggcgtgtcttttgtagactcaagcggtcaagccaaagcaaacacccctgtgctaagtgccaatgggctggacctgggtggcaaggtcatcagtaatgtgggcaaaggcacaaaagataccgacgctgccaatgtacaacagttaaacgaagtacgcaacttgttgggtcttggtaatgctggtaatgataacgctgacggcaatcaggtaaacattgccgacatcaaaaaagacccaaattcaggttcatcatctaaccgcactgtcatcaaagcaggcacggtacttggcggtaaaggtaataacgataccgaaaaacttgccactggtggtatacaagtgggcgtggataaagacggcaacgctaacggcgatttaagcaatgtttgggtcaaaacccaaaaagatggcagcaaaaaagccctgctcgccacttataacgccgcaggtcagaccaactatttgaccaacaaccccgcagaagccattgacagaataaatgaacaaggtatccgcttcttccatgtcaacgatggcaatcaagagcctgtggtacaagggcgtaacggcattgactcaagtgcctcaggcaagcactcagtggcgataggtttccaggccaaggcagatggtgaagccgccgttgccataggcagacaaacccaagcaggcaaccaatccatcgccatcggtgataacgcacaagccacgggcgatcaatccatcgccatcggtacaggcaatgtggtagcaggtaagcactctggtgccatcggcgacccaagcactgttaaggctgataacagttacagtgtgggtaataacaaccagtttaccgatgccactcaaaccgatgtctttggtgtgggcaataacatcaccgtgaccgaaagtaactcggttgccttaggttcaaactctgccatcagtgcaggcacacacgcaggcacacaagccaaaaaatctgacggcacagcaggtacaaccaccacagcaggtgcaaccggtacggttaaaggctttgctggacaaacggcggttggtgcggtctccgtgggtgcctcaggtgctgaacgccgtatccaaaatgtggcagcaggtgaggtcagtgccaccagcaccgatgcggtcaatggtagccagttgtacaaagccacccaaagcattgccaacgcaaccaatgagcttgaccatcgtatccaccaaaacgaaaataaggccaatgcagggatttcatcagcgatggcgatggcgtccatgccacaagcctacattcctggcagatccatggttaccgggggtattgccacccacaacggtcaaggtgcggtggcagtgggactgtcgaagctgtcggataatggtcaatgggtatttaaaatcaatggttcagccgatacccaaggccatgtaggggcggcagttggtgcaggttttcacttt

SEQ ID NO: 3 - protein, 1992 aa, Moraxella catarrhalis
VIGATLSGSAYAQKKDTKHIAIGEQNQPRRSGTAKADGDRAIAIGENANAQGGQAIAIGSSNKTVNGSSLDKIGTDATGQESIAIGGDVKASGDASIAIGSDDLHLLDQHGNPKHPKGTLINDLINGHAVLKEIRSSKDNDVKYRRTTASGHASTAVGAMSYAQGHFSNAFGTRATAKSAYSLAVGLAATAEGQSTIAIGSDATSSSLGAIALGAGTRAQLQGSIALGQGSVVTQSDNNSRPAYTPNTQALDPKFQATNNTKAGPLSIGSNSIKRKIINVGAGVNKTDAVNVAQLEAVVKWAKERRITFQGDDNSTDVKIGLDNTLTIKGGAETNALTDNNIGVVKEADNSGLKVKLAKTLNNLTEVNTTTLNATTTVKVGSSSSTTAELLSDSLTFTQPNTGSQSTSKTVYGVNGVKFTNNAETTAAIGTTRITRDKIGFARDGDVDEKQAPYLDKKQLKVGSVAITIDNGIDAGNKKISNLAKGSSANDAVTIEQLKAAKPTLNAGAGISVTPTEISVDAKSGNVTAPTYNIGVKTTELNSDGTSDKFSVKGSGTNNSLVTAEHLASYLNEVNRTADSALQSFTVKEEDDDDANAITVAKDTTKNAGAVSILKLKGKNGLTVATKKDGTVTFGLSQDSGLTIGKSTLNNDGLTVKDTNEQIQVGANGIKFTNVNGSNPGTGIANTARITRDKIGFAGSDGAVDTNKPYLDQDKLQVGNVKITNTGINAGGKAITGLSPTLPSIADQSSRNIELGNTIQDKDKSNAASINDILNTGFNLKNNNNPIDFVSTYDIVDFANGNATTATVTHDTANKTSKVVYDVNVDDTTIHLTGTDDNKKLGVKTTKLNKTSANGNTATNFNVNSSDEDALVNAKDIAENLNTLAKEIHTTKGTADTALQTFTVKKVDENNNADDANAITVGQKNANNQVNTLTLKGENGLNIKTDKNGTVTFGINTTSGLKAGKSTLNDGGLSIKNPTGSEQIQVGADGVKFAKVNNNGVVGAGIDGTTRITRDEIGFTGTNGSLDKSKPHLSKDGINAGGKKITNIQSGEIAQNSHDAVTGGKIYDLKTELENKISSTAKTAQNSLHEFSVADEQGNNFTVSNPYSSYDTSKTSDVITFAGENGITTKVNKGVVRVGIDQTKGLTTPKLTVGNNNGKGIVIDSQNGQNTITGLSNTLANVTNDKGSVRTTEQGNIIKDEDKTRAASIVDVLSAGFNLQGNGEAVDFVSTYDTVNFADGNATTAKVTYDDTSKTSKVVYDVNVDDTTIEVKDKKLGVKTTTLTSTGTGANKFALSNQATGDALVKASDIVAHLNTLSGDIQTAKGASQANNSAGYVDADGNKVIYDSTDNKYYQAKNDGTVDKTKEVAKDKLVAQAQTPDGTLAQMNVKSVINKEQVNDANKKQGINEDNAFVKGLEKAASDNKTKNAAVTVGDLNAVAQTPLTFAGDTGTTAKKLGETLTIKGGQTDTNKLTDNNIGVVAGTDGFTVKLAKDLTNLNSVNAGGTKIDDKGVSFVDSSGQAKANTPVLSANGLDLGGKVISNVGKGTKDTDAANVQQLNEVRNLLGLGNAGNDNADGNQVNIADIKKDPNSGSSSNRTVIKAGTVLGGKGNNDTEKLATGGIQVGVDKDGNANGDLSNVWVKTQKDGSKKALLATYNAAGQTNYLTNNPAEAIDRINEQGIRFFHVNDGNQEPVVQGRNGIDSSASGKHSVAIGFQAKADGEAAVAIGRQTQAGNQSIAIGDNAQATGDQSIAIGTGNVVAGKHSGAIGDPSTVKADNSYSVGNNNQFTDATQTDVFGVGNNITVTESNSVALGSNSAISAGTHAGTQAKKSDGTAGTTTTAGATGTVKGFAGQTAVGAVSVGASGAERRIQNVAAGEVSATSTDAVNGSQLYKATQSIANATNELDHRIHQNENKANAGISSAMAMASMPQAYIPGRSMVTGGIATHNGQGAVAVGLSKLSDNGQWVFKINGSADTQGHVGAAVGAGFHF

SEQ ID NO: 4 - protein, 2047 aa, Moraxella catarrhalis
MNHIYKVIFNKATGTFMAVAEYAKSHSTGGGSCATGQVGSVCTLSFARIAALAVLVIGATLSGSAYAQKKDTKHIAIGEQNQPRRSGTAKADGDRAIAIGENANAQGGQAIAIGSSNKTVNGSSLDKIGTDATGQESIAIGGDVKASGDASIAIGSDDLHLLDQHGNPKHPKGTLINDLINGHAVLKEIRSSKDNDVKYRRTTASGHASTAVGAMSYAQGHFSNAFGTRATAKSAYSLAVGLAATAEGQSTIAIGSDATSSSLGAIALGAGTRAQLQGSIALGQGSVVTQSDNNSRPAYTPNTQALDPKFQATNNTKAGPLSIGSNSIKRKIINVGAGVNKTDAVNVAQLEAVVKWAKERRITFQGDDNSTDVKIGLDNTLTIKGGAETNALTDNNIGVVKEADNSGLKVKLAKTLNNLTEVNTTTLNATTTVKVGSSSSTTAELLSDSLTFTQPNTGSQSTSKTVYGVNGVKFTNNAETTAAIGTTRITRDKIGFARDGDVDEKQAPYLDKKQLKVGSVAITIDNGIDAGNKKISNLAKGSSANDAVTIEQLKAAKPTLNAGAGISVTPTEISVDAKSGNVTAPTYNIGVKTTELNSDGTSDKFSVKGSGTNNSLVTAEHLASYLNEVNRTADSALQSFTVKEEDDDDANAITVAKDTTKNAGAVSILKLKGKNGLTVATKKDGTVTFGLSQDSGLTIGKSTLNNDGLTVKDTNEQIQVGANGIKFTNVNGSNPGTGIANTARITRDKIGFAGSDGAVDTNKPYLDQDKLQVGNVKITNTGINAGGKAITGLSPTLPSIADQSSRNIELGNTIQDKDKSNAASINDILNTGFNLKNNNNPIDFVSTYDIVDFANGNATTATVTHDTANKTSKVVYDVNVDDTTIHLTGTDDNKKLGVKTTKLNKTSANGNTATNFNVNSSDEDALVNAKDIAENLNTLAKEIHTTKGTADTALQTFTVKKVDENNNADDANAITVGQKNANNQVNTLTLKGENGLNIKTDKNGTVTFGINTTSGLKAGKSTLNDGGLSIKNPTGSEQIQVGADGVKFAKVNNNGVVGAGIDGTTRITRDEIGFTGTNGSLDKSKPHLSKDGINAGGKKITNIQSGEIAQNSHDAVTGGKIYDLKTELENKISSTAKTAQNSLHEFSVADEQGNNFTVSNPYSSYDTSKTSDVITFAGENGITTKVNKGVVRVGIDQTKGLTTPKLTVGNNNGKGIVIDSQNGQNTITGLSNTLANVTNDKGSVRTTEQGNIIKDEDKTRAASIVDVLSAGFNLQGNGEAVDFVSTYDTVNFADGNATTAKVTYDDTSKTSKVVYDVNVDDTTIEVKDKKLGVKTTTLTSTGTGANKFALSNQATGDALVKASDIVAHLNTLSGDIQTAKGASQANNSAGYVDADGNKVIYDSTDNKYYQAKNDGTVDKTKEVAKDKLVAQAQTPDGTLAQMNVKSVINKEQVNDANKKQGINEDNAFVKGLEKAASDNKTKNAAVTVGDLNAVAQTPLTFAGDTGTTAKKLGETLTIKGGQTDTNKLTDNNIGVVAGTDGFTVKLAKDLTNLNSVNAGGTKIDDKGVSFVDSSGQAKANTPVLSANGLDLGGKVISNVGKGTKDTDAANVQQLNEVRNLLGLGNAGNDNADGNQVNIADIKKDPNSGSSSNRTVIKAGTVLGGKGNNDTEKLATGGIQVGVDKDGNANGDLSNVWVKTQKDGSKKALLATYNAAGQTNYLTNNPAEAIDRINEQGIRFFHVNDGNQEPVVQGRNGIDSSASGKHSVAIGFQAKADGEAAVAIGRQTQAGNQSIAIGDNAQATGDQSIAIGTGNVVAGKHSGAIGDPSTVKADNSYSVGNNNQFTDATQTDVFGVGNNITVTESNSVALGSNSAISAGTHAGTQAKKSDGTAGTTTTAGATGTVKGFAGQTAVGAVSVGASGAERRIQNVAAGEVSATSTDAVNGSQLYKATQSIANATNELDHRIHQNENKANAGISSAMAMASMPQAYIPGRSMVTGGIATHNGQGAVAVGLSKLSDNGQWVFKINGSADTQGHVGAAVGAGFHF

SEQ ID NO: 5 - DNA, 6972 bp, Moraxella catarrhalis
ccatggatatgggcaggtgtgctcgcctgccgtatgatggcgatgacaccccatttgccccatatctgtacgatttgacatgtgatatgatttaacatgtgacatgatttaacattgtttaatactgttgccatcattaccataatttagtaacgcatttagtaacgcatttgtaaaaatcattgcgcccctttatgtgtatcatatgaatagaatattatgattgtatctgattattgtatcagaatggtgatgctatatgatgatgcctacgagttgatttgggttaatcactctatgatttgatatattttgaaactaatctattgacttaaatcaccatatggttataatttagcataatggtaggctttttgtaaaaatcacatcgcaatattgttctactgttactaccatgcttgaatgacgatcccaatcaccagattcattcaagtgatgtgtttgtatacgcaccatttaccctaattatttcaatcaaatgcctatgtcagcatgtatcatttttttaaggtaaaccaccatgaatcacatctataaagtcatctttaacaaagccacaggcacatttatggcagtggcagagtacgccaaatcccacagcacgggggggggtagctgtgctacagggcaagttggcagtgtatgcactctgagctttgcccgtattgccgcgctcgctgtcctcgtgatcggtgcaacgctcagtggcagtgcttatgctcaaaaaaaagataccaaacatatcgcaattggtgaacaaaaccagccaagacgctcaggcactgccaaggcggacggtgatcgagccattgctattggtgaaaatgctaacgcacagggcggtcaagccatcgccatcggtagtagtaataaaactgtcaatggaagcagtttggataagataggtaccgatgctacgggtcaagagtccatcgccatcggtggtgatgtaaaggctagtggtgatgcctcgattgccatcggtagtgatgacttacatttgcttgatcagcatggtaatcctaaacatccgaaaggtactctgattaacgatcttattaacggccatgcagtattaaaagaaatacgaagctcaaaggataatgatgtaaaatatagacgcacaaccgcaagcggacacgccagtactgcagtgggagccatgtcatatgcacagggtcatttttccaacgcctttggtacacgggcaacagctaaaagtgcctattccttggcagtgggtcttgccgccacagccgagggccaatctacaatcgctattggttctgatgcaacatctagctcgttgggagcgatagcccttggtgcaggtactcgtgctcagctacagggcagtattgccctaggtcaaggttctgttgtcactcagagtgataataattctagaccggcctatacaccaaatacccaggcactagaccccaagtttcaagccaccaataatacgaaggcgggtccactttccattggtagtaactctatcaaacgtaaaatcatcaatgtcggtgcaggtgttaataaaaccgatgcggtcaatgtggcacagctagaagcggtggtgaagtgggctaaggagcgtagaattacttttcagggtgatgataacagtactgacgtaaaaataggtttggataatactttaactattaaaggtggtgcagagaccaacgcattaaccgataataatatcggtgtggtaaaagaggctgataatagtggtctgaaagttaaacttgctaaaactttaaacaatcttactgaggtgaatacaactacattaaatgccacaaccacagttaaggtaggtagtagtagtagtactacagctgaattattgagtgatagtttaacctttacccagcccaatacaggcagtcaaagcacaagcaaaaccgtctatggcgttaatggggtgaagtttactaataatgcagaaacaacagcagcaatcggcactactcgtattaccagagataaaattggctttgctcgagatggtgatgttgatgaaaaacaagcaccatatttggataaaaaacaacttaaagtgggtagtgttgcaattaccatagacaatggcattgatgcaggtaataaaaagatcagtaatcttgccaaaggtagcagtgctaacgatgcggttaccatcgaacagctcaaagccgccaagcctactttaaacgcaggcgctggcatcagtgtcacacctactgaaatatcagttgatgctaagagtggcaatgttaccgccccaacttacaacattggcgtgaaaaccaccgagcttaacagtgatggcactagtgataaatttagtgttaagggtagtggtacgaacaatagcttagttaccgccgaacatttggcaagctatctaaatgaagtcaatcgaacggctgacagtgctctacaaagctttaccgttaaagaagaagacgatgatgacgccaacgctatcaccgtggctaaagatacgacaaaaaatgccggcgcagtcagcatcttaaaactcaaaggtaaaaacggtctaacggttgctaccaaaaaagatggtacggttacctttgggcttagccaagatagcggtctgaccattggcaaaagcaccctaaacaacgatggcttgactgttaaagataccaacgaacaaatccaagtcggtgctaatggcattaaatttactaatgtgaatggtagtaatccaggtactggcattgcaaataccgctcgcattaccagagataaaattggctttgctggttctgatggtgcagttgatacaaacaaaccttatcttgatcaagacaagctacaagttggcaatgttaagattaccaacactggcattaacgcaggtggtaaagccatcacagggctgtccccaacactgcctagcattgccgatcaaagtagccgcaacatagaactgggcaatacaatccaagacaaagacaaatccaacgctgccagcattaatgatatattaaatacaggctttaacctaaaaaataataacaaccccattgactttgtctccacttatgacattgttgactttgccaatggcaatgccaccaccgccacagtaacccatgataccgctaacaaaaccagtaaagtggtatatgatgtgaatgtggatgatacaaccattcatctaacaggcactgatgacaataaaaaacttggcgtcaaaaccaccaaactgaacaaaacaagtgctaatggtaatacagcaactaactttaatgttaactctagtgatgaagatgcccttgttaacgccaaagacatcgccgaaaatctaaacaccctagccaaggaaattcacaccaccaaaggcacagcagacaccgccctacaaacctttaccgttaaaaaggtagatgaaaataataatgctgatgacgccaacgccatcaccgtgggtcaaaagaacgcaaataatcaagtcaacaccctaacactcaaaggtgaaaacggtcttaatattaaaaccgacaaaaatggtacggttacctttggcattaacaccacaagcggtcttaaagccggcaaaagcaccctaaacgacggtggcttgtctattaaaaaccccactggtagcgaacaaatccaagtcggtgctgatggcgtgaagtttgccaaggttaataataatggtgttgtaggtgctggcattgatggcacaactcgcattaccagagatgaaattggctttactgggactaatggctcacttgataaaagcaaaccccacctaagcaaagacggcattaacgcaggtggtaaaaagattaccaacattcaatcaggtgagattgcccaaaacagccatgatgctgtgacaggcggcaagatttatgatttaaaaaccgaacttgaaaacaaaatcagcagtactgccaaaacagcacaaaactcattacacgaattctcagtagcagatgaacaaggtaataactttacggttagtaacccttactccagttatgacacctcaaagacctctgatgtcatcacctttgcaggtgaaaacggcattaccaccaaggtaaataaaggtgtggtgcgtgtgggcattgaccaaaccaaaggcttaaccacgcctaagctgaccgtgggtaataataatggcaaaggcattgtcattgacagccaaaatggtcaaaataccatcacaggactaagcaacactctagctaatgttaccaatgataaaggtagcgtacgcaccacagaacagggcaatataatcaaagacgaagacaaaacccgtgccgccagcattgttgatgtgctaagcgcaggctttaacttgcaaggcaatggtgaagcggttgactttgtctccacttatgacaccgtcaactttgccgatggcaatgccaccaccgctaaggtgacctatgatgacacaagcaaaaccagtaaagtggtctatgatgtcaatgtggatgatacaaccattgaagttaaagataaaaaacttggcgtaaaaaccaccacattgaccagtactggcacaggtgctaataaatttgccctaagcaatcaagctactggcgatgcgcttgtcaaggccagtgatatcgttgctcatctaaacaccttatctggcgacatccaaactgccaaaggggcaagccaagcgaacaactcagcaggctatgtggatgctgatggcaataaggtcatctatgacagtaccgataacaagtactatcaagccaaaaatgatggcacagttgataaaaccaaagaagttgccaaagacaaactggtcgcccaagcccaaaccccagatggcacattggctcaaatgaatgtcaaatcagtcattaacaaagaacaagtaaatgatgccaataaaaagcaaggcatcaatgaagacaacgcctttgttaaaggacttgaaaaagccgcttctgataacaaaaccaaaaacgccgcagtaactgtgggtgatttaaatgccgttgcccaaacaccgctgacctttgcaggggatacaggcacaacggctaaaaaactgggcgagactttgaccatcaaaggtgggcaaacagacaccaataagctaaccgataataacatcggtgtggtagcaggtactgatggcttcactgtcaaacttgccaaagacctaaccaatcttaacagcgttaatgcaggtggcaccaaaattgatgacaaaggcgtgtcttttgtagactcaagcggtcaagccaaagcaaacacccctgtgctaagtgccaatgggctggacctgggtggcaaggtcatcagtaatgtgggcaaaggcacaaaagataccgacgctgccaatgtacaacagttaaacgaagtacgcaacttgttgggtcttggtaatgctggtaatgataacgctgacggcaatcaggtaaacattgccgacatcaaaaaagacccaaattcaggttcatcatctaaccgcactgtcatcaaagcaggcacggtacttggcggtaaaggtaataacgataccgaaaaacttgccactggtggtatacaagtgggcgtggataaagacggcaacgctaacggcgatttaagcaatgtttgggtcaaaacccaaaaagatggcagcaaaaaagccctgctcgccacttataacgccgcaggtcagaccaactatttgaccaacaaccccgcagaagccattgacagaataaatgaacaaggtatccgcttcttccatgtcaacgatggcaatcaagagcctgtggtacaagggcgtaacggcattgactcaagtgcctcaggcaagcactcagtggcgataggtttccaggccaaggcagatggtgaagccgccgttgccataggcagacaaacccaagcaggcaaccaatccatcgccatcggtgataacgcacaagccacgggcgatcaatccatcgccatcggtacaggcaatgtggtagcaggtaagcactctggtgccatcggcgacccaagcactgttaaggctgataacagttacagtgtgggtaataacaaccagtttaccgatgccactcaaaccgatgtctttggtgtgggcaataacatcaccgtgaccgaaagtaactcggttgccttaggttcaaactctgccatcagtgcaggcacacacgcaggcacacaagccaaaaaatctgacggcacagcaggtacaaccaccacagcaggtgcaaccggtacggttaaaggctttgctggacaaacggcggttggtgcggtctccgtgggtgcctcaggtgctgaacgccgtatccaaaatgtggcagcaggtgaggtcagtgccaccagcaccgatgcggtcaatggtagccagttgtacaaagccacccaaagcattgccaacgcaaccaatgagcttgaccatcgtatccaccaaaacgaaaataaggccaatgcagggatttcatcagcgatggcgatggcgtccatgccacaagcctacattcctggcagatccatggttaccgggggtattgccacccacaacggtcaaggtgcggtggcagtgggactgtcgaagctgtcggataatggtcaatgggtatttaaaatcaatggttcagccgatacccaaggccatgtaggggcggcagttggtgcaggttttcacttttaagccataaatcgcaagattttacttaaaaatcaatctcaccatagttgtataaaacagcatcagcatcagtcatattactgatgctgatgttttttatcacttaaaccattttaccgctcaagtgattctctttcaccatgaccaaatcgccattgatcataggtaaacttattgagtaaattttatcaatgtagttgttagatatggttaaaattgtgccattgaccaaaaaatgaccgatttatcccgaaaatttctgattatgatccgttgacctgcaggtcgac

SEQ ID NO: 6 - DNA, 6141 bp, Moraxella catarrhalis
atgaatcacatctataaagtcatctttaacaaagccacaggcacatttatggcagtggcagagtacgccaaatcccacagcacgggggggggtagctgtgctacagggcaagttggcagtgtatgcactctgagctttgcccgtattgccgcgctcgctgtcctcgtgatcggtgcaacgctcagtggcagtgcttatgctcaaaaaaaagataccaaacatatcgcaattggtgaacaaaaccagccaagacgctcaggcactgccaaggcggacggtgatcgagccattgctattggtgaaaatgctaacgcacagggcggtcaagccatcgccatcggtagtagtaataaaactgtcaatggaagcagtttggataagataggtaccgatgctacgggtcaagagtccatcgccatcggtggtgatgtaaaggctagtggtgatgcctcgattgccatcggtagtgatgacttacatttgcttgatcagcatggtaatcctaaacatccgaaaggtactctgattaacgatcttattaacggccatgcagtattaaaagaaatacgaagctcaaaggataatgatgtaaaatatagacgcacaaccgcaagcggacacgccagtactgcagtgggagccatgtcatatgcacagggtcatttttccaacgcctttggtacacgggcaacagctaaaagtgcctattccttggcagtgggtcttgccgccacagccgagggccaatctacaatcgctattggttctgatgcaacatctagctcgttgggagcgatagcccttggtgcaggtactcgtgctcagctacagggcagtattgccctaggtcaaggttctgttgtcactcagagtgataataattctagaccggcctatacaccaaatacccaggcactagaccccaagtttcaagccaccaataatacgaaggcgggtccactttccattggtagtaactctatcaaacgtaaaatcatcaatgtcggtgcaggtgttaataaaaccgatgcggtcaatgtggcacagctagaagcggtggtgaagtgggctaaggagcgtagaattacttttcagggtgatgataacagtactgacgtaaaaataggtttggataatactttaactattaaaggtggtgcagagaccaacgcattaaccgataataatatcggtgtggtaaaagaggctgataatagtggtctgaaagttaaacttgctaaaactttaaacaatcttactgaggtgaatacaactacattaaatgccacaaccacagttaaggtaggtagtagtagtagtactacagctgaattattgagtgatagtttaacctttacccagcccaatacaggcagtcaaagcacaagcaaaaccgtctatggcgttaatggggtgaagtttactaataatgcagaaacaacagcagcaatcggcactactcgtattaccagagataaaattggctttgctcgagatggtgatgttgatgaaaaacaagcaccatatttggataaaaaacaacttaaagtgggtagtgttgcaattaccatagacaatggcattgatgcaggtaataaaaagatcagtaatcttgccaaaggtagcagtgctaacgatgcggttaccatcgaacagctcaaagccgccaagcctactttaaacgcaggcgctggcatcagtgtcacacctactgaaatatcagttgatgctaagagtggcaatgttaccgccccaacttacaacattggcgtgaaaaccaccgagcttaacagtgatggcactagtgataaatttagtgttaagggtagtggtacgaacaatagcttagttaccgccgaacatttggcaagctatctaaatgaagtcaatcgaacggctgacagtgctctacaaagctttaccgttaaagaagaagacgatgatgacgccaacgctatcaccgtggctaaagatacgacaaaaaatgccggcgcagtcagcatcttaaaactcaaaggtaaaaacggtctaacggttgctaccaaaaaagatggtacggttacctttgggcttagccaagatagcggtctgaccattggcaaaagcaccctaaacaacgatggcttgactgttaaagataccaacgaacaaatccaagtcggtgctaatggcattaaatttactaatgtgaatggtagtaatccaggtactggcattgcaaataccgctcgcattaccagagataaaattggctttgctggttctgatggtgcagttgatacaaacaaaccttatcttgatcaagacaagctacaagttggcaatgttaagattaccaacactggcattaacgcaggtggtaaagccatcacagggctgtccccaacactgcctagcattgccgatcaaagtagccgcaacatagaactgggcaatacaatccaagacaaagacaaatccaacgctgccagcattaatgatatattaaatacaggctttaacctaaaaaataataacaaccccattgactttgtctccacttatgacattgttgactttgccaatggcaatgccaccaccgccacagtaacccatgataccgctaacaaaaccagtaaagtggtatatgatgtgaatgtggatgatacaaccattcatctaacaggcactgatgacaataaaaaacttggcgtcaaaaccaccaaactgaacaaaacaagtgctaatggtaatacagcaactaactttaatgttaactctagtgatgaagatgcccttgttaacgccaaagacatcgccgaaaatctaaacaccctagccaaggaaattcacaccaccaaaggcacagcagacaccgccctacaaacctttaccgttaaaaaggtagatgaaaataataatgctgatgacgccaacgccatcaccgtgggtcaaaagaacgcaaataatcaagtcaacaccctaacactcaaaggtgaaaacggtcttaatattaaaaccgacaaaaatggtacggttacctttggcattaacaccacaagcggtcttaaagccggcaaaagcaccctaaacgacggtggcttgtctattaaaaaccccactggtagcgaacaaatccaagtcggtgctgatggcgtgaagtttgccaaggttaataataatggtgttgtaggtgctggcattgatggcacaactcgcattaccagagatgaaattggctttactgggactaatggctcacttgataaaagcaaaccccacctaagcaaagacggcattaacgcaggtggtaaaaagattaccaacattcaatcaggtgagattgcccaaaacagccatgatgctgtgacaggcggcaagatttatgatttaaaaaccgaacttgaaaacaaaatcagcagtactgccaaaacagcacaaaactcattacacgaattctcagtagcagatgaacaaggtaataactttacggttagtaacccttactccagttatgacacctcaaagacctctgatgtcatcacctttgcaggtgaaaacggcattaccaccaaggtaaataaaggtgtggtgcgtgtgggcattgaccaaaccaaaggcttaaccacgcctaagctgaccgtgggtaataataatggcaaaggcattgtcattgacagccaaaatggtcaaaataccatcacaggactaagcaacactctagctaatgttaccaatgataaaggtagcgtacgcaccacagaacagggcaatataatcaaagacgaagacaaaacccgtgccgccagcattgttgatgtgctaagcgcaggctttaacttgcaaggcaatggtgaagcggttgactttgtctccacttatgacaccgtcaactttgccgatggcaatgccaccaccgctaaggtgacctatgatgacacaagcaaaaccagtaaagtggtctatgatgtcaatgtggatgatacaaccattgaagttaaagataaaaaacttggcgtaaaaaccaccacattgaccagtactggcacaggtgctaataaatttgccctaagcaatcaagctactggcgatgcgcttgtcaaggccagtgatatcgttgctcatctaaacaccttatctggcgacatccaaactgccaaaggggcaagccaagcgaacaactcagcaggctatgtggatgctgatggcaataaggtcatctatgacagtaccgataacaagtactatcaagccaaaaatgatggcacagttgataaaaccaaagaagttgccaaagacaaactggtcgcccaagcccaaaccccagatggcacattggctcaaatgaatgtcaaatcagtcattaacaaagaacaagtaaatgatgccaataaaaagcaaggcatcaatgaagacaacgcctttgttaaaggacttgaaaaagccgcttctgataacaaaaccaaaaacgccgcagtaactgtgggtgatttaaatgccgttgcccaaacaccgctgacctttgcaggggatacaggcacaacggctaaaaaactgggcgagactttgaccatcaaaggtgggcaaacagacaccaataagctaaccgataataacatcggtgtggtagcaggtactgatggcttcactgtcaaacttgccaaagacctaaccaatcttaacagcgttaatgcaggtggcaccaaaattgatgacaaaggcgtgtcttttgtagactcaagcggtcaagccaaagcaaacacccctgtgctaagtgccaatgggctggacctgggtggcaaggtcatcagtaatgtgggcaaaggcacaaaagataccgacgctgccaatgtacaacagttaaacgaagtacgcaacttgttgggtcttggtaatgctggtaatgataacgctgacggcaatcaggtaaacattgccgacatcaaaaaagacccaaattcaggttcatcatctaaccgcactgtcatcaaagcaggcacggtacttggcggtaaaggtaataacgataccgaaaaacttgccactggtggtatacaagtgggcgtggataaagacggcaacgctaacggcgatttaagcaatgtttgggtcaaaacccaaaaagatggcagcaaaaaagccctgctcgccacttataacgccgcaggtcagaccaactatttgaccaacaaccccgcagaagccattgacagaataaatgaacaaggtatccgcttcttccatgtcaacgatggcaatcaagagcctgtggtacaagggcgtaacggcattgactcaagtgcctcaggcaagcactcagtggcgataggtttccaggccaaggcagatggtgaagccgccgttgccataggcagacaaacccaagcaggcaaccaatccatcgccatcggtgataacgcacaagccacgggcgatcaatccatcgccatcggtacaggcaatgtggtagcaggtaagcactctggtgccatcggcgacccaagcactgttaaggctgataacagttacagtgtgggtaataacaaccagtttaccgatgccactcaaaccgatgtctttggtgtgggcaataacatcaccgtgaccgaaagtaactcggttgccttaggttcaaactctgccatcagtgcaggcacacacgcaggcacacaagccaaaaaatctgacggcacagcaggtacaaccaccacagcaggtgcaaccggtacggttaaaggctttgctggacaaacggcggttggtgcggtctccgtgggtgcctcaggtgctgaacgccgtatccaaaatgtggcagcaggtgaggtcagtgccaccagcaccgatgcggtcaatggtagccagttgtacaaagccacccaaagcattgccaacgcaaccaatgagcttgaccatcgtatccaccaaaacgaaaataaggccaatgcagggatttcatcagcgatggcgatggcgtccatgccacaagcctacattcctggcagatccatggttaccgggggtattgccacccacaacggtcaaggtgcggtggcagtgggactgtcgaagctgtcggataatggtcaatgggtatttaaaatcaatggttcagccgatacccaaggccatgtaggggcggcagttggtgcaggttttcacttt

SEQ ID NO: 7 - protein, 2047 aa, Moraxella catarrhalis; identical to SEQ ID NO: 4

SEQ ID NO: 8 - DNA, 6159 bp, Moraxella catarrhalis
atgaatcacatctataaagtcatctttaacaaagccacaggcacatttatggccgtggcggaatatgccaaatcccacagtacgggggggggtagctgtgctacagggcaagttggcagtgtacgcactctaagctttgcccgtattgccgcgctcgctgtcctcgtgatcggtgcgacgctcaatggcagtgcttatgctcaacaaattactaccaagatcgaaattggtcaaacaaacaagataaacaacacgctgaaaggcgatgccctagcgacaggtgaagcatccattgcttttggtagtctttctaaggcacaaggctctcaagctattgctatcggtagtgtcaaaccagatcctaataatggtagtaatggtaatgtaggttcccacgccaaaggtaacgagtccatcgccatcggtggtgatgtattggctgagggtgatgcctcgattgccatcggtagtgatgacttatatttgcctaagaatcttgatctgaagaatgaatttcacaaacttattcatggccatgaaatattaaaaaaaatacaaacctcaaccgatggtaaaatcaaatatcgacgcacaagagcacaagggcacgccagtactgcagtgggagccatgtcatatgcacagggtcatttttccaacgcctttggtacatacgcaacagctgaagctgcctattccttggcagtaggtcttgccgcccaagccacaaaacaatcttcaatcgctgttggttccaatgcaaaagctaacgcgtttgcagcgacagccattggtggaaatactgtagttaatttgggtcgaggcgttgccctaggttttggttctcagatccttgatagggataataatacagatgccagtgcctatgtaccactaggtaaaacgttagcagaccagtataaagccacccgccagggtgattctacggatatattttccattggtaatagtaataataataatagcagtatcaggcgtaaaatcatcaatgtcggtgcgggttctcgggataccgatgcggtcaatgtggcacagcttaaattggtggaggaactggctaatcgtaaaattacttttaagggtgatggtgacaataatagcaatagcgtagaaagaggtttgggcaatactttaactattaaaggtgatgcacagaccaacgcattaaccgaagctaacatcggtgtggtaacagatggcaatggtctgaaagttaaacttgctaaagagctgactggattgaccagtgtctccgctaccaacaaaatcaccgttagtaataccaacaacaacaacgccgagctacaaagcggtggtttgacctttagcccaataacaggtacaaaaacagataaaaccgtctacagcattgatggattgaagtttactaatgatagtaatagtatagcaactaaaggtactactcgtattaccaaaaagaaaattggttttgctggtactaatgatggagttgatgaaagcaaaccttatcttgacaacgaaaagctaaaagttggcaacagcaccctaaacagtggtagcttgactgttaataacaccactggtaataaacaaatccaagtcggtgctaatggcattaaatttgccacagtcgctaataatgttgcaaatacctcagcaacagtcggcactgctcgtattaccgaagagaaaattggttttgctggtactaatgatggagttgatgaacaagcaccatatttggataaagaacgacttaaagtgggtcgtgttgaaattaccacagatagtggtattaatgctggtaatcacaagattaccggacttactaatggtatagcaaataccgatgcggttaccatcaaacagctcaaagacgccaagcctactttaaacgcaggcgatggcatcagtattaatagtaataacggggatctagttgatagtagtggcaatattaccaccccaacttataacattagcgtgaaaaccactaagcttaacagtaatggcaccagtggtaataataaatttagtgttagtaatgctcatgataacaatagcttagttaccgccaaagatttggcagactatctaaataaagtcaatgaaacggctgacagtgctctaccaagctttaaagtccaaaacggtgataatagcaacaacgccatcaccgtgggtaaagatacaaacggcaagaccttcaacaccttaaaactcaaaggtgaaaacggtgttaatattacgaccaatagagccacaggtacagttacctttggcattgaccaaagtaatggtctcaccacgcctaagctgaccgtgggtagcgatacaaatggtaatcgattggttattgagcaagtccctagcgctgacggtaacagcaccaaaaacatcattaaaggattgtccccaacactgcctagcattgccagtccaagtggccgcaacatagcactgggcaatacaatcgaagaaaaagacaaatccaacgctgccagcattgatgatgtgctaaatgcaggctttaacctaaaaaataatggcaaagacaaagactttgtctccacttatgacactgttgactttatcgatggcaatgccaccaccgccacagtaacttatgatgaagccaatcaaaccagtaaagtggcgtatgatgtgaatgtggatgagaaaaccattgaactgacaggcgataatggcaagaaacaacttggcgtcaaaaccatcaaactgaccgaaacaagtactaatggtaatgcaactacatttagtaccgacgatgaccatgcccttgttaaagccagtgatatcgccggcaatctaaacaccctagccgaggaaattcacaccaccaaaggcacagcaaacaccgccctacaaacctttaccgttaaaaaggtagatgaaaatgataaggctgatgacaccaacgccatcaccgtgggtaaagatggcacaagtggtaaagtcaacaccttaaaactcaaaggtaaaaacggtcttgatattaaaaccgacaaagatggtacggttacctttggcattaacacccaaagcggtcttaaagccggcgacagcaccactctaaacaacaatggcttgtctattaaaaacaccgctagtaacgaacaaatccaagtcggtgctgatggcgtgaagtttgccatggttaataatggtgttgtaggtgctggcattgatggcacaactcgcattaccagagatgaaattggctttactgggactaatggctcacttgataaaagcaaaccccacctaagcaaagacggcattaacgcaggtggtaaaaagattaccaacattcaatcaggtgagattgccaaaaacagccatgatgctgtgacaggcggcaagatttatgatttaaaaaccgaacttgaaaataaaatcagcagtactgccaaaacagcacaaaactcattacacgaattctcagtagcagatgaacaaggtaataactttacggttagtaacccttactccagttatgacacctcaaagacctctgatgtcatcacctttgcaggtgaaaacggcattaccaccaaggtaaataaaggtgtggtgcgtgtgggcattgaccaaaccaaaggcttaaccacgcctaagctgaccgtgggtaataataatggcaaaggcattgtcattaacagccaaaatggtcaaaataccatcacaggactaagcaacactctagctaatgttaccaatgataaaggtagcgtacgcaccacagaacagggcaatataatcaaagacgaagacaaaacccgtgccgccagcattgttgatgtgctaagcgcaggctttaacttgcaaggcaatggtgaagcggttgactttgtctccacttatgacaccgtcaactttgccaatggcaataccaccaccgctaaggtgacctatgatgacacaagcaaaaccagtaaagtggtctatgatgtcaatgtggatgatacaaccattgaagttaaagataaaaaacttggcgtaaaaaccaccacattgaccagtactggcacaggtgctaataaatttgccctaagcaatcaagctactggcgatgcgcttgtcaaggccagtgatatcgttgctcatctaaacaccttatctggcgacatccaaactgccaaaggggcaagccaagcgaacaactcagcaggctatgtggatgctgatggcaataaggtcatctatgacagtaccgataacaagtactatcaagccaaaaatgatggcacagttgataaaaccaaagaagttgccaaagacaaactggtcgcccaagcccaaaccccagatggcacattggctcaaatgaatgtcaaatcagtcattaacaaagaacaagtaaatgatgccaataaaaagcaaggcatcaatgaagacaacgcctttgttaaaggacttgaaaaagccgcttctgataacaaaaccaaaaacgccgcagtaactgtgggtgatttaaatgccgttgcccaaacaccgctgacctttgcaggggatacaggcacaacggctaaaaaactgggcgagactttgaccatcaaaggtgggcaaacagacaccaataagctaaccgataataacatcggtgtggtagcaggtactgatggcttcactgtcaaacttgccaaagacctaaccaatcttaacagcgttaatgcaggtggcaccaaaattgatgaaaaaggcatctcttttgtagacgcaaacggtcaagccaaagcaaacacccctgtgctaagtgccaatgggctggacctgggtggcaaggtcatcagtaatgtgggcaaaggcacaaaagataccgacgctgccaatgtacaacagttaaacgaagtacgcaacttgttgggtcttggtaatgataacgctgacggcaatcaggtaaacattgccgacatcaaaaaagacccaaattcaggttcatcatctaaccgcactgtcatcaaagcaggcacggtacttggcggtaaaggtaataacgataccgaaaaacttgccactggtggtgtacaagtgggcgtggataaagacggcaacgctaacggcgatttaagcaatgtttgggtcaaaacccaaaaagatggcagcaaaaaagccctgctcgccacttataacgccgcaggtcagaccaactatgtgaccaacaaccccgcagaagccattgacagaataaatgaacaaggtatccgcttcttccatgtcaacgatggcaatcaagagcctgtggtacaagggcgtaacggcattgactcaagtgcctcaggcaagcactcagtggcgataggtttccaggccaaggcagatggtgaagccgccgttgccataggcagacaaacccaagcaggcaaccaatccatcgccatcggtgataacgcacaagccacgggcgatcaatccatcgccatcggtacaggcaatgtggtagcaggtaagcactctggtgccatcggcgacccaagcactgttaaggctgataacagttacagtgtgggtaataacaaccagtttaccgatgccactcaaaccgatgtctttggtgtgggcaataacatcaccgtgaccgaaagtaactcggttgccttaggttcaaactctgccatcagtgcaggcacacacgcaggcacacaagccaaaaaatctgacggcacagcaggtacaaccaccacagcaggtgccacaggtacggttaaaggctttgctggacaaacggcggttggtgcggtctccgtgggtgcctcaggtgctgaacgccgtatccaaaatgtggcagcaggtgaggtcagtgccaccagcaccgatgcggtcaatggtagccagttgtacaaagccacccaaagcattgccaacgcaaccaatgagcttgaccatcgtatccaccaaaacgaaaataaagccaatgcagggatttcatcagcgatggcgatggcgtccatgccacaagcctacattcctggcagatccatggttaccgggggtattgccacccacaacggtcaaggtgcggtggcagtgggactgtcgaagctgtcggataatggtcaatgggtatttaaaatcaatggttcagccgatacccaaggccatgtaggggcggcagttggtgcaggttttcacttt

SEQ ID NO: 9 - protein, 2053 aa, Moraxella catarrhalis
MNHIYKVIFNKATGTFMAVAEYAKSHSTGGGSCATGQVGSVRTLSFARIAALAVLVIGATLNGSAYAQQITTKIEIGQTNKINNTLKGDALATGEASIAFGSLSKAQGSQAIAIGSVKPDPNNGSNGNVGSHAKGNESIAIGGDVLAEGDASIAIGSDDLYLPKNLDLKNEFHKLIHGHEILKKIQTSTDGKIKYRRTRAQGHASTAVGAMSYAQGHFSNAFGTYATAEAAYSLAVGLAAQATKQSSIAVGSNAKANAFAATAIGGNTVVNLGRGVALGFGSQILDRDNNTDASAYVPLGKTLADQYKATRQGDSTDIFSIGNSNNNNSSIRRKIINVGAGSRDTDAVNVAQLKLVEELANRKITFKGDGDNNSNSVERGLGNTLTIKGDAQTNALTEANIGVVTDGNGLKVKLAKELTGLTSVSATNKITVSNTNNNNAELQSGGLTFSPITGTKTDKTVYSIDGLKFTNDSNSIATKGTTRITKKKIGFAGTNDGVDESKPYLDNEKLKVGNSTLNSGSLTVNNTTGNKQIQVGANGIKFATVANNVANTSATVGTARITEEKIGFAGTNDGVDEQAPYLDKERLKVGRVEITTDSGINAGNHKITGLTNGIANTDAVTIKQLKDAKPTLNAGDGISINSNNGDLVDSSGNITTPTYNISVKTTKLNSNGTSGNNKFSVSNAHDNNSLVTAKDLADYLNKVNETADSALPSFKVQNGDNSNNAITVGKDTNGKTFNTLKLKGENGVNITTNRATGTVTFGIDQSNGLTTPKLTVGSDTNGNRLVIEQVPSADGNSTKNIIKGLSPTLPSIASPSGRNIALGNTIEEKDKSNAASIDDVLNAGFNLKNNGKDKDFVSTYDTVDFIDGNATTATVTYDEANQTSKVAYDVNVDEKTIELTGDNGKKQLGVKTIKLTETSTNGNATTFSTDDDHALVKASDIAGNLNTLAEEIHTTKGTANTALQTFTVKKVDENDKADDTNAITVGKDGTSGKVNTLKLKGKNGLDIKTDKDGTVTFGINTQSGLKAGDSTTLNNNGLSIKNTASNEQIQVGADGVKFAMVNNGVVGAGIDGTTRITRDEIGFTGTNGSLDKSKPHLSKDGINAGGKKITNIQSGEIAKNSHDAVTGGKIYDLKTELENKISSTAKTAQNSLHEFSVADEQGNNFTVSNPYSSYDTSKTSDVITFAGENGITTKVNKGVVRVGIDQTKGLTTPKLTVGNNNGKGIVINSQNGQNTITGLSNTLANVTNDKGSVRTTEQGNIIKDEDKTRAASIVDVLSAGFNLQGNGEAVDFVSTYDTVNFANGNTTTAKVTYDDTSKTSKVVYDVNVDDTTIEVKDKKLGVKTTTLTSTGTGANKFALSNQATGDALVKASDIVAHLNTLSGDIQTAKGASQANNSAGYVDADGNKVIYDSTDNKYYQAKNDGTVDKTKEVAKDKLVAQAQTPDGTLAQMNVKSVINKEQVNDANKKQGINEDNAFVKGLEKAASDNKTKNAAVTVGDLNAVAQTPLTFAGDTGTTAKKLGETLTIKGGQTDTNKLTDNNIGVVAGTDGFTVKLAKDLTNLNSVNAGGTKIDEKGISFVDANGQAKANTPVLSANGLDLGGKVISNVGKGTKDTDAANVQQLNEVRNLLGLGNDNADGNQVNIADIKKDPNSGSSSNRTVIKAGTVLGGKGNNDTEKLATGGVQVGVDKDGNANGDLSNVWVKTQKDGSKKALLATYNAAGQTNYVTNNPAEAIDRINEQGIRFFHVNDGNQEPVVQGRNGIDSSASGKHSVAIGFQAKADGEAAVAIGRQTQAGNQSIAIGDNAQATGDQSIAIGTGNVVAGKHSGAIGDPSTVKADNSYSVGNNNQFTDATQTDVFGVGNNITVTESNSVALGSNSAISAGTHAGTQAKKSDGTAGTTTTAGATGTVKGFAGQTAVGAVSVGASGAERRIQNVAAGEVSATSTDAVNGSQLYKATQSIANATNELDHRIHQNENKANAGISSAMAMASMPQAYIPGRSMVTGGIATHNGQGAVAVGLSKLSDNGQWVFKINGSADTQGHVGAAVGAGFHF

SEQ ID NO: 10 - DNA, 6942 bp, Moraxella catarrhalis
atgaatcacatctataaagtcatctttaacaaagccacaggcacatttatggccgtggcagagtgcgccaaatcccacagcggagggagtagcagtagtaccgcaggacaggtgggcagctctcctgtcatccgcctgactcgtgttgccacgctcgctatcctagtgatcggtgcgacgctcaatggcagtgcttatgctcaaaataatagcaagatcgcatttggtaccacaggcaacaatgacaatgcctcggctagcaatgaagcatccattgctattggtagtcttgctaaggcacatgccaatcaagctattgctatcggtggtagcaaaccagatcctcgtaatcaagcggctaatcagaaggcaggttcccacgccaaaggtaaagagtccatcgccatcggtggtgatgtactggctgagggtgatgcctcgattgccattggtagtgatgacttatatttggataggaatagcactaactctaaatatccaaatggtcttcttagcactcttattcaaaaccatacagtattacgccaaatacgagactcaaatggttctcagaaatatagacgcacagcagcagaaggacacgccagtactgcagtgggagccatggcatatgcaaagggtcattttgccaacgcctttggtacacggtcaacagctgaaggcaactattccttggcagtaggtcttaccgccaaagccgaaaaaggatatacaatcgctattggttctaatgcacaagctatcaattatggagcactagcccttggtgcagatactcgagttgatttggattacggtattgccctaggttatggttctcagatccttaataataataataataataataataaagcctatgtaccagaaggtaatgggtcaaacataaaatcgtctaaagccaccggcaatggtttattttccattggtagtagcactatcaagcgtaaaatcatcaatgtcggtgcaggttatgaggataccgatgcggtcaatgtggcacagctaaaagcggtggagaatctggctaagcgtcaaattacttttaagggtgatgataacggtactggcgttaagaaaaaactgggcgagactttaaccattaaaggtggtgagacccaagcggacaagctaaccgataataataacattggtgtggtaacagataataatactggtctgaaagttaaacttgctaaaaacctaagcggtcttgaaacagttagcaccaaaaacctaaccgccagcgagaaagttacggtaggtagtggtaataacaccgctgagctacaaagcggtggtttaacctttaccccaacaacaaatgcaagcacagacaaaaccgtctatggcactgatgggcttaagtttactgataattctaatacggcacttgaagatactactcgtatcaccaaagataaaattggttttagcaataaagctggtacagttgatgaaaacaaaccttatcttgataaagacaagctaaaagttggcaacagcaccctaaacaacggtggcttgactgttaataacaccattggtggtagcaataaacaaatccaagtcggtgctgatggcattaaatttgccgatgtgaatgttaatgtatcaaatgccgcaaaattcggcactactcgtattaccgaagaggaaattggctttgctgatgctgatggtaaagttgataaaaagtcaccatatttggataaaaaacaacttcaagtgggtggtgttaaaattaccaaagacagtggcattaatgcaggtgatcaaaagatcagtaatgttaaagatgcaacggacgataccgatgcagtcacttataaacagcttaaacaagtccaacaagacgccgacggtgccctacaaagcttctctattcgtgatgaaaaaggtcaggaatttacgattagtaacttgtattctaatggtaataccccaaatacctttgagaccatcacctttgcaggtgaaaacggcatcagtatcagcaatgacatagccaaaggtaaagtcaaagttggtattgacccaatcaatggtctcaccacgcctaagctgaccgtgggtagcgataaagatggtaaaactcaattggttattgagcaagtggctagcggtaacgacaccaaaaacatcattagaggattgtccccaacactgcctagcattaccaatgcaggtggcgtacgcaccacagaacagggcaatacaatcaccagcgacgaagacaaatccaaagccgccagtatcggtgatatattaaatacaggctttaacctaaaaaataatagcaactccgttggctttgtctccacttataacactgttgactttatcgatggcaatgccaccaccgctaaggtaacttacgatgaaaccaatcaaaccagtaaagtaacttatgatgtcaatgtggatgagaaaaccattgaactcacaggcgataatggcaagacaaacaaaattggcgtcaaaaccaccacactgaccacaacaaatgctaatggtaaagcaaccaactttagtaccaccgataacgatgcccttgttaacgccaaagacatcgccgaaaatctaaacaccctagccaaggaaattcacaccaccaaaggcacagcagacaccgccctacaaacctttaaagtcaaaaaagacggtgcaactgatgacgaaaccatcaccgtgggtaaagatggtacacaaaacggcaagaccgtcaacactctaaaactcaaaggtgaaaacggtctaacggttgctaccaataaagatggtacggttacctttggcattaacacccaaagcggtcttaaagccggcgacagcaccactctaaacaaagatggcttgtctattaaaaaccccgctagtaacgaacaaatccaagtcggtgctgatggcgtgaagtttgccaaggttgataagggtaattcaagcactggcattgatggcacaagccgtatcaccaaagatcaaattggctttactggggctaatggctcacttgataccaccaaaccccacctaaccaaagacaagcttaaagtgggtgaagttgaaattaccaacactggcattaacgcaggtggtaaaaagattaccaacattcaatcaggtgatattacccaaaacagcaatgatgctgtgacaggcggtcgggtttatgatttaaaaaccgaacttgaaagcaaaatcaacagtgctgctaaaacagcacaaaactcattacacgaattctcagtagcagatgaacaaggtaatcactttacggttagtaacccttactccagttatgacacctcaaagacctctgatgtcatcacctttgcaggtgaaaacggcattaccaccaaggtaaataaaggtgtggtgcgtgtgggcattgaccaaaccaaaggcttaaccacgcctaagctgaccgtgggtaataataatggcaaaggcattgtcattgacagtaaagatggtcaaaataccatcacaggactaagcaacactctagctaatgttaccaatgatggtgcaggacacgcactaagccaagggcttgccaatgacaccgacaaaacccgtgccgccagcattggtgatgtgctaaacgcaggctttaacttgcaaggcaatggtgaagcggttgactttgtctccacttatgacactgttgactttatcgatggcaatgccaccaccgctaaggtgacctatgatgacacaagcaaaaccagtaaagtggtctatgatgtcaatgtggataataaaaccattgaagtgacaagtgataaaaaacttggcgtcaaaaccaccacactgaccaaaacaagtgctaatggtaatgcaaccaaatttagtgccgccgatggcgatgcccttgttaaagccagtgatatcgccacccatctaaataccttggctggcgacatccaaaccgccaaaggggcaagccaagcaagcagctcagcaagctatgtggatgctgatggcaacaaggtcatctatgacagtaccgataagaagtactatcaagtcaatgacaagggtcaagtggacaaaaacaaagaagttgccaaagacaaactggtcgcccaagcccaaaccccagatggcacattggctcaaatgaatgtcaaatcagtcattaacaaagagcaagtaaatgatgccaataaaaagcaaggcatcaatgaagacaacgcctttatcaaagggcttgaaaacgccgccaaagacaccaaaaccaaaaacgccgcagtaactgtgggtgatttaaatgccgttgcccaaacaccgctgacctttgcaggggatacaggcacaacggctaaaaaactgggcgagactttgaccatcaaaggtgggcaaacagacaccaataagctaaccgataataacatcggtgtggtagcaggtactgatggcttcactgtcaaacttgccaaagacctaaccaatcttaacagcgttaatgcaggtggcaccagaattgatgaaaaaggcatctcttttgtagacgcaaacggtcaagccaaagcaaacacccctgtgctaagtgccaatgggctggacctgggtggcaaacgcatcagtaacatcggtgcagctgttgatgataacgatgcggtgaactttaagcagtttaatgaagttgccaaaacggtcaacaacctaaacaaccaaagtaactcaggtgcgtcattaccctttgtggtaaccgatgccaatggcaagcccatcaatggcaccgatggcaagccccaaaaagccatcaagggcgccgatggtaaatactatcacgccaacgccaacggcgtacctgtggacaaagatggcaagcccatcaccgatgcggacaaacttgccaatctggcagctcatggcaaaccccttgatgcaggtcatcaagtggtggcaagcctaggcggcaactcagatgccatcaccctaaccaacatcaagtccactttgccacaaattgacacaccaaacacaggtaatgccaatgcagggcaagcccaaagtctgcccagcctatcagcagcacagcaaagtaatgctgccagtgtcaaagatgtgctaaatgtaggctttaacttgcagaccaatcacaatcaagtggactttgtcaaagcctatgataccgtcaactttgtcaatggtacaggtgccgacatcacaagcgtgcgtagtgctgatggcacgatgagtaacatcaccgtcaacaccgccttagcagcgaccgatgatgatggcaatgtgcttatcaaagccaaagatggtaagttctacaaagcagacgacctcatgccaaacggctcactaaaagcaggcaaatcagccagtgatgccaaaactccaactggtctaagccttgttaaccccaatgctggtaaaggcagtacaggcgatgcagtggctcttaataacttatcaaaagcggtatttaaatccaaagatggtacaactactaccacagtaagctctgatggcatcagtatccaaggcaaagataacagcagcatcaccctaagcaaagatgggctgaatgtaggcggtaaggtcatcagcaatgtgggtaaaggcacaaaagacaccgacgctgccaatgtacaacagttaaacgaagtacgcaacttgttgggtcttggtaatgctggtaatgataacgctgacggcaatcaggtaaacattgccgacatcaaaaaagacccaaattcaggttcatcatctaaccgcactgtcatcaaagcaggcacggtacttggcggtaaaggtaataacgataccgaaaaacttgccactggtggtgtacaagtgggcgtggataaagacggcaacgctaacggcgatttaagcaatgtttgggtcaaaacccaaaaagatggcagcaaaaaagccctgctcgccacttataacgccgcaggtcagaccaactatttgaccaacaaccccgcagaagccattgacagaataaatgaacaaggtatccgcttcttccatgtcaacgatggcaatcaagagcctgtggtacaagggcgtaacggcattgactcaagtgcctcaggcaagcactcagtggcgataggtttccaggccaaggcagatggtgaagccgccgttgccataggcagacaaacccaagcaggcaaccaatccatcgccatcggtgataacgcacaagccacgggcgatcaatccatcgccatcggtacaggcaatgtggtaacaggtaagcactctggtgccatcggcgacccaagcactgttaaggctgataacagttacagtgtgggtaataacaaccagtttatcgatgccactcagaccgatgtctttggtgtgggcaataacatcaccgtgaccgaaagtaactcggttgccttaggttcaaactctgccatcagtgcaggcacacacgcaggcacacaagccaaaaaatctgacggcacagcaggtacaaccaccacagcaggtgcaacaggtacggttaaaggctttgctggacaaacggcggttggtgcggtctccgtgggtgcctcaggtgctgaacgccgtatccaaaatgtggcagcaggtgaggtcagtgccaccagcaccgatgcggtcaatggtagccagttgtacaaagccacccaaggcattgccaacgcaaccaatgagcttgaccatcgtatccaccaaaacgaaaataaagccaatgcagggatttcatcagcgatggcgatggcgtccatgccacaagcctacattcctggcagatccatggttaccgggggtattgccacccacaacggtcaaggtgcggtggcagtgggactgtcgaagctgtcggataatggtcaatgggtatttaaaatcaatggttcagccgatacccaaggccatgtaggggcggcagttggtgcaggttttcacttt

SEQ ID NO: 11 - protein, 2314 aa, Moraxella catarrhalis
MNHIYKVIFNKATGTFMAVAECAKSHSGGSSSSTAGQVGSSPVIRLTRVATLAILVIGATLNGSAYAQNNSKIAFGTTGNNDNASASNEASIAIGSLAKAHANQAIAIGGSKPDPRNQAANQKAGSHAKGKESIAIGGDVLAEGDASIAIGSDDLYLDRNSTNSKYPNGLLSTLIQNHTVLRQIRDSNGSQKYRRTAAEGHASTAVGAMAYAKGHFANAFGTRSTAEGNYSLAVGLTAKAEKGYTIAIGSNAQAINYGALALGADTRVDLDYGIALGYGSQILNNNNNNNNKAYVPEGNGSNIKSSKATGNGLFSIGSSTIKRKIINVGAGYEDTDAVNVAQLKAVENLAKRQITFKGDDNGTGVKKKLGETLTIKGGETQADKLTDNNNIGVVTDNNTGLKVKLAKNLSGLETVSTKNLTASEKVTVGSGNNTAELQSGGLTFTPTTNASTDKTVYGTDGLKFTDNSNTALEDTTRITKDKIGFSNKAGTVDENKPYLDKDKLKVGNSTLNNGGLTVNNTIGGSNKQIQVGADGIKFADVNVNVSNAAKFGTTRITEEEIGFADADGKVDKKSPYLDKKQLQVGGVKITKDSGINAGDQKISNVKDATDDTDAVTYKQLKQVQQDADGALQSFSIRDEKGQEFTISNLYSNGNTPNTFETITFAGENGISISNDIAKGKVKVGIDPINGLTTPKLTVGSDKDGKTQLVIEQVASGNDTKNIIRGLSPTLPSITNAGGVRTTEQGNTITSDEDKSKAASIGDILNTGFNLKNNSNSVGFVSTYNTVDFIDGNATTAKVTYDETNQTSKVTYDVNVDEKTIELTGDNGKTNKIGVKTTTLTTTNANGKATNFSTTDNDALVNAKDIAENLNTLAKEIHTTKGTADTALQTFKVKKDGATDDETITVGKDGTQNGKTVNTLKLKGENGLTVATNKDGTVTFGINTQSGLKAGDSTTLNKDGLSIKNPASNEQIQVGADGVKFAKVDKGNSSTGIDGTSRITKDQIGFTGANGSLDTTKPHLTKDKLKVGEVEITNTGINAGGKKITNIQSGDITQNSNDAVTGGRVYDLKTELESKINSAAKTAQNSLHEFSVADEQGNHFTVSNPYSSYDTSKTSDVITFAGENGITTKVNKGVVRVGIDQTKGLTTPKLTVGNNNGKGIVIDSKDGQNTITGLSNTLANVTNDGAGHALSQGLANDTDKTRAASIGDVLNAGFNLQGNGEAVDFVSTYDTVDFIDGNATTAKVTYDDTSKTSKVVYDVNVDNKTIEVTSDKKLGVKTTTLTKTSANGNATKFSAADGDALVKASDIATHLNTLAGDIQTAKGASQASSSASYVDADGNKVIYDSTDKKYYQVNDKGQVDKNKEVAKDKLVAQAQTPDGTLAQMNVKSVINKEQVNDANKKQGINEDNAFIKGLENAAKDTKTKNAAVTVGDLNAVAQTPLTFAGDTGTTAKKLGETLTIKGGQTDTNKLTDNNIGVVAGTDGFTVKLAKDLTNLNSVNAGGTRIDEKGISFVDANGQAKANTPVLSANGLDLGGKRISNIGAAVDDNDAVNFKQFNEVAKTVNNLNNQSNSGASLPFVVTDANGKPINGTDGKPQKAIKGADGKYYHANANGVPVDKDGKPITDADKLANLAAHGKPLDAGHQVVASLGGNSDAITLTNIKSTLPQIDTPNTGNANAGQAQSLPSLSAAQQSNAASVKDVLNVGFNLQTNHNQVDFVKAYDTVNFVNGTGADITSVRSADGTMSNITVNTALAATDDDGNVLIKAKDGKFYKADDLMPNGSLKAGKSASDAKTPTGLSLVNPNAGKGSTGDAVALNNLSKAVFKSKDGTTTTTVSSDGISIQGKDNSSITLSKDGLNVGGKVISNVGKGTKDTDAANVQQLNEVRNLLGLGNAGNDNADGNQVNIADIKKDPNSGSSSNRTVIKAGTVLGGKGNNDTEKLATGGVQVGVDKDGNANGDLSNVWVKTQKDGSKKALLATYNAAGQTNYLTNNPAEAIDRINEQGIRFFHVNDGNQEPVVQGRNGIDSSASGKHSVAIGFQAKADGEAAVAIGRQTQAGNQSIAIGDNAQATGDQSIAIGTGNVVTGKHSGAIGDPSTVKADNSYSVGNNNQFIDATQTDVFGVGNNITVTESNSVALGSNSAISAGTHAGTQAKKSDGTAGTTTTAGATGTVKGFAGQTAVGAVSVGASGAERRIQNVAAGEVSATSTDAVNGSQLYKATQGIANATNELDHRIHQNENKANAGISSAMAMASMPQAYIPGRSMVTGGIATHNGQGAVAVGLSKLSDNGQWVFKINGSADTQGHVGAAVGAGFHF

SEQ ID NO: 12 - DNA, 6259 bp, Moraxella catarrhalis
atgatcggtgcaacgctcagtggcagtgcttatgctcaaaaaaaagataccaaacatatcgcaattggtgaacaaaaccagccaagacgctcaggcactgccaaggcggacggtgatcgagccattgctattggtgaaaatgctaacgcacagggcggtcaagccatcgccatcggtagtagtaataaaactgtcaatggaagcagtttggataagataggtaccgatgctacgggtcaagagtccatcgccatcggtggtgatgtaaaggctagtggtgatgcctcgattgccatcggtagtgatgacttacatttgcttgatcagcatggtaatcctaaacatccgaaaggtactctgattaacgatcttattaacggccatgcagtattaaaagaaatacgaagctcaaaggataatgatgtaaaatatagacgcacaaccgcaagcggacacgccagtactgcagtgggagccatgtcatatgcacagggtcatttttccaacgcctttggtacacgggcaacagctaaaagtgcctattccttggcagtgggtcttgccgccacagccgagggccaatctacaatcgctattggttctgatgcaacatctagctcgttgggagcgatagcccttggtgcaggtactcgtgctcagctacagggcagtattgccctaggtcaaggttctgttgtcactcagagtgataataattctagaccggcctatacaccaaatacccaggcactagaccccaagtttcaagccaccaataatacgaaggcgggtccactttccattggtagtaactctatcaaacgtaaaatcatcaatgtcggtgcaggtgttaataaaaccgatgcggtcaatgtggcacagctagaagcggtggtgaagtgggctaaggagcgtagaattacttttcagggtgatgataacagtactgacgtaaaaataggtttggataatactttaactattaaaggtggtgcagagaccaacgcattaaccgataataatatcggtgtggtaaaagaggctgataatagtggtctgaaagttaaacttgctaaaactttaaacaatcttactgaggtgaatacaactacattaaatgccacaaccacagttaaggtaggtagtagtagtagtactacagctgaattattgagtgatagtttaacctttacccagcccaatacaggcagtcaaagcacaagcaaaaccgtctatggcgttaatggggtgaagtttactaataatgcagaaacaacagcagcaatcggcactactcgtattaccagagataaaattggctttgctcgagatggtgatgttgatgaaaaacaagcaccatatttggataaaaaacaacttaaagtgggtagtgttgcaattaccatagacaatggcattgatgcaggtaataaaaagatcagtaatcttgccaaaggtagcagtgctaacgatgcggttaccatcgaacagctcaaagccgccaagcctactttaaacgcaggcgctggcatcagtgtcacacctactgaaatatcagttgatgctaagagtggcaatgttaccgccccaacttacaacattggcgtgaaaaccaccgagcttaacagtgatggcactagtgataaatttagtgttaagggtagtggtacgaacaatagcttagttaccgccgaacatttggcaagctatctaaatgaagtcaatcgaacggctgacagtgctctacaaagctttaccgttaaagaagaagacgatgatgacgccaacgctatcaccgtggctaaagatacgacaaaaaatgccggcgcagtcagcatcttaaaactcaaaggtaaaaacggtctaacggttgctaccaaaaaagatggtacggttacctttgggcttagccaagatagcggtctgaccattggcaaaagcaccctaaacaacgatggcttgactgttaaagataccaacgaacaaatccaagtcggtgctaatggcattaaatttactaatgtgaatggtagtaatccaggtactggcattgcaaataccgctcgcattaccagagataaaattggctttgctggttctgatggtgcagttgatacaaacaaaccttatcttgatcaagacaagctacaagttggcaatgttaagattaccaacactggcattaacgcaggtggtaaagccatcacagggctgtccccaacactgcctagcattgccgatcaaagtagccgcaacatagaactgggcaatacaatccaagacaaagacaaatccaacgctgccagcattaatgatatattaaatacaggctttaacctaaaaaataataacaaccccattgactttgtctccacttatgacattgttgactttgccaatggcaatgccaccaccgccacagtaacccatgataccgctaacaaaaccagtaaagtggtatatgatgtgaatgtggatgatacaaccattcatctaacaggcactgatgacaataaaaaacttggcgtcaaaaccaccaaactgaacaaaacaagtgctaatggtaatacagcaactaactttaatgttaactctagtgatgaagatgcccttgttaacgccaaagacatcgccgaaaatctaaacaccctagccaaggaaattcacaccaccaaaggcacagcagacaccgccctacaaacctttaccgttaaaaaggtagatgaaaataataatgctgatgacgccaacgccatcaccgtgggtcaaaagaacgcaaataatcaagtcaacaccctaacactcaaaggtgaaaacggtcttaatattaaaaccgacaaaaatggtacggttacctttggcattaacaccacaagcggtcttaaagccggcaaaagcaccctaaacgacggtggcttgtctattaaaaaccccactggtagcgaacaaatccaagtcggtgctgatggcgtgaagtttgccaaggttaataataatggtgttgtaggtgctggcattgatggcacaactcgcattaccagagatgaaattggctttactgggactaatggctcacttgataaaagcaaaccccacctaagcaaagacggcattaacgcaggtggtaaaaagattaccaacattcaatcaggtgagattgcccaaaacagccatgatgctgtgacaggcggcaagatttatgatttaaaaaccgaacttgaaaacaaaatcagcagtactgccaaaacagcacaaaactcattacacgaattctcagtagcagatgaacaaggtaataactttacggttagtaacccttactccagttatgacacctcaaagacctctgatgtcatcacctttgcaggtgaaaacggcattaccaccaaggtaaataaaggtgtggtgcgtgtgggcattgaccaaaccaaaggcttaaccacgcctaagctgaccgtgggtaataataatggcaaaggcattgtcattgacagccaaaatggtcaaaataccatcacaggactaagcaacactctagctaatgttaccaatgataaaggtagcgtacgcaccacagaacagggcaatataatcaaagacgaagacaaaacccgtgccgccagcattgttgatgtgctaagcgcaggctttaacttgcaaggcaatggtgaagcggttgactttgtctccacttatgacaccgtcaactttgccgatggcaatgccaccaccgctaaggtgacctatgatgacacaagcaaaaccagtaaagtggtctatgatgtcaatgtggatgatacaaccattgaagttaaagataaaaaacttggcgtaaaaaccaccacattgaccagtactggcacaggtgctaataaatttgccctaagcaatcaagctactggcgatgcgcttgtcaaggccagtgatatcgttgctcatctaaacaccttatctggcgacatccaaactgccaaaggggcaagccaagcgaacaactcagcaggctatgtggatgctgatggcaataaggtcatctatgacagtaccgataacaagtactatcaagccaaaaatgatggcacagttgataaaaccaaagaagttgccaaagacaaactggtcgcccaagcccaaaccccagatggcacattggctcaaatgaatgtcaaatcagtcattaacaaagaacaagtaaatgatgccaataaaaagcaaggcatcaatgaagacaacgcctttgttaaaggacttgaaaaagccgcttctgataacaaaaccaaaaacgccgcagtaactgtgggtgatttaaatgccgttgcccaaacaccgctgacctttgcaggggatacaggcacaacggctaaaaaactgggcgagactttgaccatcaaaggtgggcaaacagacaccaataagctaaccgataataacatcggtgtggtagcaggtactgatggcttcactgtcaaacttgccaaagacctaaccaatcttaacagcgttaatgcaggtggcaccaaaattgatgacaaaggcgtgtcttttgtagactcaagcggtcaagccaaagcaaacacccctgtgctaagtgccaatgggctggacctgggtggcaaggtcatcagtaatgtgggcaaaggcacaaaagataccgacgctgccaatgtacaacagttaaacgaagtacgcaacttgttgggtcttggtaatgctggtaatgataacgctgacggcaatcaggtaaacattgccgacatcaaaaaagacccaaattcaggttcatcatctaaccgcactgtcatcaaagcaggcacggtacttggcggtaaaggtaataacgataccgaaaaacttgccactggtggtatacaagtgggcgtggataaagacggcaacgctaacggcgatttaagcaatgtttgggtcaaaacccaaaaagatggcagcaaaaaagccctgctcgccacttataacgccgcaggtcagaccaactatttgaccaacaaccccgcagaagccattgacagaataaatgaacaaggtatccgcttcttccatgtcaacgatggcaatcaagagcctgtggtacaagggcgtaacggcattgactcaagtgcctcaggcaagcactcagtggcgataggtttccaggccaaggcagatggtgaagccgccgttgccataggcagacaaacccaagcaggcaaccaatccatcgccatcggtgataacgcacaagccacgggcgatcaatccatcgccatcggtacaggcaatgtggtagcaggtaagcactctggtgccatcggcgacccaagcactgttaaggctgataacagttacagtgtgggtaataacaaccagtttaccgatgccactcaaaccgatgtctttggtgtgggcaataacatcaccgtgaccgaaagtaactcggttgccttaggttcaaactctgccatcagtgcaggcacacacgcaggcacacaagccaaaaaatctgacggcacagcaggtacaaccaccacagcaggtgcaaccggtacggttaaaggctttgctggacaaacggcggttggtgcggtctccgtgggtgcctcaggtgctgaacgccgtatccaaaatgtggcagcaggtgaggtcagtgccaccagcaccgatgcggtcaatggtagccagttgtacaaagccacccaaagcattgccaacgcaaccaatgagcttgaccatcgtatccaccaaaacgaaaataaggccaatgcagggatttcatcagcgatggcgatggcgtccatgccacaagcctacattcctggcagatccatggttaccgggggtattgccacccacaacggtcaaggtgcggtggcagtgggactgtcgaagctgtcggataatggtcaatgggtatttaaaatcaatggttcagccgatacccaaggccatgtaggggcggcagttggtgcaggttttcacttttaagccataaatcgcaagattttacttaaaaatcaatctcaccatagttgtataaaacagcatcagcatcagtcatattactgatgctgatgttttttatcacttaaaccattttaccgctcaagtgattctctttcaccatgaccaaatcgccattgatcataggtaaacttattgagtaaattttatcaatgtagttgttagatatggttaaaattgtgccattgaccaaaaaatgaccgatttatcccgaaaatttctgattatgatccgttgacctgca

SEQ ID NO: 13 - protein, 1992 aa, Moraxella catarrhalis
MIGATLSGSAYAQKKDTKHIAIGEQNQPRRSGTAKADGDRAIAIGENANAQGGQAIAIGSSNKTVNGSSLDKIGTDATGQESIAIGGDVKASGDASIAIGSDDLHLLDQHGNPKHPKGTLINDLINGHAVLKEIRSSKDNDVKYRRTTASGHASTAVGAMSYAQGHFSNAFGTRATAKSAYSLAVGLAATAEGQSTIAIGSDATSSSLGAIALGAGTRAQLQGSIALGQGSVVTQSDNNSRPAYTPNTQALDPKFQATNNTKAGPLSIGSNSIKRKIINVGAGVNKTDAVNVAQLEAVVKWAKERRITFQGDDNSTDVKIGLDNTLTIKGGAETNALTDNNIGVVKEADNSGLKVKLAKTLNNLTEVNTTTLNATTTVKVGSSSSTTAELLSDSLTFTQPNTGSQSTSKTVYGVNGVKFTNNAETTAAIGTTRITRDKIGFARDGDVDEKQAPYLDKKQLKVGSVAITIDNGIDAGNKKISNLAKGSSANDAVTIEQLKAAKPTLNAGAGISVTPTEISVDAKSGNVTAPTYNIGVKTTELNSDGTSDKFSVKGSGTNNSLVTAEHLASYLNEVNRTADSALQSFTVKEEDDDDANAITVAKDTTKNAGAVSILKLKGKNGLTVATKKDGTVTFGLSQDSGLTIGKSTLNNDGLTVKDTNEQIQVGANGIKFTNVNGSNPGTGIANTARITRDKIGFAGSDGAVDTNKPYLDQDKLQVGNVKITNTGINAGGKAITGLSPTLPSIADQSSRNIELGNTIQDKDKSNAASINDILNTGFNLKNNNNPIDFVSTYDIVDFANGNATTATVTHDTANKTSKVVYDVNVDDTTIHLTGTDDNKKLGVKTTKLNKTSANGNTATNFNVNSSDEDALVNAKDIAENLNTLAKEIHTTKGTADTALQTFTVKKVDENNNADDANAITVGQKNANNQVNTLTLKGENGLNIKTDKNGTVTFGINTTSGLKAGKSTLNDGGLSIKNPTGSEQIQVGADGVKFAKVNNNGVVGAGIDGTTRITRDEIGFTGTNGSLDKSKPHLSKDGINAGGKKITNIQSGEIAQNSHDAVTGGKIYDLKTELENKISSTAKTAQNSLHEFSVADEQGNNFTVSNPYSSYDTSKTSDVITFAGENGITTKVNKGVVRVGIDQTKGLTTPKLTVGNNNGKGIVIDSQNGQNTITGLSNTLANVTNDKGSVRTTEQGNIIKDEDKTRAASIVDVLSAGFNLQGNGEAVDFVSTYDTVNFADGNATTAKVTYDDTSKTSKVVYDVNVDDTTIEVKDKKLGVKTTTLTSTGTGANKFALSNQATGDALVKASDIVAHLNTLSGDIQTAKGASQANNSAGYVDADGNKVIYDSTDNKYYQAKNDGTVDKTKEVAKDKLVAQAQTPDGTLAQMNVKSVINKEQVNDANKKQGINEDNAFVKGLEKAASDNKTKNAAVTVGDLNAVAQTPLTFAGDTGTTAKKLGETLTIKGGQTDTNKLTDNNIGVVAGTDGFTVKLAKDLTNLNSVNAGGTKIDDKGVSFVDSSGQAKANTPVLSANGLDLGGKVISNVGKGTKDTDAANVQQLNEVRNLLGLGNAGNDNADGNQVNIADIKKDPNSGSSSNRTVIKAGTVLGGKGNNDTEKLATGGIQVGVDKDGNANGDLSNVWVKTQKDGSKKALLATYNAAGQTNYLTNNPAEAIDRINEQGIRFFHVNDGNQEPVVQGRNGIDSSASGKHSVAIGFQAKADGEAAVAIGRQTQAGNQSIAIGDNAQATGDQSIAIGTGNVVAGKHSGAIGDPSTVKADNSYSVGNNNQFTDATQTDVFGVGNNITVTESNSVALGSNSAISAGTHAGTQAKKSDGTAGTTTTAGATGTVKGFAGQTAVGAVSVGASGAERRIQNVAAGEVSATSTDAVNGSQLYKATQSIANATNELDHRIHQNENKANAGISSAMAMASMPQAYIPGRSMVTGGIATHNGQGAVAVGLSKLSDNGQWVFKINGSADTQGHVGAAVGAGFHF

SEQ ID NO: 14 - DNA, 23 bp, Moraxella catarrhalis
gatgcctacgagttgatttgggt

SEQ ID NO: 15 - DNA, 23 bp, Moraxella catarrhalis
gagcgttgcaccgatcacgagga

SEQ ID NO: 16 - DNA, 26 bp, Moraxella catarrhalis
cactagcctttacatcaccaccgatg

SEQ ID NO: 17 - DNA, 35 bp, Moraxella catarrhalis
aaggtaaacccatatgaatcacatctataaagtca

SEQ ID NO: 18 - DNA, 22 bp, Moraxella catarrhalis
gcttctagctgtgccacattga

SEQ ID NO: 19 - DNA, 33 bp, Moraxella catarrhalis
cgctcgctgtccatatgatcggtgcaacgctca

SEQ ID NO: 20 - DNA, 23 bp, Moraxella catarrhalis
gaccctgtgcatatgacatggct

SEQ ID NO: 21 - DNA, 22 bp, Moraxella catarrhalis
ccttggcatcaatcgtggcaca

SEQ ID NO: 22 - DNA, 24 bp, Moraxella catarrhalis
ttacctgcatcaatgccattgtct

SEQ ID NO: 23 - DNA, 20 bp, Moraxella catarrhalis
ctgaggtgaatacaactaca

SEQ ID NO: 24 - DNA, 24 bp, Moraxella catarrhalis
catcagaggtctttgaggtgtcat

SEQ ID NO: 25 - DNA, 25 bp, Moraxella catarrhalis
catcaccgtgggtcaaaagaacgca

SEQ ID NO: 26 - DNA, 22 bp, Moraxella catarrhalis
gatgtcggcaatgtttacctga

SEQ ID NO: 27 - DNA, 30 bp, Moraxella catarrhalis
ccacattgaccagtactggcacaggtgcta

SEQ ID NO: 28 - DNA, 24 bp, Moraxella catarrhalis
acctatgatcaatggcgatttggt

SEQ ID NO: 29 - DNA, 30 bp, Moraxella catarrhalis
aaagatcatatggttacctttggcattaac

SEQ ID NO: 30 - DNA, 27 bp, Moraxella catarrhalis
gtcatctttcatatggccacaggcaca

SEQ ID NO: 31 - DNA, 28 bp, Moraxella catarrhalis
acatttatgcatatggcagagtacgcca

SEQ ID NO: 32 - DNA, 30 bp, Moraxella catarrhalis
gctacagggcatatgggcagtgtatgcact

(19) United States
(12) Patent Application Publication
(10) Pub. No.: US 2005/0249747 A1
(43) Pub. Date: Nov. 10, 2005

(54) Recombinant high molecular weight major outer membrane protein of Moraxella

(76) Inventors: Sheena M. Loosmore, Aurora (CA); Ken Sasaki, Willowdale (CA); Yan Ping Yang, Willowdale (CA); Michel H. Klein, Willowdale (CA)

Correspondence Address: Robert Yoshida, Aventis Pasteur, Inc.;Intellectual Property - Knerr Building, One Discovery Drive, Swiftwater, PA, 18370, US

(21) Appl. No.: 11/013,759

(22) Filed: Dec. 16, 2004

Related U.S. Application Data
(63) Continuation of application No. 09/361,619, filed on Jul. 27, 1999, now abandoned.

(51) Int. Cl. C07H 21/04; C12N 15/09; A61K 39/02; C07K 14/195; C12N 15/74; C12N 1/21

(52) U.S. Cl. 424/190.1; 435/69.3; 435/320.1; 435/252.3; 530/350; 536/23.7

(57) ABSTRACT

An isolated and purified outer membrane protein of a Moraxella strain, particularly M. catarrhalis, having a molecular mass of about 200 kDa, is provided by recombinant means. The about 200 kDa outer membrane protein as well as nucleic acid molecules encoding the same are useful in diagnostic applications and immunogenic compositions, particularly for in vivo administration to a host to confer protection against disease caused by a bacterial pathogen that produces the about 200 kDa outer membrane protein or produces a protein capable of inducing antibodies in a host specifically reactive with the about 200 kDa outer membrane protein.

### FIELD OF INVENTION

The present invention relates to the field of immunology and is particularly concerned with outer membrane proteins from Moraxella, methods of recombinant production thereof, genes encoding such proteins and uses thereof.

### BACKGROUND OF THE INVENTION

Otitis media is the most common illness of early childhood with approximately 70% of all children suffering at least one bout of otitis media before the age of seven. Chronic otitis media can lead to hearing, speech and cognitive impairment in children. It is caused by bacterial infection with Streptococcus pneumoniae (approximately 50%), non-typable Haemophilus influenzae (approximately 30%) and Moraxella (Branhamella) catarrhalis (approximately 20%). In the United States alone, treatment of otitis media costs between one and two billion dollars per year for antibiotics and surgical procedures, such as tonsillectomies, adenoidectomies and insertion of tympanostomy tubes. Because otitis media occurs at a time in life when language skills are developing at a rapid pace, developmental disabilities specifically related to learning and auditory perception have been documented in youngsters with frequent otitis media.

M. catarrhalis mainly colonizes the respiratory tract and is predominantly a mucosal pathogen. Studies using cultures of middle ear fluid obtained by tympanocentesis have shown that M. catarrhalis causes approximately 20% of cases of otitis media (ref. 1—Throughout this application, various references are referred to in parenthesis to more fully describe the state of the art to which this invention pertains. Full bibliographic information for each citation is found at the end of the specification, immediately preceding the claims. The disclosures of these references are hereby incorporated by reference into the present disclosure).

The incidence of otitis media caused by M. catarrhalis is increasing. As ways of preventing otitis media caused by pneumococcus and non-typable H. influenzae are developed, the relative importance of M. catarrhalis as a cause of otitis media can be expected to further increase.

M. catarrhalis is also an important cause of lower respiratory tract infections in adults, particularly in the setting of chronic bronchitis and emphysema (refs. 2, 3, 4, 5, 6, 7, and 8). M. catarrhalis also causes sinusitis in children and adults (refs. 9, 10. 11, 12, and 13) and occasionally causes invasive disease (refs. 14, 15, 16, 17, 18, and 19).

Like other Gram-negative bacteria, the outer membrane of M. catarrhalis consists of phospholipids, lipopolysaccharide (LPS), and outer membrane proteins (OMPs). Eight of the M. catarrhalis OMPs have been identified as major components. These are designated by letters A to H, beginning with OMP A which has a molecular mass of 98 kDa to OMP H which has a molecular mass of 21 kDa (ref. 20).

Recently, Klingman and Murphy purified and characterized a high molecular-weight outer membrane protein of M. catarrhalis (ref. 21). The apparent molecular mass of this protein varies from 350 kDa to 720 kDa as judged by sodium dodecyl sulfate-polyacrylamide gel electrophoresis (SDS-PAGE). This protein appears to be an oligomer of much smaller proteins or subunits thereof of molecular mass about 120 to 140 kDa and is antigenically conserved among strains of Moraxella.

Helminen et al also identified a protein of molecular mass of about 300 to 400 kDa, named UspA, that was reported to be present on the surface of Moraxella (ref. 22).

In WO 96/34960 and U.S. Pat. No. 5,808,024, assigned to the assignee hereof and the disclosure of which is incorporated herein by reference, there is described a new protein of M. catarrhalis which had an apparent molecular mass of about 200 kDa. Western blot analysis using antiserum raised against the 200 kDa protein suggested that this protein was different from the large UspA protein (>300 kDa), reported by the two groups in refs. 21 and 22. Recently, the gene sequences encoding two related proteins, UspA1 and UspA2, have been published (ref. 23). A sequence comparison between the two genes encoding the UspA proteins and the gene encoding the 200 kDa protein confirmed that the 200 kDa protein is different from either of the UspA1 and UspA2 proteins.

Fitzgerald et al (ref. 29) have identified a 200 kDa protein associated with haemagglutination. Transmission electron microcopy studies (ref. 30) showed that the 200 kDa protein associated with haemagglutination is present on the outer fibrillar layer of M. catarrhalis. Recently, a non-clumping variant of strain 4223 was prepared by serial passaging and it was observed that the non-clumping variant had reduced expression of both UspA and a 200 kDa protein that is not UspA (ref. 31). It is possible that this 200 kDa protein is the same as that described in WO 96/34960 and herein.

The 200 kDa protein described herein has been detected in most, but not all, strains of Moraxella catarrhalis, which have been isolated from various sources, including otitis media (OM), sputum, nasopharynx, expectorate and bronchial secretions. Table 1A below contains a listing of M. catarrhalis strains tested, their source and whether or not the 200 kDa protein is expressed.

M. catarrhalis infection may lead to serious disease. It would be advantageous to provide recombinant means for providing large quantities of 200 kDa outer membrane protein of M. catarrhalis strains and genes encoding such proteins from various M. catarrhalis strains for use as antigens in immunogenic preparations including vaccines, carriers for other antigens and immunogens and the generation of diagnostic reagents.

### SUMMARY OF THE INVENTION

The present invention is directed towards the provision of a recombinantly-produced purified and isolated outer membrane protein of Moraxella catarrhalis and other Moraxella strains, having an apparent molecular mass of about 200 kDa, as well as genes encoding the same from various strains of Moraxella catarrhalis.

In one aspect of the present invention, there is provided an isolated and purified nucleic acid molecule having (a) a nucleotide sequence set forth in FIG. 3, 4 or 5 (SEQ ID Nos: 5, 6, 8, 9, 11, 12) for Moraxella catarrhalis strains 4223, Q8 and LES-1 respectively or the complementary sequence thereto; (b) a nucleotide sequence encoding an about 200 kDa outer membrane protein of a strain of Moraxella catarrhalis and having the derived amino acid sequence shown in FIG. 3, 4 or 5 (SEQ ID Nos: 7, 10, 13) for Moraxella catarrhalis strains 4223, Q8 and LES-1 respectively; and (c) a nucleotide sequence encoding an about 200 kDa outer membrane protein of another strain of Moraxella catarrhalis which is characterized by a tract of consecutive G nucleotides which is 3 or a multiple thereof in length, an ATG start codon about 80 to 90 bp upstream of said tract and said tract being located between about amino acids 25 and 35 encoded by the nucleotide sequence.

The another strain of Moraxella catarrhalis in (c) is a strain as identified in Table 1A other than strains 4223, Q8 and LES-1 and expressing an about 200 kDa protein.

In another aspect of the invention, there is provided (a) a nucleotide sequence set forth in FIG. 8 (SEQ ID No: 12) for a 5′-truncation of the gene encoding an about 200 kDa outer membrane protein of Moraxella catarrhalis strain 4223; (b) a nucleotide sequence encoding the derived amino acid sequence set forth in FIG. 9 (SEQ ID No: 13) for a N-terminal truncation of an about 200 kDa outer membrane protein of Moraxella catarrhalis strain 4223; and (c) a nucleotide sequence encoding a 5′-truncation of a gene encoding an about 200 kDa outer membrane protein of another strain of Moraxella catarrhalis and being capable of expressing the corresponding N-terminally truncated about 200 kDa outer membrane protein from E. coli.

A further aspect of the invention providing an isolated and purified nucleic acid molecule which is a contiguous Nde I-Pst I fragment of SEQ ID No: 5.

The invention, in an additional aspect, provides a vector for transforming a host comprising a nucleic acid molecule as provided herein, which may be a plasmid vector. The plasmid vector may be one which has the identifying characteristics of pKS348 (ATCC 203,529) or pKS294 (ATCC 203,528). The plasmid vector also may be one having the identifying characteristics of pQWE or pQWF.

A further aspect of the invention provides a host cell, such as E. coli, transformed by a vector provided herein and expressing an about 200 kDa protein of a strain of Moraxella catarrhalis or an approximately C-terminal half thereof. The invention further provides, in an additional aspect, a recombinant about 200 kDa outer membrane protein of a strain of Moraxella catarrhalis or an approximately C-terminal half thereof producible by the transformed host provided herein.

The recombinant about 200 kDa outer membrane protein or an approximately C-terminal half thereof may be formulated into an immunogenic composition, which may be formulated as a vaccine for in vivo administration to protect against disease caused by Moraxella catarrhalis, which may be provided in combination with a targeting molecule for delivery to specific cells of the immune system, formulated as a microparticle, capsule or liposome preparation, and may further comprise an adjuvant.

The invention, in a further aspect, includes a method of inducing protection against disease caused by Moraxella catarrhalis by administering to a susceptible host, which may be a human, an effective amount of the immunogenic composition provided herein.

In an additional aspect, the invention provides a method for the production of an about 200 kDa outer membrane protein of a strain of Moraxella catarrhalis or an approximately C-terminal half thereof, which comprises:
transforming a host cell, such as E. coli, with a vector as provided herein,
growing the host cell to express the encoded about 200 kDa protein or an approximately C-terminal half thereof, and
isolating and purifying the expressed about 200 kDa protein or an approximately C-terminal half thereof.

The encoded about 200 kDa protein may be expressed in inclusion bodies. The isolation and purification of the about 200 kDa protein may be effected by:
disrupting the grown transformed cells to produce supernatant and the inclusion bodies,
solubilizing the inclusion bodies to produce a solution of the recombinant about 200 kDa protein,
chromotographically purifying the solution of recombinant about 200 kDa protein free from contaminating proteins, and
isolating the purified recombinant about 200 kDa protein.

### BRIEF DESCRIPTION OF DRAWINGS

FIG. 1 shows restriction maps of subclones of a gene encoding the 200 kDa outer membrane protein of M. catarrhalis from λEMBL3 clone 8II and the location of PCR primers used to amplify the 5%-region of the gene. The open reading frame of the about 200 kDa outer membrane protein is indicated by the shaded box. The numbers in parenthesis are approximate sizes of DNA inserts in plasmids. Restrictions sites are Sal: SalI, N: NcoI, B: BglII, K: KpnI, Xb: XbaI, Xh: XhoI, RV: EcoRV;

FIG. 2 shows the nucleotide sequence (SEQ ID No: 1—entire sequence, SEQ ID No: 2—coding sequence) of the gene encoding the about 200 kDa outer membrane protein of M. catarrhalis strain 4223, as determined from λEMBL3 clone 8II, and deduced amino acid sequence (SEQ ID No: 3—identified GTG start codon, SEQ ID No: 4—putative ATG start codon shaded) of the about 200 kDa outer membrane protein. A ten-G nucleotide segment of the 5′-UTR is identified by underlining. An ATG start codon for the same sequence but with a nine-G nucleotide segment is identified by a shaded box (see FIG. 3);

FIG. 3 shows the nucleotide sequence (SEQ ID No: 5—entire sequence, SEQ ID No: 6—coding sequence) of the gene encoding the about 200 kDa outer membrane protein of M. catarrhalis strain 4223, as determined from PCR-amplified genomic DNA of strain 4223 and the deduced amino acid sequence (SEQ ID No: 7) of the corresponding about 200 kDa outer membrane protein. A nine-G nucleotide segment of the sequence corresponding to the 10-G nucleotide segment of FIG. 2, is identified by underlining. The GTG start codon identified in FIG. 2 is identified by a light box;

FIG. 4 shows the nucleotide sequence (SEQ ID No: 8) of the gene encoding the about 200 kDa outer membrane protein of M. catarrhalis strain Q8 and the deduced amino acid sequence (SEQ ID No: 9) of the corresponding about 200 kDa outer membrane protein. A nine-G nucleotide segment is identified by underlining;

FIG. 5 shows the nucleotide sequence (SEQ ID No: 10) of the gene encoding the about 200 kDa outer membrane protein of M. catarrhalis strain LES-I and the deduced amino acid sequence (SEQ ID No: 11) of the corresponding about 200 kDa outer membrane protein. A three-G nucleotide segment is identified by underlining;

FIG. 6 contains an alignment of the amino acid sequence (in single letter code) of the about 200 kDa proteins of M. catarrhalis strain 4223 (SEQ ID No: 7), Q8 (SEQ ID No: 9) and LES-I (SEQ ID No: 11). The alignments of the sequences were made using BLAST and manual methods and are compared to the 4223 sequence. Gaps in the sequence where no corresponding or related amino acid exists are designated by “−” while identical amino acids are designed by “.”;

FIG. 7 shows the restriction sites of the M. catarrhalis strain 4223 derived 200 kDa protein gene as well as the identity of various plasmids containing partial or full length 200 kDa genes;

FIG. 8 shows the nucleotide sequence (SEQ ID No: 12) and deduced amino acid sequence (SEQ ID No: 13) of the 5′-truncated gene encoding the M56 200 kDa protein of M. catarrhalis strain 4223 contained in pKS348;

FIGS. 9A and 9B contain a schematic of the procedure for producing plasmid pKS294 expressing the full length 200 kDa protein of M. catarrhalis strain 4223;

FIG. 10 is a schematic of the procedure for producing plasmid pKS348 expressing the N-truncated M56 r200 kDa protein of M. catarrhalis strain 4223;

FIG. 11 shows a schematic procedure for the purification of recombinantly-produced 200 kDa protein from E. coli;

FIG. 12 shows SDS-PAGE analysis of the expression of M56 r200 kDa protein gene from E. coli. M. catarrhalis strain 4223 lysate was run as a positive control (a) and uninduced KS358 cultured overnight was run as a negative control (b). In each lane, 20 μg of total protein was loaded;

FIG. 13 shows the SDS-PAGE analysis of the purification of the M56 r200 kDa protein according to the scheme of FIG. 11. Lane 1, E. coli whole cells; Lane 2, soluble proteins after 50 mM Tris/NaCl, pH8, extraction; Lane 3, soluble proteins after Tris/Triton X-100/EDTA extraction; Lane 4, soluble proteins after Tris/OG extraction; Lane 5, pellet after Tris/OG extraction; Lanes 6, 7, purified 200 kDa protein;

FIG. 14 shows the anti-M56 r200 kDa protein antibody titers obtained in mice. Mice were immunized on day 1, day 29 and day 43 with 0.3 μg, 1 μg, 3 μg or 10 μg of the purified M56 r200 kDa protein in adjuvant. Antisera were obtained on days 14, 28, 42 and 56 and anti-M56 r200 kDa protein IgG titers were determined. The reactive titers of antisera were defined as the reciprocal of the dilution consistently showing a two-fold increasing in absorbance over that obtained with the pre-bleed serum sample collected on day 0;

FIG. 15 shows the anti-M56 r200 kDa antibody titers in guinea pigs. Guinea pigs were immunized and antisera were analyzed according to the protocol of FIG. 14;

FIG. 16 shows the location of PCR primers used to amplify a DNA fragments carrying portions of the 200 kDa protein gene from chromosomal DNA of M. catarrhalis strain RH408, a spontaneous mutant of strain 4223 which does not produce the 200 kDa protein;

FIG. 17 is a partial nucleotide and derived amino acid sequence for the 200 kDa protein of M. catarrhalis strain 4223, indicating by arrows the locations of the initial amino acid of the respective three truncations ALA₁₂, VAL¹⁹ and GLY³⁹;

FIG. 18 shows schematic diagrams for two 3′ half clones of the 4223 200 kDa gene. Clone pQWE contains a fusion between the 5′ end of the 200 kDa gene and the 3′ half of the gene. Clone pQWE contains the 3′ half of the gene alone. The location of the PCR primers used to generate pQWF is indicated.

FIG. 19 is a construction diagram for producing plasmid pQWE expressing a C-terminal portion of the 200 kDa protein of M. catarrhalis strain 4223 fused to the N-terminus; and

FIG. 20 is a construction diagram for producing plasmid pQWF expressing a C-terminal portion of the 200 kDa protein of M. catarrhalis strain 4223.

### GENERAL DESCRIPTION OF THE INVENTION

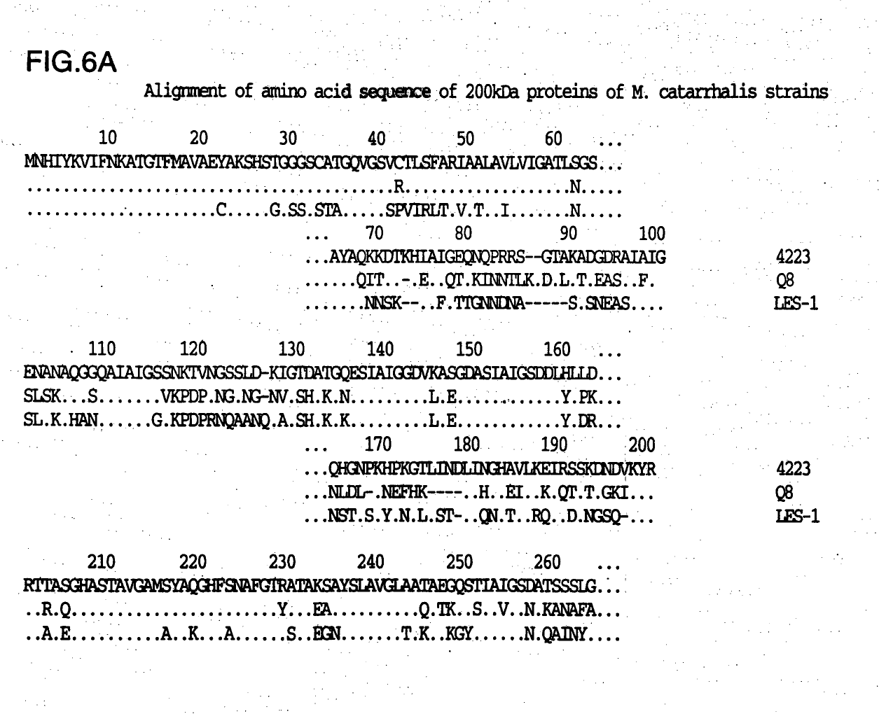

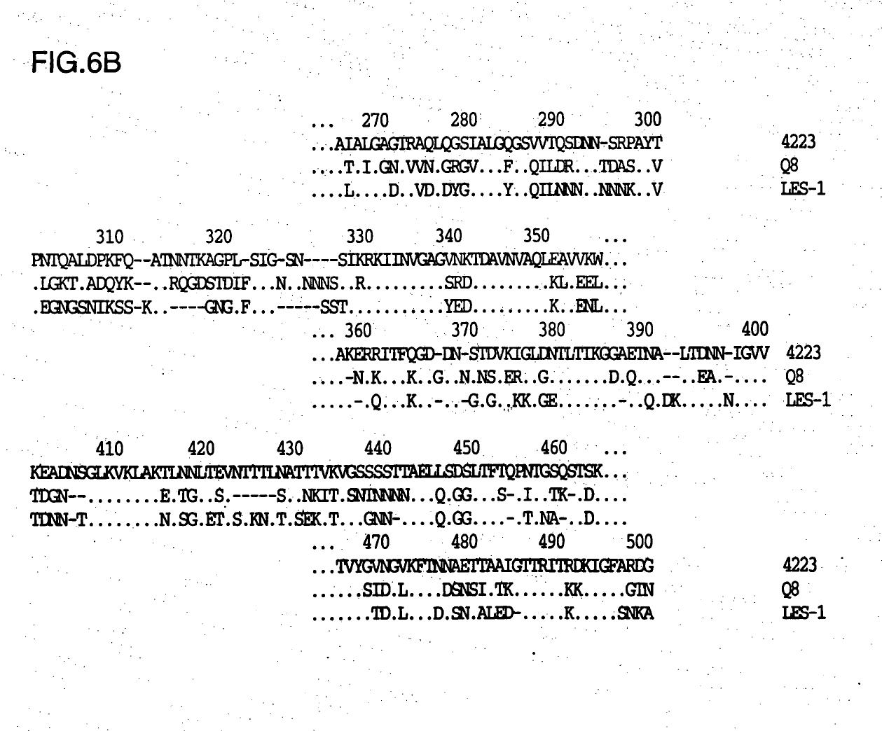

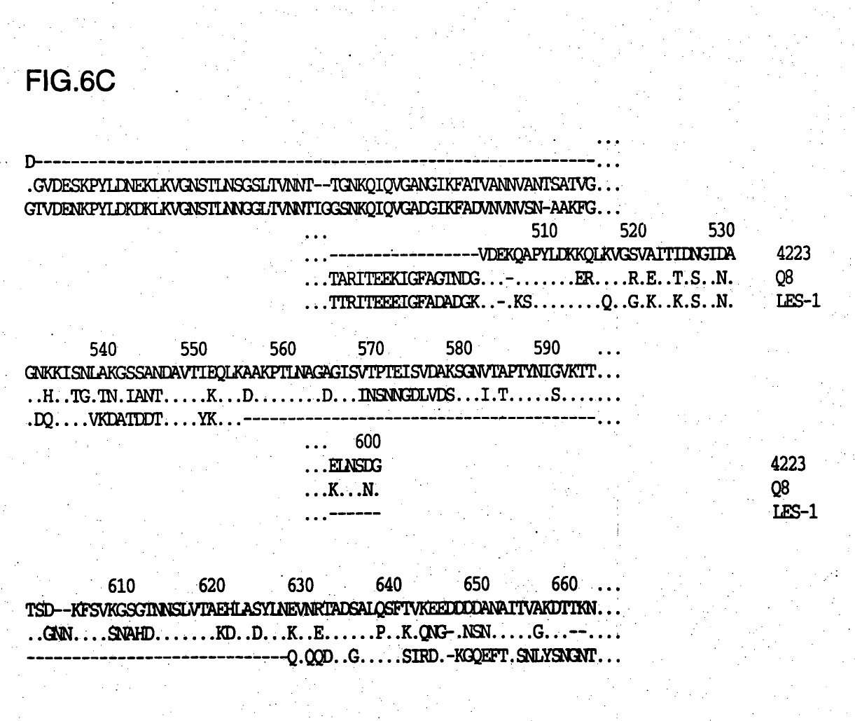

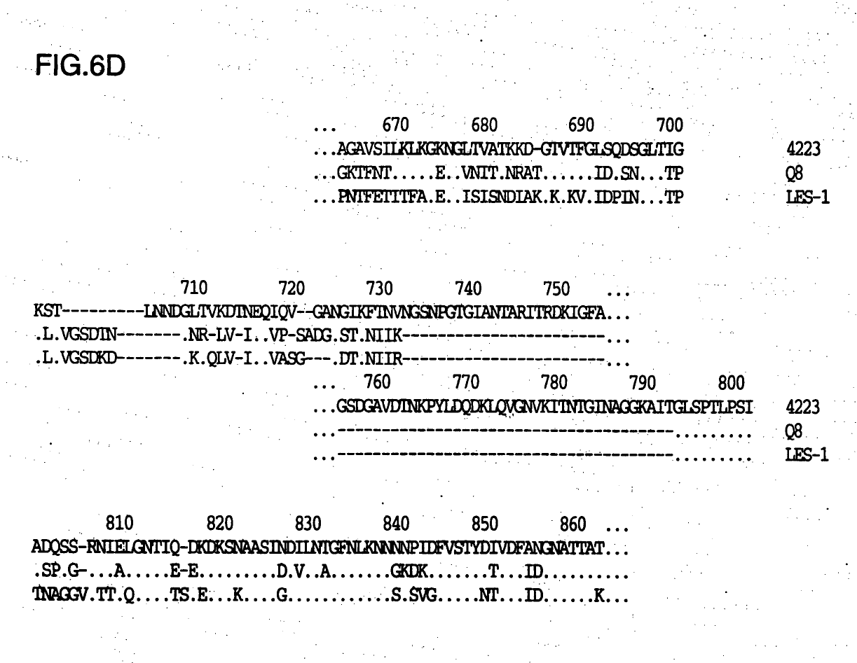

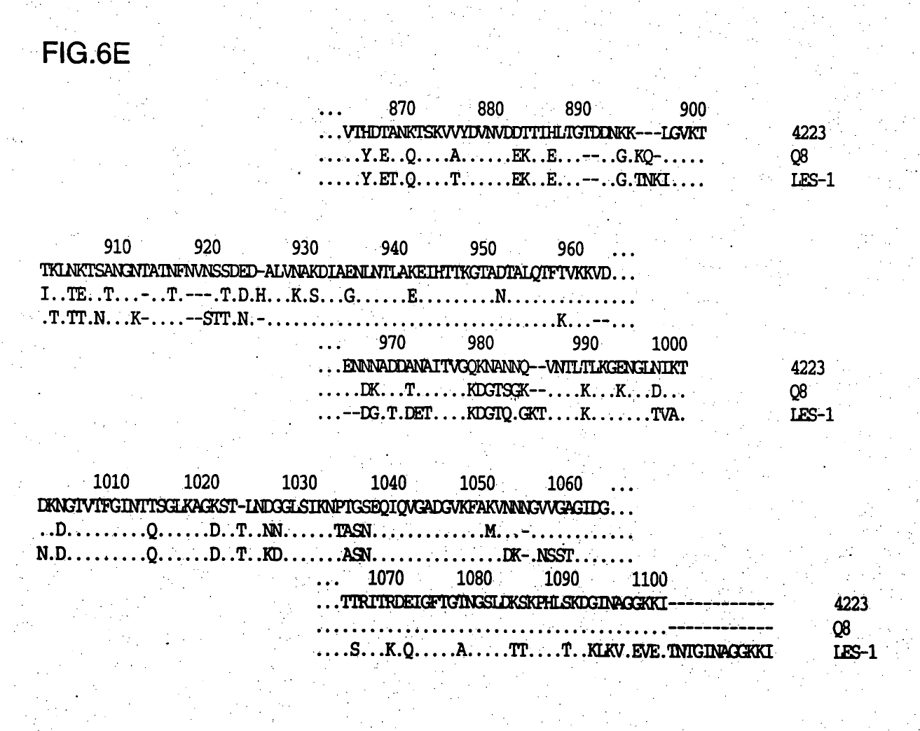

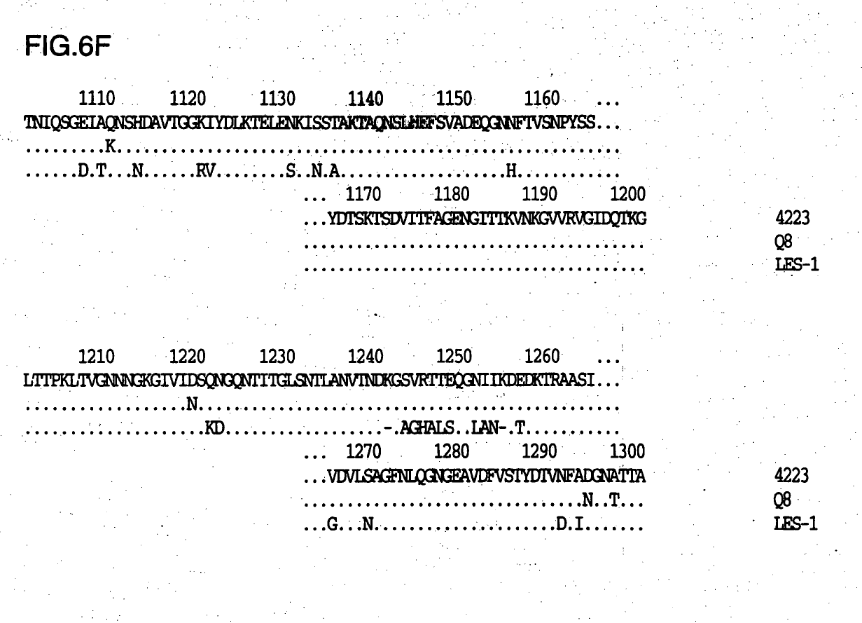

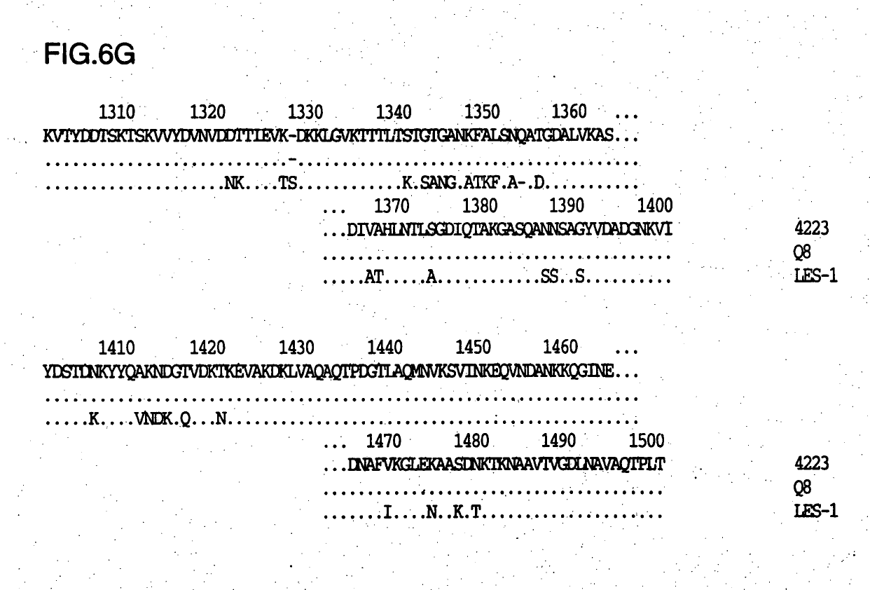

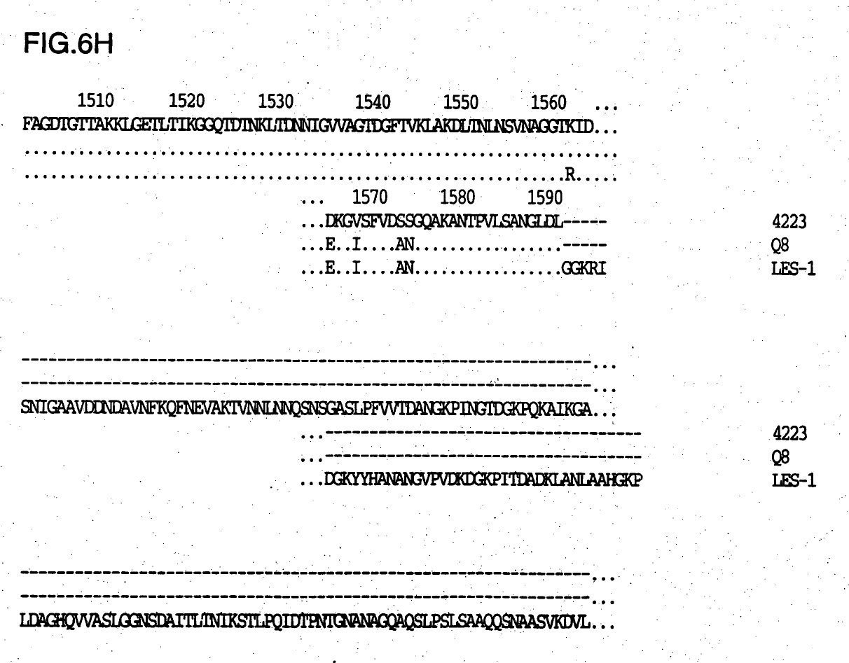

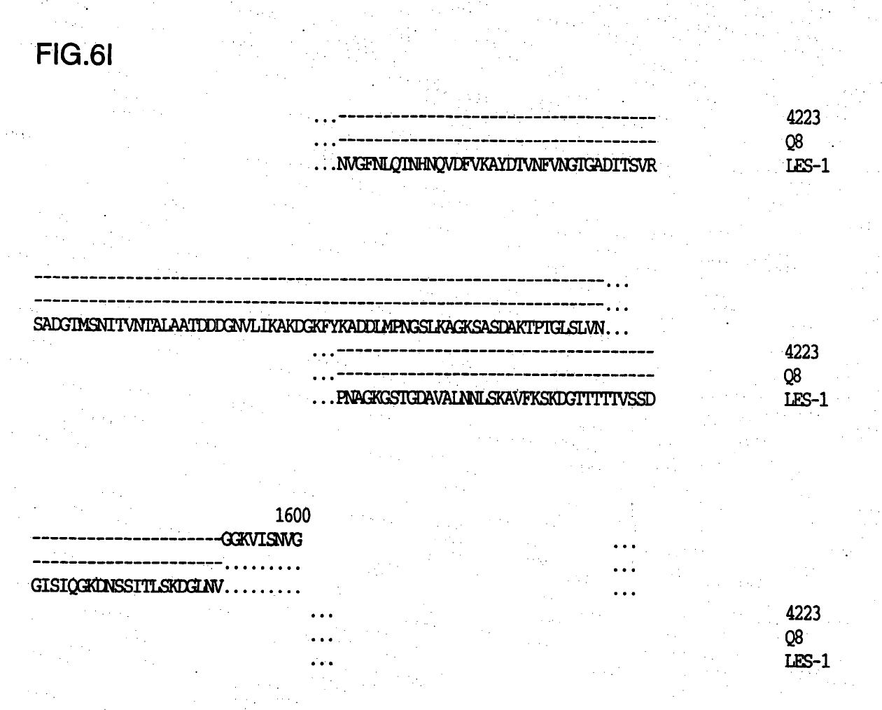

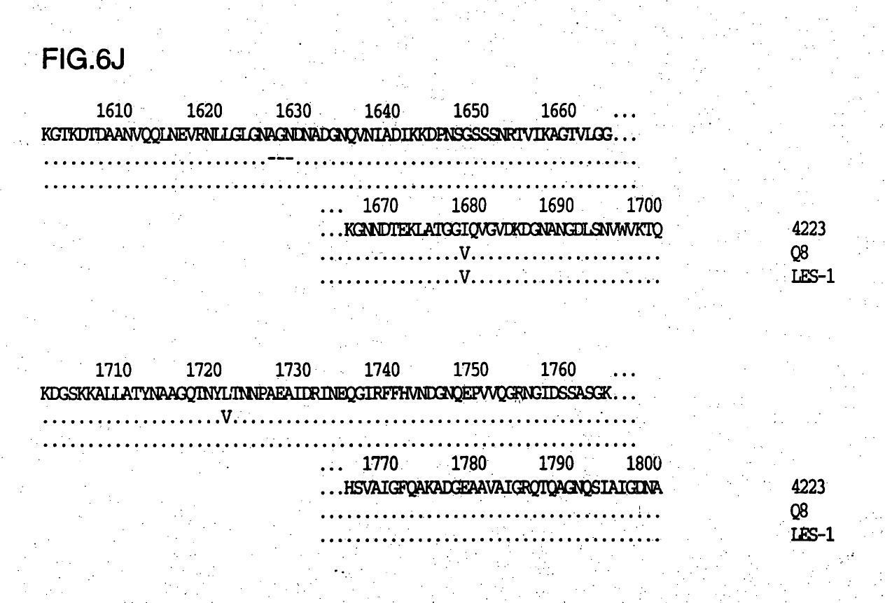

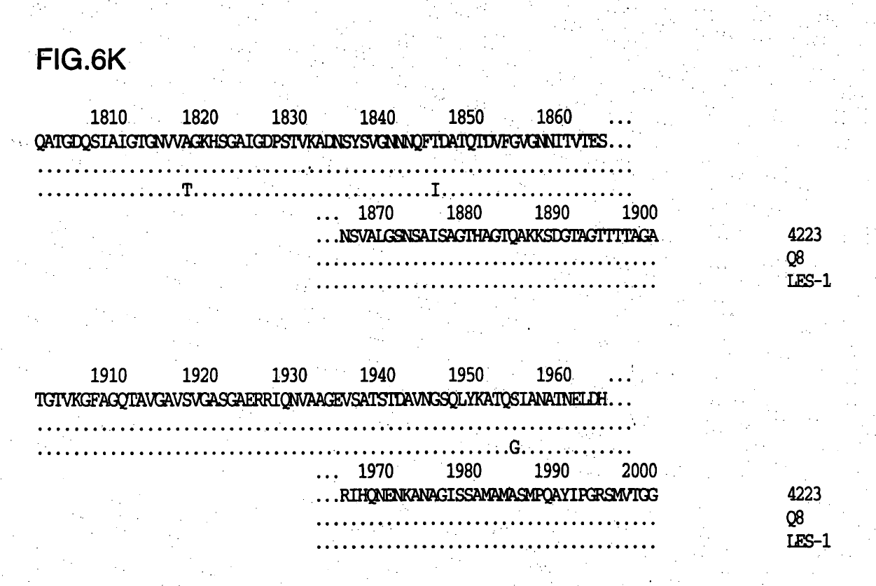

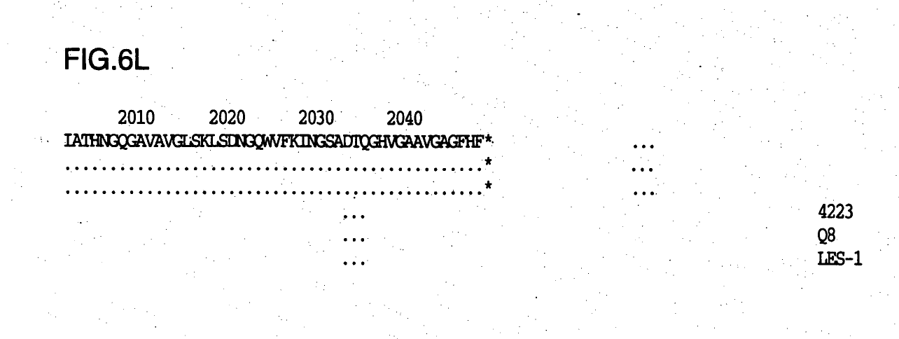

In WO 96/34960 (FIG. 6), the sequence of a cloned gene from M. catarrhalis 4223 encoding an about 200 kDa protein, was described. The open reading frame was predicted to start at a GTG codon. Sequence analysis of 200 kDa genes from additional strains, suggested that a slightly longer open reading frame was more generally found. A re-examination of the sequence from the lambda phage-derived 200 kDa gene confirmed the GTG start codon and an upstream stretch of 10 G nucleotides in a G tract. However, when sequence analysis was performed on 4223 genomic PCR-amplified subclones, the longer open reading frame was found starting from an ATG codon. The G-tract was found to contain 9 G nucleotides in the chromosomal gene. An additional G nucleotide had been inserted during cloning from the phage library. Analysis of the 5′ end of the 200 kDa gene from 24 strains suggests that the number of G nucleotides in the G tract acts as regulator of expression.

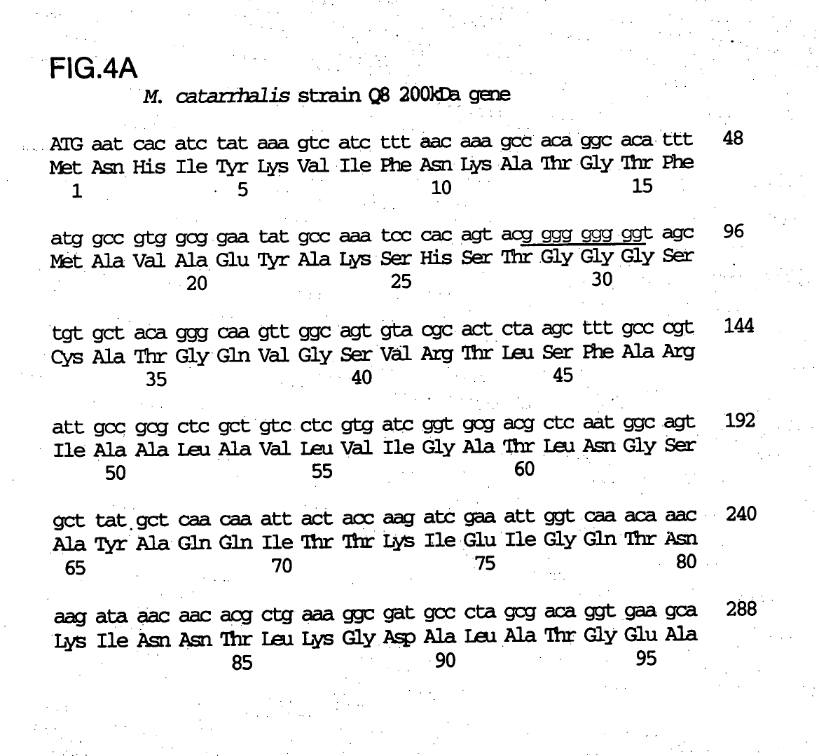

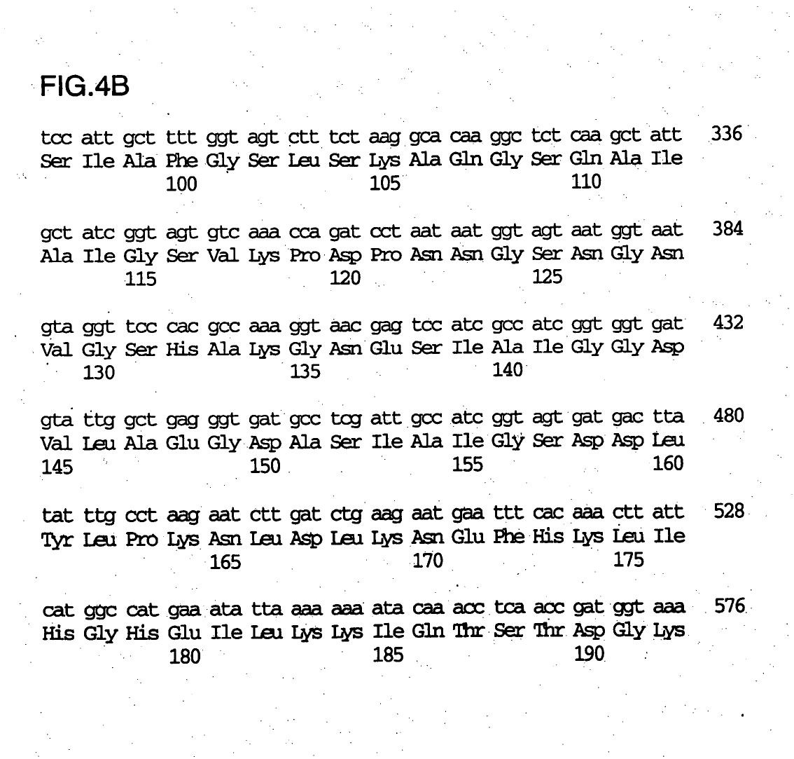

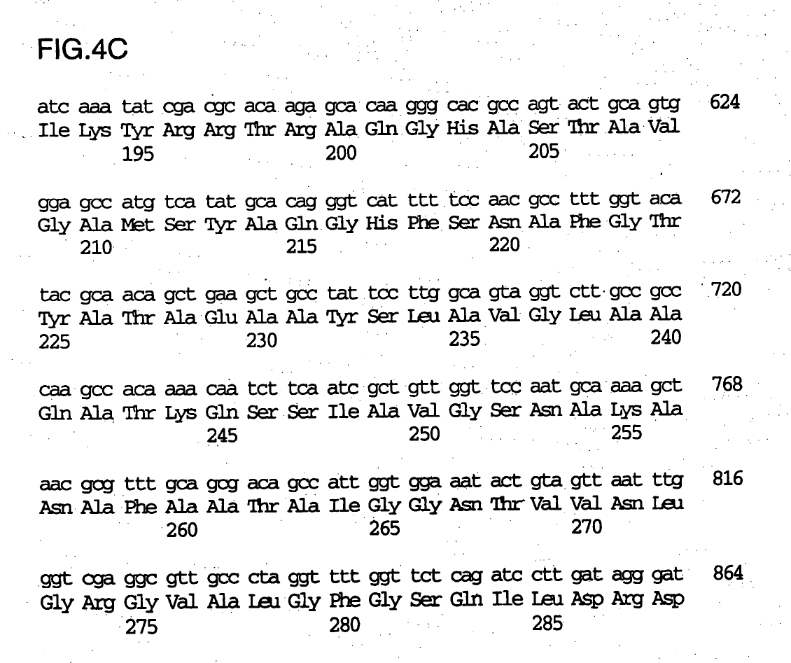

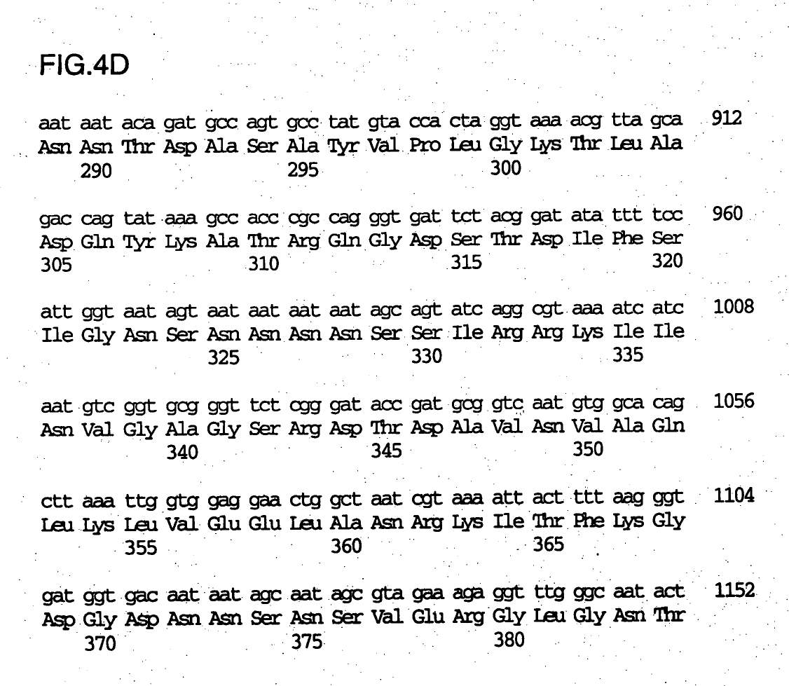

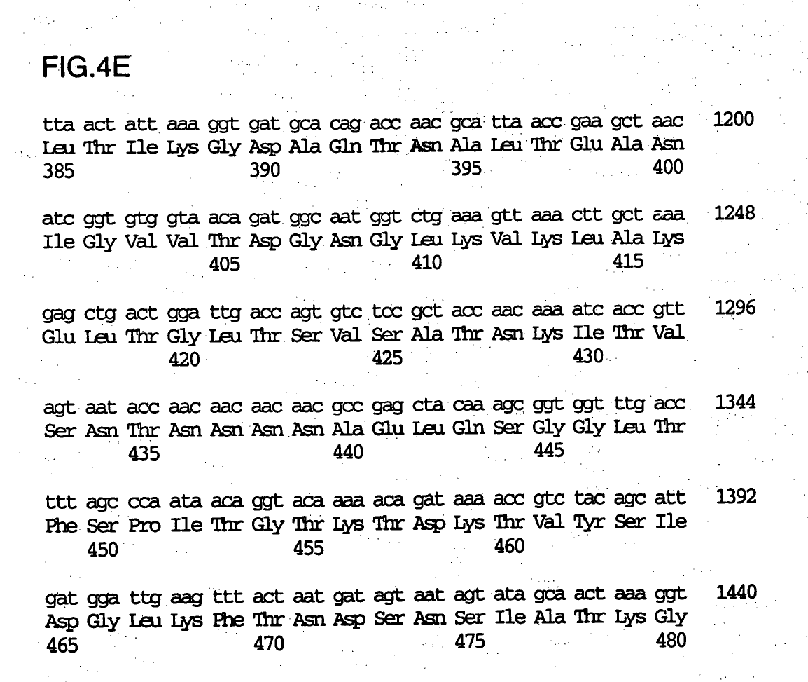

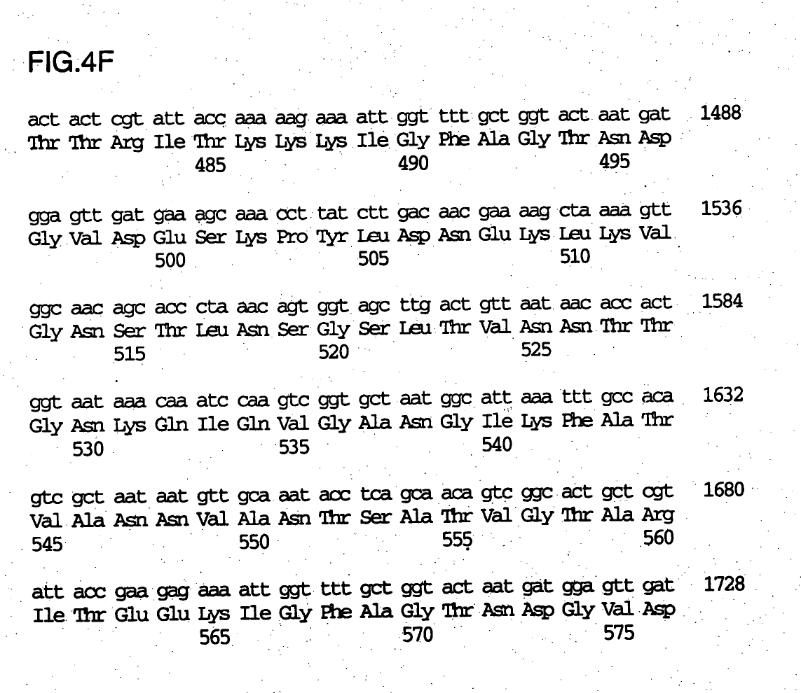

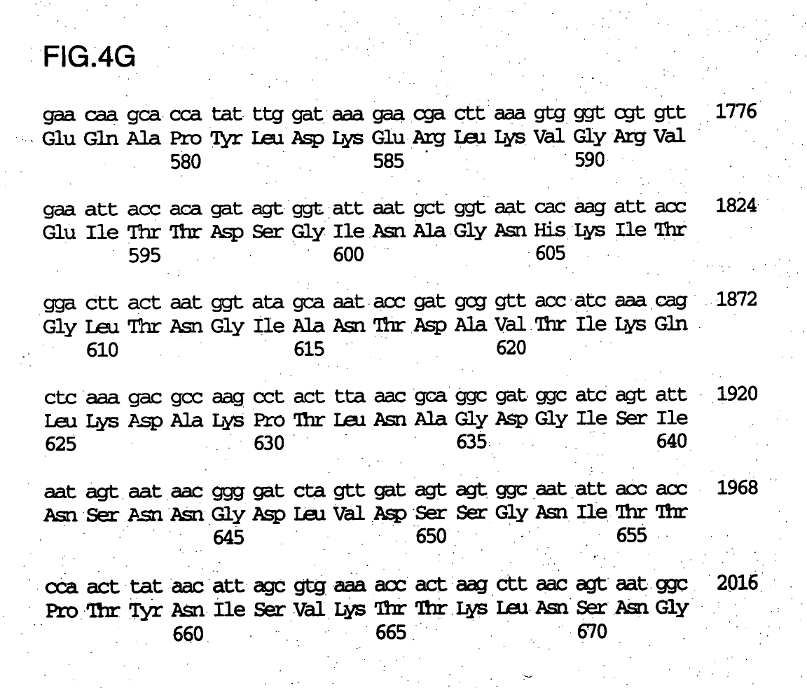

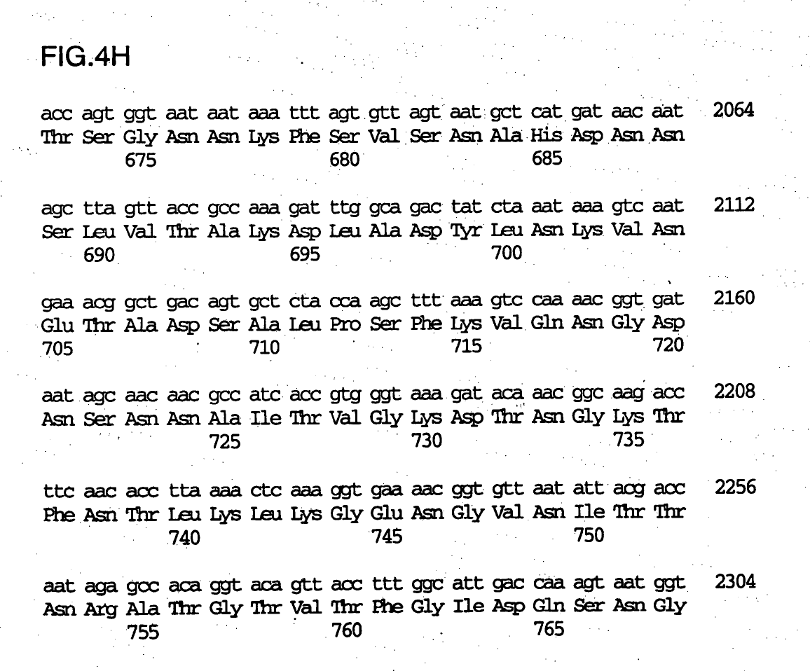

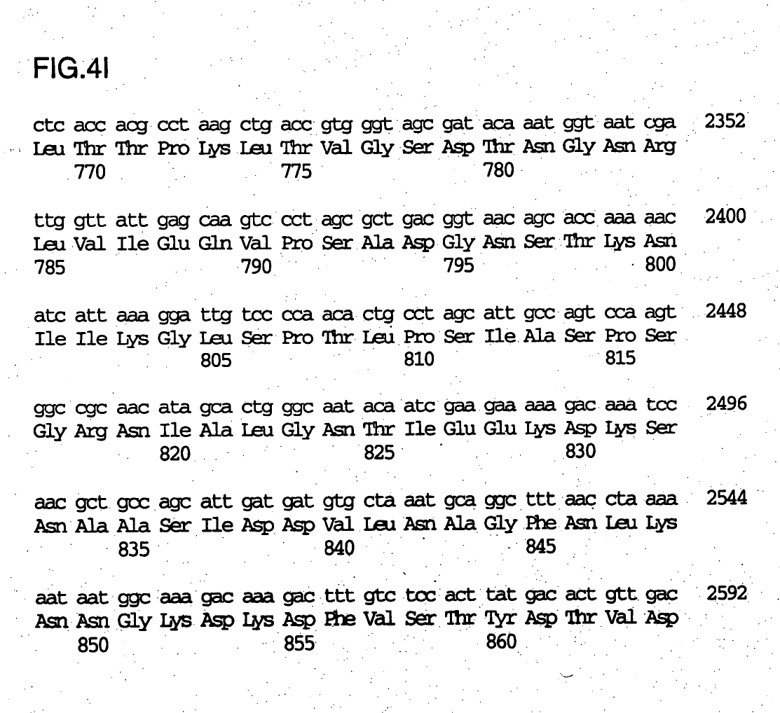

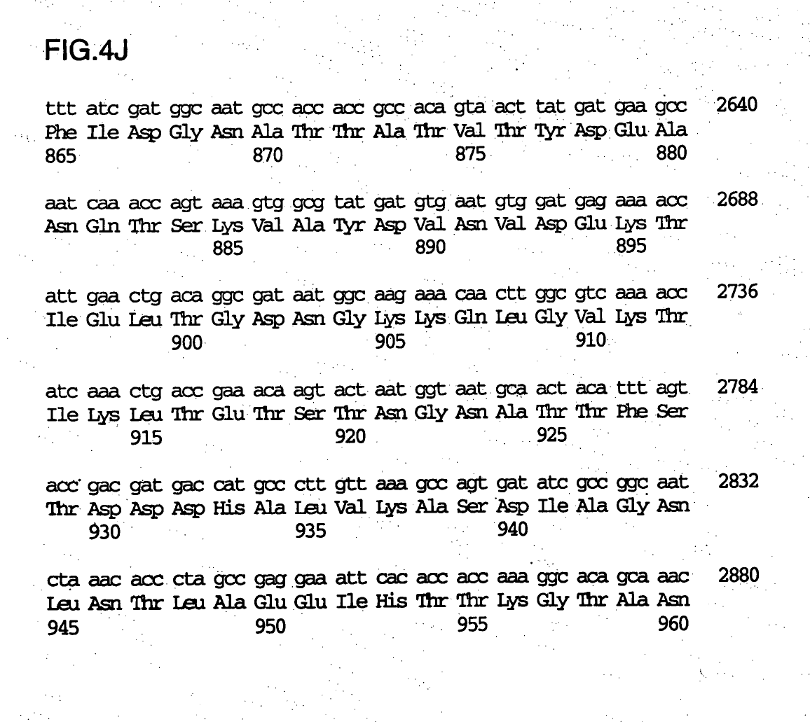

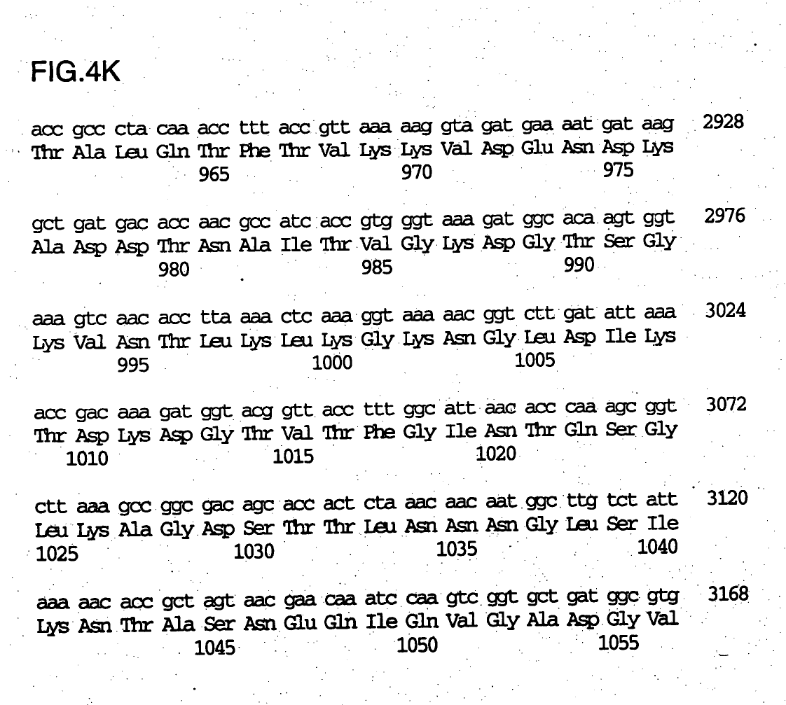

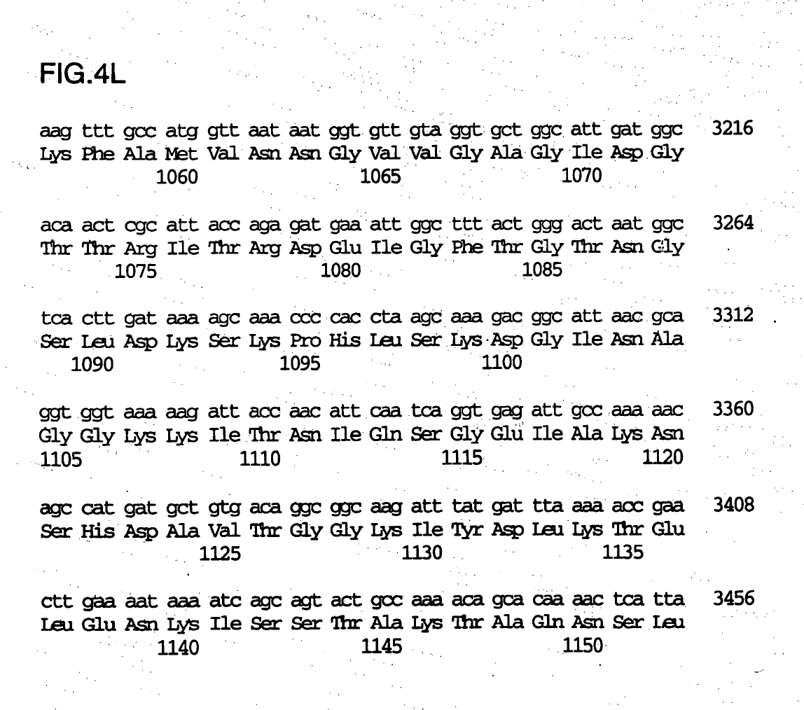

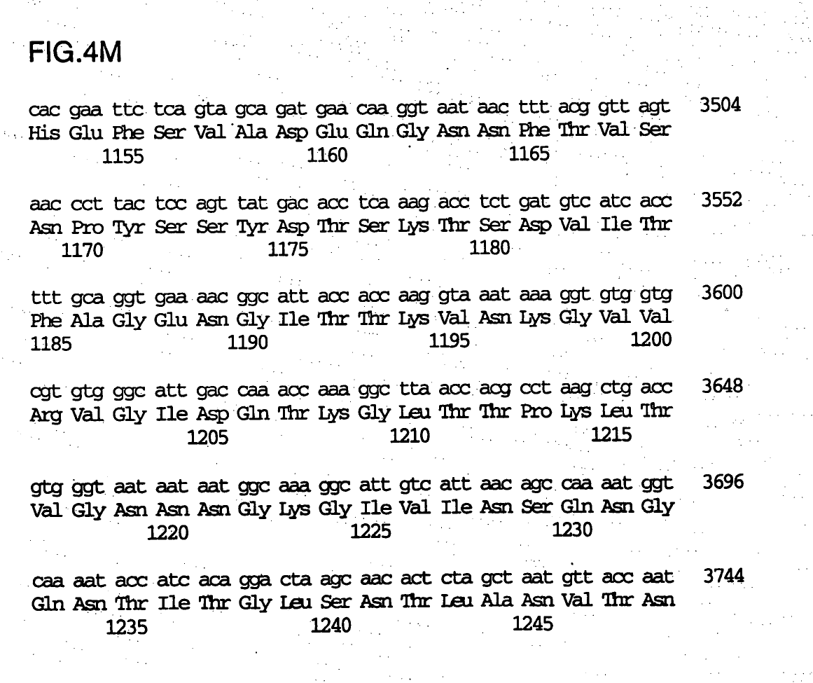

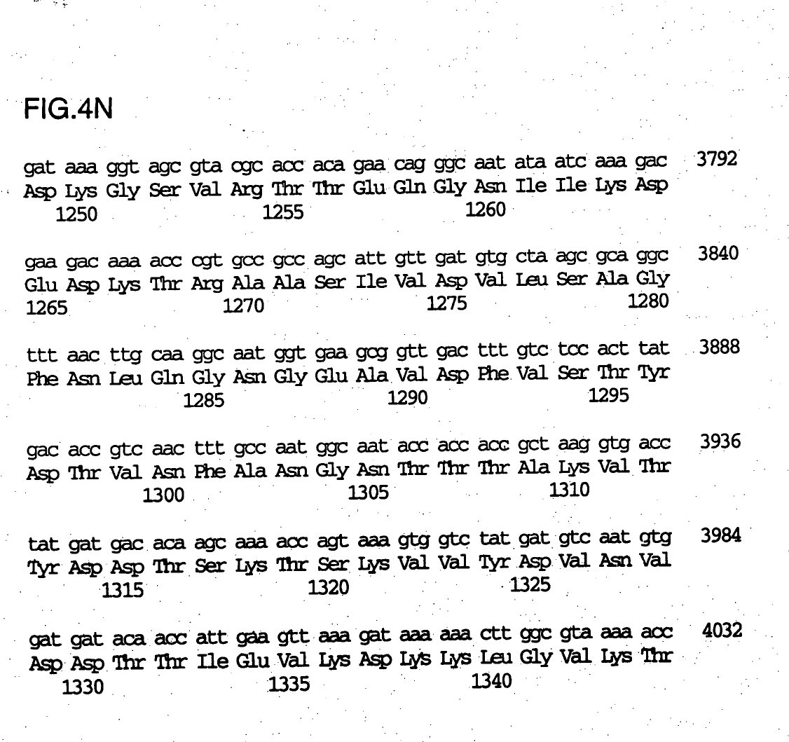

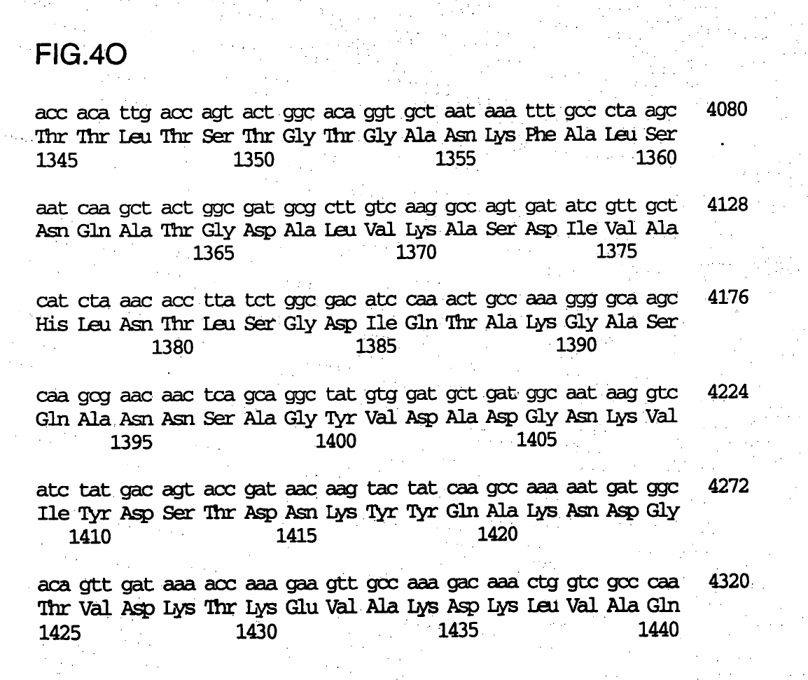

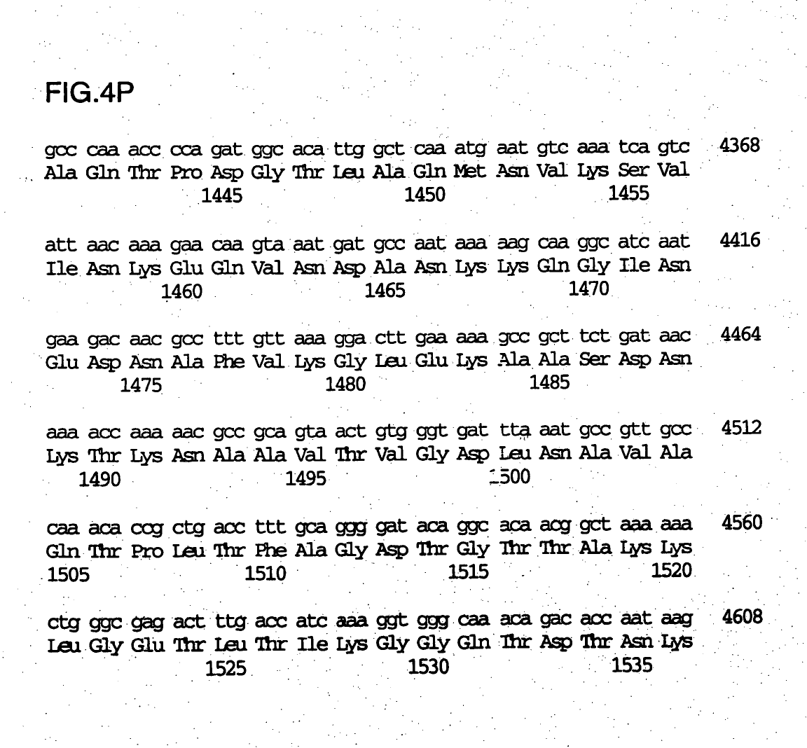

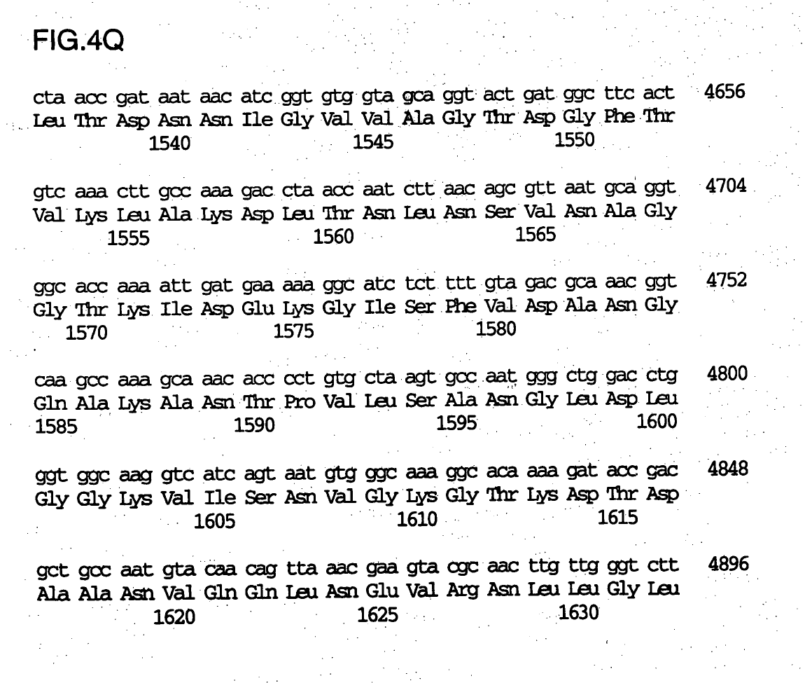

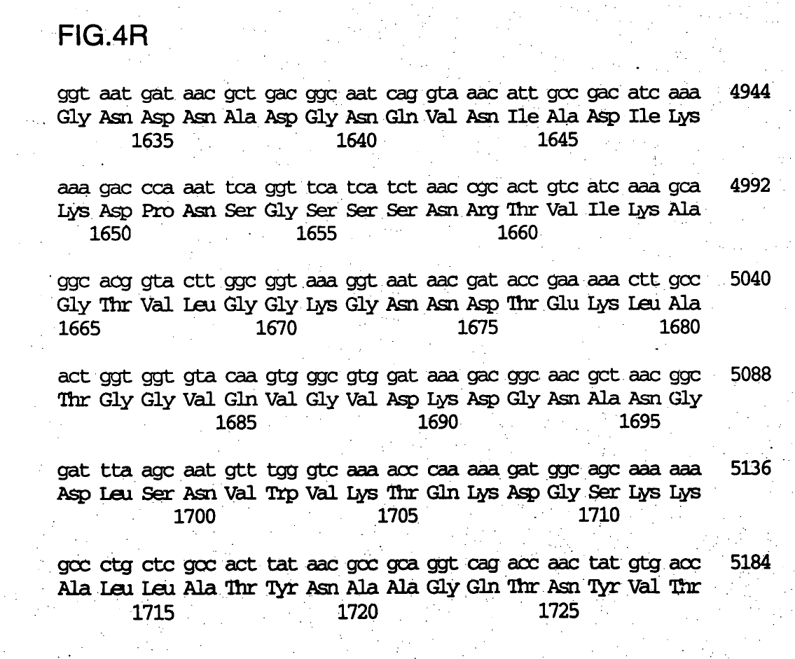

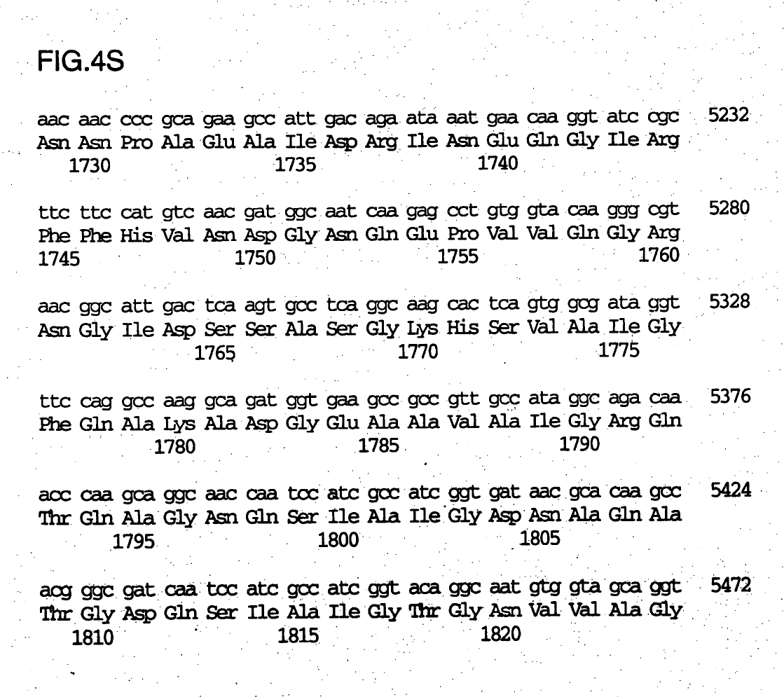

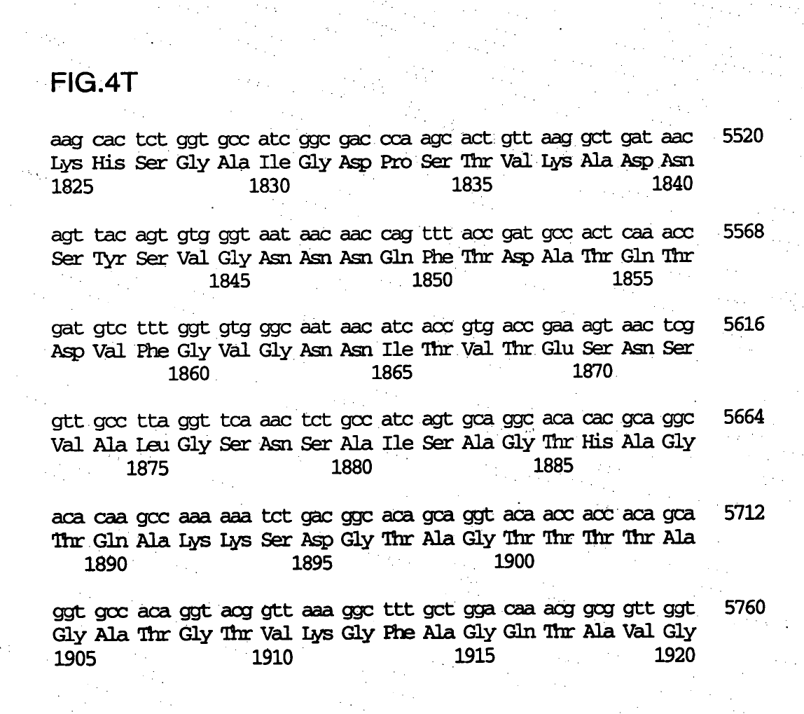

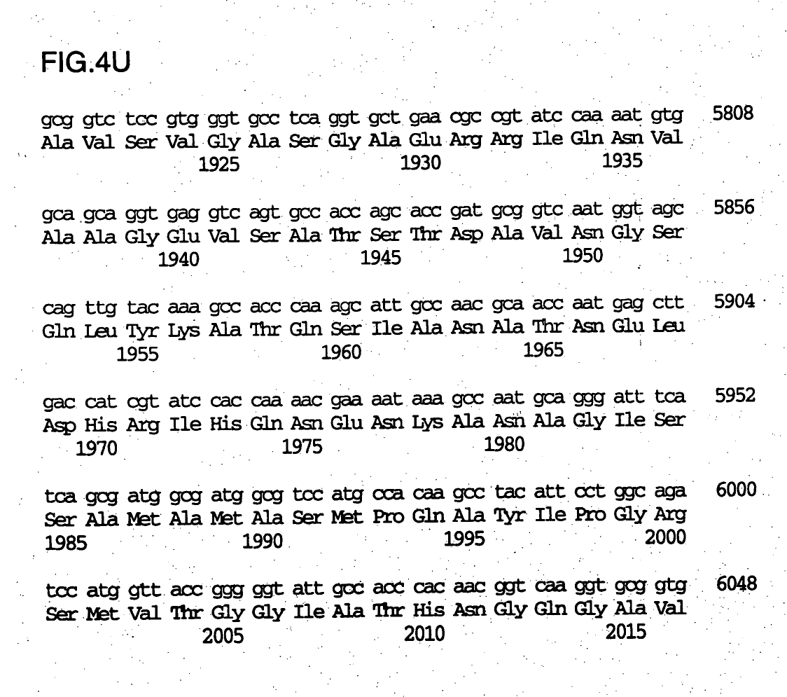

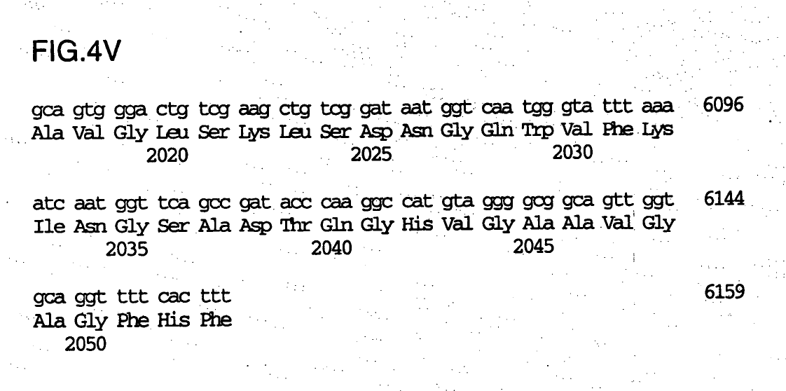

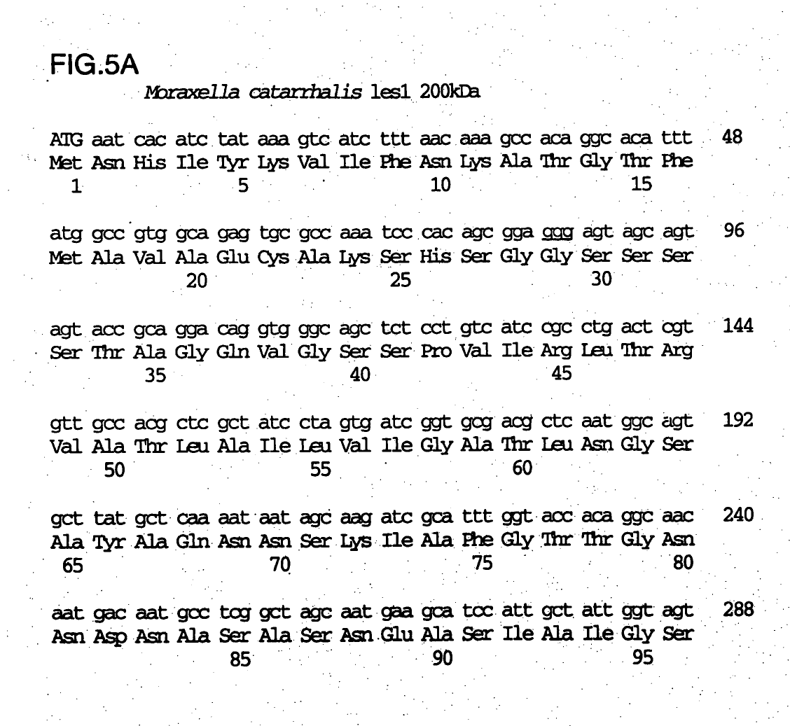

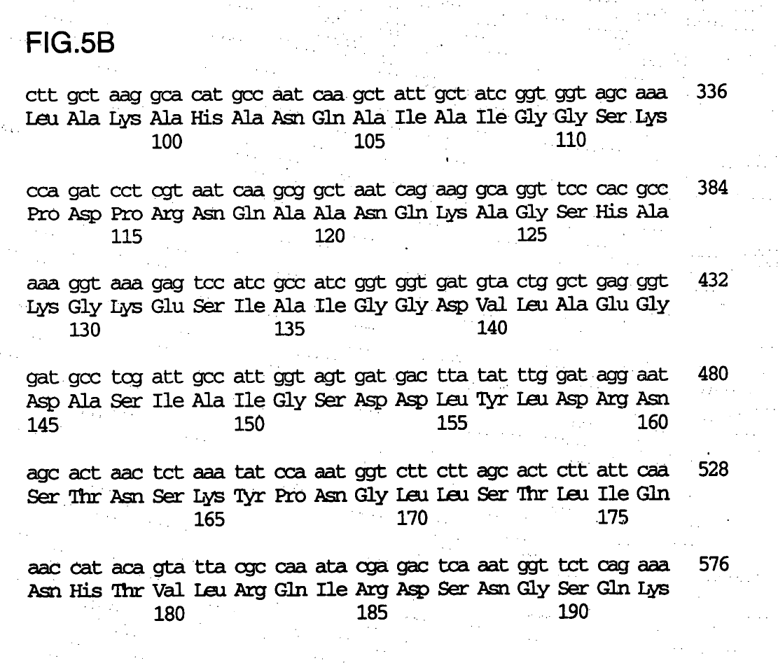

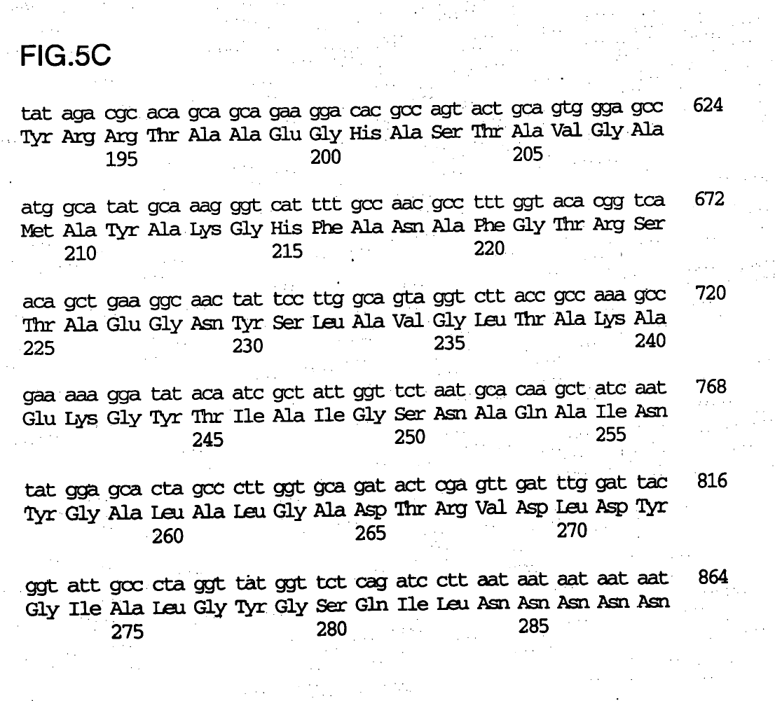

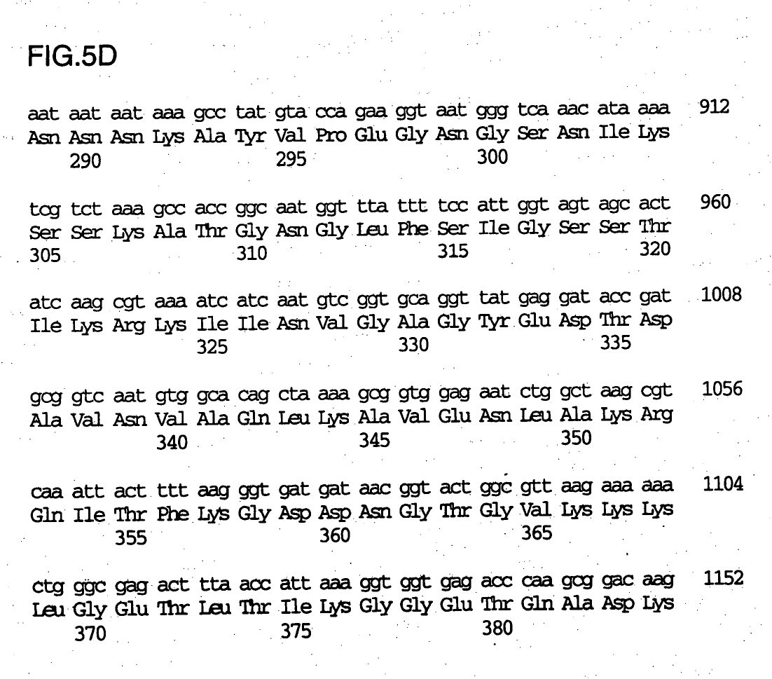

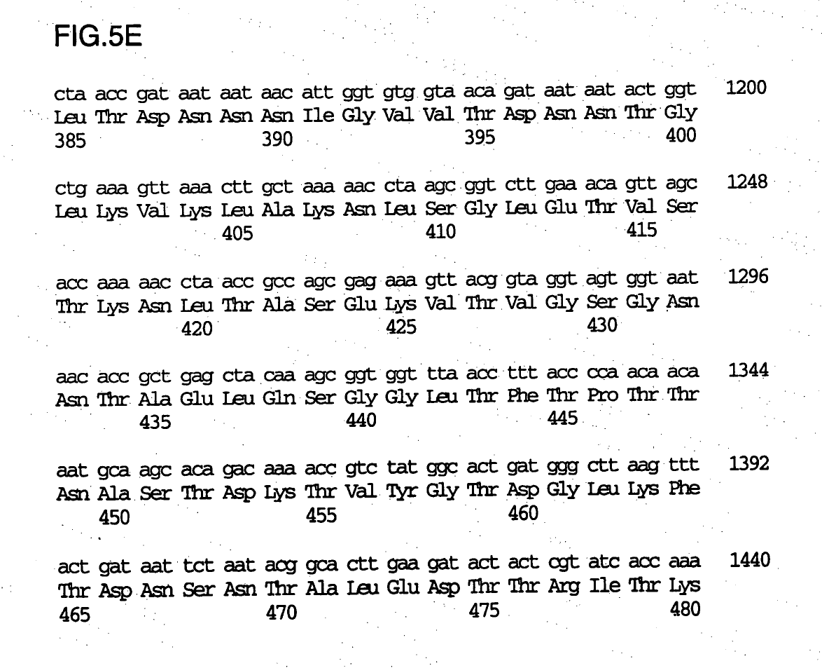

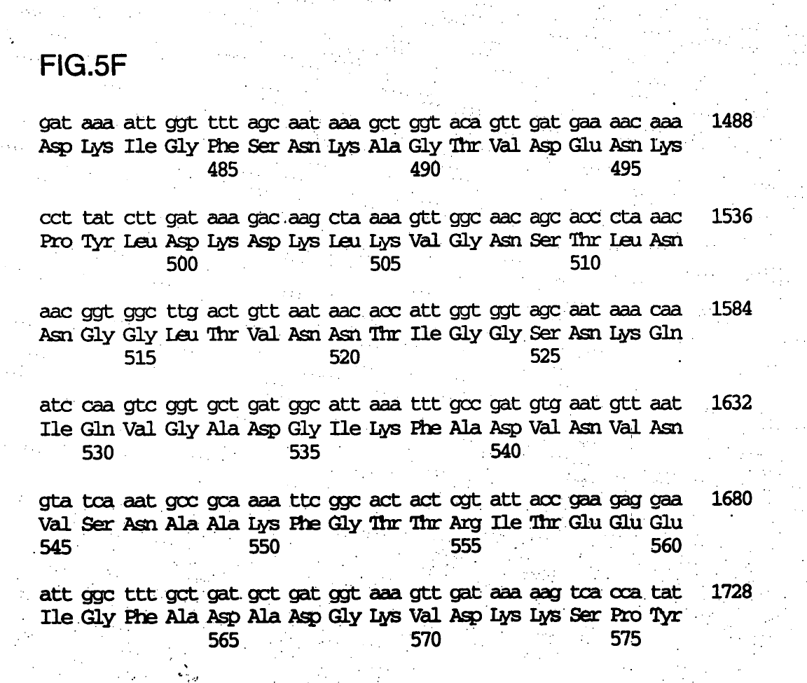

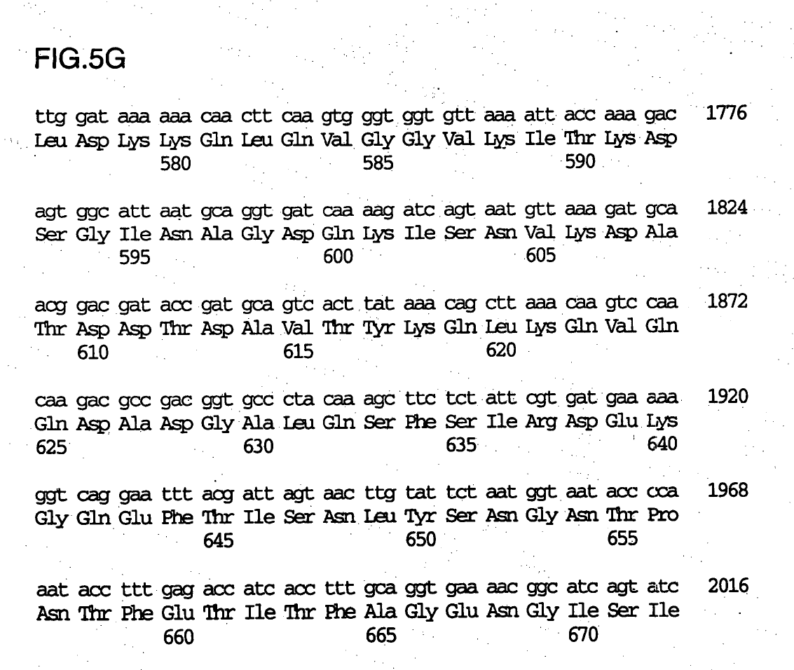

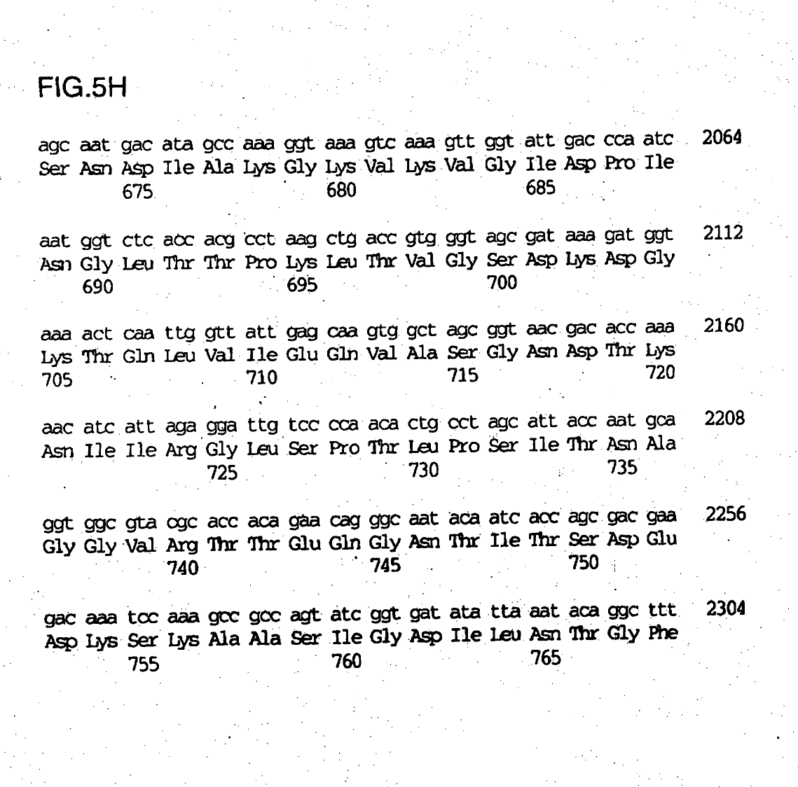

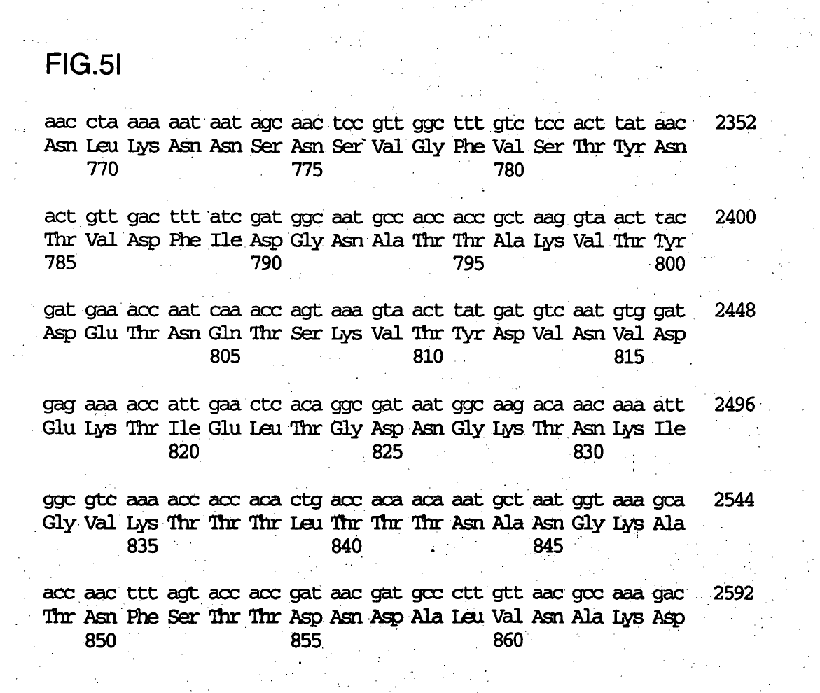

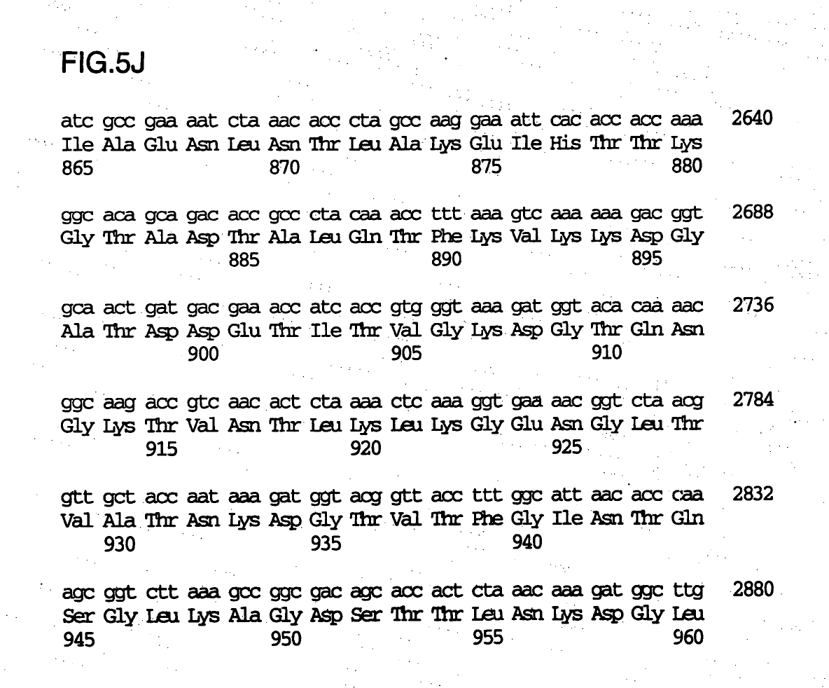

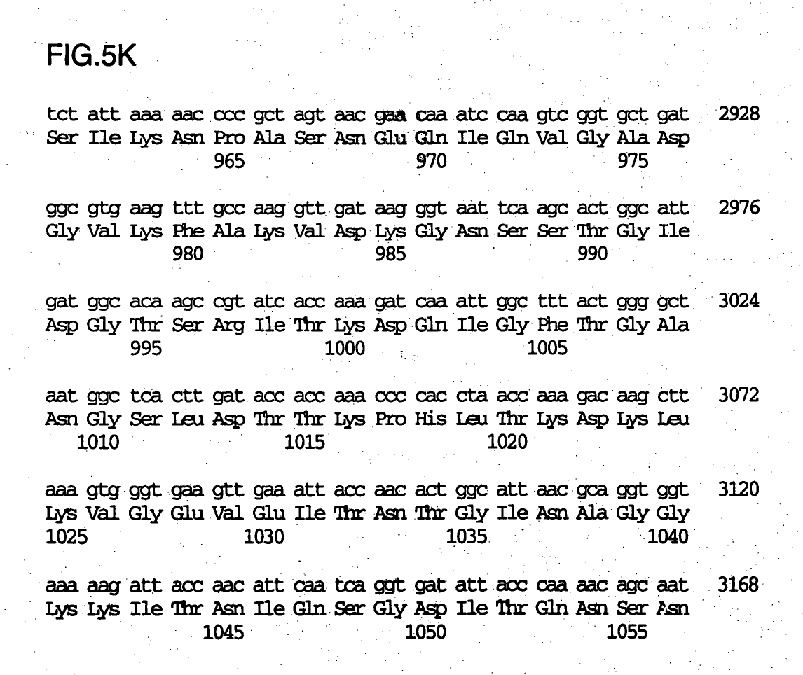

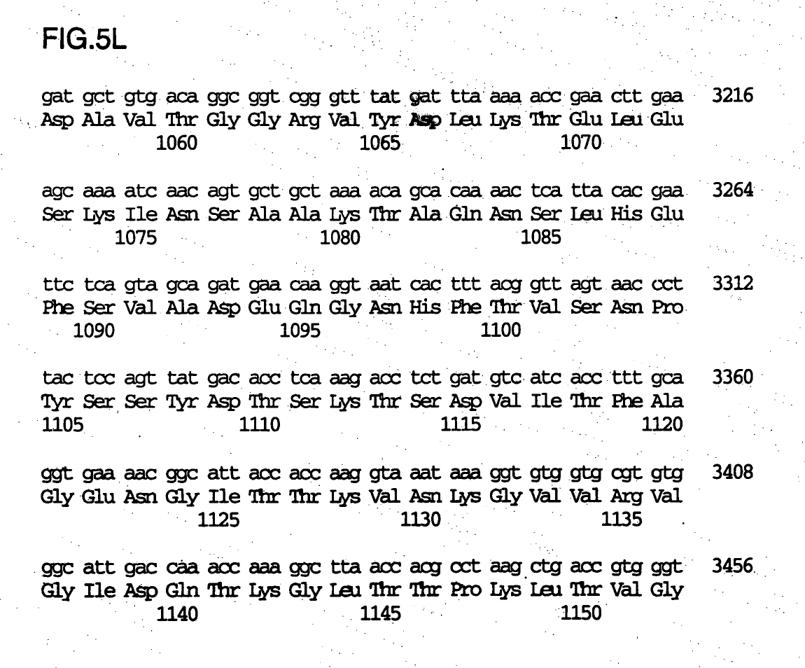

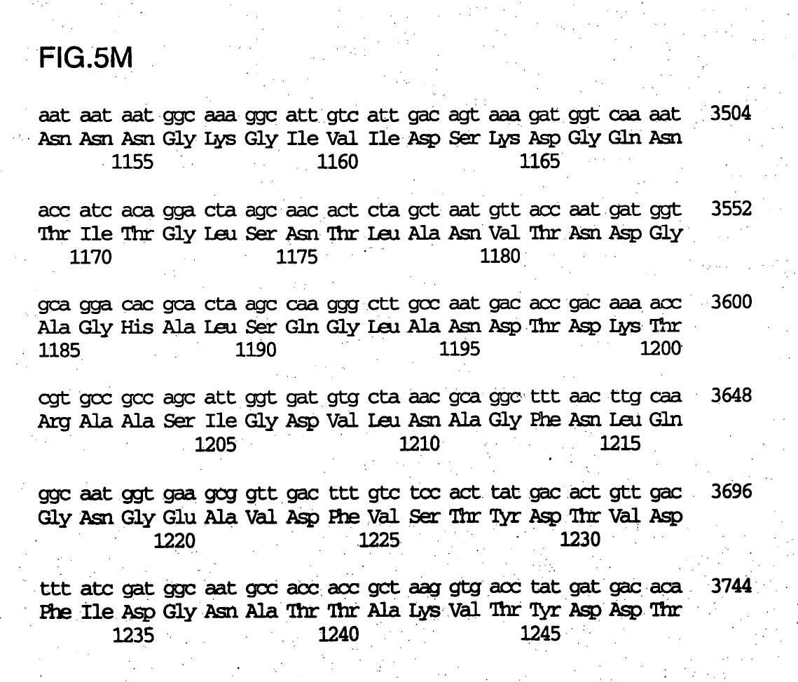

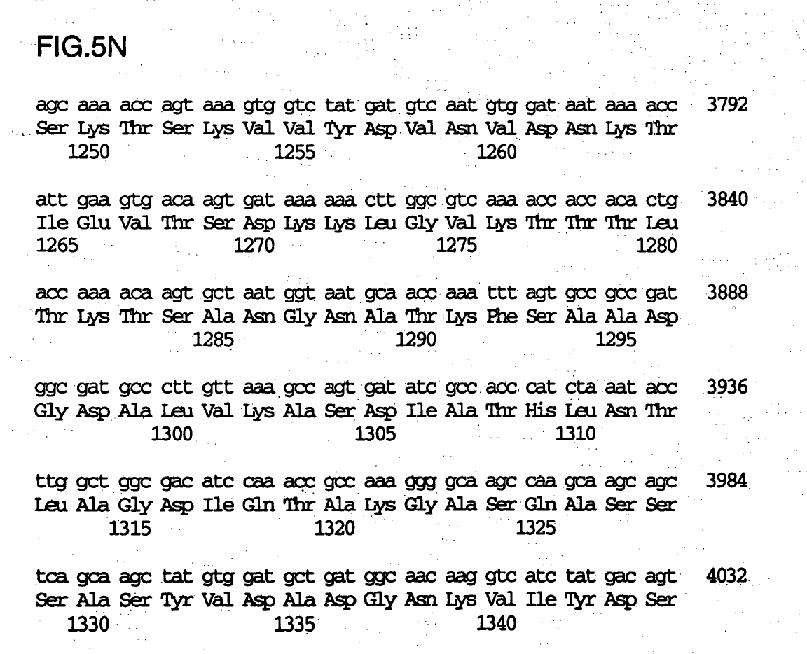

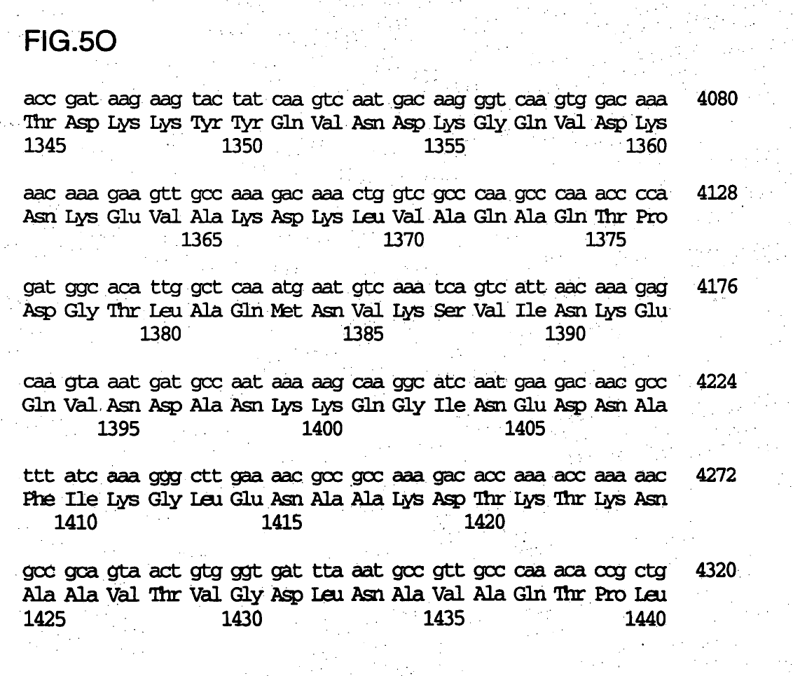

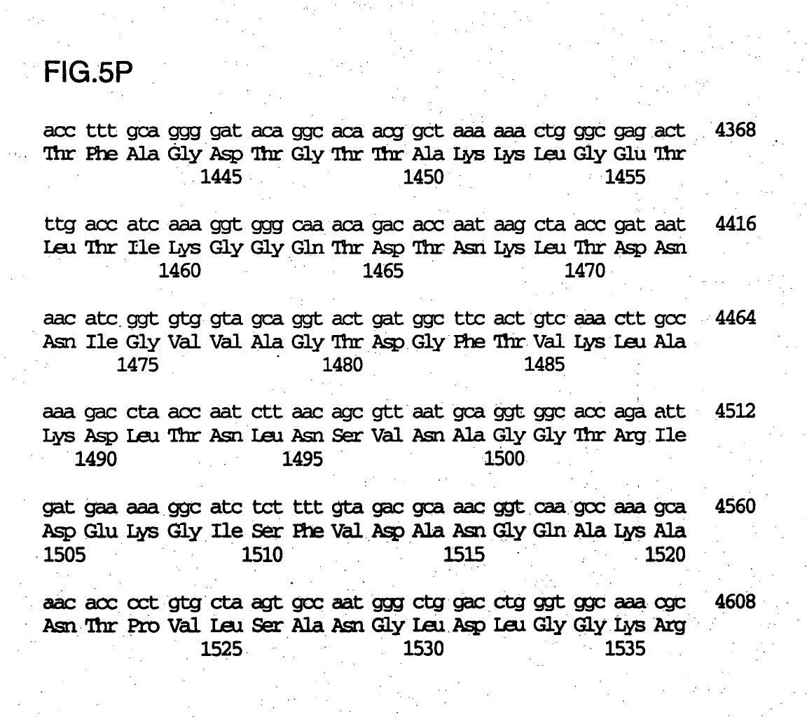

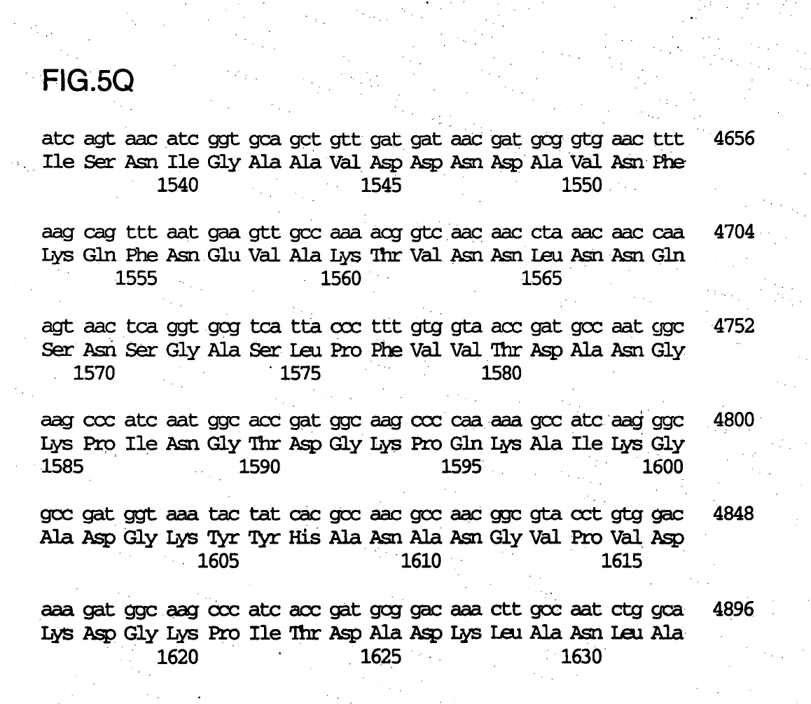

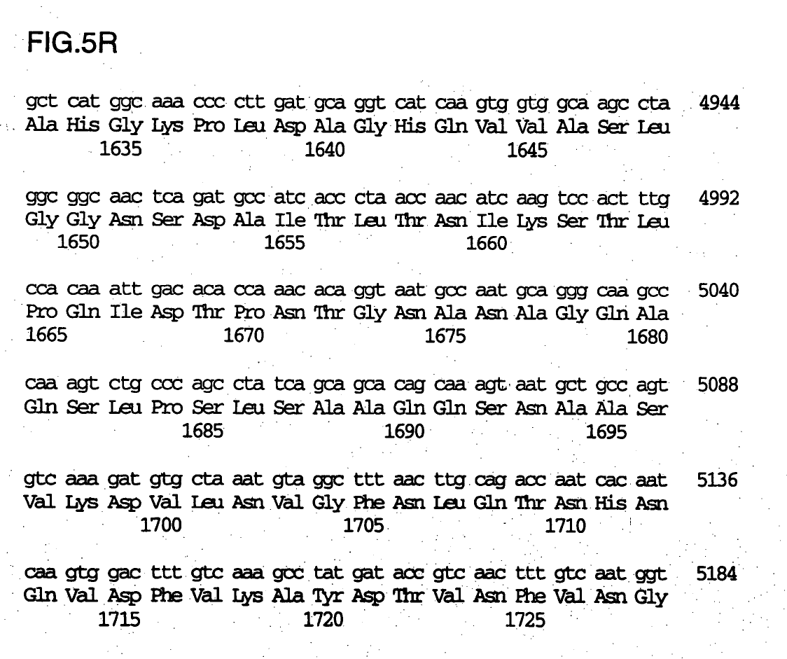

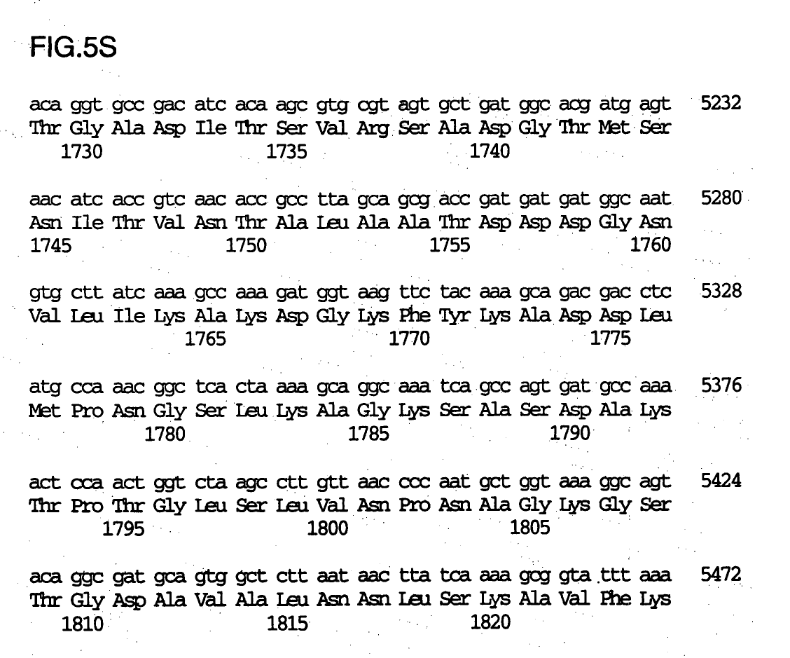

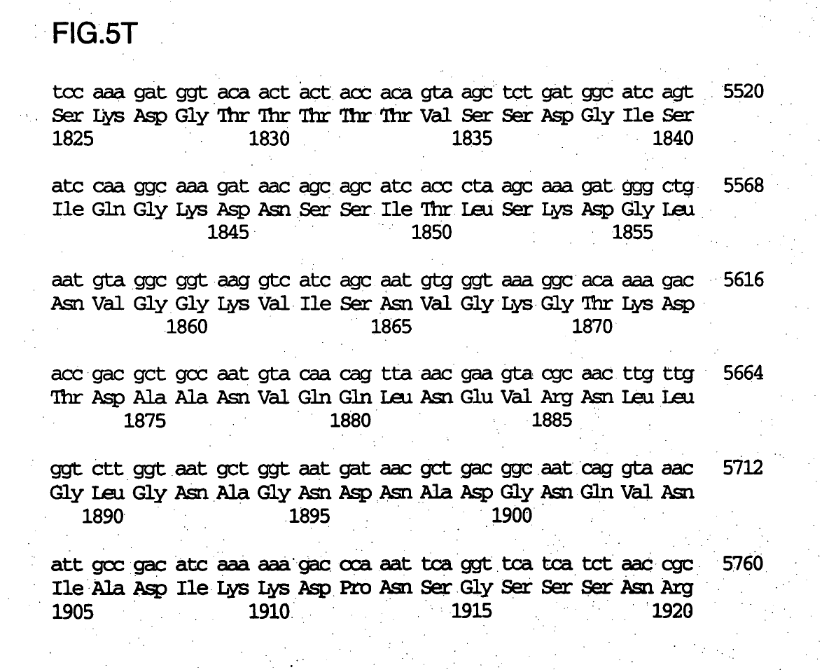

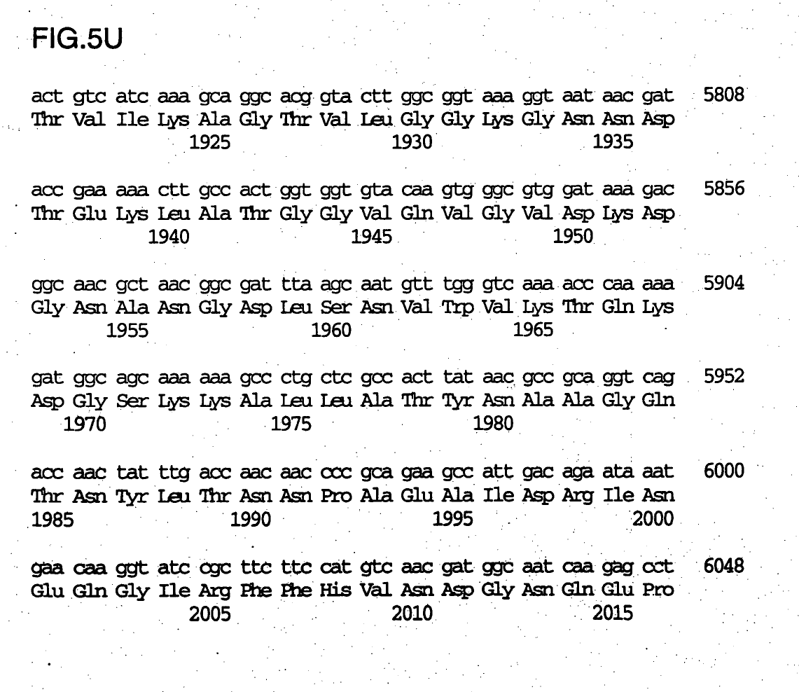

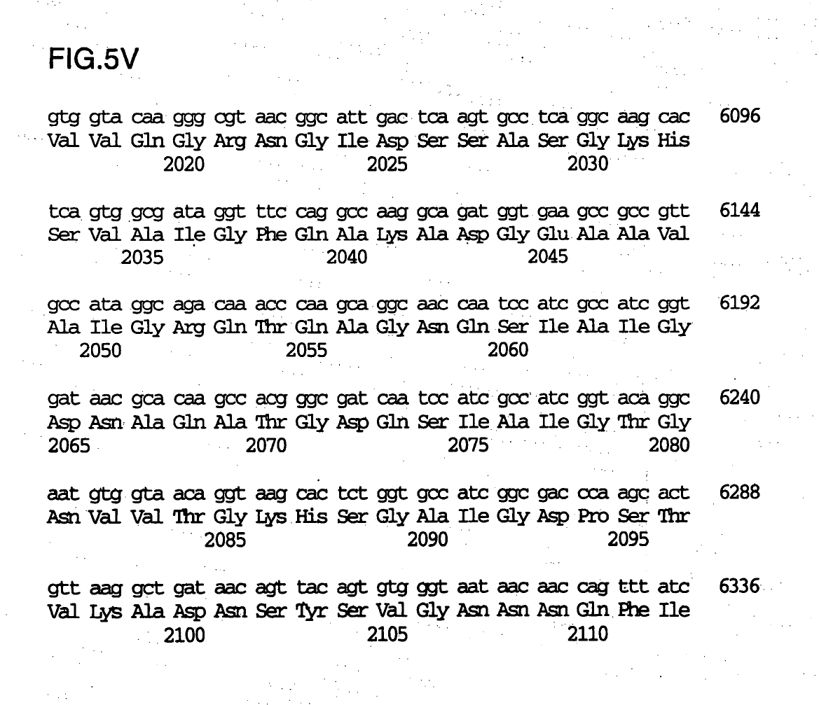

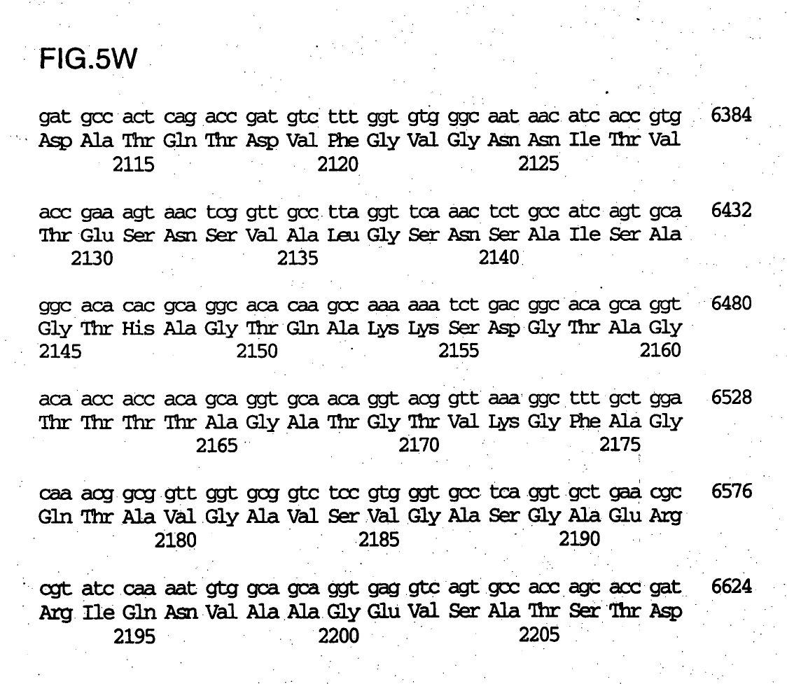

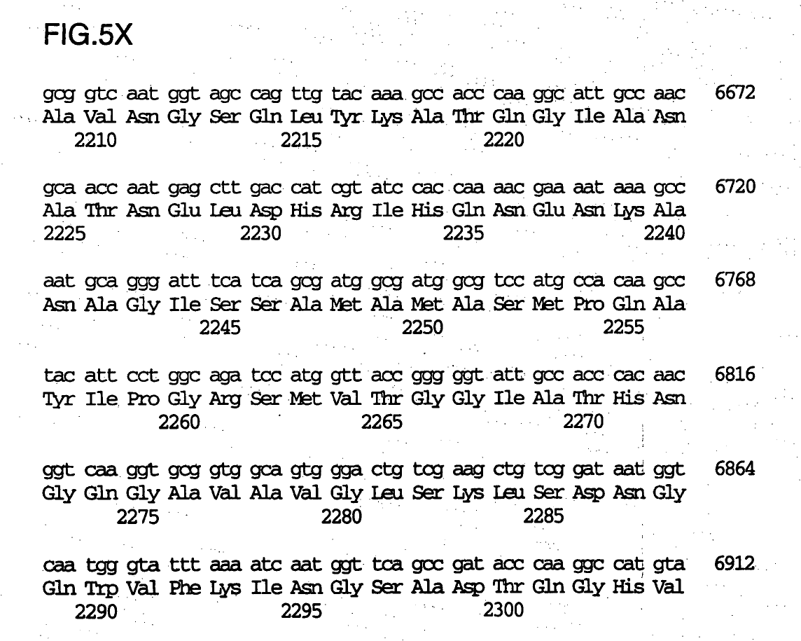

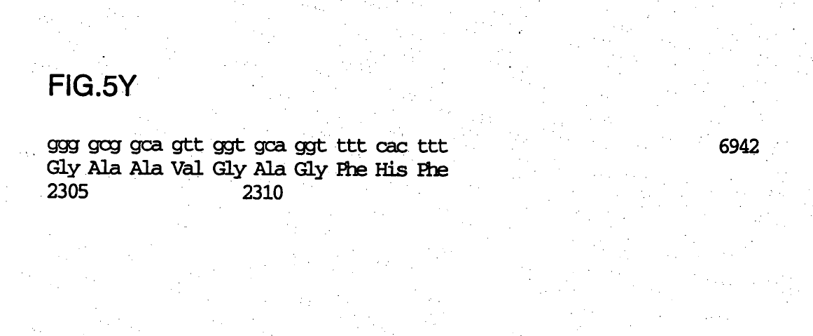

Utilizing the techniques described herein, the genes encoding the about 200 kDa protein from M. catarrhalis strains Q8 and LES-1 have been cloned and sequenced. FIGS. 4 and 5 show respectively the nucleotide and derived amino acid sequences. An amino acid sequence comparison of the derived amino acid sequences of the 200 kDa protein from the three strains of M. catarrhalis is contained in FIG. 6.

Based on the sequence information, a plasmid (pKS294) was constructed that contained the full-length 200 kDa protein gene of strain 4223 starting at the ATG codon, under control of the bacteriophage T7 promoter. However, even a basal level of expression of the full-length gene from the ATG was lethal to E. coli. Deletion of a 165 bp 5′ fragment of the 200 kDa coding region greatly reduced the toxicity of the resultant protein to E. coli. Plasmid pKS348 contains the T7 promoter transcriptionally driving a 200 kDa protein gene which starts at amino acid residue 56. The V56 codon was changed to M56. The M56 r200 kDa protein was produced and the purified protein was used to generate guinea pig antiserum.

In WO 96/34960, a bactericidal antibody assay was described that was used to demonstrate that anti-200 kDa antibody was bactericidal for M. catarrhalis. The assay was used herein to demonstrate broad bactericidal antibody activity against heterologous clinical isolates from different geographical locations, by anti-M56 r200 kDa antibody. A single anti-M56 r200 kDa antibody was lytic for 62% of strains tested.

The 200 kDa protein was originally identified as a putative adhesin when its presence was detected in a clumping strain, but not a non-clumping derivative. In order to determine whether it were truly an adhesin, an in vitro adherence assay was developed in which the inhibition of binding by antibody between M. catarrhalis and epithelial cells was measured. Using this assay, anti-M56 r200 kDa antibody was capable of inhibiting adherence of the homologous strain by 48%, demonstrating that the 200 kDa protein was an adhesin. When an additional 25 strains of M. catarrhalis were assayed, 21 were found to have reduced adherence to epithelial cells in the presence of anti-M56 r200 kDa antibody. 19 of these strains had not been killed by the same antibody. Thus, a single anti-M56 r200 kDa antibody was capable of killing or blocking adherence of 91% of the strains tested.

The sequence comparison for the 200 kDa gene from three strains of M. catarrhalis showed that the C-terminal half of the protein was quite conserved. Strain LES-1 contained an insert of about 300 amino acids. Thus, based upon the C-terminal region, the strains may be divided into two families depending upon whether they contained the insert 4223 and Q8 formed one family while LES-1 formed the other. The carboxy terminal halves (3′ halves) of the 4223 or LES-1 200 kDa genes were expressed in E. coli with good yields and the purified carboxy terminal half of the proteins were used to generate antibodies. When tested in the bactericidal antibody assay, these antisera were bactericidal, as seen in Table 1B.

It is clearly apparent to one skilled in the art, that the various embodiments of the present invention have many applications in the fields of vaccination, diagnosis, treatment of Moraxella infections, and in the generation of immunological reagents. A further non-limiting discussion of such uses is further presented below.

1. Vaccine Preparation and Use

Immunogenic compositions, including those suitable to be used as vaccines, may be prepared from the about 200 kDa outer membrane protein as disclosed herein, as well as immunological fragments and fusions thereof, which may be purified from the bacteria or which may be produced recombinantly. The vaccine elicits an immune response in a subject which produces antibodies, including anti-200 kDa outer membrane protein antibodies and antibodies that are opsonizing or bactericidal. Should the vaccinated subject be challenged by Moraxella or other bacteria that produce proteins capable of producing antibodies that specifically recognize 200 kDa outer membrane protein, the antibodies bind to and inactivate the bacterium. Furthermore, opsonizing or bactericidal anti-200 kDa outer membrane protein antibodies may also provide protection by alternative mechanisms.

Immunogenic compositions including vaccines may be prepared as injectables, as liquid solutions or emulsions. The about 200 kDa outer membrane protein may be mixed with pharmaceutically acceptable excipients which are compatible with the about 200 kDa outer membrane protein. Such excipients may include, water, saline, dextrose, glycerol, ethanol, and combinations thereof. The immunogenic compositions and vaccines may further contain auxiliary substances, such as wetting or emulsifying agents, pH buffering agents, or adjuvants to enhance the effectiveness thereof. Immunogenic compositions and vaccines may be administered parenterally, by injection subcutaneously or intramuscularly. Alternatively, the immunogenic compositions formed according to the present invention, may be formulated and delivered in a manner to evoke an immune response at mucosal surfaces. Thus, the immunogenic composition may be administered to mucosal surfaces by, for example, the nasal or oral (intragastric) routes. Alternatively, other modes of administration including suppositories and oral formulations may be desirable. For suppositories, binders and carriers may include, for example, polyalkalene glycols or triglycerides. Oral formulations may include normally employed incipients such as, for example, pharmaceutical grades of saccharine, cellulose and magnesium carbonate. These compositions can take the form of solutions, suspensions, tablets, pills, capsules, sustained release formulations or powders and contain about 1 to 95% of the about 200 kDa outer membrane protein. The immunogenic preparations and vaccines are administered in a manner compatible with the dosage formulation, and in such amount as will be therapeutically effective, protective and immunogenic. The quantity to be administered depends on the subject to be treated, including, for example, the capacity of the individual's immune system to synthesize antibodies, and if needed, to produce a cell-mediated immune response. Precise amounts of active ingredient required to be administered depend on the judgement of the practitioner. However, suitable dosage ranges are readily determinable by one skilled in the art and may be of the order of micrograms of the about 200 kDa outer membrane protein. Suitable regimes for initial administration and booster doses are also variable, but may include an initial administration followed by subsequent administrations. The dosage may also depend on the route of administration and will vary according to the size of the host.

The immunogenic preparations including vaccines may comprise as the immunostimulating material a nucleotide vector comprising at least a portion of the gene encoding the about 200 kDa protein, or the at least a portion of the gene may be used directly for immunization.

The concentration of the about 200 kDa outer membrane antigen in an immunogenic composition according to the invention is in general about 1 to 95%. A vaccine which contains antigenic material of only one pathogen is a monovalent vaccine. Vaccines which contain antigenic material of several pathogens are combined vaccines and also belong to the present invention. Such combined vaccines contain, for example, material from various pathogens or from various strains of the same pathogen, or from combinations of various pathogens.

Immunogenicity can be significantly improved if the antigens are co-administered with adjuvants, commonly used as 0.05 to 0.1 percent solution in phosphate-buffered saline. Adjuvants enhance the immunogenicity of an antigen but are not necessarily immunogenic themselves. Adjuvants may act by retaining the antigen locally near the site of administration to produce a depot effect facilitating a slow, sustained release of antigen to cells of the immune system. Adjuvants can also attract cells of the immune system to an antigen depot and stimulate such cells to elicit immune responses.

Immunostimulatory agents or adjuvants have been used for many years to improve the host immune responses to, for example, vaccines. Intrinsic adjuvants, such as lipopolysaccharides, normally are the components of the killed or attenuated bacteria used as vaccines. Extrinsic adjuvants are immunomodulators which are typically non-covalently linked to antigens and are formulated to enhance the host immune responses. Thus, adjuvants have been identified that enhance the immune response to antigens delivered parenterally. Some of these adjuvants are toxic, however, and can cause undesirable side-effects, making them unsuitable for use in humans and many animals. Indeed, only aluminum hydroxide and aluminum phosphate (collectively commonly referred to as alum) are routinely used as adjuvants in human and veterinary vaccines. The efficacy of alum in increasing antibody responses to diphtheria and tetanus toxoids is well established and a HBsAg vaccine has been adjuvanted with alum. While the usefulness of alum is well established for some applications, it has limitations. For example, alum is ineffective for influenza vaccination and inconsistently elicits a cell mediated immune response.

A wide range of extrinsic adjuvants can provoke potent immune responses to antigens. These include saponins complexed to membrane protein antigens (immune stimulating complexes), pluronic polymers with mineral oil, killed mycobacteria in mineral oil, Freund's complete adjuvant, bacterial products, such as muramyl dipeptide (MDP) and lipopolysaccharide (LPS), as well as lipid A, and liposomes.

To efficiently induce humoral immune responses (HIR) and cell-mediated immunity (CMI), immunogens are typically emulsified in adjuvants. Many adjuvants are toxic, inducing granulomas, acute and chronic inflammations (Freund's complete adjuvant) FCA, cytolysis (saponins and Pluronic polymers) and pyrogenicity, arthritis and anterior uveitis (LPS and MDP). Although FCA is an excellent adjuvant and widely used in research, it is not licensed for use in human or veterinary vaccines because of its toxicity.

Desirable characteristics of ideal adjuvants include:
(1) lack of toxicity;
(2) ability to stimulate a long-lasting immune response;
(3) simplicity of manufacture and stability in long-term storage;
(4) ability to elicit both CMI and HIR to antigens administered by various routes, if required;
(5) synergy with other adjuvants;
(6) capability of selectively interacting with populations of antigen presenting cells (APC);
(7) ability to specifically elicit appropriate T_{H}1 or T_{H}2 cell-specific immune responses; and
(8) ability to selectively increase appropriate antibody isotype levels (for example, IgA) against antigens.

U.S. Pat. No. 4,855,283 granted to Lockhoff et al on Aug. 8, 1989 which is incorporated herein by reference thereto, teaches glycolipid analogues including N-glycosylamides, N-glycosylureas and N-glycosylcarbamates, each of which is substituted in the sugar residue by an amino acid, as immuno-modulators or adjuvants. Thus, Lockhoff et al. (U.S. Pat. No. 4,855,283 and ref. 27) reported that N-glycolipid analogs displaying structural similarities to the naturally-occurring glycolipids, such as glycosphospholipids and glycoglycerolipids, are capable of eliciting strong immune responses in both herpes simplex virus vaccine and pseudorabies virus vaccine. Some glycolipids have been synthesized from long chain-alkylamines and fatty acids that are linked directly with the sugars through the anomeric carbon atom, to mimic the functions of the naturally occurring lipid residues.

U.S. Pat. No. 4,258,029 granted to Moloney, assigned to the assignee hereof and incorporated herein by reference thereto, teaches that octadecyl tyrosine hydrochloride (OTH) functioned as an adjuvant when complexed with tetanus toxoid and formalin inactivated type I, II and III poliomyelitis virus vaccine. Also, Nixon-George et al. (ref. 24), reported that octadecyl esters of aromatic amino acids complexed with a recombinant hepatitis B surface antigen, enhanced the host immune responses against hepatitis B virus.

Lipidation of synthetic peptides has also been used to increase their immunogenicity. Thus, Wiesmuller (ref. 25) describes a peptide with a sequence homologous to a foot-and-mouth disease viral protein coupled to an adjuvant tripalmityl-S-glyceryl-cysteinylserylserine, being a synthetic analogue of the N-terminal part of the lipoprotein from Gram negative bacteria. Furthermore, Deres et al. (ref. 26) reported in vivo priming of virus-specific cytotoxic T lymphocytes with synthetic lipopeptide vaccine which comprised of modified synthetic peptides derived from influenza virus nucleoprotein by linkage to a lipopeptide, N-palmityl-S-2,3-bis(palmitylxy)-(2RS)-propyl-[R]-cysteine (TPC).

2. Immoassays

The about 200 kDa outer membrane protein of the present invention is useful as an immunogen for the generation of anti-200 kDa outer membrane protein antibodies, as an antigen in immunoassays including enzyme-linked immunosorbent assays (ELISA), RIAs and other non-enzyme linked antibody binding assays or procedures known in the art for the detection of anti-bacterial, anti-Moraxella, and anti-200 kDa outer membrane protein antibodies. In ELISA assays, the about 200 kDa outer membrane protein is immobilized onto a selected surface, for example, a surface capable of binding proteins such as the wells of a polystyrene microtiter plate. After washing to remove incompletely adsorbed about 200 kDa outer membrane protein, a nonspecific protein such as a solution of bovine serum albumin (BSA) that is known to be antigenically neutral with regard to the test sample may be bound to the selected surface. This allows for blocking of nonspecific adsorption sites on the immobilizing surface and thus reduces the background caused by nonspecific bindings of antisera onto the surface.

The immobilizing surface is then contacted with a sample, such as clinical or biological materials, to be tested in a manner conducive to immune complex (antigen/antibody) formation. This may include diluting the sample with diluents, such as solutions of BSA, bovine gamma globulin (BGG) and/or phosphate buffered saline (PBS)/Tween. The sample is then allowed to incubate for from 2 to 4 hours, at temperatures such as of the order of about 20° to 37° C. Following incubation, the sample-contacted surface is washed to remove non-immunocomplexed material. The washing procedure may include washing with a solution, such as PBS/Tween or a borate buffer. Following formation of specific immunocomplexes between the test sample and the bound about 200 kDa outer membrane protein, and subsequent washing, the occurrence, and even amount, of immunocomplex formation may be determined by subjecting the immunocomplex to a second antibody having specificity for the first antibody. If the test sample is of human origin, the second antibody is an antibody having specificity for human immunoglobulins and in general IgG. To provide detecting means, the second antibody may have an associated activity such as an enzymatic activity that will generate, for example, a colour development upon incubating with an appropriate chromogenic substrate. Quantification may then be achieved by measuring the degree of colour generation using, for example, a visible spectrophotometer.

3. Use of Sequences as Hybridization Probes

The nucleotide sequences of the present invention, comprising the sequence of the about 200 kDa protein gene, now allow for the identification and cloning of the about 200 kDa protein gene from any species of Moraxella.

The nucleotide sequences comprising the sequence of the about 200 kDa protein gene of the present invention are useful for their ability to selectively form duplex molecules with complementary stretches of other about 200 kDa protein genes. Depending on the application, a variety of hybridization conditions may be employed to achieve varying degrees of selectivity of the probe toward the other genes. For a high degree of selectivity, relatively stringent conditions are used to form the duplexes, such as low salt and/or high temperature conditions, such as provided by 0.02 M to 0.15 M NaCl at temperatures of between about 50° C. to 70° C. For some applications, less stringent hybridization conditions are required such as 0.15 M to 0.9 M salt, at temperatures ranging from between about 20° C. to 55° C. Hybridization conditions can also be rendered more stringent by the addition of increasing amounts of formamide, to destabilize the hybrid duplex. Thus, particular hybridization conditions can be readily manipulated, and will generally be a method of choice depending on the desired results. In general, convenient hybridization temperatures in the presence of 50% formamide are: 42° C. for a probe which is 95 to 100% homologous to the target fragment, 37° C. for 90 to 95% homology and 32° C. for 85 to 90% homology.

In a clinical diagnostic embodiment, the nucleic acid sequences of the about 200 kDa protein genes of the present invention may be used in combination with an appropriate means, such as a label, for determining hybridization. A wide variety of appropriate indicator means are known in the art, including radioactive, enzymatic or other ligands, such as avidin/biotin and digoxigenin-labelling, which are capable of providing a detectable signal. In some diagnostic embodiments, an enzyme tag such as urease, alkaline phosphatase or peroxidase, instead of a radioactive tag may be used. In the case of enzyme tags, colorimetric indicator substrates are known which can be employed to provide a means visible to the human eye or spectrophotometrically, to identify specific hybridization with samples containing about 200 kDa protein gene sequences.

The nucleic acid sequences of the about 200 kDa protein genes of the present invention are useful as hybridization probes in solution hybridizations and in embodiments employing solid-phase procedures. In embodiments involving solid-phase procedures, the test DNA (or RNA) from samples, such as clinical samples, including exudates, body fluids (e.g., serum, amniotic fluid, middle ear effusion, sputum, bronchoalveolar lavage fluid) or even tissues, is adsorbed or otherwise affixed to a selected matrix or surface. The fixed, single-stranded nucleic acid is then subjected to specific hybridization with selected probes comprising the nucleic acid sequences of the about 200 kDa protein encoding genes or fragments or analogs thereof of the present invention under desired conditions. The selected conditions will depend on the particular circumstances based on the particular criteria required depending on, for example, the G+C contents, type of target nucleic acid, source of nucleic acid, size of hybridization probe etc. Following washing of the hybridization surface so as to remove non-specifically bound probe molecules, specific hybridization is detected, or even quantified, by means of the label. It is preferred to select nucleic acid sequence portions which are conserved among species of Moraxella. The selected probe may be at least 18 bp and may be in the range of about 30 to 90 bp.

4. Expression of the about 200 kDa Protein Gene

Plasmid vectors containing replicon and control sequences which are derived from species compatible with the host cell may be used for the expression of the genes encoding the about 200 kDa protein in expression systems. The vector ordinarily carries a replication site, as well as marking sequences which are capable of providing phenotypic selection in transformed cells. For example, E. coli may be transformed using pBR322 which contains genes for ampicillin and tetracycline resistance and thus provides an easy means for identifying transformed cells. The plasmids or phage, must also contain, or be modified to contain, promoters which can be used by the host cell for expression of its own proteins.

In addition, phage vectors containing replicon and control sequences that are compatible with the host can be used as a transforming vector in connection with these hosts. For example, the phage in lambda GEM™-11 may be utilized in making recombinant phage vectors which can be used to transform host cells, such as E. coli LE392.

Promoters commonly used in recombinant DNA construction include the β-lactamase (penicillinase) and lactose promoter systems and other microbial promoters, such as the T7 promoter system as described in U.S. Pat. No. 4,952,496. Details concerning the nucleotide sequences of promoters are known, enabling a skilled worker to ligate them functionally with genes. The particular promoter used will generally be a matter of choice depending upon the desired results. Hosts that are appropriate for expression of the about 200 kDa protein genes, fragments, analogs or variants thereof, may include E. coli, Bacillus species, Haemophilus, fungi, yeast, Bordetella, or the baculovirus expression system may be used.

In accordance with this invention, it is preferred to make the protein by recombinant methods, particularly when the naturally occurring about 200 kDa protein as purified from a culture of a species of Moraxella may include trace amounts of toxic materials or other contaminants. This problem can be avoided by using recombinantly produced protein in heterologous systems which can be isolated from the host in a manner to minimize contaminants in the purified material. Particularly desirable hosts for expression in this regard include Gram positive bacteria which do not have LPS and are, therefore, endotoxin free. Such hosts include species of Bacillus and may be particularly useful for the production of non-pyrogenic about 200 kDa protein, fragments or analogs thereof.

### Biological Deposits

Certain plasmids that contain portions and full-length of the gene having the open reading frame of the gene encoding the about 200 kDa outer membrane protein of M. catarrhalis strain 4223 that are described and referred to herein have been deposited with the America Type Culture Collection (ATCC) located at 10801 University Blvd., Manassas, Va. 20110-2209, U.S.A., pursuant to the Budapest Treaty and pursuant to 37 CFR 1.808 and prior to the filing of this application.

Samples of the deposited plasmids will become available to the public upon grant of a patent based upon this United States patent application or relevant precursor applications. The invention described and claimed herein is not to be limited in scope by plasmids deposited, since the deposited embodiment is intended only as an illustration of the invention. Any equivalent or similar plasmids that encode similar or equivalent antigens as described in this application are within the scope of the invention.

| Plasmid | ATCC Designation | Date Deposited |
|---|---|---|
| pKS47 | 97,111 | Apr. 7, 1995 |
| pKS5 | 97,110 | Apr. 7, 1995 |
| pKS9 | 97,114 | Apr. 18, 1995 |
| pKS294 | 203,528 | Dec. 17, 1998 |
| pKS348 | 203,529 | Dec. 17, 1998 |

### EXAMPLES

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitations.

Methods of molecular genetics, protein biochemistry, and immunology used but not explicitly described in this disclosure and these Examples are amply reported in the scientific literature and are well within the ability of those skilled in the art.

### Example 1

This Example describes the cloning of a gene encoding the M. catarrhalis 200 kDa outer membrane protein.

A M. catarrhalis genomic library in phage lambda EMBL3 was prepared as described in Example 9 of U.S. Pat. No. 5,808,024 and WO 96/34960 and was screened using guinea pig anti-200 kDa protein antiserum. A lambda phage clone 8II, which expressed an about 200 kDa protein, was confirmed by immunoblotting of the phage lysate using the about 200 kDa outer membrane-specific antiserum.

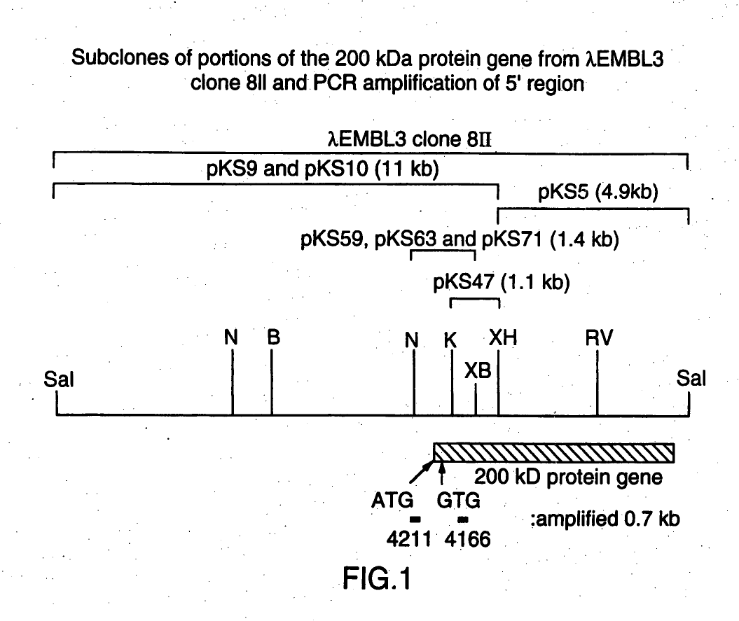

Plate lysate cultures of this recombinant phage were prepared. The DNA was extracted from the plate lysates using a Wizard Lambda Preps DNA Purification System (Promega Corp, Madison, Wis.) according to the manufacturer's instructions. This phage clone carried a DNA insert of about 16 kb in size (the restriction map for which is shown in FIG. 1). The phage DNA was digested with a mixture of the restriction enzymes SalI and XhoI, and separated by agarose gel electrophoresis. Two DNA bands, approximately 5 kb and 11 kb in size, respectively, were cut out from the gel and extracted using a Geneclean kit (BIO 101 Inc., LaJolla, Calif.) according to the manufacturer's direction.

The smaller 5 kb fragment was ligated into a plasmid vector, pBluescript II SK+/−(Stratagene Cloning Systems, LaJolla, Calif.), which had been previously digested with SalI and XhoI, to produce plasmid pKS5. The larger 11 kb fragment was ligated into a plasmid vector, pSP72 (Promega Corp., Madison, Wis.), digested with SalI and XhoI, to produce plasmid pKS9. Both ligated plasmids were used to transform E. coli, strain DH5α.

The lambda phage DNA was also digested with a mixture of XhoI and KpnI and the approximately 1.1 kb fragment was isolated after agarose gel separation as described above. This 1.1 kb fragment was ligated into a plasmid vector, pGEM-7Zf(+) (Promega Corp., Madison, Wis.), to produce plasmid pKS47.

### Example 2

This Example describes the isolation of chromosomal DNA from M. catarrhalis for use in PCR amplification.

M. catarrhalis was cultured in 25 ml of BHI broth overnight and centrifuged at 5,000 rpm for 10 min. The bacteria pellet was suspended in 10 ml of 10 mM Tris/HCl (pH 8.0) containing 100 mM EDTA and mixed with RNaseA (final concentration: 100 μg/ml) and lysozyme (final concentration: 1 mg/ml). After incubation on ice for 10 min and at room temperature for 50 min, the suspension was gently mixed with 1 ml of 10% SDS and then heated at 65° C. for 20 min. The suspension was mixed with proteinase K (final concentration: 200 μg/ml) and incubated at 50° C. for 1 h. The suspension was gently mixed with 10 ml chloroform on a nutator for 15 min and centrifuged at 5,000 rpm for 10 min. The upper phase was slowly removed with a wide-bore pipette and mixed with 10 ml of Tris-saturated phenol and 10 ml of chloroform on a nutator. After centrifugation at 5,000 rpm for 10 min, the upper phase was re-extracted with a mixture of Tris-saturated phenol and chloroform, again, and then extracted with chloroform, and then twice dialyzed against 1M NaCl at 4° C. and twice against TE buffer (pH 8.0) at 4° C.

### Example 3

This Example describes subcloning and sequence analysis of fragments of the 200 kDa protein gene from M. catarrhalis strain 4223.

The procedures used to produce a phage λEMBL3 clone 8II, and its subclones, pKS5, pKS9 and pKS47, are described in U.S. Pat. No. 5,808,024 and WO 96/34960. pKS10 was constructed from the λEMBL3 clone 8II exactly as described for pKS9. pKS59 and pKS63 were constructed by insertion of a 1.4 kb XbaI-NcoI fragment of pKS9 into pGEM5Z(+) that had been digested with NcoI and SpeI. pKS71 was made by insertion of the same 1.4 kb XbaI-NcoI fragment, isolated from the λEMBL3 clone 8II into pGEM5Z(+). Sequence analysis confirmed that all three plasmids, pKS59, pKS63 and pKS71, carried identical DNA fragments. FIG. 1 shows partial restriction maps for the plasmids.

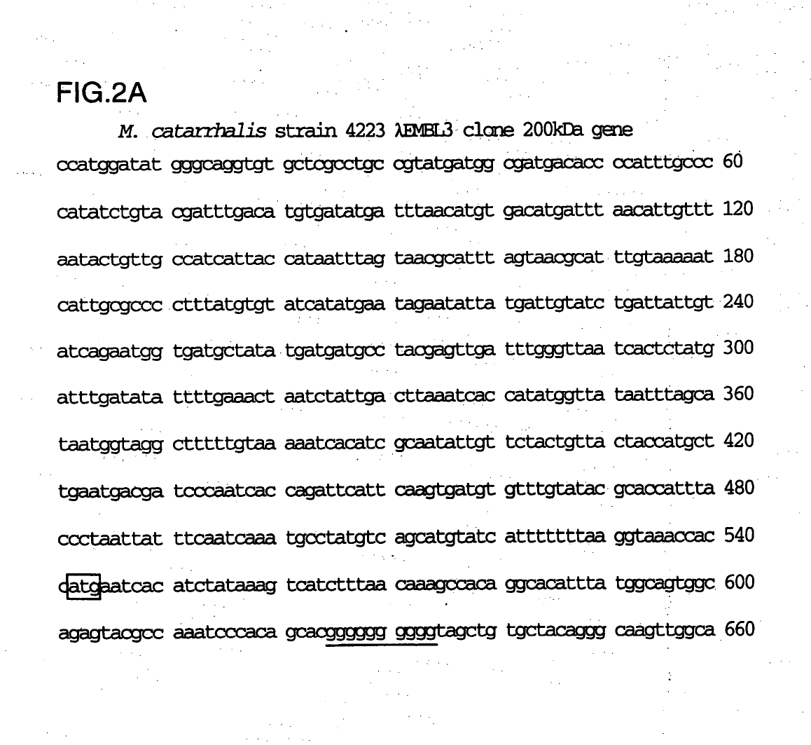

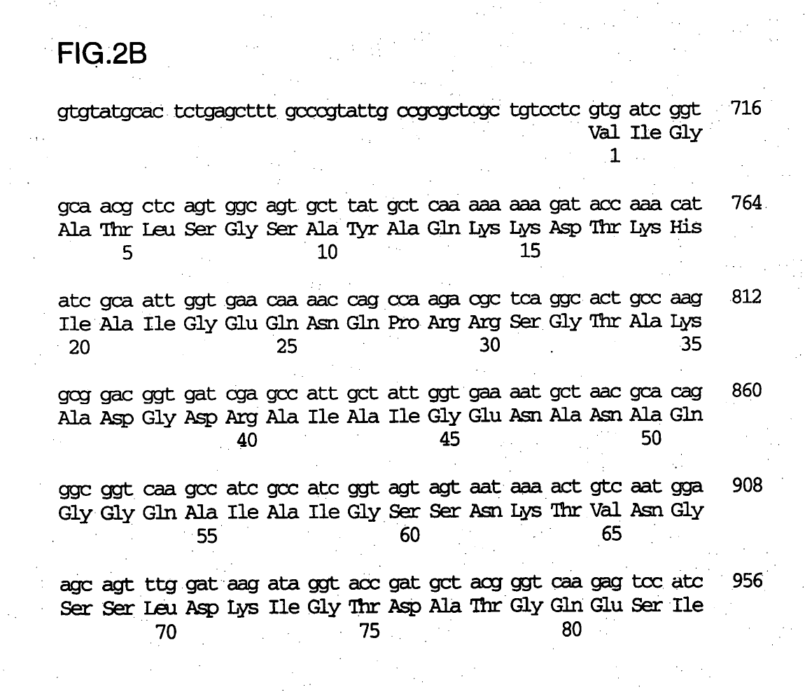

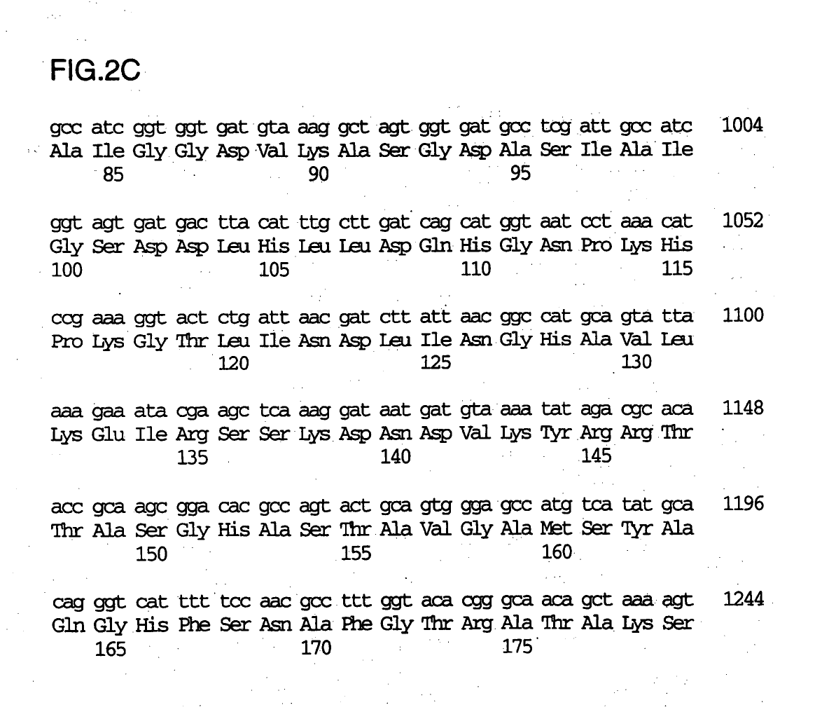

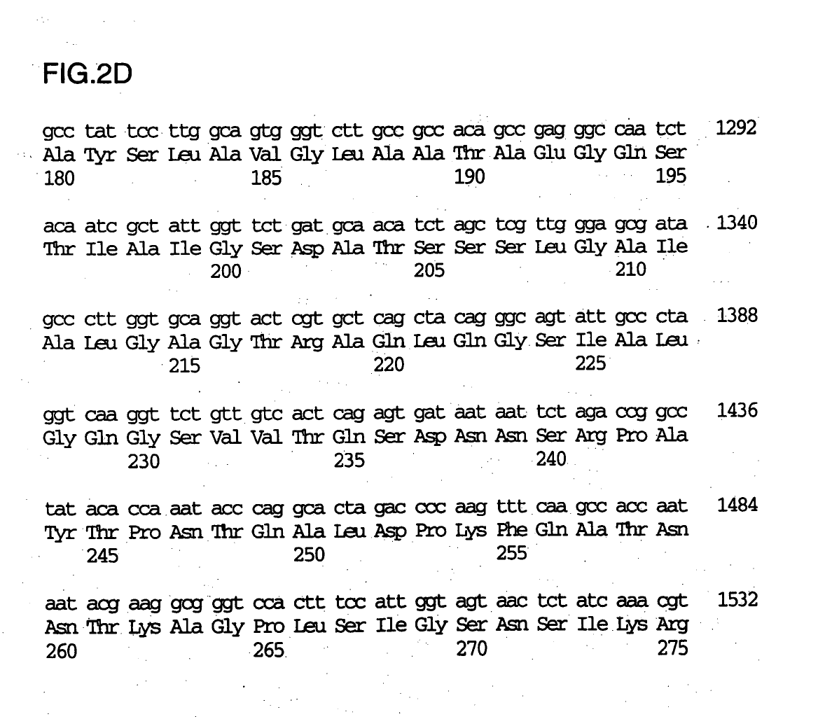

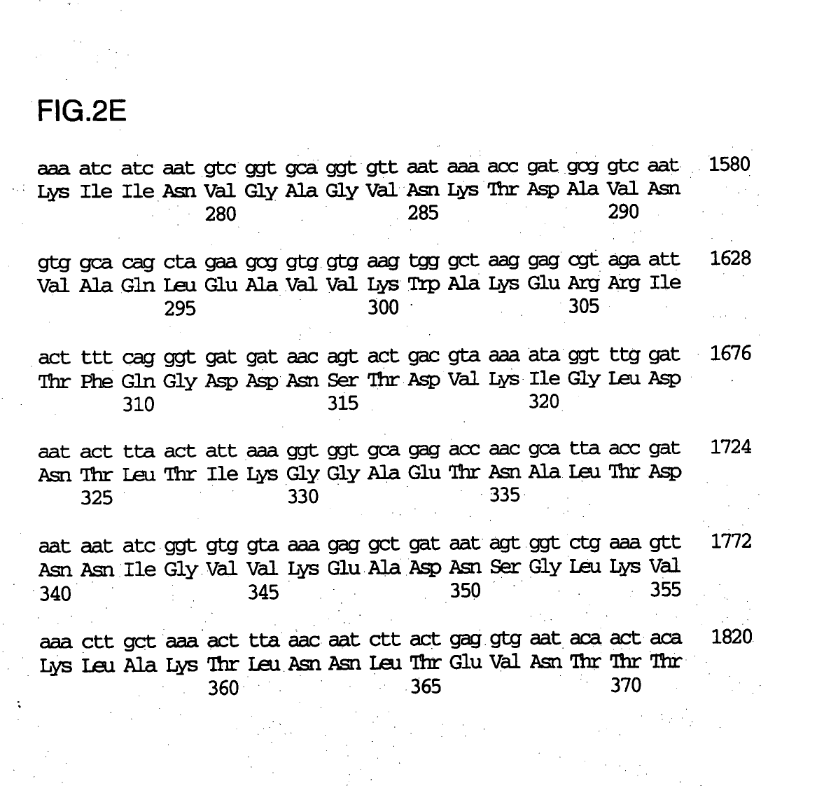

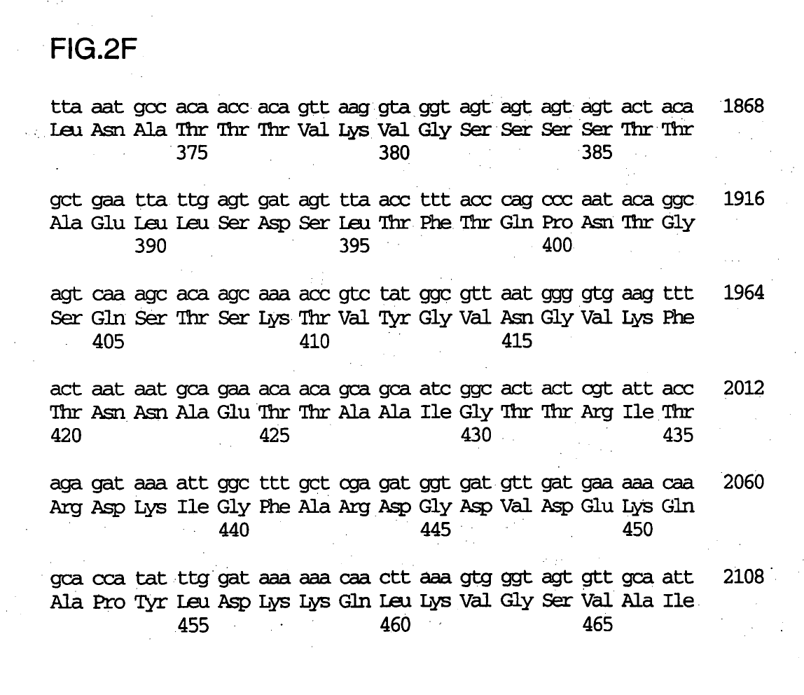

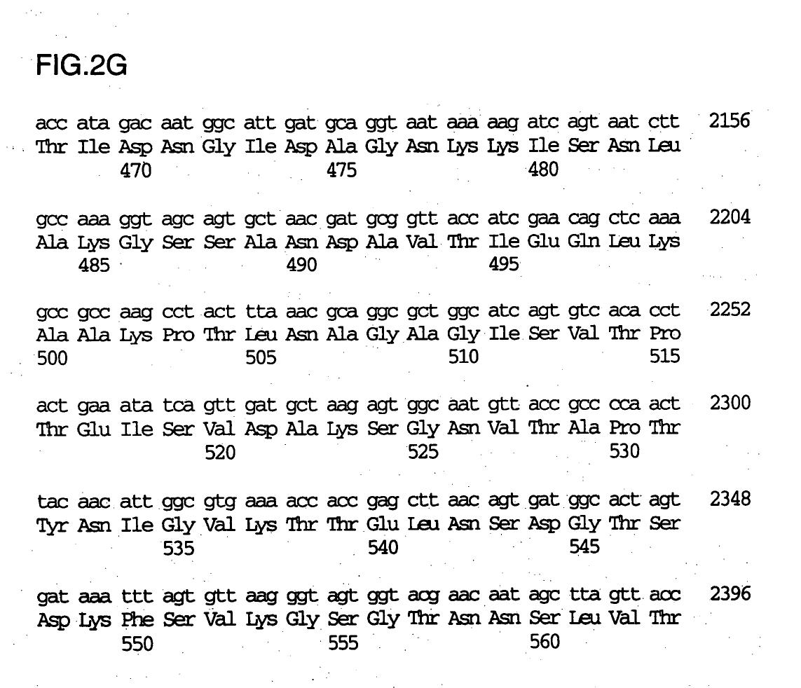

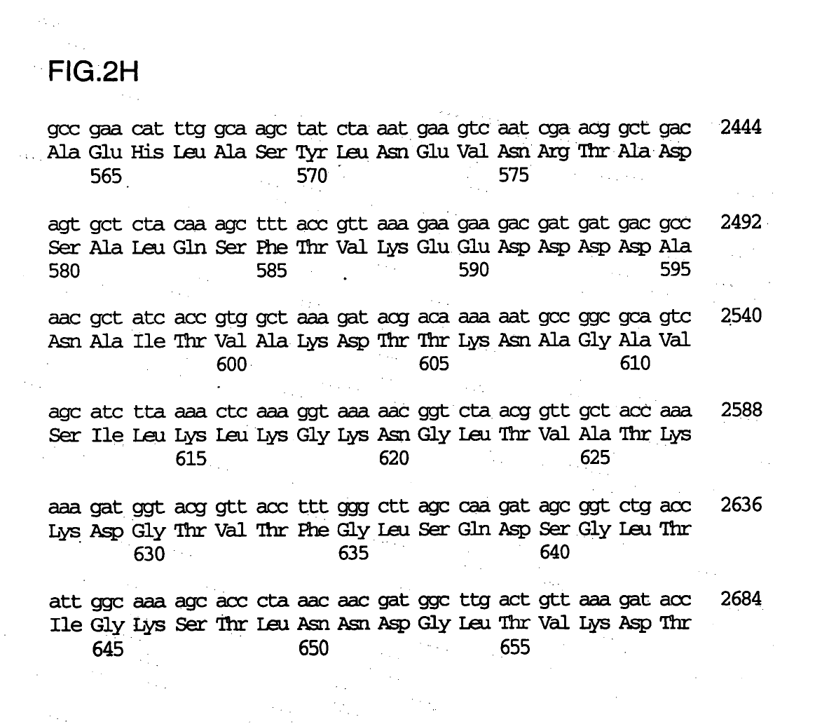

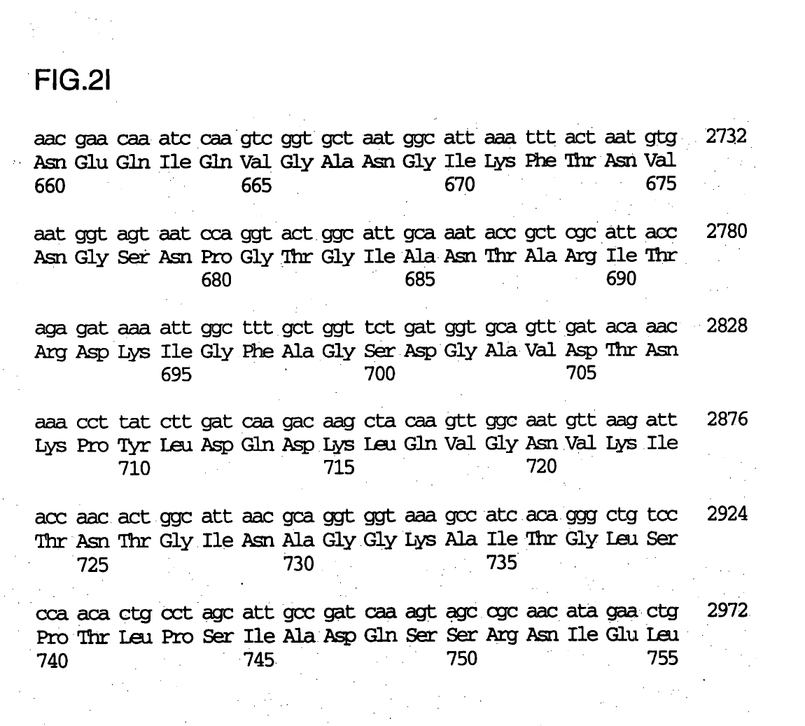

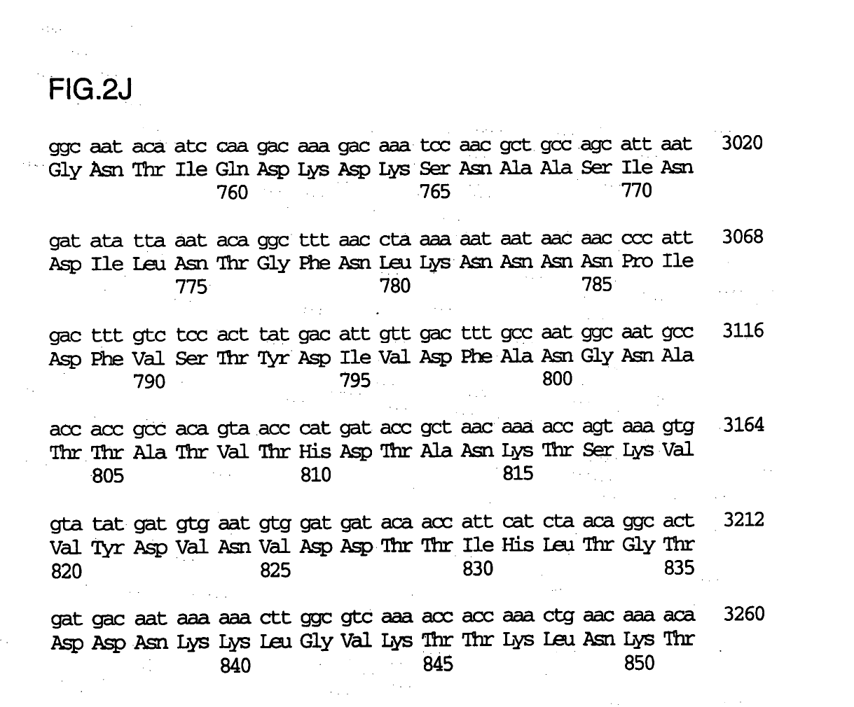

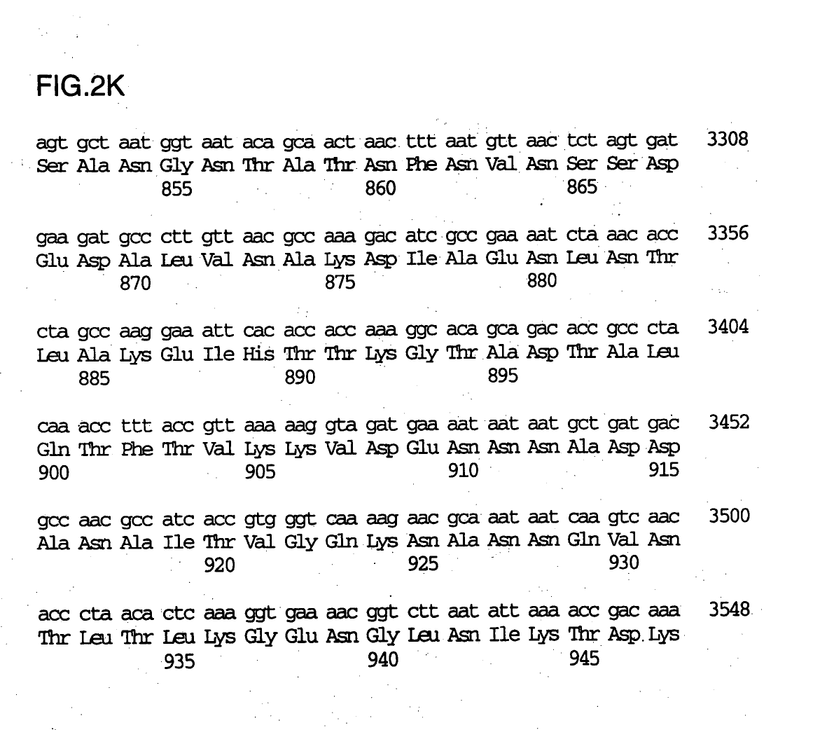

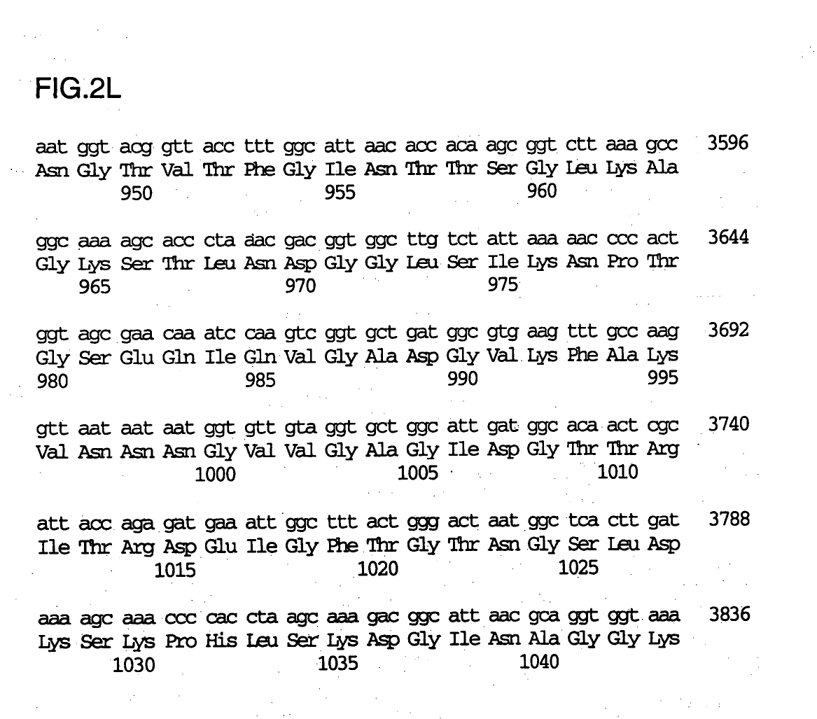

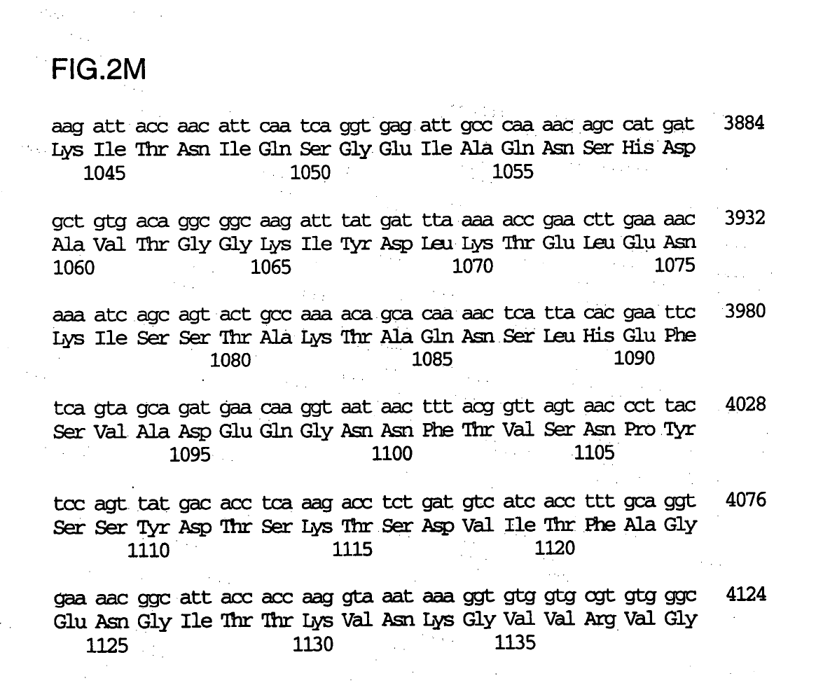

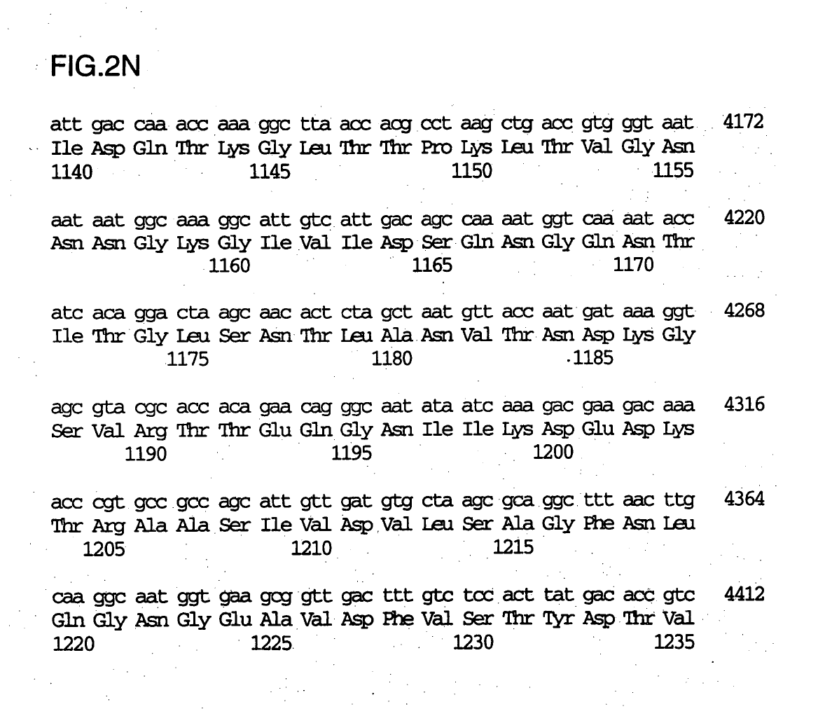

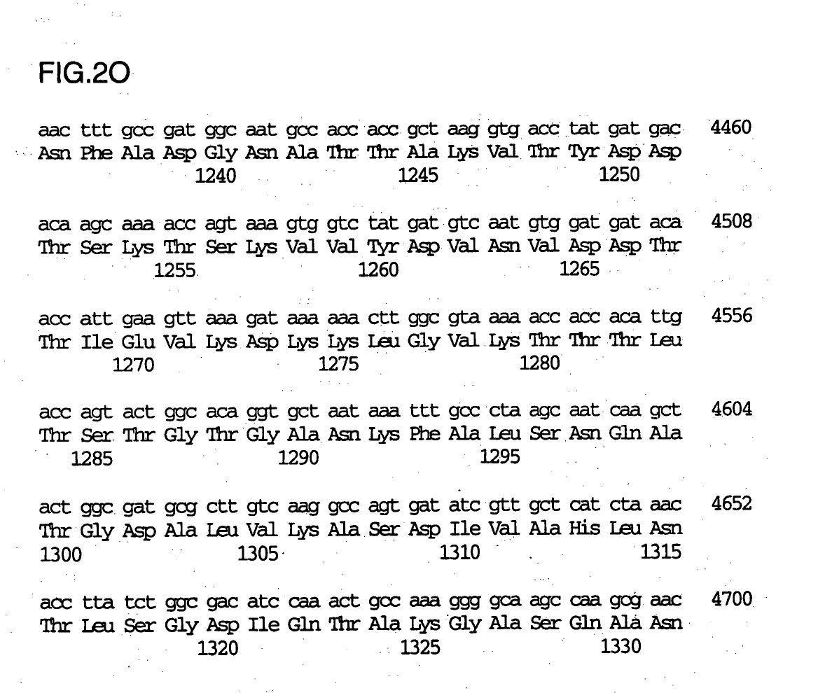

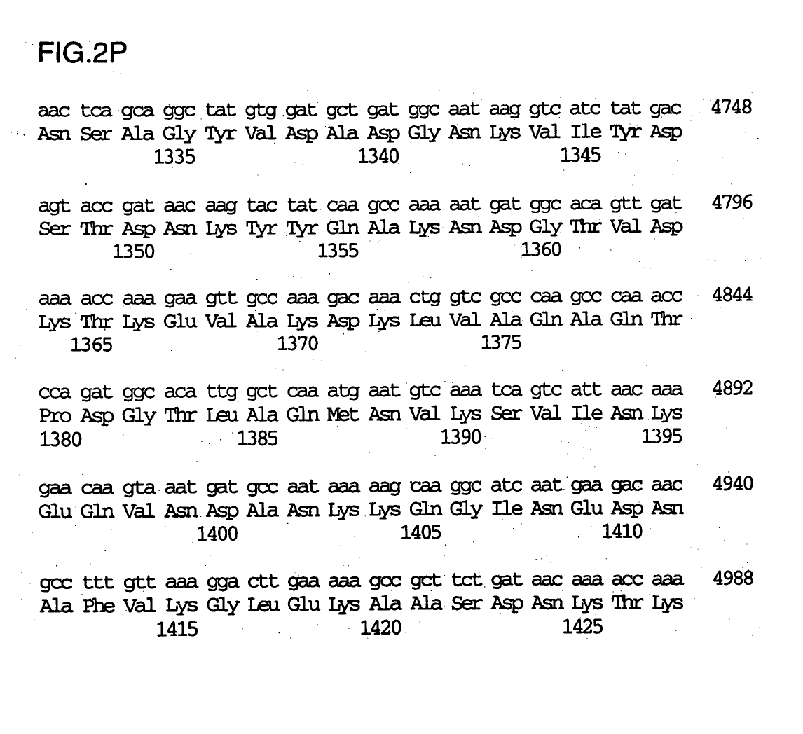

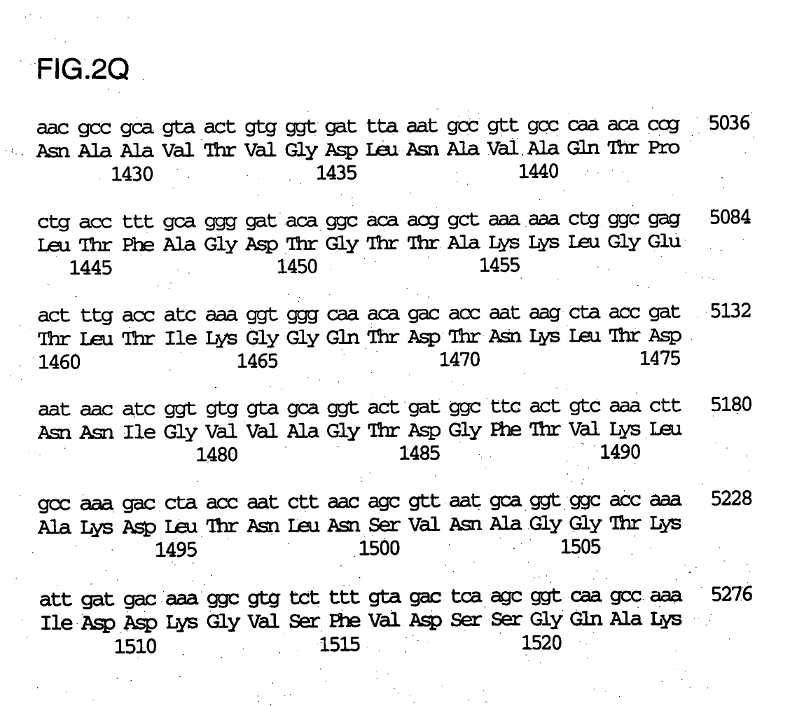

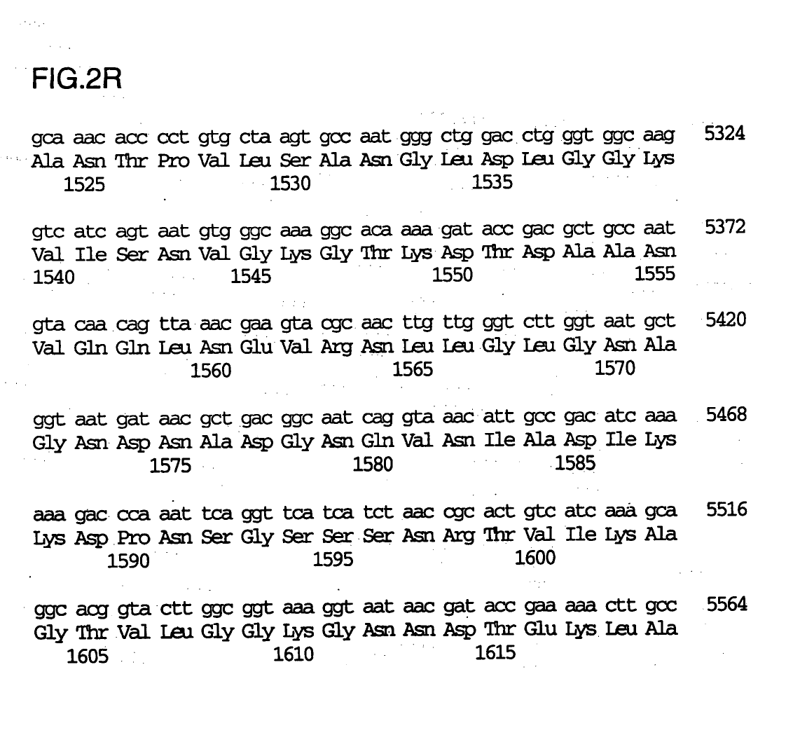

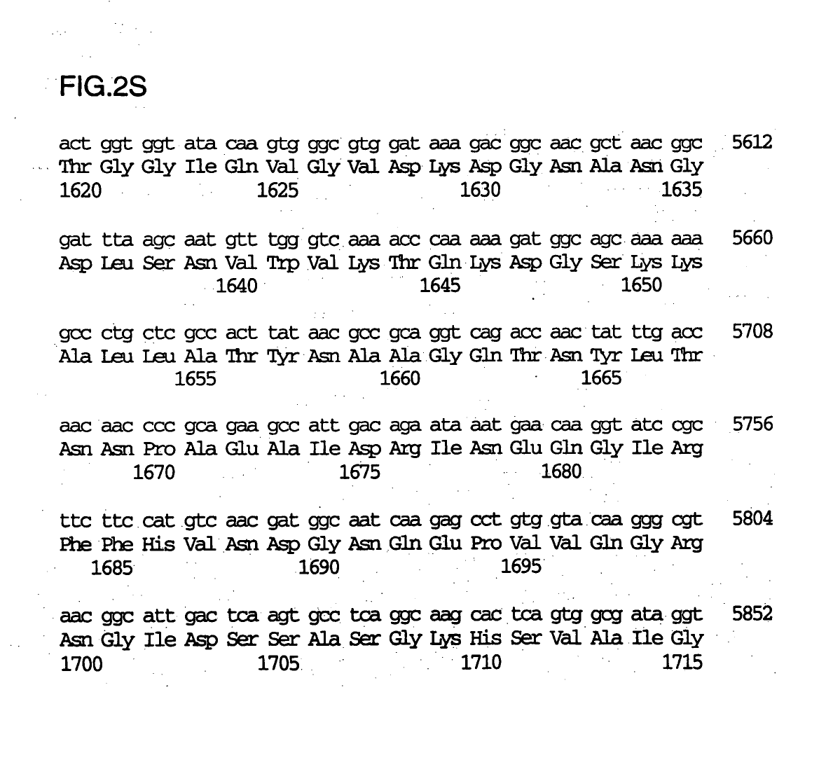

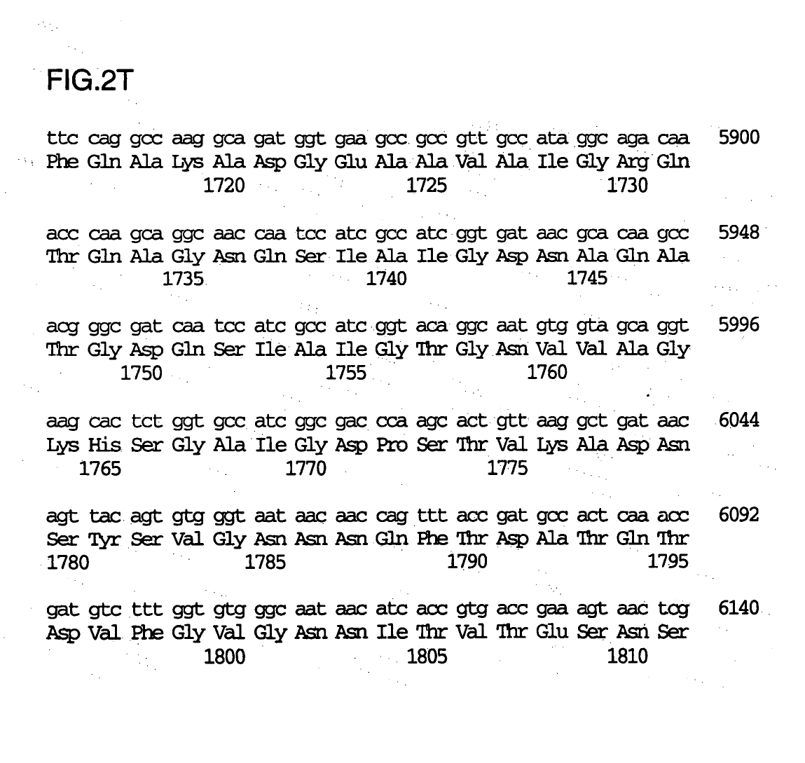

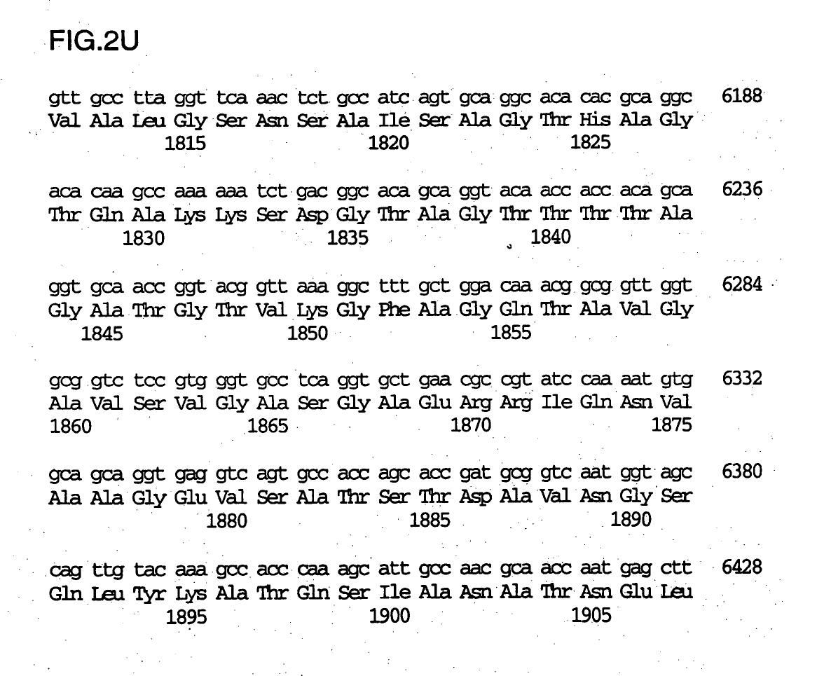

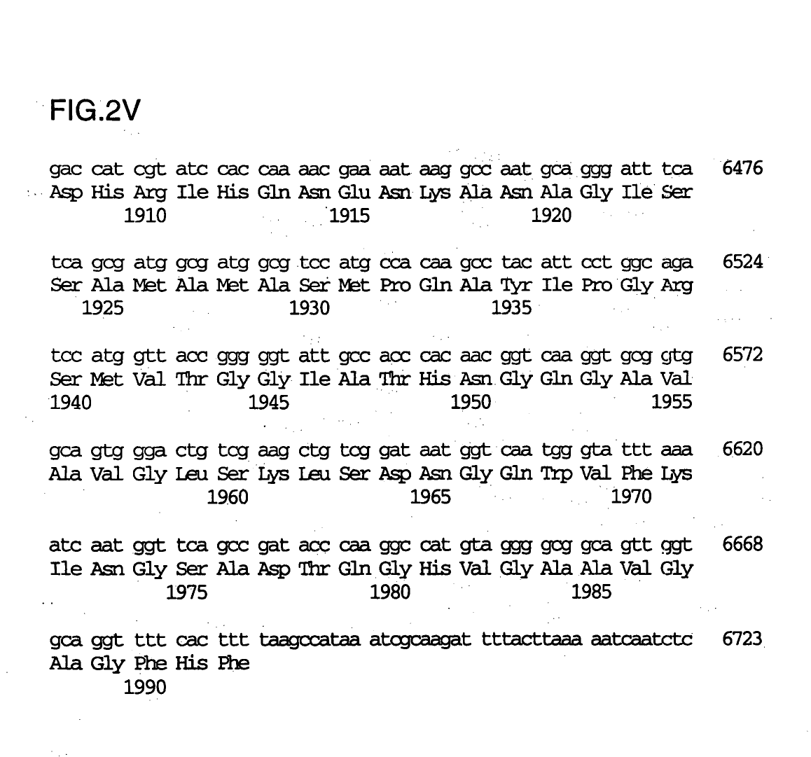

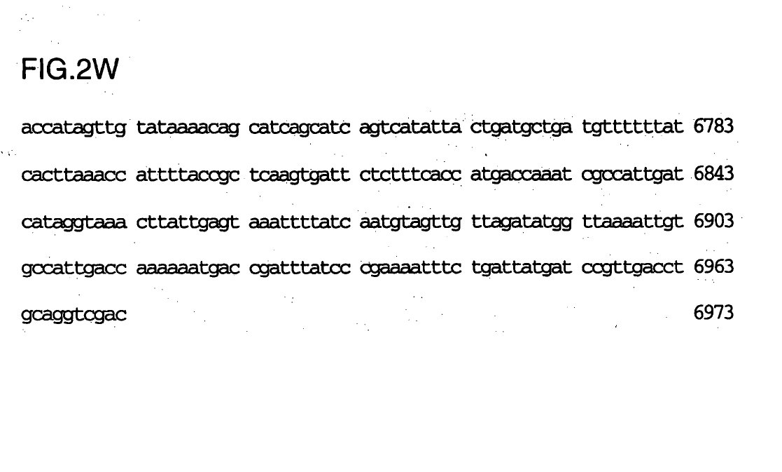

The full sequence of the 200 kDa gene locus from the λDNA clone was described in U.S. Pat. No. 5,808,024 and WO 96/34960 and is shown in FIG. 2. There is a tract of 10 consecutive G nucleotides between position 623 and 632 in clones derived from the λ library. The first possible start codon is, therefore, located at nucleotides 706 to 708 and is a GTG encoding a valine, boxed lightly in FIG. 2. A series of strains expressing a 200 kDa gene, were identified by immunoblot analysis and the 5′ end of their 200 kDa genes was PCR amplified and sequenced. A summary of the findings is shown in Table 5 wherein the expression level of the gene appeared to be related to the number of G nucleotides in the tract and for those strains within higher expression levels, the start codon was an ATG upstream of the GTG codon identified from the 4223 λ clones. Based upon these findings, the sequence of the 5′ end of the 200 kDa gene from strain 4223 was re-examined.

Plasmids pKS9 and pKS10 were directly derived from the λ clone. The subclones pKS59 and pKS63 were derived from pKS9 whereas pKS71 contained the same fragment derived directly from the λ clone. All of these plasmids contained 10 G nucleotides in the G tract, as described previously. To determine whether the λ clone contained an extra G nucleotide or the strain itself contained an aberrant gene, PCR amplification of the region was performed from chromosomal DNA preparations and from the λ subclones. The data in Table 3 show that PCR fragments of the λ subclones all contained 10 G nucleotides. The data in Table 4, however, demonstrate that PCR fragments derived directly from chromosomal DNA, contain 9 G nucleotides in the tract. When the single extra G nucleotide is removed from the 200 kDa sequence of strain 4223, the open reading frame is extended in the 5′ direction to start from an ATG codon 156 nucleotides earlier, at positions 541 to 543 in FIG. 2. This new start codon corresponds to that suggested for the 200 kDa genes sequenced from other strains and summarized in Table 5.

### Example 4

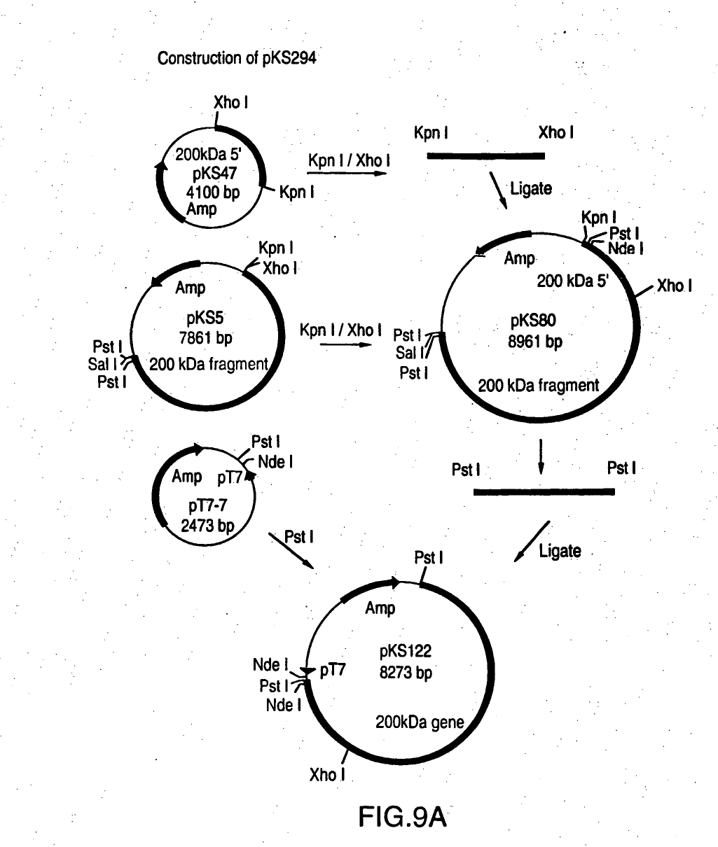

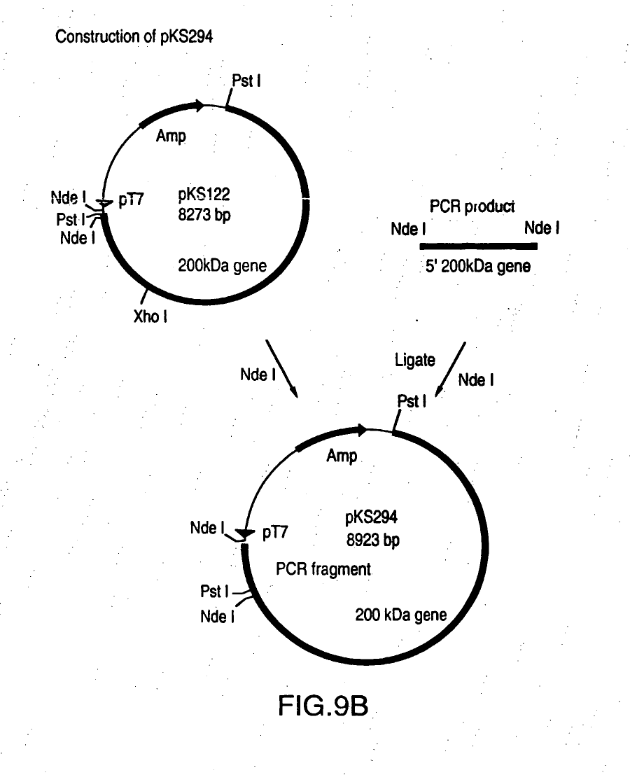

This Example describes the construction of the full length 200 kDa protein gene from M. catarrhalis strain 4223. The construction scheme is shown in FIG. 9.

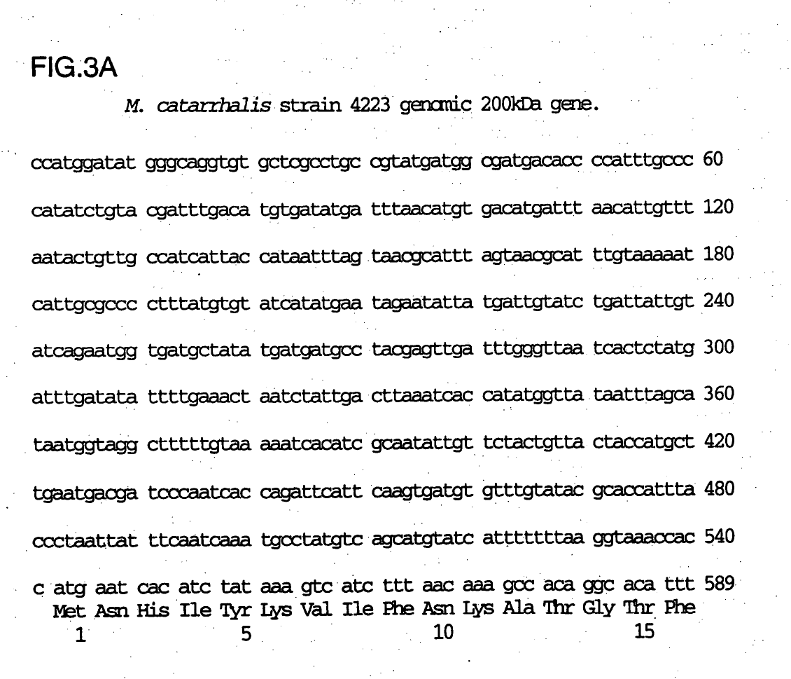

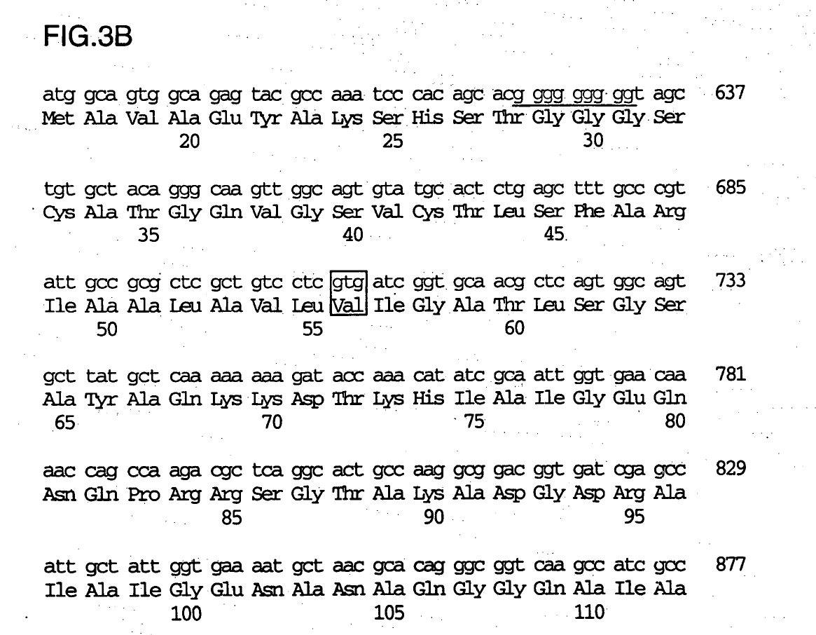

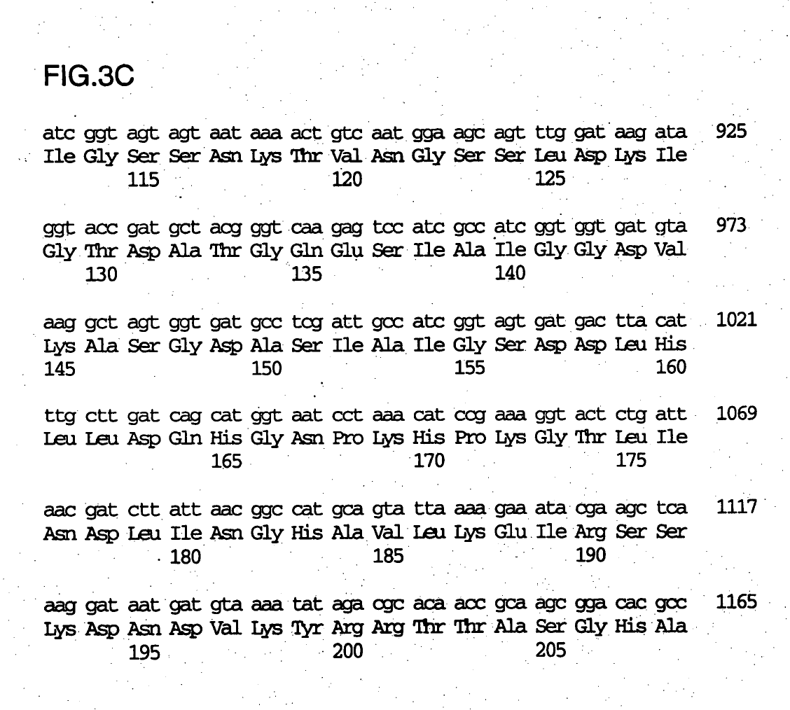

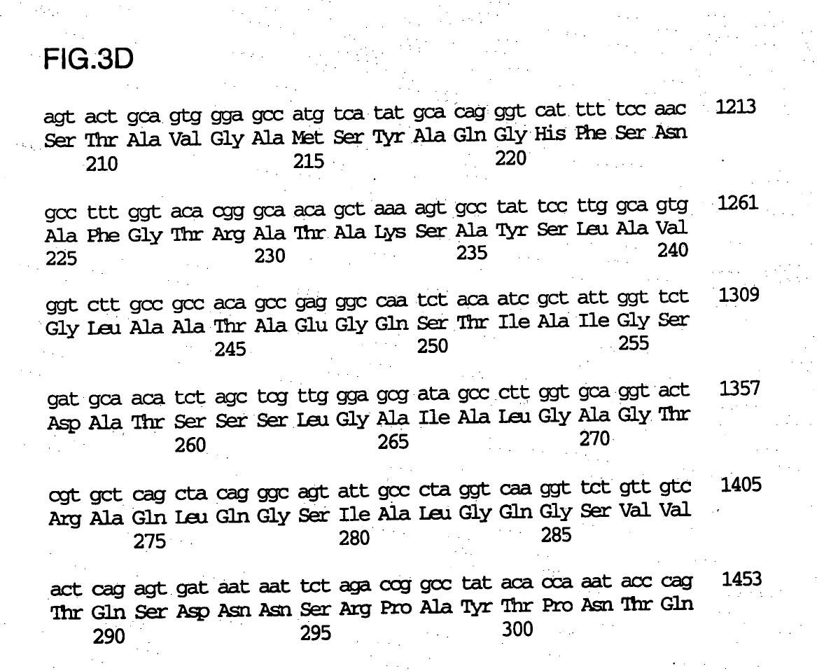

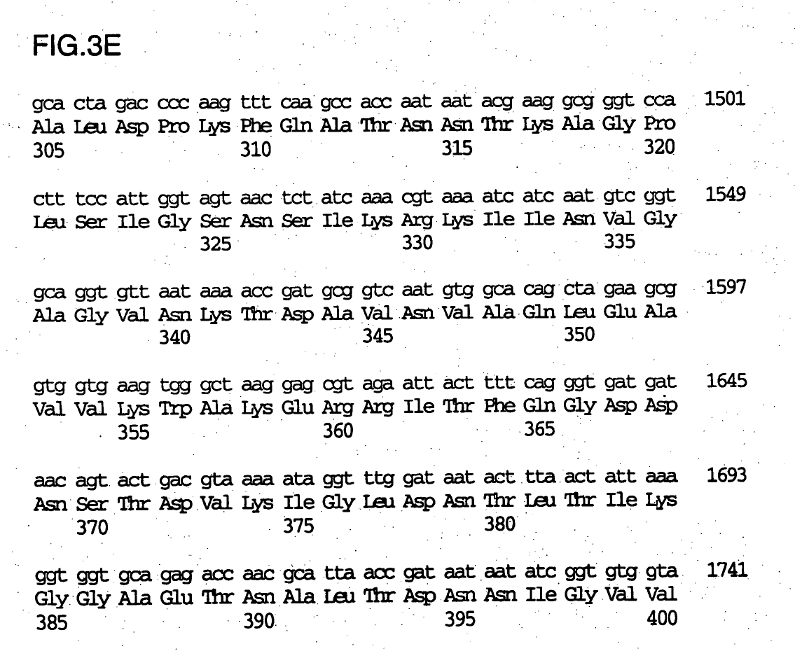

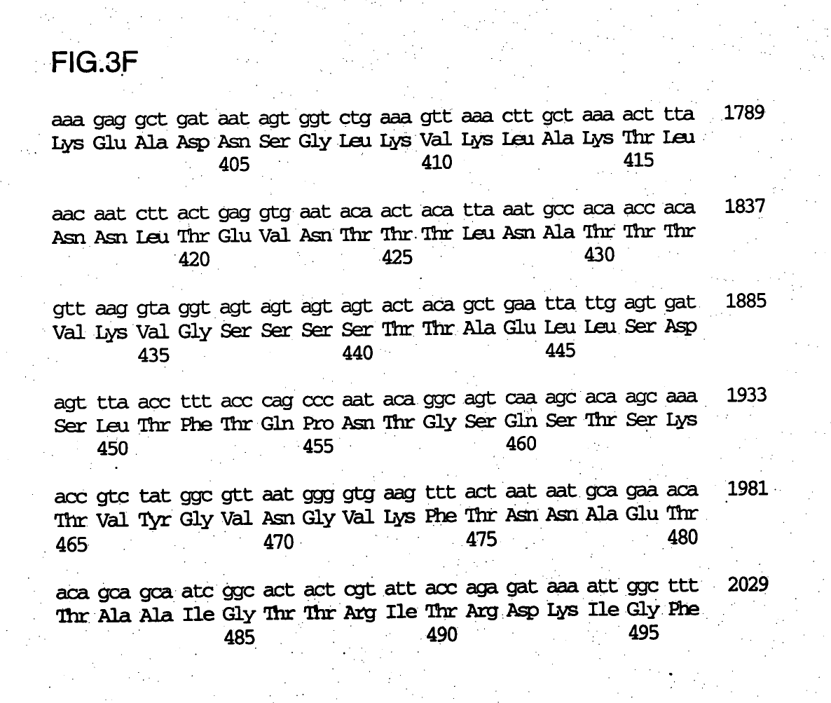

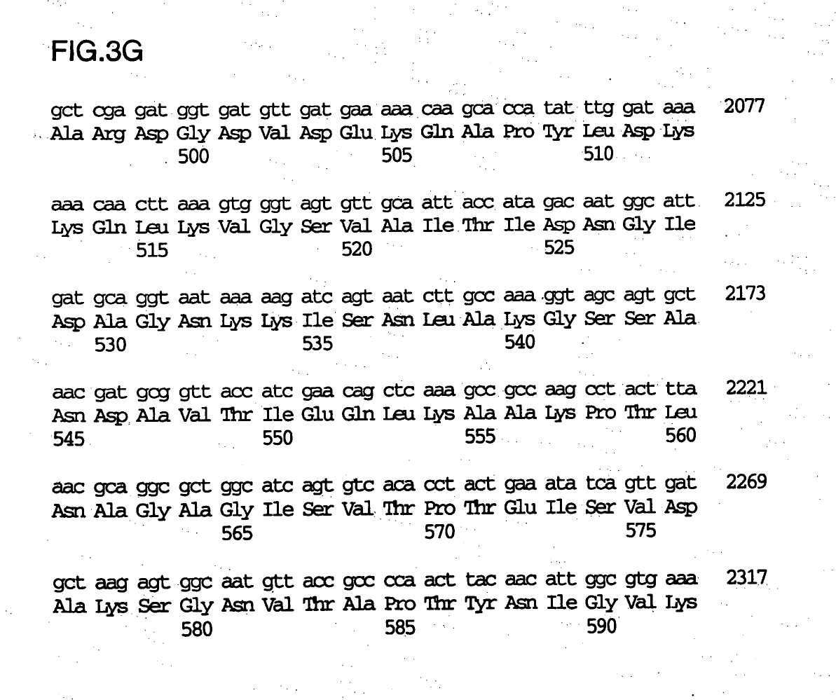

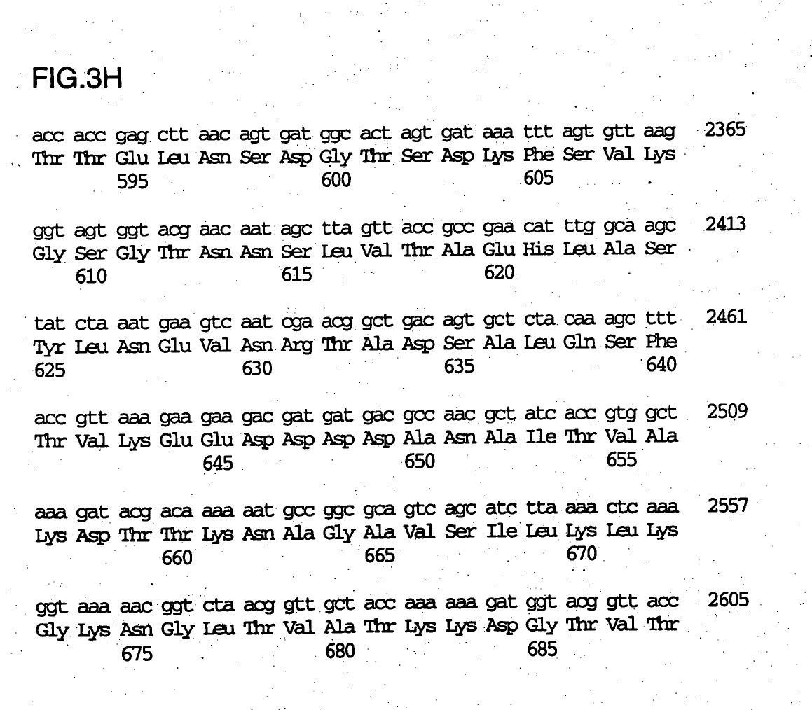

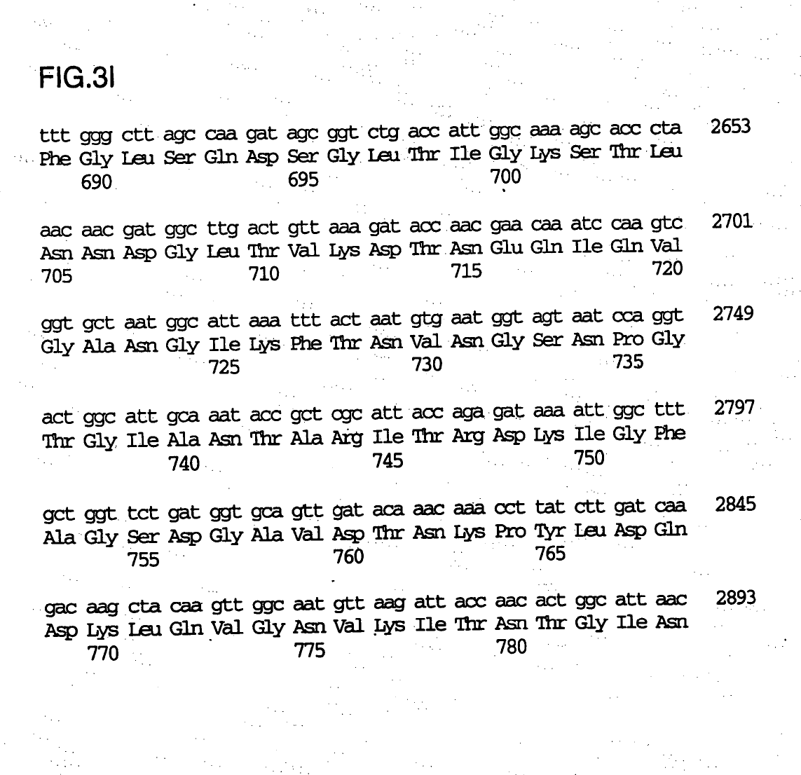

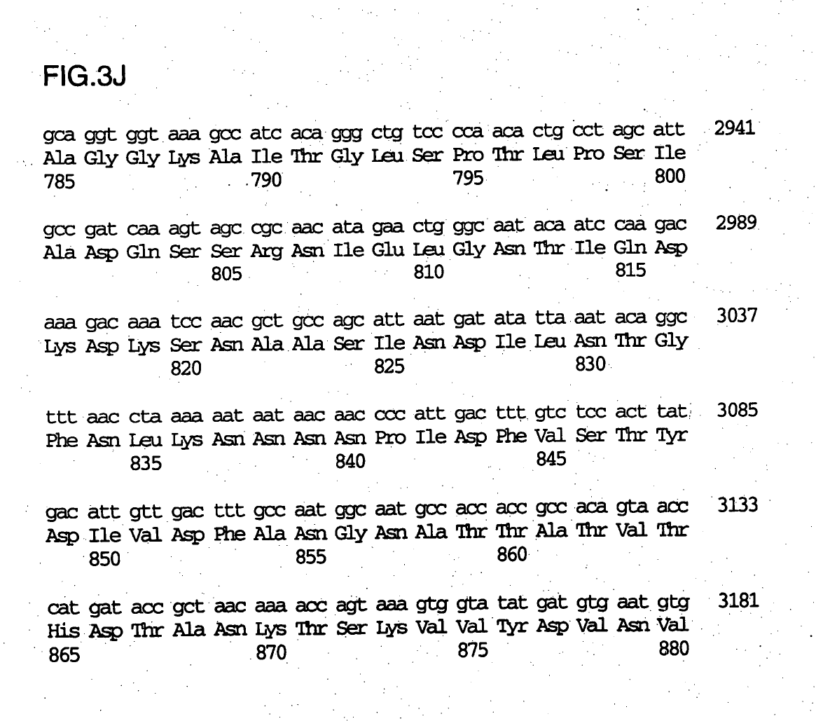

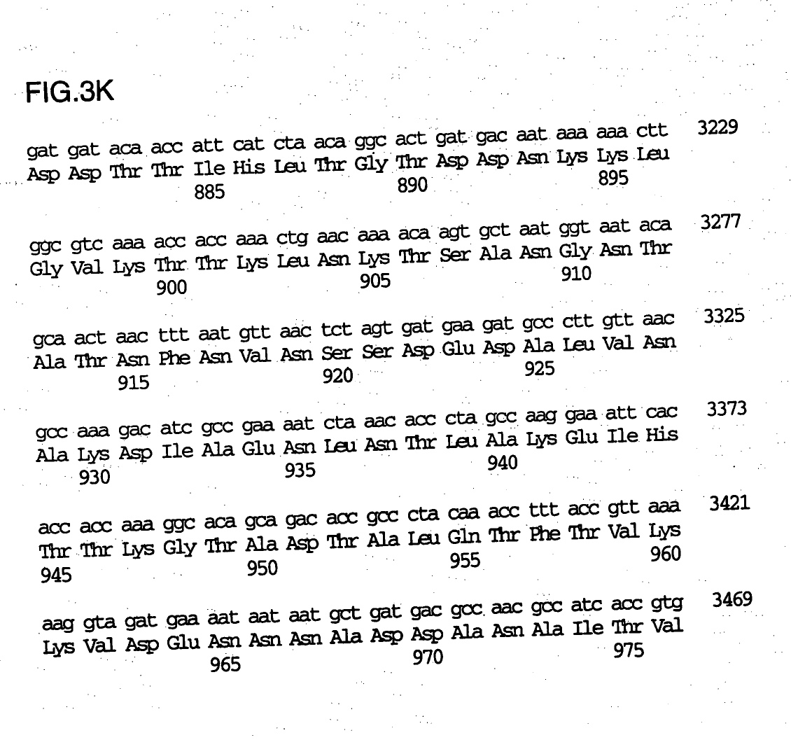

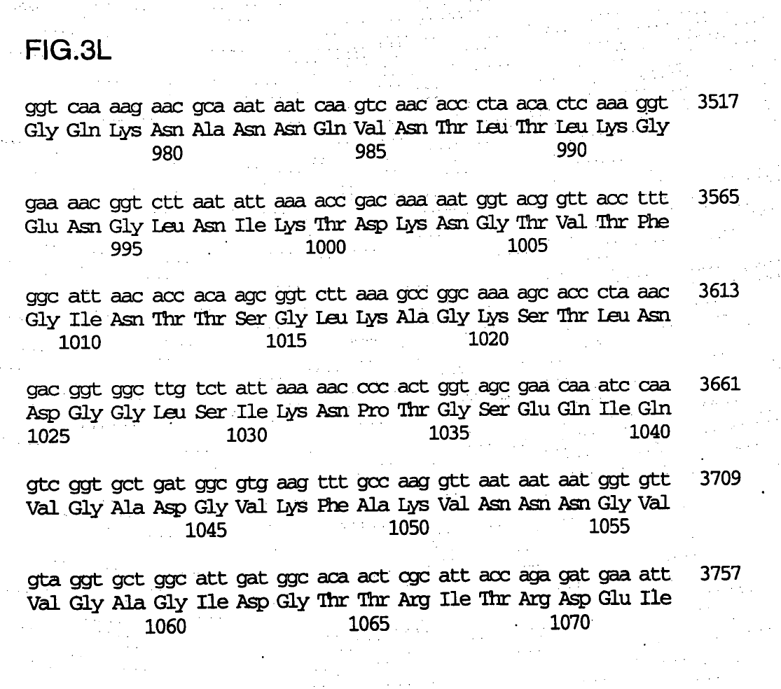

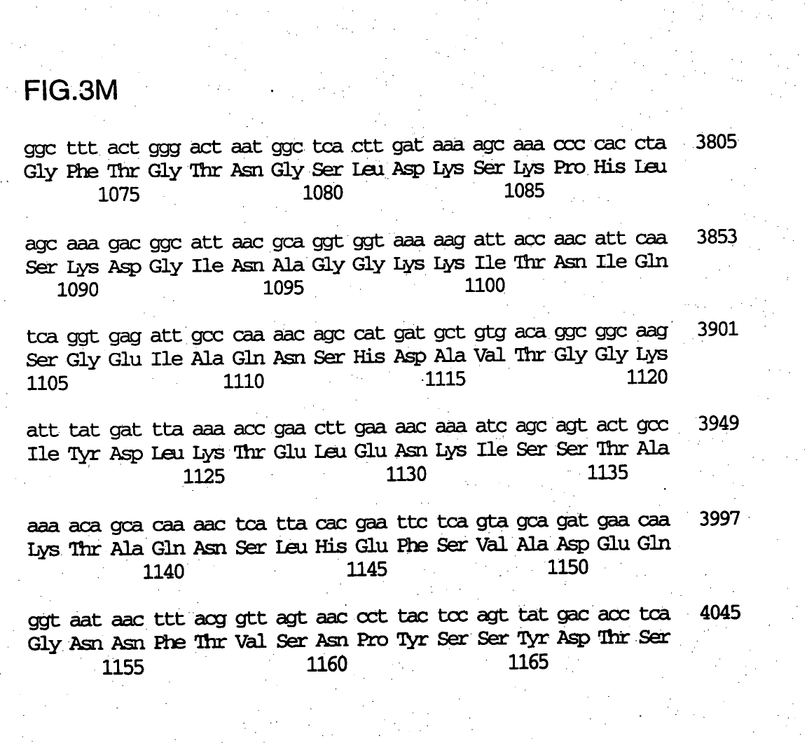

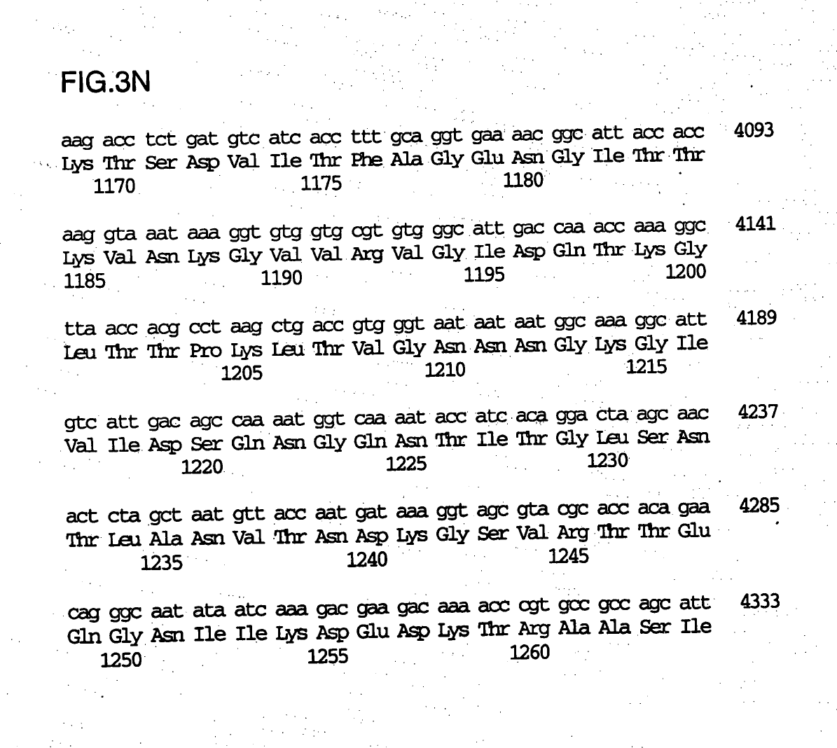

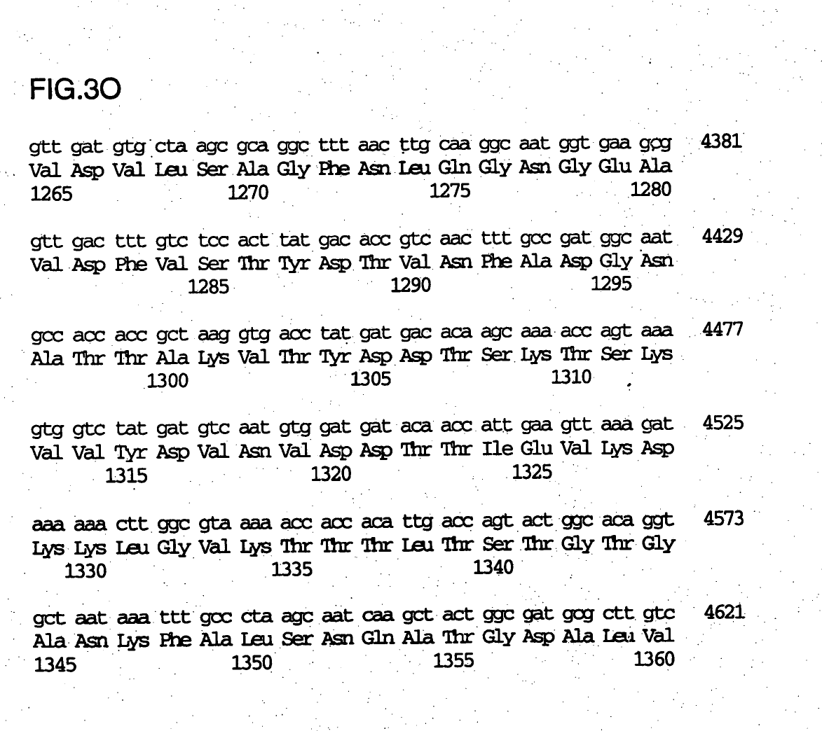

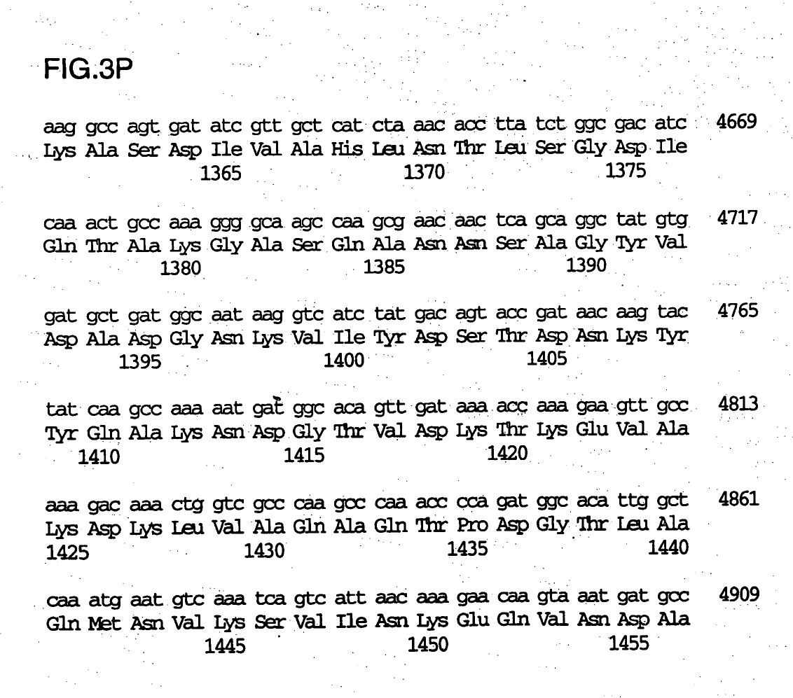

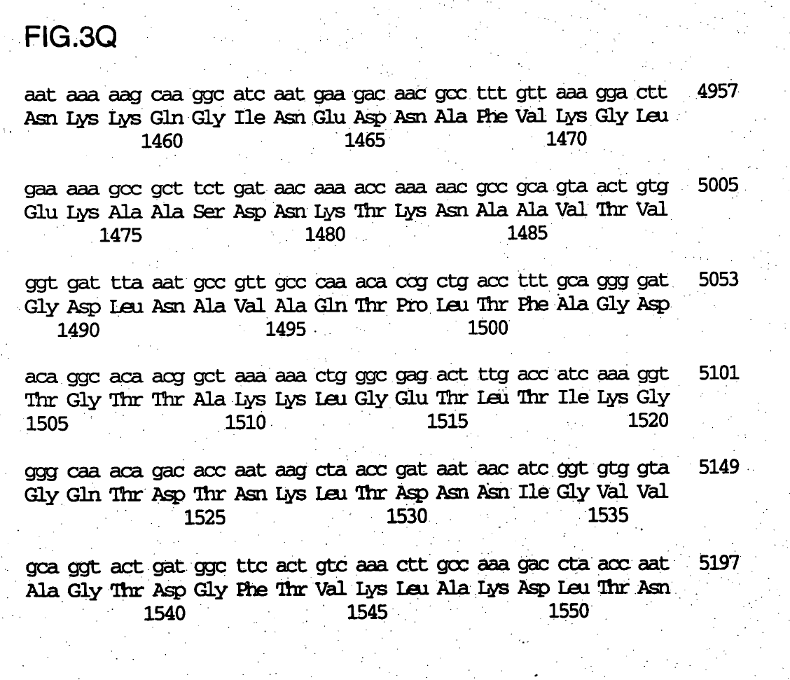

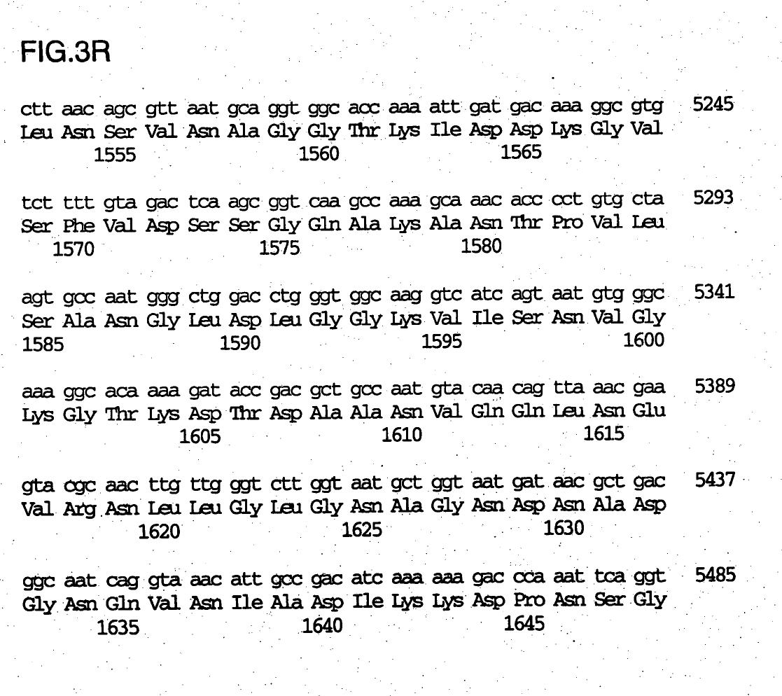

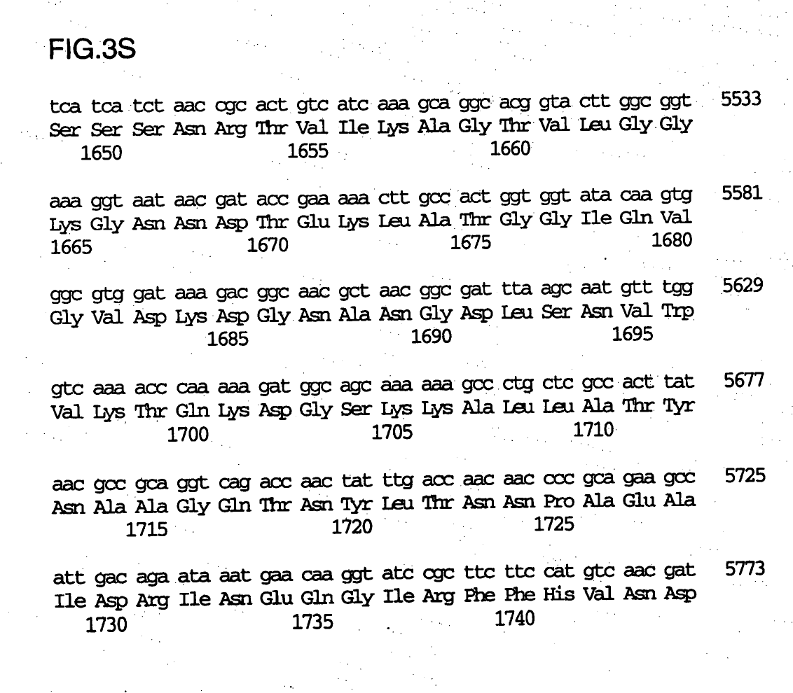

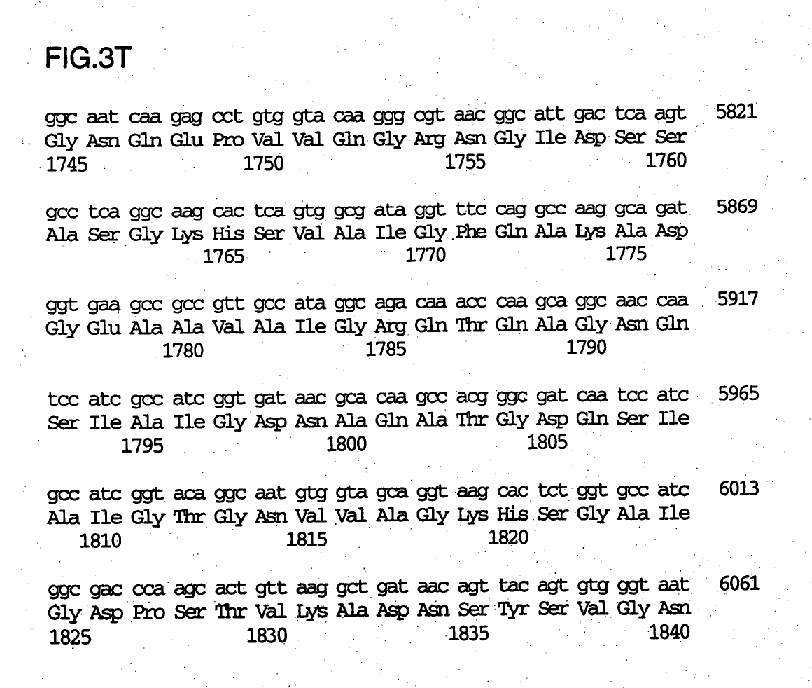

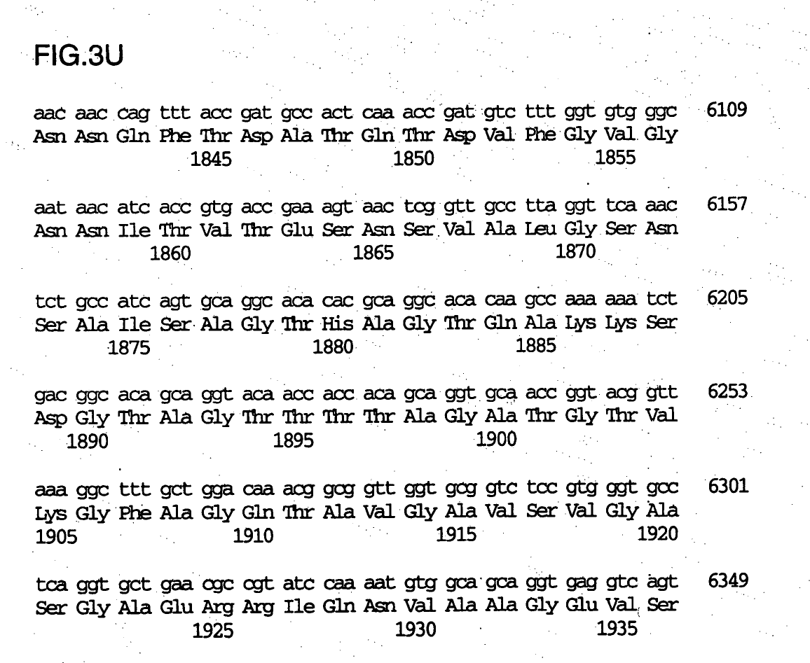

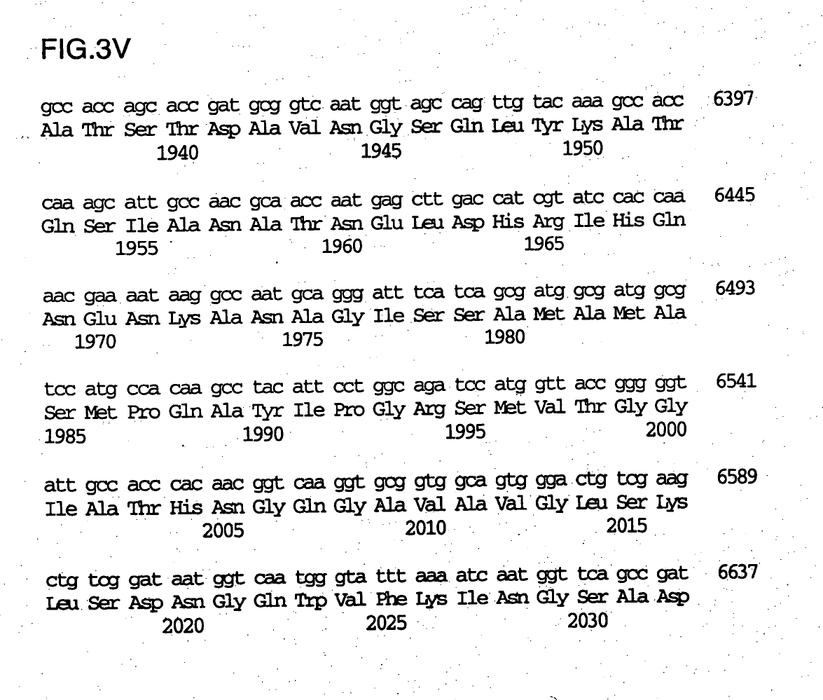

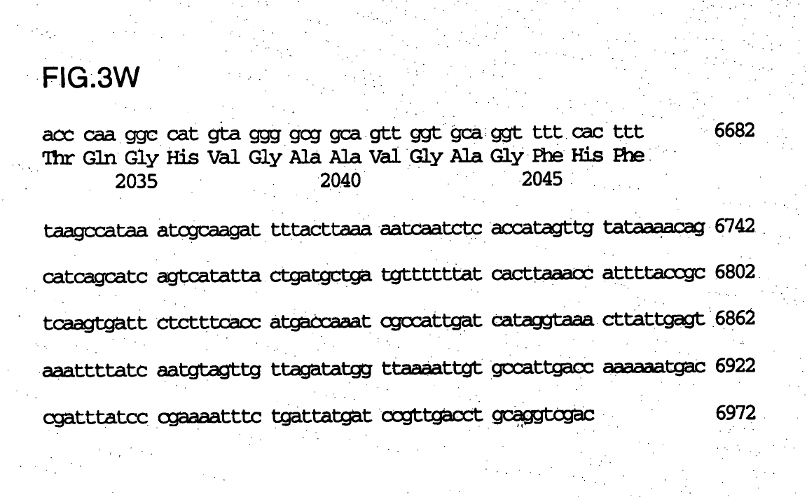

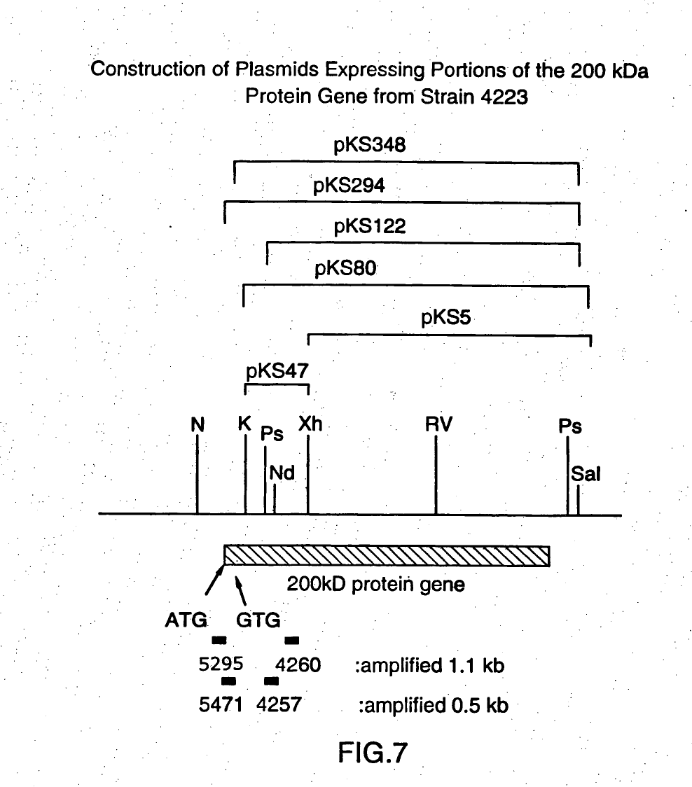

The full-length 200 kDa protein gene was constructed from the new ATG start codon identified by analysis of the chromosomally derived DNA as described in Example 3 and shown in FIG. 3. pKS47 was digested with XhoI and KpnI and separated by agarose gel electrophoresis. The 1.1 kb fragment was isolated from the gel and inserted into pKS5, which had previously been digested with the same two enzymes and purified to form pKS80. An about 5.8 kb PstI fragment from pKS80 was inserted into pT7-7 vector (ref. 28) that had been digested with PstI and dephosphorylated. The orientation of the insert was determined by restriction enzyme analysis and pKS122 was chosen for further construction (see FIG. 7).

The 5′ region of the 200 kDa protein gene was amplified from strain 4223 chromosomal DNA. PCR reactions were performed using Taq Plus or Tsg Plus enzyme (Sangon Ltd., Scarborough, Ont., Canada) and a Perkin Elmer DNA Thermocycler (Perkin Elmer Cetus, Foster City, Calif., USA). The lower PCR reaction mixture (50 μl) contained 5 μl of 10× buffer, 0.4 mM each of four deoxynucleotide triphosphates (Perkin Elmer, Foster City, Calif., USA) and 1 to 2 μM each of two primers. The upper PCR reaction mixture (50 μM) contained 5 μl of 10× buffer, 0.5 to 1 μl of Taq Plus or Tsg Plus enzyme, and template DNA. The lower and upper mixtures were separated by a layer of AmpliWax PCR Gem50 (Perkin Elmer, Foster City, Calif., USA) before heating cycles started. The thermocycling condition employed for the provision of PCR products in the construction of various plasmids are set forth in Table 11 below. The PCR products were purified using a QIAquick PCR purification kit (Qiagen Inc., Mississauga, Ont., Canada). The purified PCR products were sequenced on both strands directly and/or after cloning in appropriate vectors using an Applied Biosystem sequencer.

The 5′ primer (designated 5295.KS) was designed, so that it contained the first possible translation start codon, ATG, and its flanking sequences with a mutation to introduce an NdeI site at the ATG. The 3′ primer (designated 4260.KS) was based upon the non-coding strand in the region about 1 kb downstream from the ATG start codon. (The nucleic acid sequences and SEQ ID's of the PCR primers utilized herein are identified in Table 10). The PCR-product was digested with NdeI and an approximately 650 bp DNA fragment was gel purified and inserted into pKS122, which had previously been linearized with NdeI and dephosphorylated.

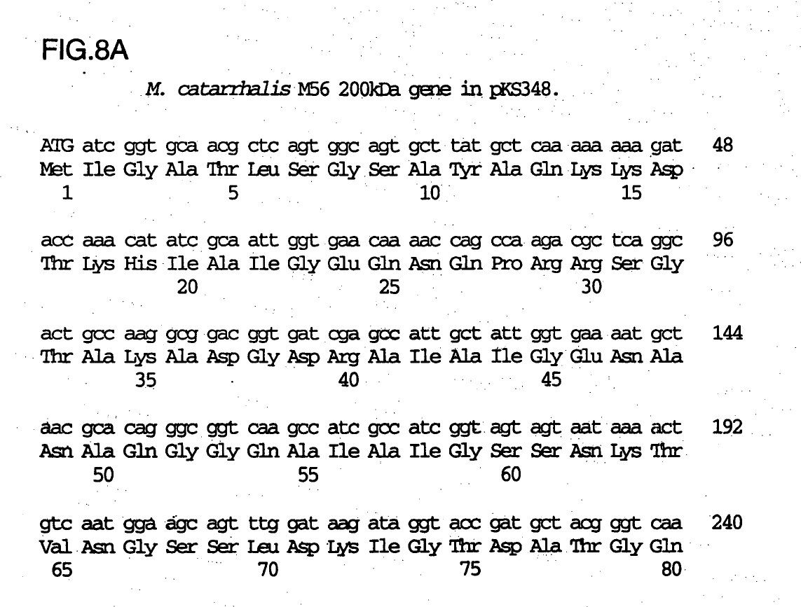

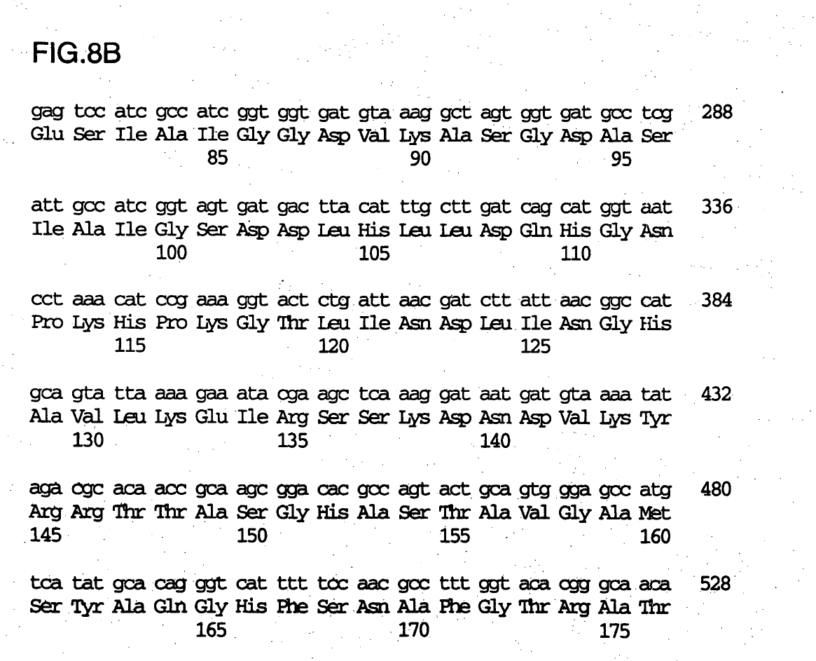

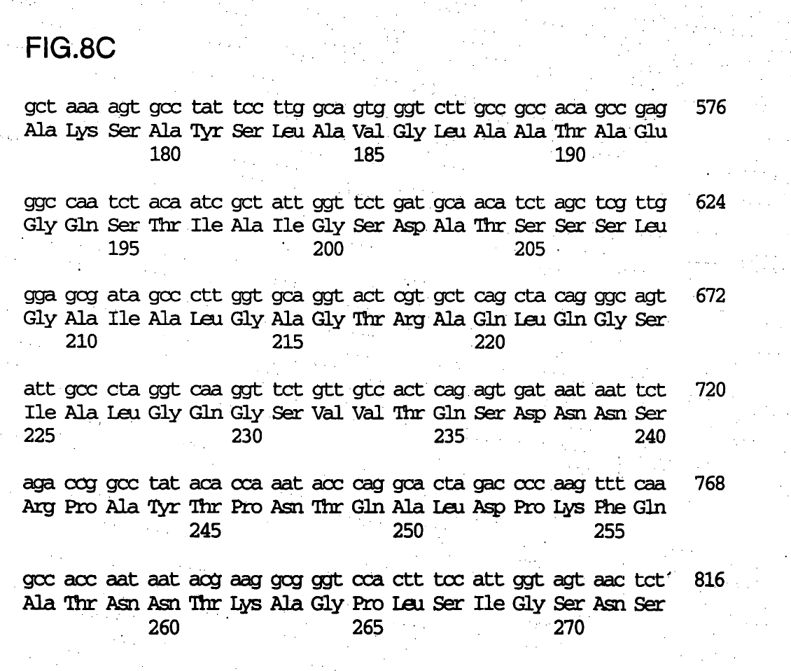

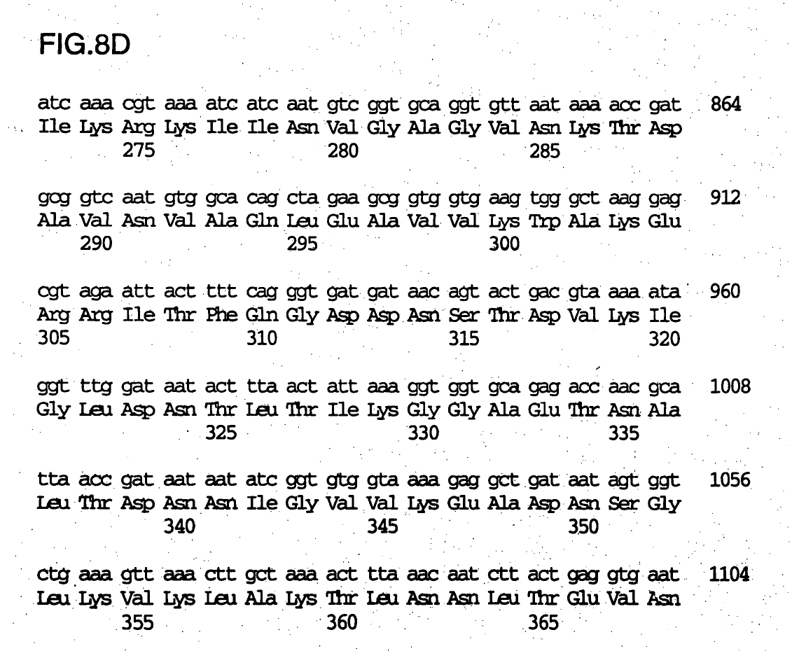

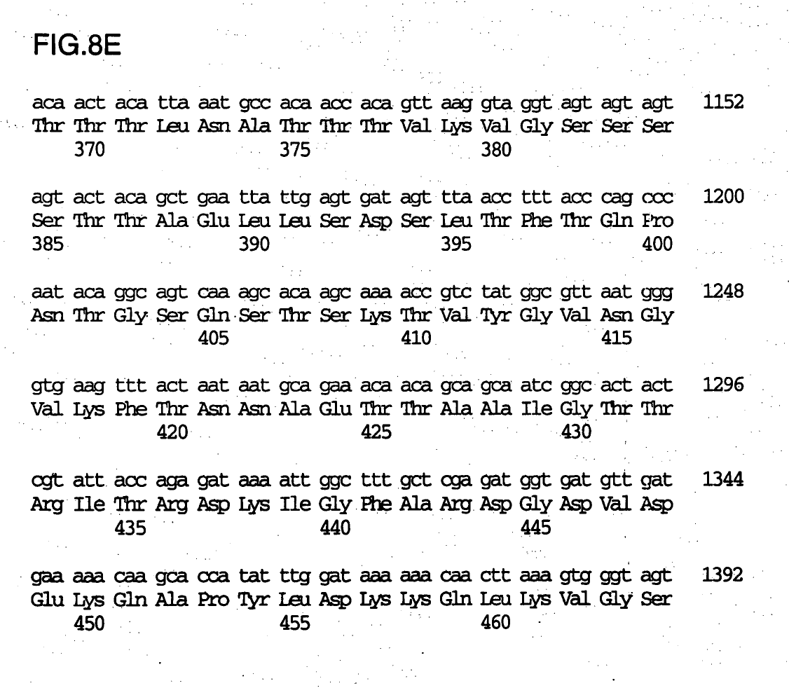

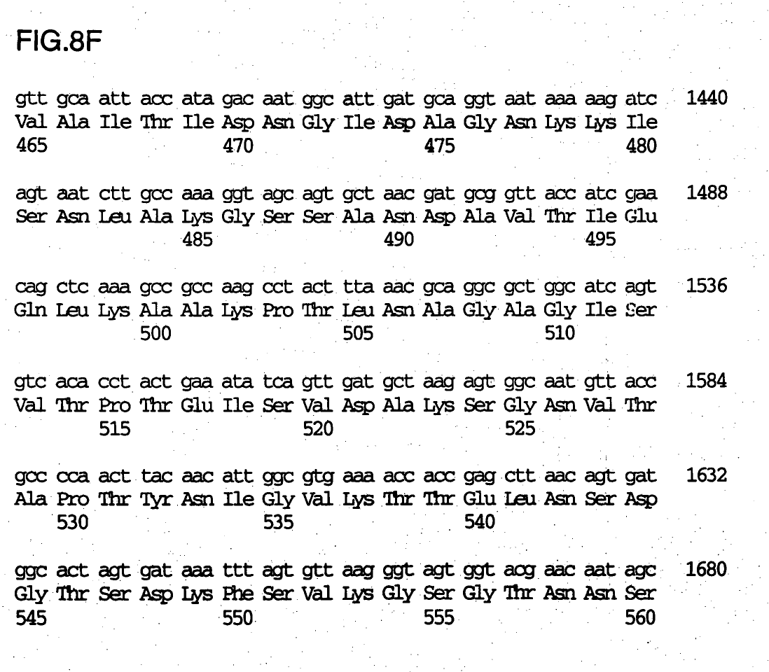

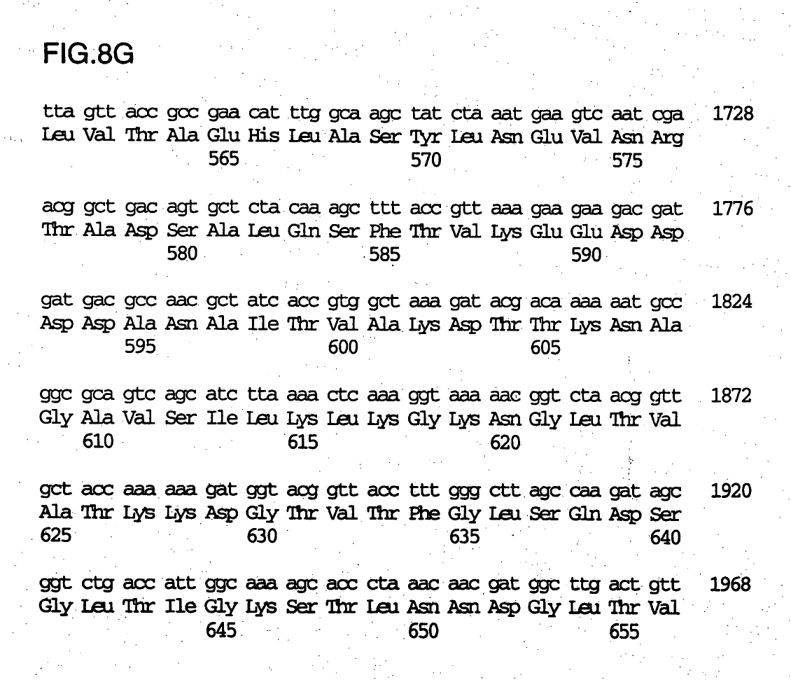

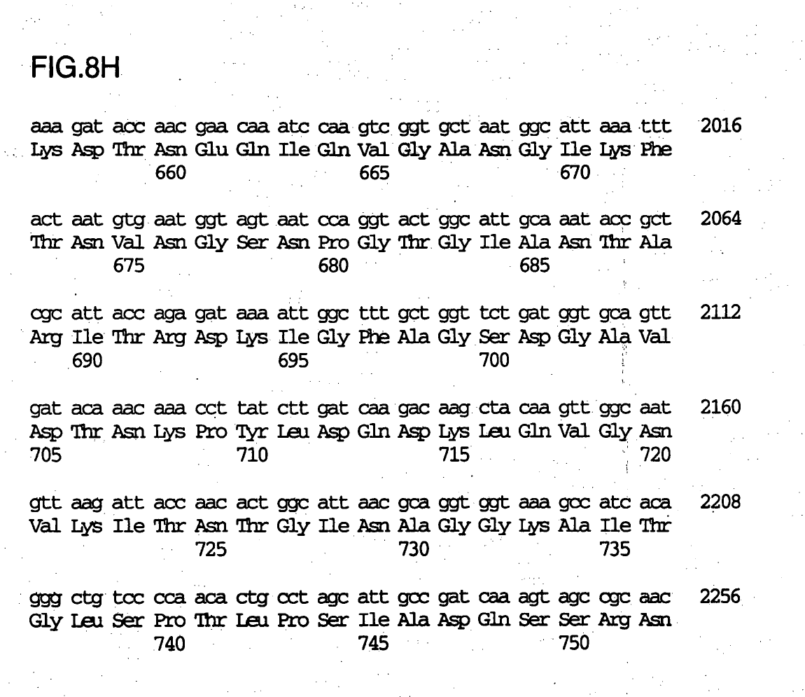

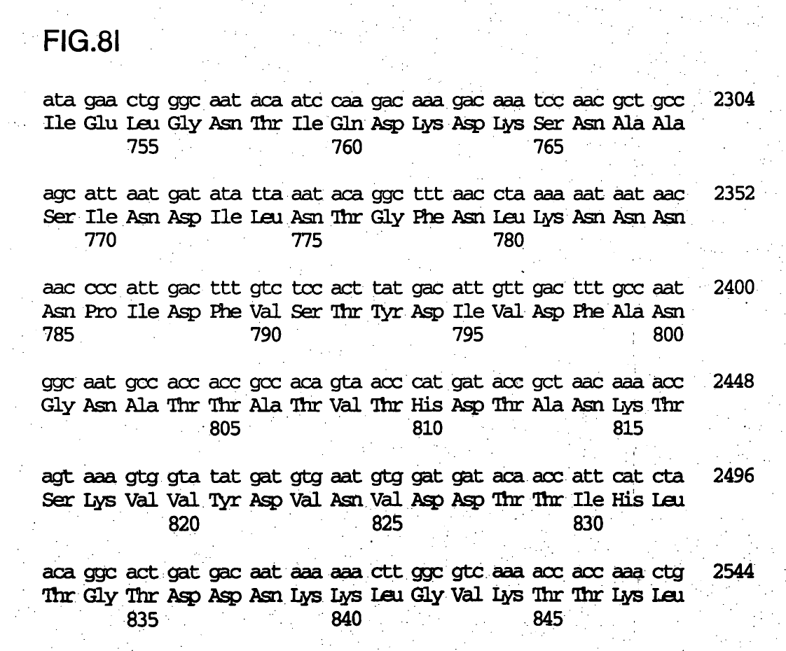

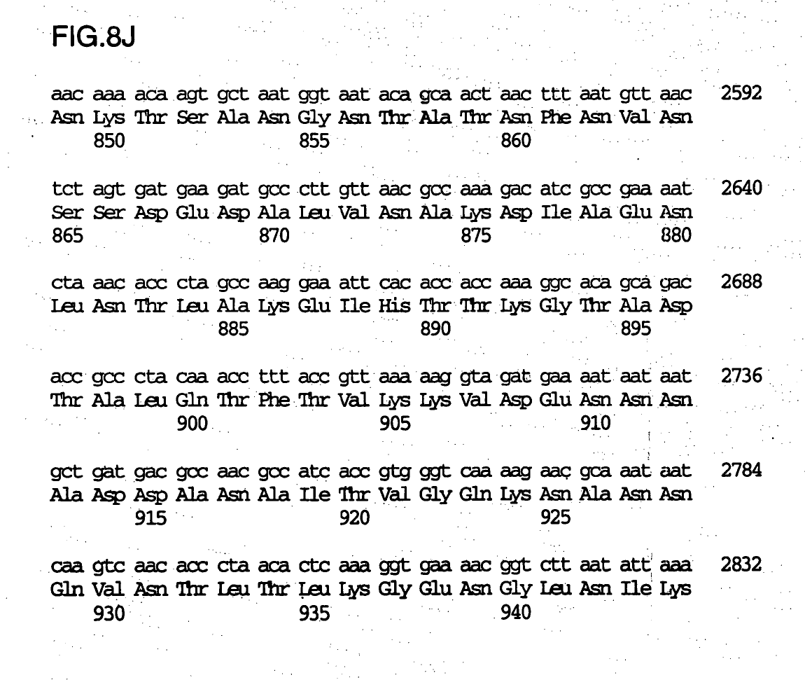

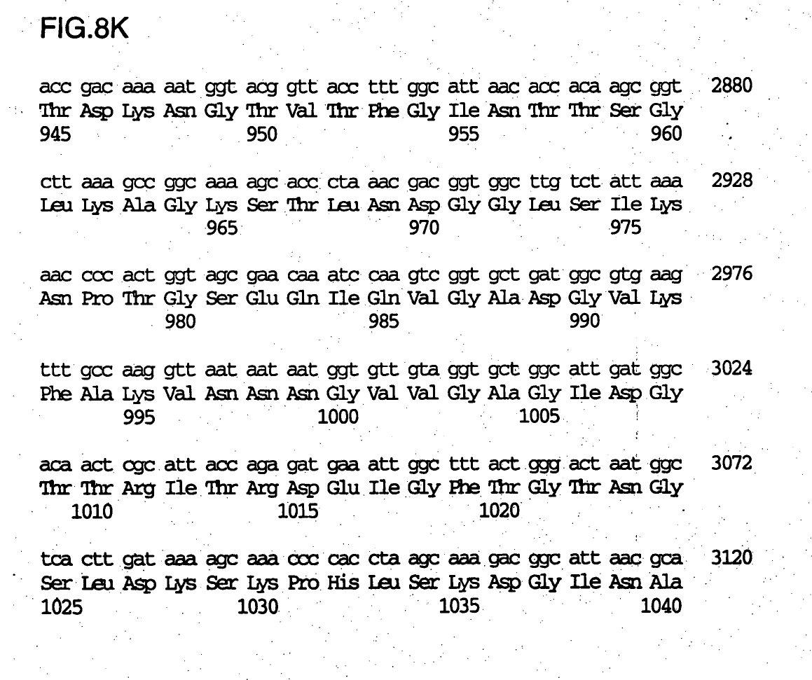

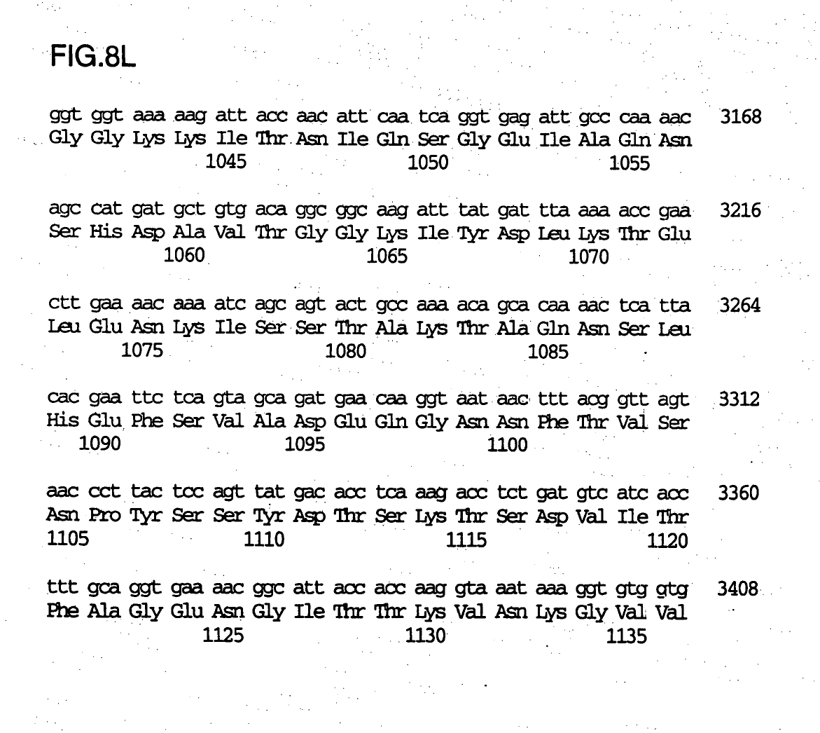

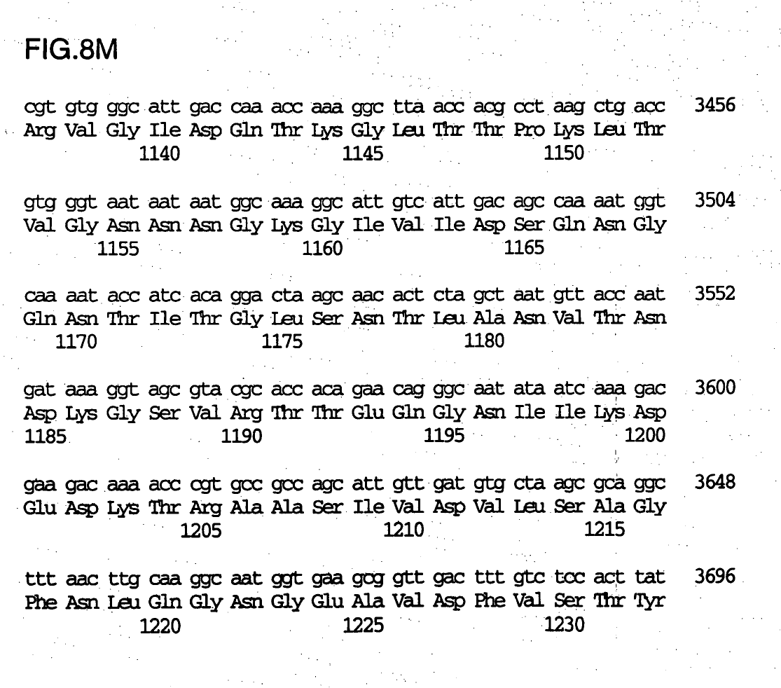

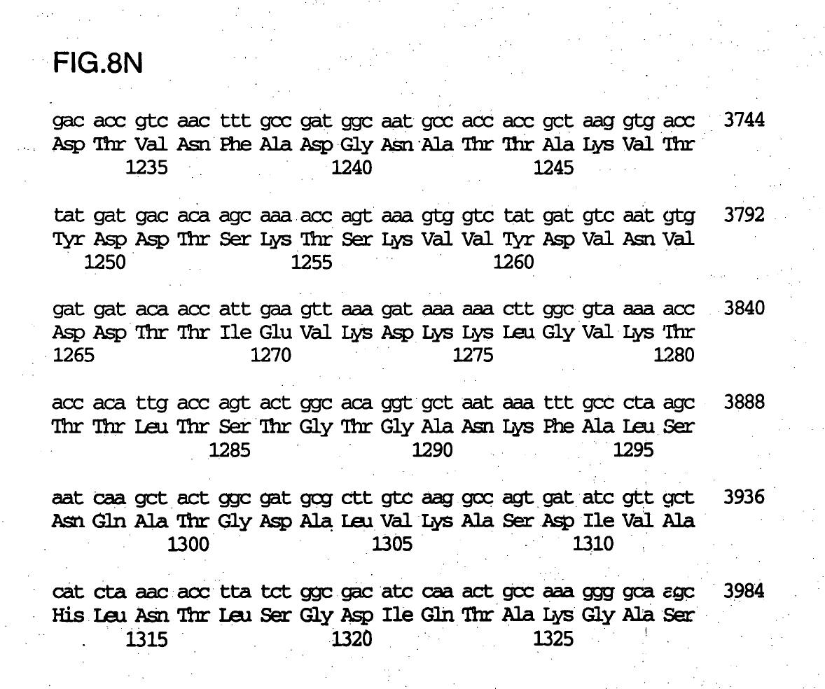

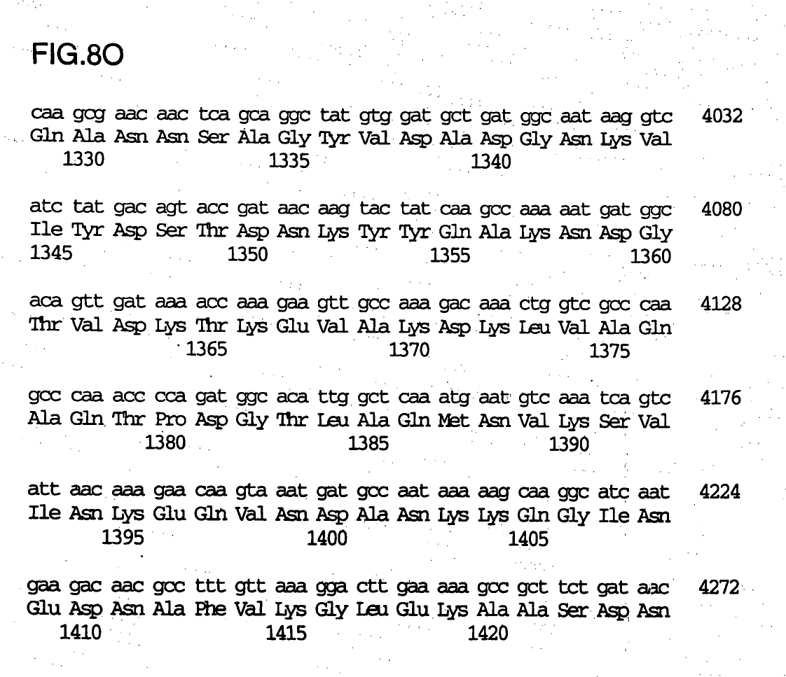

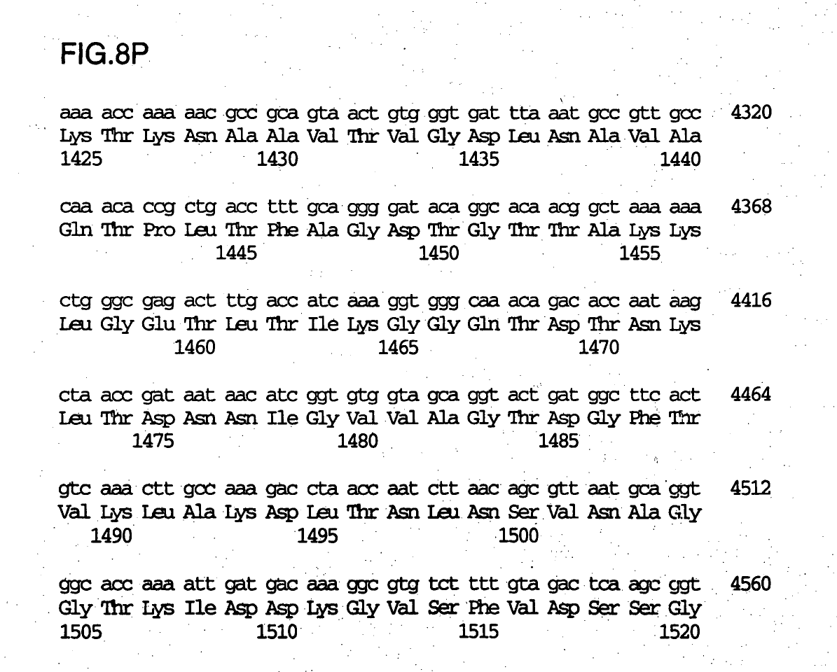

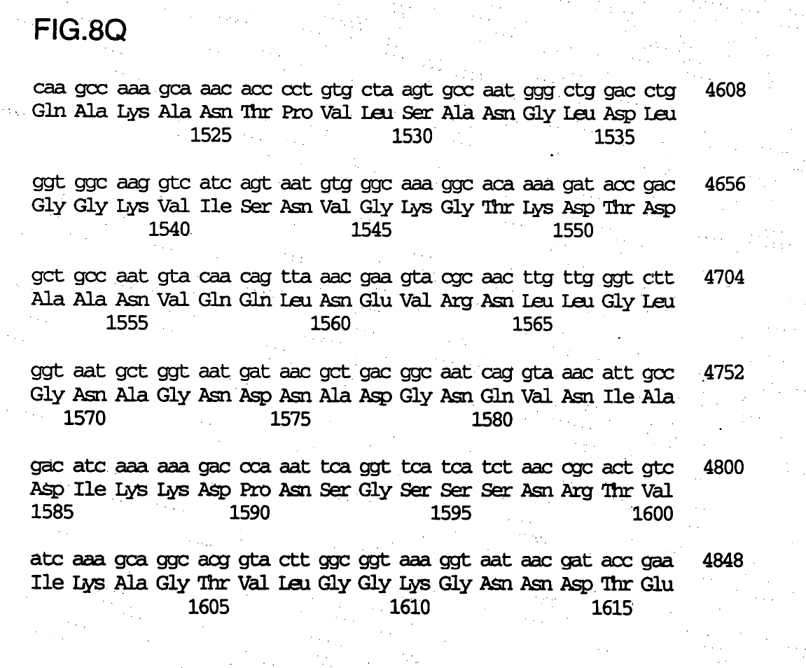

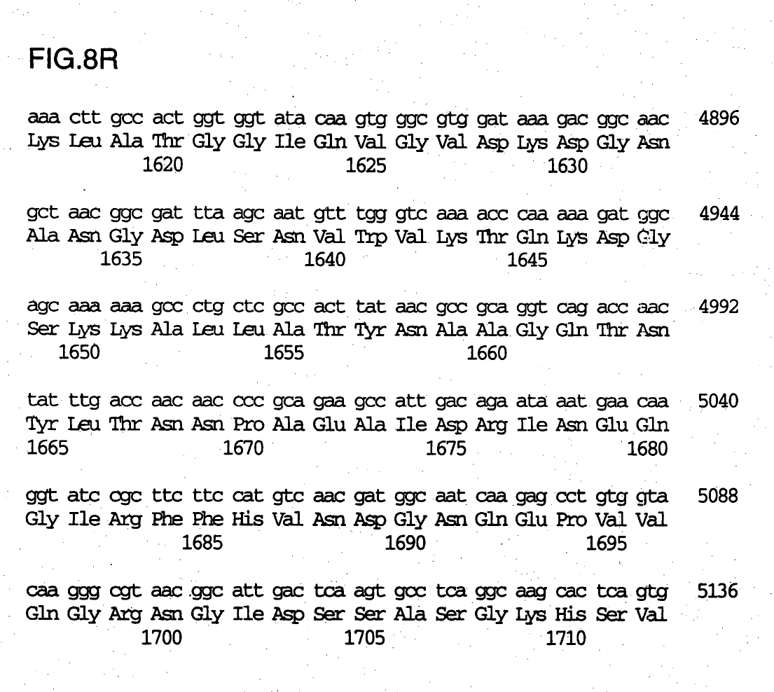

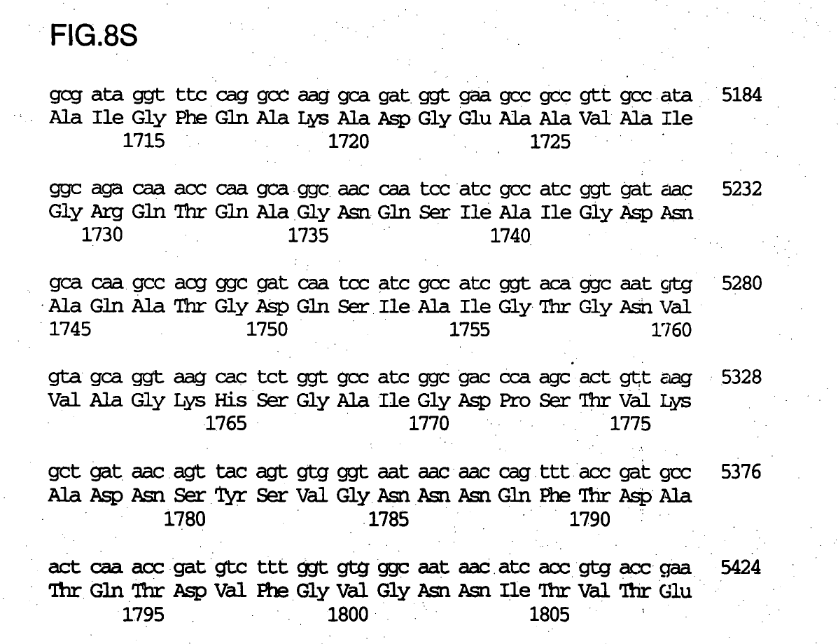

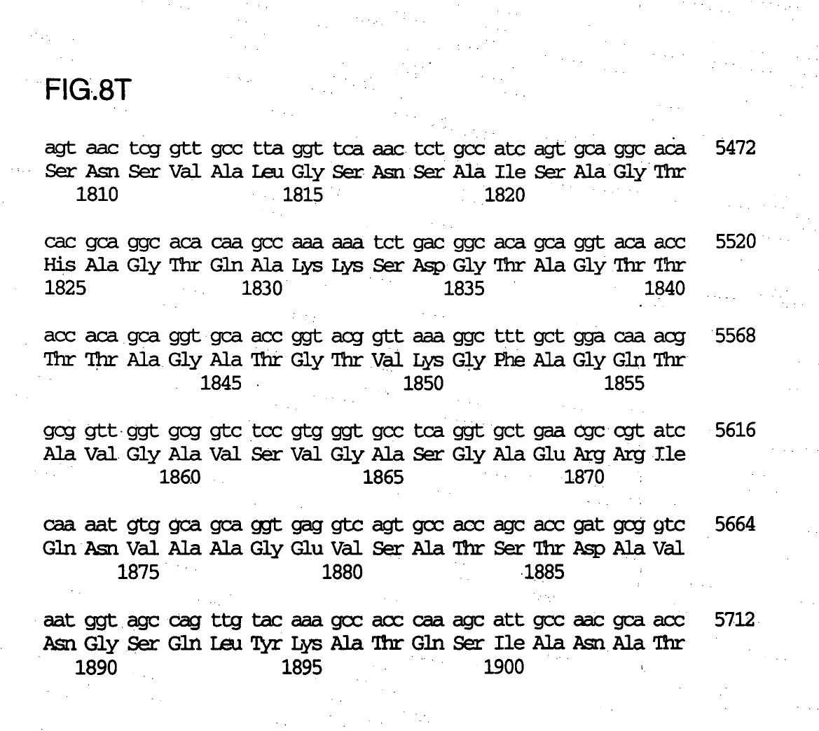

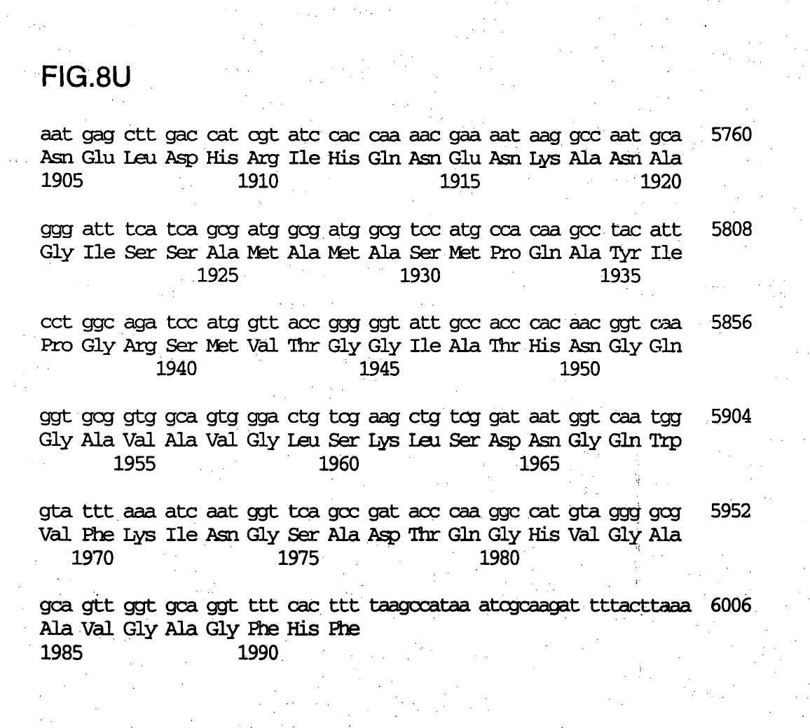

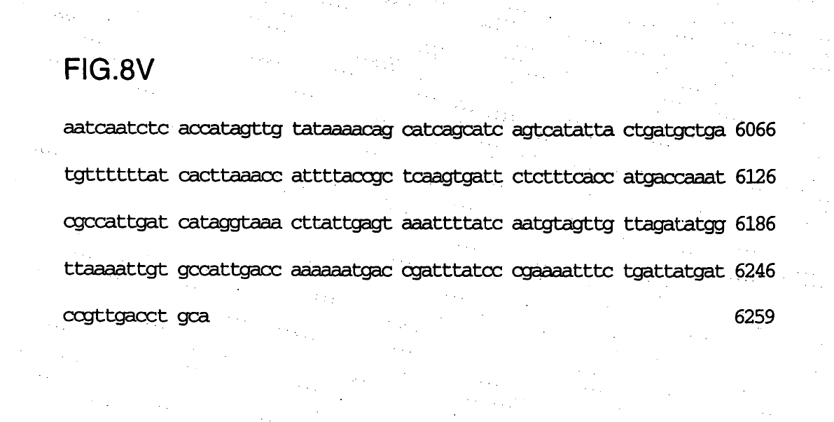

The new construct, designated pKS294 (FIG. 8), was confirmed by restriction enzyme analyses and by sequencing of the PCR-amplified DNA and its joint regions. The number of G nucleotides in the G tract was nine, and the open reading frame continued from the newly found translation start codon, ATG, to the remaining portion of 200 kDa protein gene in pKS122. pKS294, therefore, carried the correct, full-length 200 kDa protein gene from Moraxella catarrhalis strain 4223. During construction of pKS294, E. coli strain DH5α was used for transformation and plasmid analyses.

### Example 5

This Example describes the cloning and sequence analysis of genes encoding the 200 kDa protein from additional M. catarrhalis clinical isolates.

A panel of M. catarrhalis clinical isolates was analysed by immunoblot with guinea pig anti-200 kDa antibody, as described in U.S. Pat. No. 5,808,024 and WO 96/34960. From these analyses, it was evident that there is size heterogeneity among the 200 kDa proteins from various strains. In order to assess the possible genetic heterogeneity, representative strains were chosen for gene cloning. Strain Q8 is a naturally occurring relatively non-clumping strain that produces a 200 kDa protein of about the same size as the 4223-derived protein. Strain LES-1 produces a larger 200 kDa protein. These strains were also selected based upon bactericidal antibody data as illustrated in Table 1. The 200 kDa genes were cloned from these two strains of M. catarrhalis and sequenced.

The nucleotide and derived amino acid sequences of the 200 kDa genes from strains Q8 and LES-1 are shown in FIGS. 4 and 5 respectively. An alignment of the amino acid sequences with the 4223-derived sequence is shown in FIG. 6. As can be seen, the first 68 residues of the N-terminus are quite conserved, especially between strains 4223 and Q8. In addition, the final 456 residues of the C-terminus are nearly identical among the three strains. The remainder of the sequence has regions of high homology and significant diversity, including an insert of more than 300 residues for strain LES-1.

The N-terminal sequence of the 200 kDa proteins is homologous to the H. influenzae Hia and Hsf proteins, as well as other high molecular weight proteins or adhesins, such as AIDA (ref. 33).

The C-terminal region also has some homology to H. influenzae Hia and Hsf proteins as do some stretches of internal sequence. There is also some homology in the C-terminal region to UspA (ref. 23). A further indication of the relatedness of this family of proteins, is the finding that guinea pig anti-200 kDa antibody raised to gel-purified native protein was able to recognize recombinant Hia protein by immunoblot. This data has been described in copending U.S. patent application Ser. No. 09/268,347 (Hia) filed Mar. 16, 1999, assigned to the assignee hereof and the disclosure of which is incorporated herein by reference.

### Example 6

This Example shows the expression of the full-length about 200 kDa protein from pKS294.

E. coli strain, BL21(DE3)/pLysS was transformed by electroporation with pKS294, prepared as described in Example 4, for the expression study of the full-length 200 kDa protein gene.

The product of the pKS294 construct was found to be toxic to the host E. coli. At room temperature, the BL21(DE3)/pLysS transformants grew very slowly on LB-agar plates containing ampicillin (Amp) and chloramphenicol (Cm) and at 37° C., no transformants were detected. When the transformants which grew at room temperature, were cultured overnight at 30° C. on BHI agar containing the two antibiotics and glucose, they grew well, producing colonies with a normal size. However, when these clones were cultured overnight in liquid medium at 30° C., subcultured into broth without glucose, and then induced by addition of IPTG, no recombinant protein was found on Western blot using anti-200 kDa protein serum. When the cells cultured overnight were examined before subculturing, a small quantity of recombinant 200 kDa protein was detected by SDS-PAGE stained with Coomassie Blue and by Western blot, showing that the gene was expressed during the overnight culture.

When E. coli strain, DH5α, which cannot express the gene under the control of a T7 promoter, was transformed with pKS294, the transformants grew well at 37° C. both on LB-agar and in LB-broth containing the antibiotics. These results suggest that the gene product is very toxic to host E. coli, and that even a basal level of expression of the full-length 200 kDa protein gene from the ATG is lethal to E. coli.

M. catarrhalis strain LES-1 also produced similar toxicity in E. coli when the full length 200 kDa protein was expressed.

### Example 7

This Example describes the deletion of a short 5′-sequence from the strain 4223 or strain LES-1 200 kDa protein gene and expression of the truncated genes producing a M56 r200 kDa product.

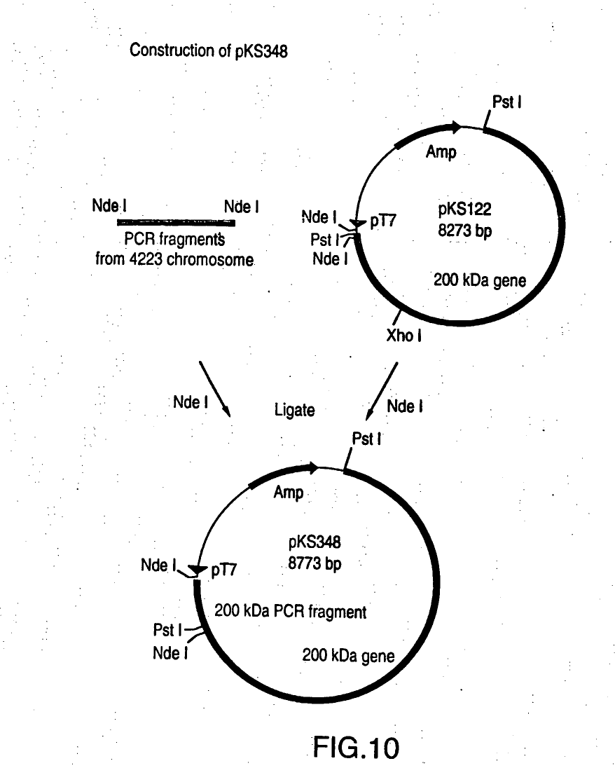

The deletion of a short 5′ region from the 4223 200 kDa protein gene is shown in FIG. 10 and was performed using a similar approach as described in Example 4. An about 500 bp 5′ region of the 200 kDa gene was PCR amplified from strain 4223 using primers 5471.KS and 4257.KS (Table 8) from chromosomal DNA. The 5′ primer (designated 5471.KS) was based upon the region surrounding the previously identified GTG downstream start codon. In primer 5471.KS, the flanking regions around the GTG codon were incorporated and the GTG was mutated to ATG with further mutations used to introduce an NdeI site incorporating the new ATG. Using numbering from the full-length 200 kDa protein, the new start codon would be M56 replacing the previous V56 codon. The 3′ primer (designated 4257.KS) was based upon the non-coding strand located about 500 bp downstream from the GTG codon in the 200 kDa protein gene. The PCR-product was digested with NdeI, purified using a QIAquick PCR purification kit (Qiagen Inc., Mississauga, Ont.), and inserted into NdeI digested and dephosphorylated pKS122 to provide pKS348 (see FIG. 7). Plasmid pKS348 was confirmed by restriction enzyme analyses and by sequencing of the PCR-amplified DNA piece and its joint regions. The nucleotide sequence (SEQ ID No: 12) and the deduced amino acid sequence (SEQ ID No: 13) for the 5′-truncation contained in pKS348 are shown in FIG. 8. A similar N-terminal truncated 200 kDa gene from strain LES-1 was generated in the same manner and was designated pKS444.

A single colony of E. coli, BL21(DE3)/pLysS, (KS358) which carried pKS348, was suspended in 5 ml of BHI broth containing Amp (100 μM), Cm (50 μM) and 0.4% of glucose, and cultured overnight at 37° C. To study the kinetics of expression, 2.5 ml of the overnight culture was added to 250 ml of LB (Luria-Bertani) broth containing Amp (100 μM) and Cm (50 μM), and grown with shaking at 37° C. to A₆₀₀=0.33 to 0.36. Another 0.3 ml of the overnight culture was added to 30 mL of LB broth containing Amp (100 μM) and Cm (50 μM) and grown with shaking at 37° C. to A₆₀₀=0.26 to 0.44. Gene expression from the cultures was induced by addition of IPTG (final concentration: 4 mM). The bacteria were grown and harvested at different time points by centrifugation. The expression of the 200 kDa protein gene in the culture was confirmed by SDS-PAGE analysis using Coomassie Blue staining and by Western blot analysis using guinea pig anti-200 kDa protein serum, as described in U.S. Pat. No. 5,808,024 and WO 96/34960.

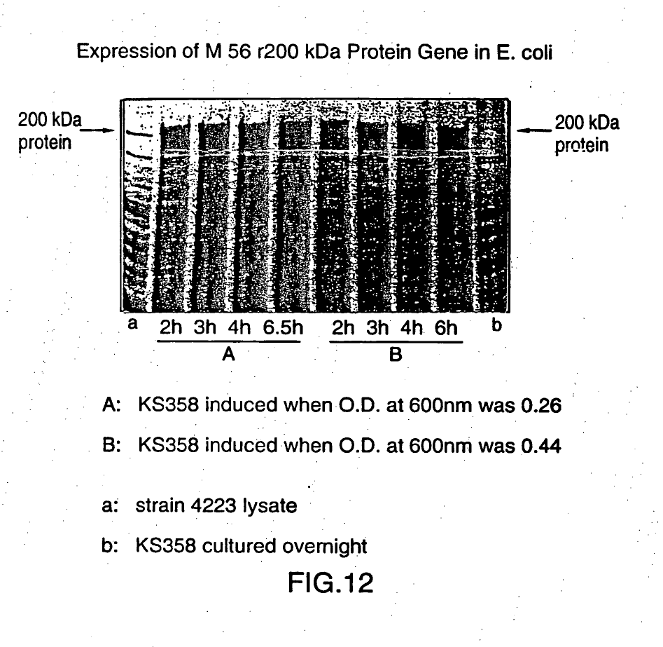

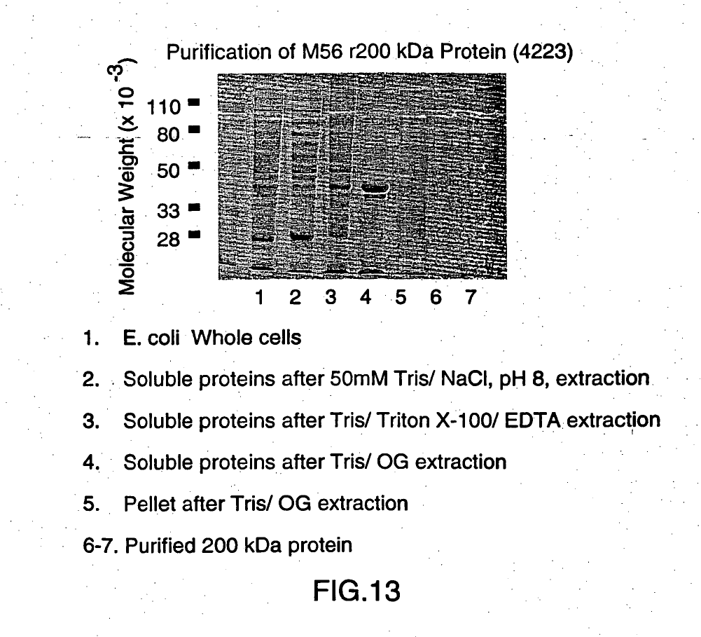

When E. coli BL21(DE3)/pLysS was transformed with pKS348, transformants grew well even on LB agar plates and in LB broth containing antibiotics at 37° C. After induction with IPTG, these clones produced a large amount of the N-terminally truncated r200 kDa protein which was clearly seen by SDS-PAGE Coomassie Blue stain, as shown in FIG. 12.

The bacterial culture induced at A₆₀₀=0.26 produced slightly more truncated r200 kDa protein than the culture induced when the OD reading was 0.44. The largest amount of truncated r200 kDa protein was seen at 3 hr after induction. Similar results were observed for the M56 r200 kDa expression from strain LES-1.

### Example 8

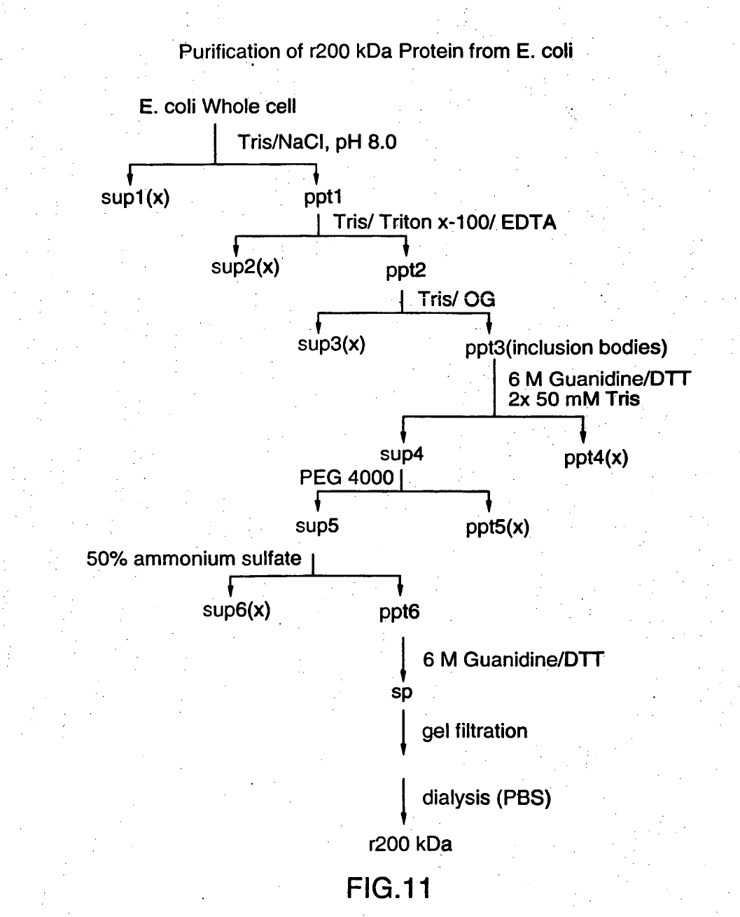

This Example describes the purification of the M56 r200 kDa proteins from strain 4223 or LES-1, according to the procedure shown in FIG. 11.

E. coli cell pellets were obtained from 500 ml culture prepared as described in Example 7, by centrifugation and were resuspended in 50 ml of 50 mM Tris-HCl, pH 8.0, containing 0.1 M NaCl, and disrupted by sonication. The sonicate was centrifuged at 20,000×g for 30 min. and the resultant supernatant (sup1) was discarded. The pellet (ppt1) was extracted, in 50 ml of 50 mM Tris-HCl, pH 8.0 containing 0.5% Triton X-100 and 10 mM EDTA, then centrifuged at 20,000×g for 30 min. and the supernatant (sup2) was discarded. The pellet (ppt2) was further extracted in 50 ml of 50 mM Tris-HCl, pH 8.0, containing 1% octylglucoside, then centrifuged at 20,000×g for 30 min. and the supernatant (sup3) was discarded.

The resultant pellet (ppt3) contained the inclusion bodies. The pellet was solubilized in 6 ml of 50 mM Tris-HCl, pH 8.0, containing 6 M guanidine and 5 mM DTT. Twelve ml of 50 mM Tris-HCl, pH 8.0 was added, the mixture centrifuged at 20,000×g for 30 min, and the pellet (ppt4) discarded. The supernatant (sup4) was precipitated by adding polyethylene glycol (PEG) 4000 at a final concentration of 5% and incubated at 4° C. for 30 min. The resultant pellet (ppt5) was removed by centrifugation at 20,000×g for 30 min. The supernatant was then precipitated by (NH₄)₂SO₄ at 50% saturation at 4° C. overnight. After the addition of (NH₄)₂SO₄, the solution underwent phase separation with protein going to the upper phase (as judged by the cloudiness of the layer). The upper phase was collected, then subjected to centrifugation at 20,000×g for 30 min. The resultant pellet was collected and dissolved in 2 ml of 50 mM Tris-HCl, pH 8.0, containing 6 M guanidine and 5 mM DTT. The clear solution was purified on a Superdex 200 gel filtration column equilibrated in 50 mM Tris-HCl, pH 8.0, containing 2 M guanidine HCl. The fractions were analysed by SDS-PAGE and those containing the purified r200 kDa were pooled. The pooled fraction was concentrated 5 to 10 fold using a centriprep 30 and then dialysed overnight at 4° C. against PBS, and centrifuged at 20,000×g for 30 min to clarify.

The protein remained soluble under these conditions and glycerol was added to the M56 r200 kDa preparation at a final concentration of 20% for storage at −20° C. (FIG. 12). The average yield of the purified M56 r200 kDa protein is about 10 mg L⁻¹ culture. The purified protein was used for the immunization of animals, as described below.

The procedure of this Example 8 and was repeated for M. catarrhalis strain LES-1 and a corresponding r200 kDa protein was produced. The N-terminal truncated M56 r200 kDa protein from strain LES-1 gave approximately the same recovery of purified protein as described above for strain 4223.

### Example 9

This Example illustrates the immunogenicity of the M56 r200 kDa protein.

The immunogenicity of M56 r200 kDa, prepared as described in Example 8, was examined using mice and guinea pigs. Groups of five BALB/c mice (Charles River, Quebec) were immunized sub-cutaneously (s.c.) on days 1, 29 and 43 with 0.3, 1.3 and 10 μg of 4223 M56 r200 kDa antigen, prepared as described in Example 8, in the presence AlPO₄ (1.5 mg per dose). Blood samples were collected on days 0, 14, 28, 42 and 56.

Groups of five guinea pigs (Charles River, Quebec) were immunized i.m. on days 1, 29 and 43 with 25, 50 and 100 μg of 4223 M56 r200 kDa antigen prepared as described in Example 8, in the presence AlPO₄ (1.5 mg per dose). Blood samples were collected on days 0, 14, 28, 42 and 56.

Anti-M56 r200 kDa IgG titers were determined by antigen-specific enzyme-linked immunosorbent assays (EIAs). Microtiter wells (Nunc-MAXISORP, Nunc, Denmark) were coated with 50 μL of protein antigen 0.2 μg mL⁻¹) The reagents used in the assays were as follows: affinity-purified F(ab′)2 fragments of goat anti-mouse IgG (Fc-specific) conjugated to horseradish peroxidase (Jackson ImmunoResearch Labs, Mississauga, Ontario); affinity-purified guinea pig anti-IgG antibody (1 μg ml⁻¹)(prepared by the inventors); and affinity-purified F(ab′)2 fragment of goat anti-guinea pig IgG (H+L) antibodies conjugated to horseradish peroxidase (HRP) (Jackson ImmunoResearch Laboratories) used as a reporter. The reactions were developed using tetramethylbenzidine (TMB/H₂O₂, ADI, Mississauga, Ontario) and absorbancies were measured at 450 nm (using 540 nm as a reference wavelength) in a Flow Multiskan MCC microplate reader (ICN Biomedicals, Mississauga, Ontario). The reactive titer of an antiserum was defined as the reciprocal of the dilution consistently showing a two-fold increase in absorbance over that obtained with the pre-bleed serum sample.

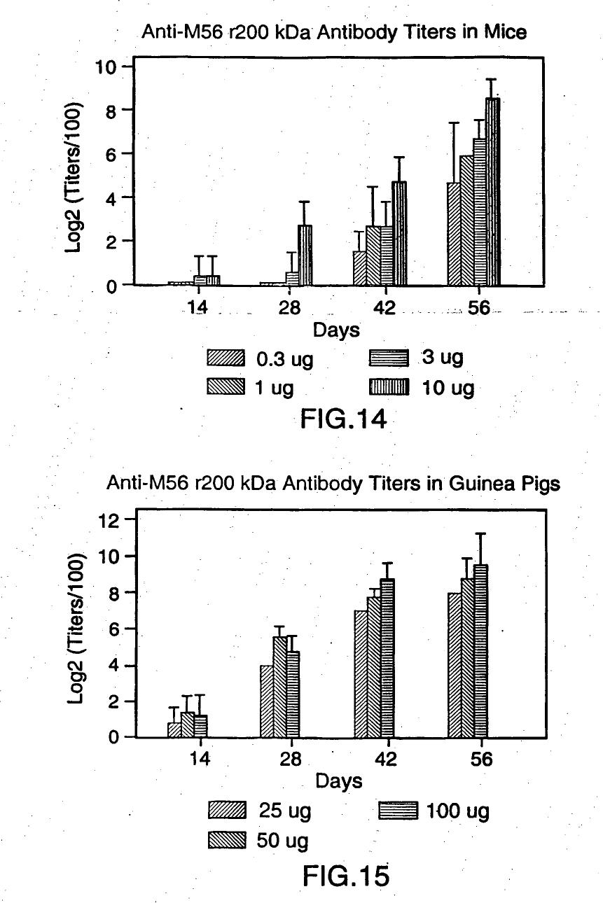

The mice generated dose-dependent anti-M56 r200 kDa antibody responses, as shown in FIG. 14. These results clearly show that the protein remained immunogenic after inclusion bodies extraction, solubilization and purification. Only a slight difference in the antibody titers were found for the higher dose range tested in guinea pigs (FIG. 15), indicating that the amount of antigen used was nearly at saturation.

### Example 10

This Example describes the generation of hyper-immune sera against the M56 r200 kDa proteins in rabbits and guinea pigs.

To generate hyper-immune sera against M56 r200 kDa proteins, groups of two rabbits and two guinea pigs (Charles River, Quebec) were immunized intramuscularly (i.m.) on day 1 with a 5 μg dose of purified M56 r200 kDa protein, prepared as described in Example 8, emulsified in complete Freund's adjuvant (CFA). Animals were boosted on days 14 and 29 with the same dose of protein emulsified in incomplete Freund's adjuvant (IFA). Blood samples were taken on day 42 for analyzing the anti-M56 r200 kDa antibody titers and bactericidal activities. Anti-r200 kDa IgG titers were determined by antigen-specific enzyme-linked immunosorbent assays (EIAs), as described in Example 9. The results obtained in the two animals using r200 kDa protein from strains 4223 and LES-1 are illustrated in Table 6.

### Example 11

This Example describes a bactericidal antibody assay.

The bactericidal antibody activity of guinea pig anti-M56 r200 kDa sera from 4223 or LES-1 protein prepared as described in Example 10 against various strains of M. catarrhalis was estimated using a viability plating assay. Each test strain of M. catarrhalis was cultured overnight in brain heart infusion (BHI) broth (Difco Laboratories, Detroit, Mich.) at 37° C. The overnight culture was subcultured into 10 ml BHI broth, and grown to an absorbance at 578 nm of 0.5. The number of bacteria at A₅₇₈=0.5 changes from strain to strain. Therefore, several ten-fold dilutions of each strain were used in order to achieve 100 to 300 colonies per plate for the preimmune serum group. Bacteria were diluted in Veronal buffered saline (VBS, pH 7.6) containing 140 mM NaCl, 93 mM NaHCO₃, 2 mM Na-barbiturate, 4 mM barbituric acid, 0.5 mM MgCl₂.6H₂O, 0.4 mM CaCl₂.2H₂O, and 0.1% bovine serum albumin. Guinea pig anti-M56 r200 kDa serum and pre-immune control serum were heated at 56° C. for 30 min. to inactivate endogenous complement. Serum and antiserum were diluted in VBS, and placed on ice.

Twenty-five μl of diluted pre-immune serum or test antiserum were added to the wells of a 96-well Nunclon microtitre plate (Nunc, Roskilde, Denmark). Twenty-five μl of diluted bacterial cells were added to each of the wells. A guinea pig complement (BioWhittaker, Walkerville, Md.) was diluted 1:10 in VBS, and 25 μl portions were added to each well. The plates were incubated for 60 min, gently shaking at 70 rpm on a rotary platform. Fifty μl of each reaction mixture were plated onto Mueller Hinton agar plates (Becton-Dickinson, Cockeysville, Md.). The plates were incubated at 37° C. for 24 hours, and then left at room temperature for a further 24 hours. The number of colonies per plate was counted, and average values of colonies per plate were estimated from duplicate pairs.

When pre-immune serum plates were compared with PBS control plates (no serum), pre-immune serum had no bactericidal effect on the homologous strain 4223. Therefore, it was assumed that the number of colonies per plate on pre-immune serum plates represented 100% viability for each strain and percent bactericidal killing was calculated as follows:

$$100 \% - [ \frac{\begin{matrix}average number of colonies per plate \\ in anti \text{-} r200 kDa antiserum group \times 100\end{matrix}}{\begin{matrix}average number of colonies per plate \\ in pre \text{-} immune serum group\end{matrix}} ] \%$$

When the bactericidal antibody activity of the 4223 anti-M56 r200 kDa antiserum was examined against the homologous strain (Table 7), 50% killing was observed at a serum dilution between 1/512 and 1/1024, showing that the antiserum raised against M56 r200 kDa protein possesses bactericidal antibody activity. Next, the bactericidal antibody activity of the antiserum was tested at a dilution of 1/64 against a total of 55 different strains, which were isolated from otitis media patients in various geographical locations (Table 1B). The antiserum raised against the M56 r200 kDa protein from strain 4223 showed more than 30% bactericidal antibody activity against 38 out of 56 (68%) strains examined. When LES-1 anti-M56 r200 kDa antibody was tested in the bactericidal antibody assay, 36/55 (65%) strains were killed, including 11 strains that were not killed by the 4223 anti-M56 r200 kDa antibody. Only six strains out of 55 strains examined were not killed by either one of the two antisera. These results indicate that the 200 kDa protein is a very good candidate for inclusion in an otitis media vaccine.

### Example 12

This Example describes the inhibition of binding of M. catarrhalis strains to either Chang or Hep-2 epithelial cells by 4223 anti-M56 r200 kDa serum.

The 200 kDa protein had previously been proposed to be an adhesin on the basis of its apparent absence from a spontaneous non-clumping variant of strain 4223. This strain, obtained by serial passaging of culture supernatants, was designated RH408 and is described in WO 96/34960. Electron microcopy also suggested that the 200 kDa protein was an adhesin. The sequence homology demonstrated between the M. catarrhalis 200 kDa proteins and other high molecular weight adhesins from different organisms, also suggested that it was an adhesin. Based upon these observations, an assay was developed to try to demonstrate that anti-r200 kDa antibody could block adherence between M. catarrhalis and epithelial cells, thus identifying it definitively as an adhesin.

On day 1, 24 well tissue culture plates were seeded with approximately 3×10⁵ Chang cells per well to achieve a confluent monolayer following overnight incubation at 37° C. in the presence of 5% CO₂. M. catarrhalis 4223 or Q8 was cultured in 10 ml of BHI broth at 37° C. for 18 hr, shaking at 200 rpm.

On day 2, bacterial cultures were pelleted by centrifugation at 3500 rpm for 10 min, and washed with 10 ml of PBS. After a centrifugation as above, each pellet was resuspended in 2 ml of DMEM supplemented with 10% FBS and 2 mM glutamine. The bacteria cultures were diluted 1/10 in the supplemented DMEM to OD of approximately 1.8 at 578 nm. Confluent monolayers of Chang cells were washed once with 1 ml of PBS per well, and 0.5 ml of 10% BSA in PBS was added to each well as a blocking agent. Plates were incubated at 37° C. for 30 min and monolayers were washed twice with PBS as above.

A guinea pig anti-4223 MS6 r200 kDa antiserum, prepared as described in Example 10 and pooled pre-immune guinea pig sera were heated at 56° C. for 30 min to inactivate endogenous complement. Equal volumes of appropriately diluted antisera and bacteria were mixed, and 200 μl of the mixture were added into each well. Examples of antiserum dilutions tested included 1/4, 1/16 and 1/64. The plate was incubated at 37° C. for 1 hr, with gentle shaking. The plate was carefully washed four times with 1 ml of PBS per well to remove the bacteria. To each well, 100 μl of trypsin were added, and the plate was incubated at 37° C. for 5 min. After inactivation of trypsin by addition of 900 μl Dulbecco's Minimal Essential Medium (DMEM) to each well, the cells were resuspended by pipetting up and down several times.

Ten-fold dilutions of resuspended cells were prepared in a new 96-well plate. Fifty μl each of the 1×10⁻², 1×10⁻³, 1×10⁻⁴ and 1×10⁻⁵ diluted samples were plated on a Mueller-Hinton agar plate. Plates were incubated at 37° C. overnight, and then left at room temperature for a further 24 hours. The number of colonies per plate was counted for the estimation of the total bound bacteria.

Dilution plating was also carried out for each bacterial strain, to estimate bacterial concentrations and to calculate the total amount of bacteria added to each well. It was assumed that the number of bacteria bound to tissue culture cells in the presence of pre-immune sera represented 100% optimal binding for each assay, and 0% inhibition. Therefore, in order to calculate the percent inhibition of the antiserum, we used the following formula:

$$\% inhibition = 100 - [ \frac{\begin{matrix}total bacteria bound in 4223 \\ anti \text{-} r200 kDa antiserum samples\end{matrix}}{\begin{matrix}total bacteria bound in \\ pre \text{-} immune sera samples\end{matrix}} \times 100 ]$$

When the guinea pig 4223 anti-M56 r200 kDa protein serum was examined for the inhibition of binding of strain 4223 to Chang cells (Table 8), inhibition of 98%, 92% and 83% was observed at antiserum dilutions of 1/4, 1/16 and 1/64, respectively. With the heterologous strain Q8, the inhibition of binding to the tissue culture cells was estimated to be 77%, 82% and 55% at antiserum dilutions of 1/4, 1/16 and 1/64, respectively. The results clearly showed that anti-M56 r200 kDa protein serum inhibited the binding of M. catarrhalis to cultured human epithelial cells.

Having demonstrated that 4223 anti-M56 r200 kDa antibody could block adherence of M. catarrhalis strains 4223 or Q8 to Chang epithelial cells in a dose-dependent manner, the studies were extended to other strains. Of particular interest, were those strains that were not killed by anti-M56 r200 kDa antisera in the bactericidal antibody assay. To perform the in vitro adherence assay on several strains, a single antibody dilution of 1/16 was used. The data for inhibition of in vitro adherence to Hep-2 cells is summarized in Table 9. The procedure for the Hep-2 epithelial cells was identical to the Chang cell procedure described above. The 4223 anti-M56 r200 kDa antibody effectively blocked adherence of the homologous strain by 48%. Strain RH408 does not express the 200 kDa gene and in the assay, antibody inhibited adherence of RH408 to 9%. This would be assumed to be a background level. Of 20 strains tested, 16 were inhibited at rates higher than 9%. Among these strains were 19 strains that had not been killed by the 4223 anti-M56 r200 kDa antibody.

To summarize and as shown in Tables 1, 8 and 9, in our collection of 89 strains of Moraxella catarrhalis, 80 express 200 kDa. Of 57 strains tested with 4223 anti-M56 r200 kDa antibody in the bactericidal antibody assay, 39 were killed (58%). An additional 15 strains were inhibited from binding to epithelial cells by the same antibody for a total of 54 strains (95%), against which a single antibody was effective. These data demonstrate the very high potential of r200 kDa proteins as vaccine antigens.

### Example 13

This Example describes the sequence analysis of the 200 kDa protein gene from M. catarrhalis strain RH408, the non-clumping variant of 4223 described in WO 96/34960.

As described in Example 4 and Table 5, it appeared that the number of G nucleotides in the G tract had a regulatory function on the expression of the 200 kDa gene. M. catarrhalis strain 4223 and its non-clumping derivative RH408 appeared to differ only in the expression of the 200 kDa gene. The 200 kDa gene from strain RH408 was subcloned and sequenced and its sequence compared to the parental gene from strain 4223.

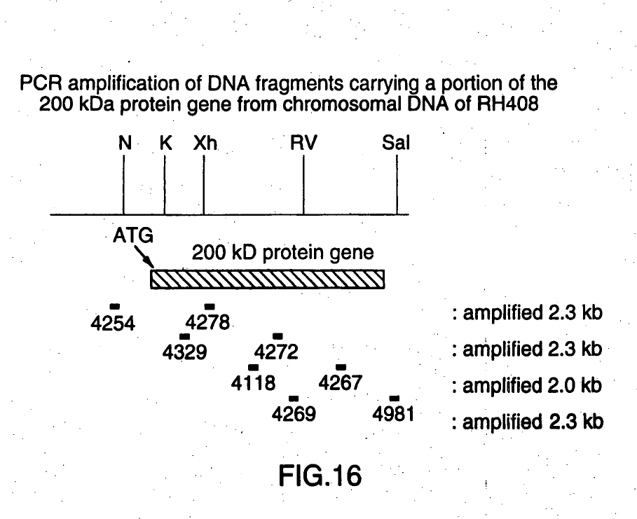

Four partially overlapping fragments of the 200 kDa protein gene were PCR amplified from strain M. catarrhalis RH408, using primers illustrated in FIG. 16 and Table 10, under the conditions set out in Table 11. The combined sequences of the four PCR products covered approximately 6.5 kb including the entire 200 kDa protein gene and its flanking regions. When the sequence of the 6.5 kb fragment was compared with the sequence of the same region from its parent strain 4223, the only difference was the number of G nucleotides in the G tract. As described in Example 4, the correct number of G nucleotides in the G tract was nine. However, the number G nucleotides in the G tract of RH408 was only eight.

This result, along with the analysis of this region in 24 other strains of M. catarrhalis (Table 5) strongly suggests that the number of G nucleotides in the G tract controls the expression of the 200 kDa gene in M. catarrhalis strains. Similar mechanisms of transcriptional control are found for other bacterial genes, such as the N. gonorrhehoae Pilc gene (ref. 32).

### Example 14

This Example describes the generation of additional N-terminal truncated r200 kDa proteins and expression studies.

As described in Example 6, the full-length r200 kDa protein appeared to be toxic to E. coli and could not be expressed under normal induction conditions. The M56 r200 kDa proteins were readily expressed, as described in Example 7, and were subsequently shown to be highly promising vaccine candidates in in vitro assays (Examples 11 and 12). The expression of r200 kDa proteins of intermediate length and their properties was studied.

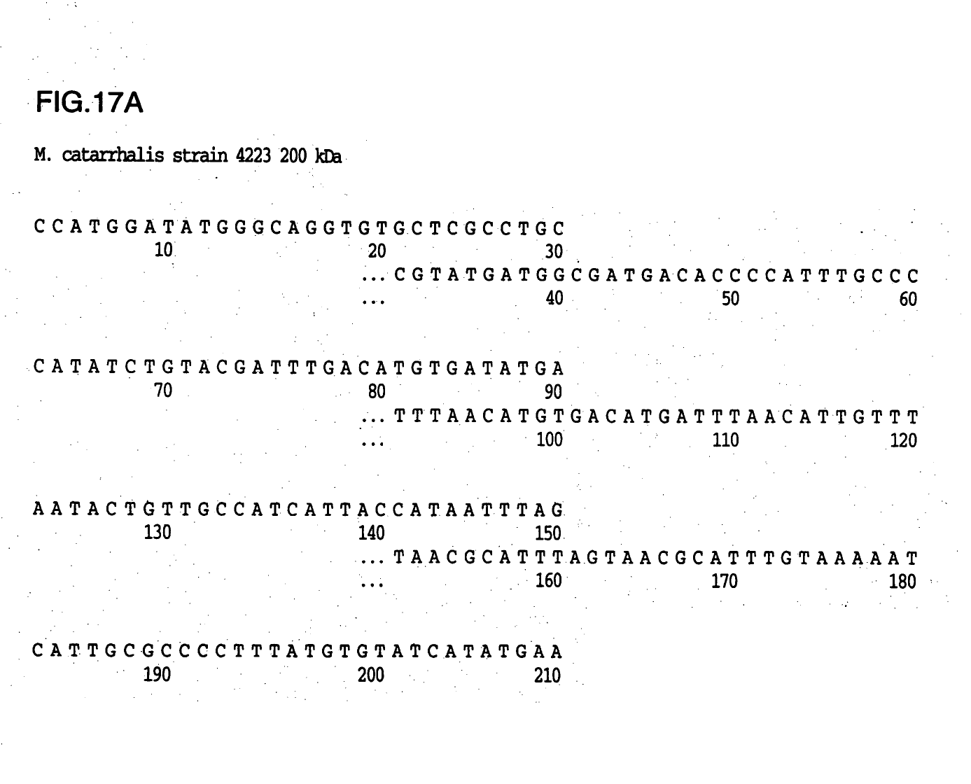

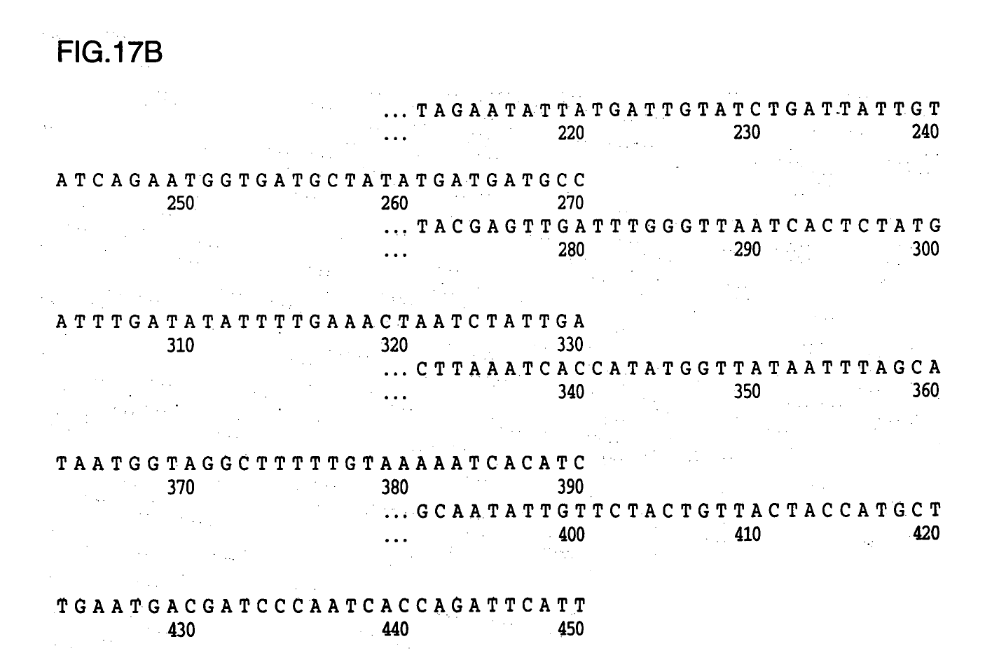

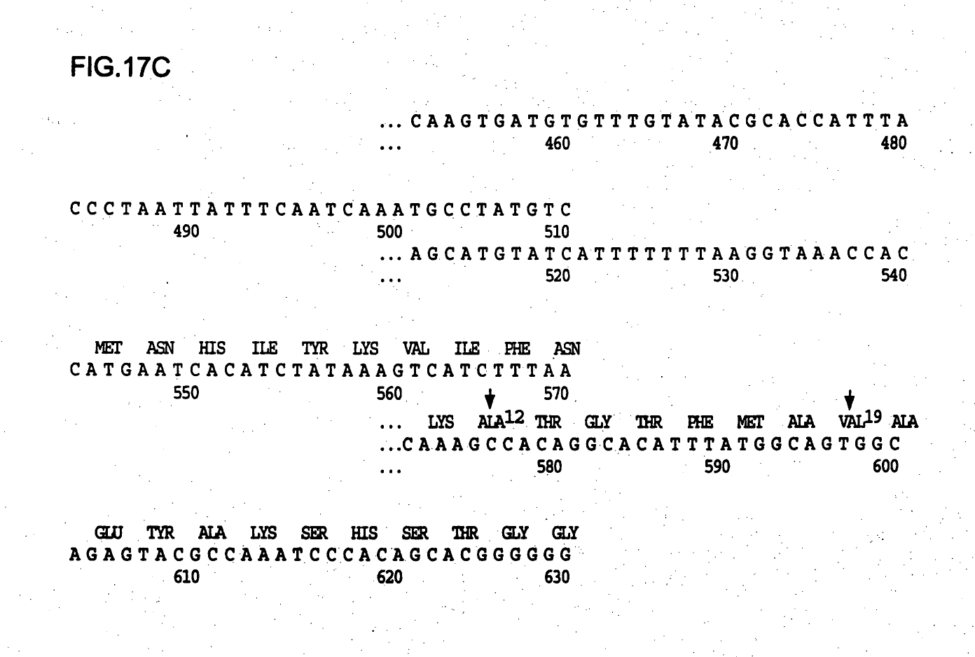

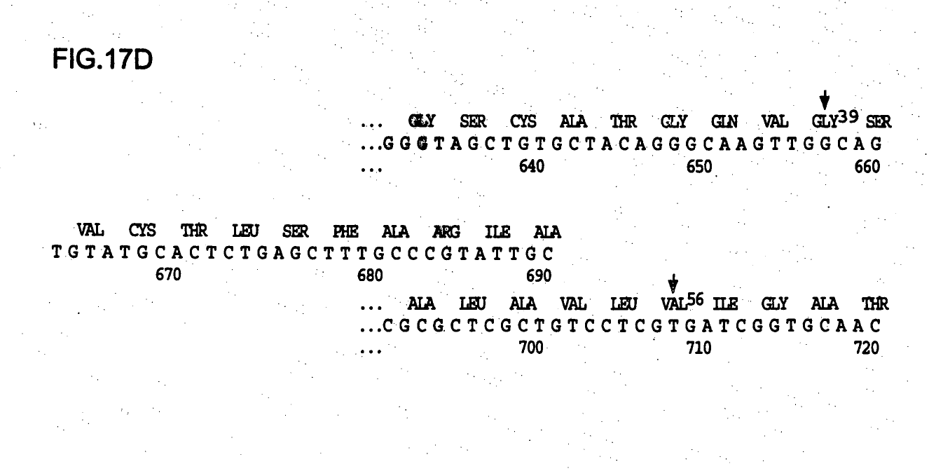

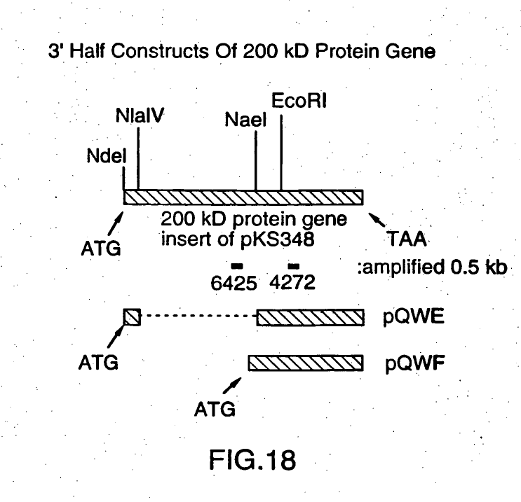

Three additional N-terminal truncated 200 kDa genes were constructed from the 4223 200 kDa gene using the procedures described in Example 7. The sites of truncation were chosen based upon and are illustrated in FIG. 17. The arrows in FIG. 17 indicate the sites of truncation, namely ALA¹², VAL¹⁹ and GLY³⁹, each modified to MET. A 5′ fragment up to an internal site was PCR amplified using primers illustrated in Table 8. For the ALA¹² truncation, the primers were 5′ 6242.ks and 3′ 4257.ks, for the VAL¹⁹ truncation, the primers were 5′ 6243.ks and 3′ 4257.ks and for the GL4³⁹ truncation, the primers were 5′ 6244.ks and 3′ 4257.ks (Table 10). The amplification conditions were the same as those used for pKS348 (Table 11). The PCR products were restricted with NdeI and ligated into the NdeI sites of pKS348 for expression. While some expression of r200 kDa was obtained with each of the N-terminal truncations, the level did not approach the levels obtained using pKS348.

### Example 15

This Example illustrates the construction of plasmids pQWE and pQWF expressing C-terminal fragments of the 200 kDa gene.

As shown in the amino acid comparison of FIG. 6, the carboxy half of the 200 kDa protein in quite conserved, the main difference being a large approximately 300 amino acid residue insert in strain LES-1. Since so much cross-reactivity for the anti-M56 r200 kDa antisera had been observed, the conserved carboxy half of the protein was expressed.

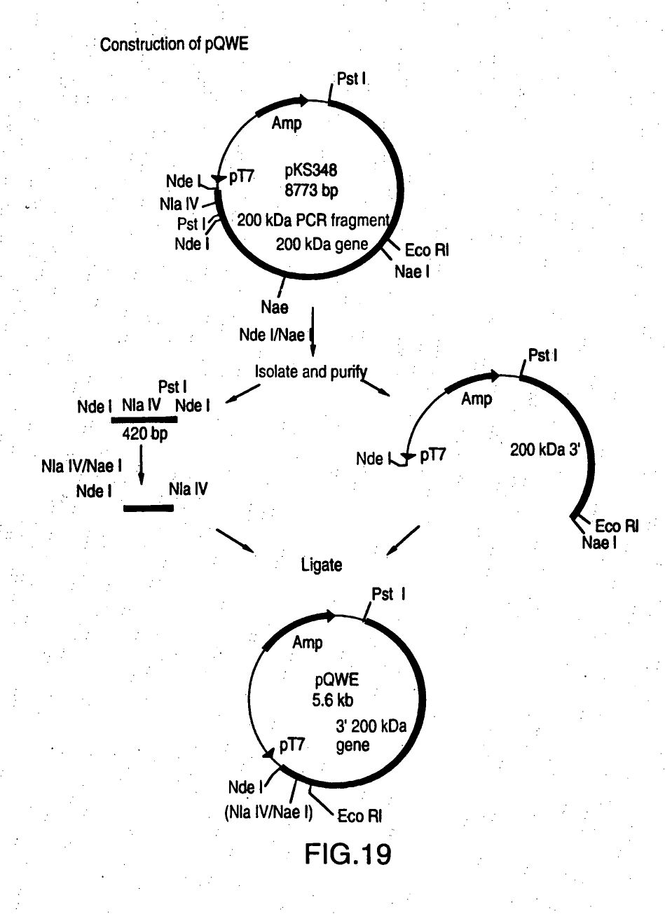

Plasmid pKS348 prepared as described in Example 7 was digested with restriction enzymes, Nde I and Nae I, producing four fragments. The approximately 5.8 kb Nde I/Nae I fragment containing the T7 promoter, ampicillin antibiotic resistance marker and the 3′ end of the 200 kDa gene was agarose gel purified. The approximately 480 bp Nde I/Nde I fragment containing the 5′ end of the 200 kDa gene was also gel purified. This approximately 480 bp fragment was then restriction digested with the enzymes Nla IV and Pst I and the Nde I/Nla IV fragment ligated to the previously isolated 5.8 kb Nde I/Nae I fragment to produce plasmid pQWE, as illustrated in FIG. 19. This plasmid construct contained a 200 kba gene with the Nla IV to Nae I fragment deleted. This plasmid construct resulted, upon expression as described in Example 7, in a fusion 200 kDa protein containing a very short piece of the 5′ end and the 3′ half of the 200 kDa protein.

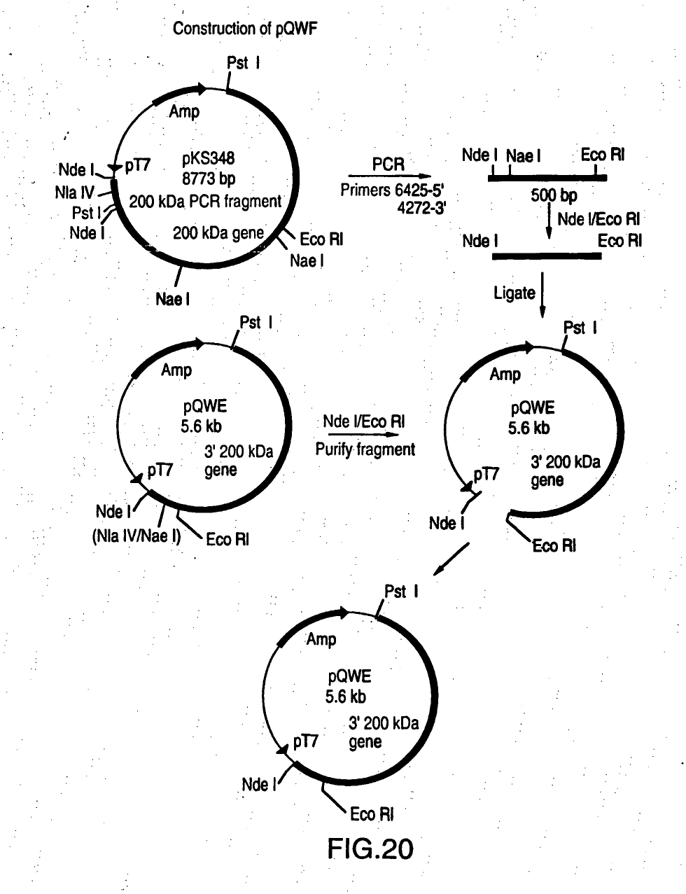

An approximately 500 bp fragment around the Eco RI site in the 200 kDa gene from plasmid pKS348 was PCR amplified utilizing a 5′ oligonucleotide, 6425.KS and a 3′ oligonucleotide 4272.KS (Table 10) using the conditions outlined in Table 11. The 5′ oligonucleotide was synthesized with an ATG translational start codon and a Nde I restriction site, while the 3′ oligonucleotide was synthesized with an Eco RI site. The approximately 500 bp PCR fragment was the restriction digested with the enzymes Nde I and Eco RI. Plasmid pQWE, prepared as described above, was restriction digested with Nde I and Eco RI as illustrated in FIG. 20, and this larger fragment agarose gel purified. The Nde I/Eco RI PCR fragment was then ligated into the isolated Nde I/Eco RI fragment from pQWE, to produce plasmid pQWF. This construct expresses a 5′ truncated 200 kDa protein, having only the 3′ half of this protein from the region about 40 bp upstream of the Nde I site to the 3′ end.

The constructs pQWE and pQWF, prepared as described above and as illustrated in FIGS. 19 and 20, were expressed in E. coli strain BL21(DE3)/pLysS as described in Example 7. The C-terminal half proteins were obtained at levels of expression approximately twice those achieved using pKS348. Corresponding constructs were prepared from strain LES-1 and produced comparable results.

Antiserum was raised against the C-terminal half of 200 kDa protein produced from construct pQWE following the procedure of Example 10 and was employed in the bactericidal assay described in Example 11. As may be seen in Table 1B the antiserum showed more than 30% of killing against 30 out of 31 strains which were killed by the bactericidal assay using antiserum raised against the product from pKS348.

### SUMMARY OF THE DISCLOSURE

In summary of this disclosure nucleotide sequences encoding an about 200 kDa outer membrane protein from several strains of Moraxella catarrhalis are described along with recombinant production of such protein. Modifications are possible within the scope of this invention.

TABLE 1A
Examination of 200 kDa protein in M. catarrhalis strains

| | | | EXPRESSION OF |
|---|---|---|---|
| | ANATOMICAL | | 200 kDa |
| STRAIN | ORIGIN | SOURCE | PROTEIN |
| 4223 | MID. EAR | T. F. | +++ |
| | FLUID | MURPHY | |
| RH408 | MUTANT | | − |
| | OF 4223 | | |
| 3 | SPUTUM | T. F. | − |
| | | MURPHY | |
| 56 | SPUTUM | T. F. | − |
| | | MURPHY | |
| 135 | MID. EAR | T. F. | +++ |
| | FLUID | MURPHY | |
| 585 | BACTEREMIA | T. F. | + |
| | | MURPHY | |
| 5191 | MID. EAR | T. F. | +++ |
| | FLUID | MURPHY | |
| 8185 | NASO- | T. F. | +++ |
| | PHARYNX | MURPHY | |
| M2 | SPUTUM | T. F. | +++ |
| | | MURPHY | |
| M5 | SPUTUM | T. F. | − |
| | | MURPHY | |
| ATCC25240 | | ATCC | − |
| H-04 | OTITIS | G. D. | +++ |
| | | CAMPBELL | |
| H-12 | ″ | G. D. | − |
| | | CAMPBELL | |
| PO-34 | ″ | G. D. | +++ |
| | | CAMPBELL | |
| PO-51 | ″ | G. D. | +++ |
| | | CAMPBELL | |
| E-07 | ″ | G. D. | +++ |
| | | CAMPBELL | |
| E-22 | ″ | G. D. | +++ |
| | | CAMPBELL | |
| E-23 | ″ | G. D. | +++ |
| | | CAMPBELL | |
| E-24 | ″ | G. D. | +++ |
| | | CAMPBELL | |
| M-02 | ″ | G. D. | +++ |
| | | CAMPBELL | |
| M-20 | ″ | G. D. | +++ |
| | | CAMPBELL | |
| M-29 | ″ | G. D. | +++ |
| | | CAMPBELL | |
| M-32 | ″ | G. D. | +++ |
| | | CAMPBELL | |
| M-35 | ″ | G. D. | +++ |
| | | CAMPBELL | |
| Q-2 | EXPECTO- | M. G. | + |
| | RATION | BERGERON | |
| Q-6 | EXPECTO- | M. G. | − |
| | RATION | BERGERON | |
| Q-8 | EXPECTO- | M. G. | +++ |
| | RATION | BERGERON | |
| Q-9 | EXPECTO- | M. G. | − |
| | RATION | BERGERON | |
| Q-10 | EXPECTO- | M. G. | +++ |
| | RATION | BERGERON | |
| Q-11 | EXPECTO- | M. G. | +++ |
| | RATION | BERGERON | |
| Q-12 | EXPECTO- | M. G. | − |
| | RATION | BERGERON | |
| R-1 | BRONCHIAL | M. G. | + |
| | SECRETIONS | BERGERON | |
| R-2 | BRONCHIAL | M. G. | − |
| | SECRETIONS | BERGERON | |
| R-4 | OTITIS | M. G. | +++ |
| | | BERGERON | |
| R-5 | ″ | M. G. | +++ |
| | | BERGERON | |
| R-6 | ″ | M. G. | +++ |
| | | BERGERON | |
| R-7 | ″ | M. G. | +++ |
| | | BERGERON | |
| N-209 | BLOOD | M. G. | +++ |
| | | BERGERON | |
| VH-1 | OTITIS | V. | +++ |
| | | HOWIE | |
| VH-2 | ″ | V. | +++ |
| | | HOWIE | |
| VH-3 | ″ | V. | +++ |
| | | HOWIE | |
| VH-4 | ″ | V. | +++ |
| | | HOWIE | |
| VH-5 | ″ | V. | +++ |
| | | HOWIE | |
| VH-6 | ″ | V. | +++ |
| | | HOWIE | |
| VH-7 | ″ | V. | +++ |
| | | HOWIE | |
| VH-8 | ″ | V. | +++ |
| | | HOWIE | |
| VH-9 | ″ | V. | +++ |
| | | HOWIE | |
| VH-10 | ″ | V. | +++ |
| | | HOWIE | |
| VH-11 | ″ | V. | +++ |
| | | HOWIE | |
| VH-12 | ″ | V. | +++ |
| | | HOWIE | |
| VH-13 | ″ | V. | +++ |
| | | HOWIE | |
| VH-14 | ″ | V. | +++ |
| | | HOWIE | |
| VH-15 | ″ | V. | +++ |
| | | HOWIE | |
| VH-16 | ″ | V. | +++ |
| | | HOWIE | |
| VH-17 | ″ | V. | +++ |
| | | HOWIE | |
| VH-18 | ″ | V. | +++ |
| | | HOWIE | |
| VH-19 | ″ | V. | +++ |
| | | HOWIE | |
| VH-20 | ″ | V. | +++ |
| | | HOWIE | |
| VH-23 | ″ | V. | +++ |
| | | HOWIE | |
| VH-24 | ″ | V. | +++ |
| | | HOWIE | |
| VH-25 | ″ | V. | +++ |
| | | HOWIE | |
| VH-26 | ″ | V. | +++ |
| | | HOWIE | |
| VH-27 | ″ | V. | +++ |
| | | HOWIE | |
| VH-28 | ″ | V. | +++ |
| | | HOWIE | |
| VH-29 | ″ | V. | +++ |
| | | HOWIE | |
| VH-30 | ″ | V. | +++ |
| | | HOWIE | |
| LES1 | OTITIS | L. S. | +++ |
| | | STENFORS | |
| LES2 | ″ | L. S. | +++ |
| | | STENFORS | |
| LES4 | ″ | L. S. | +++ |
| | | STENFORS | |
| LES5 | ″ | L. S. | +++ |
| | | STENFORS | |
| LES6 | ″ | L. S. | +++ |
| | | STENFORS | |
| LES7 | ″ | L. S. | +++ |
| | | STENFORS | |
| LES8 | ″ | L. S. | +++ |
| | | STENFORS | |
| LES9 | ″ | L. S. | +++ |
| | | STENFORS | |
| LES10 | ″ | L. S. | +++ |
| | | STENFORS | |
| LES11 | ″ | L. S. | +++ |
| | | STENFORS | |
| LES12 | ″ | L. S. | +++ |
| | | STENFORS | |
| LES13 | ″ | L. S. | +++ |
| | | STENFORS | |
| LES16 | ″ | L. S. | +++ |
| | | STENFORS | |
| LES17 | ″ | L. S. | +++ |
| | | STENFORS | |
| LES21 | ″ | L. S. | +++ |
| | | STENFORS | |
| 30607 | OTITIS | C. W. | +++ |
| | | FORD | |
| CJ1 | ″ | C. | +++ |
| | | JOHNSON | |
| CJ3 | ″ | C. | +++ |
| | | JOHNSON | |
| CJ4 | ″ | C. | +++ |
| | | JOHNSON | |
| CJ7 | ″ | C. | +++ |
| | | JOHNSON | |
| CJ8 | ″ | C. | +++ |
| | | JOHNSON | |
| CJ9 | ″ | C. | +++ |
| | | JOHNSON | |
| CJ11 | ″ | C. | +++ |
| | | JOHNSON | |

Bacteria were lysed and proteins were separated on SDS-PAGE gels. The expression of 200 kDa protein was examined by Coomassie Blue staining and by Western blot using anti-200 kDa protein guinea pig serum.

TABLE 1B
Bactericidal assay results against Moraxella catarrhalis using
antisera raised against recombinant M56 200 kDa protein
from strains 4223 and LES1, and recombinant C-terminal
half of 200 kDa protein from strain 4223.

| | | Killed by anti- | |
|---|---|---|---|
| | Killed by anti- | C-terminal half | Killed by anti- |
| | M56 200 kDa | of 200 kDa from | M56 200 kDa |
| STRAIN | from 4223 | 4223 | from LES1 |
| 4223 | ++ | ++ | − |
| 135 | ++ | ++ | ++ |
| H-04 | ++ | ++ | ? |
| H-12* | − | NT | − |
| PO-34 | − | NT | ++ |
| PO-51 | − | NT | − |
| E-07 | − | NT | ++ |
| E-22 | ++ | ++ | − |
| E-24 | − | NT | − |
| M-02 | ++ | ++ | ++ |
| M-20 | ++ | + | − |
| M-29 | ++ | ++ | ++ |
| M-32 | ++ | ++ | ++ |
| M-35 | ++ | ++ | ++ |
| R4 | − | NT | ++ |
| R5 | ++ | ++ | ++ |
| R6 | ++ | + | + |
| R7 | ++ | NT | ? |
| Q8** | ++ | + | NT |
| VH-1 | ++ | NT | ++ |
| VH-2 | ++ | NT | ++ |
| VH-4 | − | NT | ++ |
| VH-5 | ++ | ++ | − |
| VH-7 | ++ | + | ? |
| VH-8 | ++ | ++ | ++ |
| VH-9 | − | NT | ++ |
| VH-10 | ++ | ++ | ++ |
| VH-13 | − | NT | − |
| VH-15 | ++ | ++ | ++ |
| VH-17 | − | NT | − |
| VH-19 | ++ | ++ | ++ |
| VH-20 | + | + | ++ |
| VH-23 | + | NT | ++ |
| VH-24 | ++ | ++ | − |
| VH-25 | − | NT | ++ |
| VH-26 | − | NT | ++ |
| VH-27 | − | NT | − |
| VH-28 | + | NT | − |
| VH-29 | ++ | ++ | ++ |
| VH-30 | − | NT | ++ |
| LES1 | − | NT | ++ |
| LES2 | ++ | ++ | + |
| LES4 | + | NT | ++ |
| LES5 | − | NT | ++ |
| LES9 | ++ | ++ | ++ |
| LES11 | + | + | + |
| LES12 | − | NT | ? |
| LES13 | − | NT | ++ |
| LES16 | + | ++ | ++ |
| LES17 | ++ | ++ | − |
| LES21 | ++ | ++ | − |
| 30607 | + | NT | ++ |
| CJ1 | ++ | ++ | ++ |
| CJ3 | ++ | − | ++ |
| CJ4 | ++ | ++ | ++ |
| CJ7 | ++ | ++ | ++ |
| CJ8 | ++ | ++ | ? |

*This strain does not produce 200 kDa protein.
**This is the only non-otitis media strain (isolated from expectorate) in this Table.
++: Killed more than 60% (>60%),
+: killed between 30% and 60%,
−: killed 30% or less,
NT: not tested,
?: the results not tested.

TABLE 2
The number of G nucleotides in the G tract of
the 200 kDa protein gene determined by sequencing
of subcloned genes from a λEMBL3 clone.

| Plasmid* | Number of G's |
|---|---|
| pKS10 | 10 |
| pKS59 | 10 |
| PKS63 | 10 |
| PKS71 | 10 |

*pKS10 and pKS71 carried a DNA insert directly subcloned from a λEMBL3 clone. pKS59 and pKS63 carried a subcloned DNA fragment, pKS9, which was a subclone from an λEMBL3 clone. pKS59, pKS63 and pKS71 carried identical DNA inserts.

TABLE 3
The number of G nucleotides in the G tract of the 200
kDa protein gene amplified by PCR from subcloned genes

| Primers | Template DNA | Number of G's |
|---|---|---|
| 4211 and 4213 | pKS9 | 10 |
| 4211 and 4213 | pKS10 | 10 |
| 4211 and 4213 | pKS71 | 10 |

* pKS9, pKS10 and pKS71, which contain a 5′ fragment of the 200 kDa protein gene, were independently subcloned from the λEMBL3 clone.

TABLE 4
The number of G nucleotides in the G tract of the 200 kDa protein
gene amplified by PCR from chromosomal DNA of strain 4223

| Primers | Template | Number of G |
|---|---|---|
| 4211 and 4166 | 4223B | 9 |
| 4211 and 4213 | 4223B | 9 |
| 4211 and 4213 | 4223R | 9 |

* The template chromosomal DNAs, 4223B and 4223R, were independently prepared from M. catarrhalis strain 4223.

TABLE 5
The number of G nucleotides in the G tract
in different strains of M. catarrhalis

| | | Number of strains | |
|---|---|---|---|
| Expression | Number of G | examined | Possible start codon |
| +++ | 3 | 1 | ATG |
| +++ | 6 | 7 | ATG |
| +++ | 9 | 7 | ATG |
| + | 10 | 3 | GTG |
| − | 7 | 3 | GTG |
| − | 8 | 2 | GTG |
| − | 9 | 1* | ATG |
| Total | | 24 | |

*The 200 kDa protein gene of this strain was prematurely terminated by a stop codon.

TABLE 6
Anti-M56 r200 kDa antibody titers in guinea pig and rabbit sera

ANTIBODY TITERS

| | Against M56 r200 | Against M56 r200 |
|---|---|---|
| ANTISERA | kDa (4223) | kDa (LES-1) |
| Gp anti-r200 kDa (4223) | 204,800 | 102,400 |
| | 409,600 | 409,600 |
| Gp anti-r200 kDa (LES1) | 204,800 | 1,638,400 |
| | 102,400 | 1,638,400 |
| Rb anti-r200 kDa (4223) | 102,400 | 102,400 |
| | 102,400 | 102,400 |
| Rb anti-r200 kDa (LES1) | 25,600 | 204,800 |
| | 102,400 | 409,600 |

TABLE 7
Killing of M. catarrhalis strain 4223 by the bactericidal antibody
activity of guinea pig anti-M56 r200 kDa protein serum

Serum dilution

| | 1/64 | 1/128 | 1/256 | 1/512 | 1/1024 |
|---|---|---|---|---|---|
| Killing % | 97% | 95% | 95% | 80% | 38% |

* The guinea pig antiserum was raised against M56 r200 kDa protein from strain 4223, and the bactericidal antibody activity of the serum at various dilutions were examined against the strain 4223.

TABLE 8
Inhibition of the binding of M. catarrhalis strains to
Chang cells by guinea pig anti-M56 r200 kDa protein serum

| Strain | 1/4 | 1/16 | 1/64 |
|---|---|---|---|
| 4223 | 98% | 92% | 83% |
| Q8 | 77% | 82% | 55% |

* The guinea pig antiserum was raised against M56 r200 kDa protein from strain 4223.

TABLE 9
Inhibition of in vitro adherence of Moraxella catarrhalis to
Hep-2 cells by antiserum raised against recombinant
200 kDa protein from strain 4223

| STRAIN | Inhibition |
|---|---|
| 4223* | +++ |
| PO-34 | +++ |
| PO-51 | ++ |
| E-07 | ++ |
| R4 | ++ |
| VH-4 | ++ |
| VH-9 | − |
| VH-13 | + |
| VH-17 | ++ |
| VH-23 | ++ |
| VH-25 | ++ |
| VH-26 | +++ |
| VH-27 | + |
| VH-28 | +++ |
| LES1 | ++ |
| LES4 | − |
| LES12 | − |
| LES13 | − |
| 30607 | + |

+++: Inhibition was 30% or higher,
++: Inhibition was 20% to 30%,
+: Inhibition was 15% to 20%,
−: Inhibition was lower than 15%.
*This strain is the positive control, and the only strain in this Table, which was killed by the bactericidal activity of anti-recombinant 200 kDa protein serum.

TABLE 10

Nucleotide sequences of primers used for PCR
amplifications

| | | SEQ ID |
|---|---|---|
| PRIMER | NUCLEOTIDE SEQUENCE | NO: |
| 4211.KS | GATGCCTACGAGTTGATTTGGGT | 14 |
| 4213.KS | GAGCGTTGCACCGATCACGAGGA | 15 |
| 4166.KS | CACTAGCCTTTACATCACCACCGATG | 16 |
| 5295.KS | AAGGTAAACCCATATGAATCACATCTATAAAGTCA | 17 |
| 4260.KS | GCTTCTAGCTGTGCCACATTGA | 18 |
| 5471.KS | CGCTCGCTGTCCATATGATCGGTGCAACGCTCA | 19 |
| 4257.KS | GACCCTGTGCATATGACATGGCT | 20 |
| 4254.KS | CCTTGGCATCAATCGTGGCACA | 21 |
| 4278.KS | TTACCTGCATCAATGCCATTGTCT | 22 |
| 4329.KS | CTGAGGTGAATACAACTACA | 23 |
| 4272.KS | CATCAGAGGTCTTTGAGGTGTCAT | 24 |
| 4118.KS | CATCACCGTGGGTCAAAAGAACGCA | 25 |
| 4267.KS | GATGTCGGCAATGTTTACCTGA | 26 |
| 4269.KS | CCACATTGACCAGTACTGGCACAGGTGCTA | 27 |
| 4981.KS | ACCTATGATCAATGGCGATTTGGT | 28 |
| 6425.KS | AAAGATCATATGGTTACCTTTGGCATTAAC | 29 |
| 6242 | GTCATCTTTCATATGGCCACAGGCACA | 30 |
| 6243 | ACATTTATGCATATGGCAGAGTACGCCA | 31 |
| 6244 | GCTACAGGGCATATGGGCAGTGTATGCACT | 32 |

TABLE 11
PCR Cycle Conditions

| | |
|---|---|
| 1. | For the construction of pKS294, oligonucleotides 5295 |
| | and 4260 and of pKS348, oligonucleotides 5471 and 4257: |
| | 95° C. for 2 min → 95° C. for 1 min, 60° C. for |
| | 30 sec, 72° C. for 1 min (10 cycles) → 95° C. for 1 min, |
| | 62° C. for 30 sec, 72° C. for 1 min (20 cycles with |
| | extension of 1 sec/cycle) → 72° C. for 10 min → 4° C. |
| 2. | For the construction of pQWF, oligonucleotides 6425 and |
| | 4272: |
| | 95° C. for 2 min → 95° C. for 1 min, 60° C. for |
| | 30 sec, 72° C. for 1 min (10 cycles) → 95° C. for 1 min, |
| | 60° C. for 30 sec, 72° C. for 1 min (20 cycles with |
| | extension of 1 sec/cycle) → 72° C. for 10 min → 4° C. |
| 3. | For the amplification of 700 bp fragment for sequencing |
| | the G-nucleotide tract from different strains, |
| | oligonucleotides 4211 and 4166. |
| | 95° C. for 2 min → 95° C. for 1 min, 60° C. for 1 min, |
| | 72° C. for 2 min (10 cycles) → 95° C. for 1 min, 60° C. |
| | for 1 min, 72° C. for 2 min (20 cycles with extension of |
| | 5 sec/cycle) → 72° C. for 10 min → 4° C. |
| 4. | For sequencing 200 kDa protein from M. catarrhalis |
| | strain RH408, |
| | (a) oligonucleotides 4254 and 4278; 4118 and 4267; and |
| | 4269 and 4981: |
| | 95° C. for 2 min → 95° C. for 1 min, 62° C. for 30 sec, |
| | 72° C. for 1 min (10 cycles) → 95° C. for 1 min, 62° C. |
| | for 30 sec, 72° C. for 1 min (20 cycles with extension of 2 |
| | sec/cycle) → 72° C. for 10 min → 4° C. |
| | (b) oligonucleotides 4329 and 4272 |
| | 95° C. for 2 min → 95° C. for 1 min, 58° C. for 30 sec, |
| | 72° C. for 1 min 30 sec (10 cycles) → 95° C. for 1 min, |
| | 58° C. for 30 sec, 72° C. for 1 min 30 sec (20 cycles |
| | with extension of 1 sec/cycle) → 72° C. for 10 min → 4° C. |

### REFERENCES

1. Van Hare, G. F., P. A. Shurin, C. D. Marchant, N. A. Cartelli, C. E. Johnson, D. Fulton, S. Carlin, and C. H. Kim. Acute otitis media caused by Branhamella catarrhalis: biology and therapy. (1987) Rev. Infect. Dis. 9:16-27.
2. Chapman, A. J., D. M. Musher, S. Jonsson, J. E. Clarridge, and R. J. Wallace. 1985. Development of bactericidal antibody during Branhamella catarrhalis infection. J. Infect. Dis. 151:878-882.
3. Hager, H., A. Verghese, S. Alvarez, and S. L. Berk. 1987. Branhamella catarrhalis respiratory infections. Rev. Infect. Dis. 9:1140-1149.
4. McLeod, D. T., F. Ahmad, M. J. Croughan, and M. A. Calder. 1986. Bronchopulmonary infection due to M. catarrhalis. Clinical features and therapeutic response. Drugs 31(Suppl. 3):109-112.
5. Nicotra, B., M. Rivera, J. I. Luman, and R. J. Wallace. 1986. Branhamella catarrhalis as a lower respiratory tract pathogen in patients with chronic lung disease. Arch. Intern. Med. 146:890-893.
6. Ninane, G., J. Joly, and M. Kraytman. 1978. Bronchopulmonary infection due to Branhamella catarrhalis: 11 cases assessed by transtracheal puncture. Br. Med. Jr. 1:276-278.
7. Srinivasan, G., M. J. Raff, W. C. Templeton, S. J. Givens, R. C. Graves, and J. C. MeI. 1981. Branhamella catarrhalis pneumonia. Report of two cases and review of the literature. Am. Rev. Respir. Dis. 123:553-555.
8. West, M., S. L. Berk, and J. K. Smith. 1982. Branhamella catarrhalis pneumonia. South. Med. J. 75:1021-1023.
9. Brorson, J-E., A. Axelsson, and S. E. Holm. 1976. Studies on Branhamella catarrhalis (Neisseria catarrhalis) with special reference to maxillary sinusitis. Scan. J. Infect. Dis. 8:151-155.
10. Evans, F. O., Jr., J. B. Sydnor, W. E. C. Moore, G. R. Moore, J. L. Manwaring, A. H. Brill, R. T. Jackson, S. Hanna, J. S. Skaar, L. V. Holdeman, G. S. Fitz-Hugh, M. A. Sande, and J. M. Gwaltney, Jr. 1975. Sinusitis of the maxillary antrum. N. Engl. J. Med. 293:735-739.
11. Tinkelman, D. G., and H. J. Silk. 1989. Clinical and bacteriologic features of chronic sinusitis in children. Am. J. Dis. Child. 143:938-942.
12. Wald, E. R., C. Byers, N. Guerra, M. Casselbrant, and D. Beste. 1989. Subacute sinusitis in children. J. Pediatr. 115:28-32.
13. Wald, E. R., G. J. Milmoe, A. Bowen, J. Ledesma-Medina, N. Salamon, and C. D. Bluestone. 1981. Acute maxillary sinusitis in children. N. Engl. J. Med. 304:749-754.
14. Christensen, J. J., and B. Bruun. 1985. Bacteremia caused by a beta-lactamase producing strain of Branhamella catarrhalis. Acta. Pathol. Microbiol. Immunol. Scand. Sect. B 93:273-275.
15. Craig, D. B., and P. A. Wehrle. 1983. Branhamella catarrhalis septic arthritis. J. Rheumatol. 10:985-986.
16. Gray, L. D., R. E. Van Scoy, J. P. Anhalt, and P. K. W. Yu. 1989. Wound infection caused by Branhamella catarrhalis. J. Clin. Microbiol. 27:818-820.
17. Guthrie, R., K. Bakenhaster, R. Nelson, and R. Woskobnick. 1988. Branhamella catarrhalis sepsis: a case report and review of the literature. J. Infect. Dis. 158:907-908.
18. Hiroshi, S., E. J. Anaissie, N. Khardori, and G. P. Bodey. 1988. Branhamella catarrhalis septicemia in patients with leukemia. Cancer 61:2315-2317.
19. O'Neill, J. H., and P. W. Mathieson. 1987. Meningitis due to Branhamella catarrhalis. Aust. N. Z. J. Med. 17:241-242.
20. Murphy, T. F. 1989. The surface of Branhamella catarrhalis: a systematic approach to the surface antigens of an emerging pathogen. Pediatr. Infect. Dis. J. 8:S75-S77.
21. Klingman, K. L., and T. F. Murphy. 1994. Purification and characterization of a high-molecular-weight outer membrane protein of Moraxella (Branhamella) catarrhalis. Infect. Immun. 62:1150-1155.
22. Helminen, M. E., I. Maciver, J. L. Latimer, J. Klesney-Tait, L. D. Cope, M. Paris, G. H. McCracken, Jr., and E. J. Hansen. 1994. A large, antigenically conserved protein on the surface of Moraxella catarrhalis is a target for protective antibodies. J. Infect. Dis. 170:867-872.
23. Aebi, C., I. Maciver, J. L. Latimer, L. D. Cope, M. K. Stevens, S. E. Thomas, G. H. McCracken, Jr., and E. J. Hansen. 1997. A protective epitope of Moraxella catarrhalis is encoded by two different genes. Infect. Immun. 65:4367-4377.
24. Nixon-George et al. The adjuvant effect of Stearyl Tyrosine on a recombinant subunit hepatitis B surface antigen. (1990), J. Immunology 144:4798-4802.
25. Wiesmuller (1989), Novel low-molecular weight synthetis vaccine against foot-and-mouth disease containing a potent B-cell and macrophage activator, Vaccine 8:29-33.
26. Deres et al. (1989), Nature 342:561.
27. Lockhoff, O. Glycolipids as Immunomodulators: Synthesis and Properties. 1991. Chem. Int. Ed. Engl. 30:1611-1620.
28. Taber and Richardson, 1985, PNAS 82(4):1074-8.
29. Fitzgerald et al, FEMS Immunol. & Med. Microbiol. 18:209-216, 1997.
30. Fitzgerald et al, FEMS Immunol. & Med Microbiol. 23:57-66, 1999.
31. Kyd et al, J. Med. Microbiology, 47:159-168, 1998.
32. Johson et al, 1997, EMBO J. 10(2):477-488.
33. Benz, I. and Schmidt, M. A., 1992. Mol Microbiol 6:1539-1546.

1-6. (canceled)

7. A vector for transforming a host which has the identifying characteristics of pKS348 (ATCC 203529) shown in FIG. 10 or pKS294 (ATCC 203528) shown in FIG. 9.

8. A vector for transforming a host which has the identifying characteristics of pQWE shown in FIG. 19 or pQWF shown in FIG. 20.

9-23. (canceled)
